SEQUENCE LISTING (88 sequences)

SEQ ID NO: 1 - protein, 191 aa, Artificial Sequence
  BHA10-2 HA10
METPAQLLFLLLLWLPDTTGHVVKTATQGEVNVTGVIPLTTTPTGSANKSKPYYTGEHAKATGNCPIWVKTPLKLANGTKYGSAGSATQEAINKITKNLNSLSELEVKNLQRLSGASDETHNEILELDEKVDDLRADTISSQIELAVLLSNEGIINSEDEGTGGGYIPEAPRDGQAYVRKDGEWVLLSTFL

SEQ ID NO: 2 - protein, 160 aa, Artificial Sequence
  BHA10-3BHA10-2
METPAQLLFLLLLWLPDTTGHVVKTATQGEVNVTGVIPLTTTPTGSANKSKPYYTGEHAKATGNCPIWVKTPLKLANGTKYGSAGSATQEAINKITKNLNSLSELEVKNLQRLSCASDETHNCILELDEKVDDLRADTISSLIELAVLLSNEGIINSEDE

SEQ ID NO: 3 - protein, 260 aa, Artificial Sequence
  NIHGen6HASS-TM
METPAQLLFLLLLWLPDTTGDTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDQGTGGILAIYSTVASSLVLLVSLGAISFWMCSNGSLQCRICI

SEQ ID NO: 4 - protein, 266 aa, Artificial Sequence
  NIHGen6HASS-TM2
METPAQLLFLLLLWLPDTTGDTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 5 - protein, 248 aa, Artificial Sequence
  NIHGen6HASS
METPAQLLFLLLLWLPDTTGDTICIGYHANNSTDTVDTVLEKNVTVTHSVNLGSGLRMVTGLRNIPQRETRGLFGAIAGFIEGGWTGMVDGWYGYHHQNEQGSGYAADQKSTQNAINGITNMVNSVIEKMGSGGSGTDLAELLVLLLNERTLDFHDSNVKNLYEKVKSQLKNNAKEIGNGCFEFYHKCNNECMESVKNGTYDYPKYSEESKLNREKIDPGSGYIPEAPRDGQAYVRKDGEWVLLSTFL

SEQ ID NO: 6 - protein, 566 aa, Artificial Sequence
  ConH1
MKAKLLVLLCAFTATDADTICIGYHANNSTDTVDTVLEKNVTVTHSVNLLEDSHNGKLCKLKGIAPLQLGKCNIAGWILGNPECESLISKRSWSYIVETPNSENGTCYPGDFADYEELREQLSSVSSFERFEIFPKESSWPNHNVTKGVTAACSHAGKSSFYRNLLWLTEKNGSYPKLSKSYVNNKEKEVLVLWGVHHPSNITDQRTLYQNENAYVSVVSSHYNRRFTPEIAKRPKVRGQAGRINYYWTLLEPGDTIIFEANGNLIAPWYAFALSRGFGSGIITSNAPMHECDTKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 7 - protein, 566 aa, Artificial Sequence
  ConH3
MKTIIALSYIFCLVFAQKLPGNDNSTATLCLGHHAVPNGTLVKTITNDQIEVTNATELVQSSSTGRICDSPHRILDGTNCTLIDALLGDPHCDGFQNKEWDLFVERSKAYSNCYPYDVPDYASLRSLVASSGTLEFNNEGFNWTGVTQNGGSSACKRGSDKSFFSRLNWLHKLKYKYPALNVTMPNNDKFDKLYIWGVHHPSTDSDQTSLYVQASGRVTVSTKRSQQTVIPNIGSRPWVRGLSSRISIYWTIVKPGDILLINSTGNLIAPRGYFKIRSGKSSIMRSDAPIGTCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAIDQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 8 - protein, 566 aa, Artificial Sequence
  MRK_pH1_Con
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 9 - protein, 529 aa, Artificial Sequence
  MRK_pH1_Con
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQ

SEQ ID NO: 10 - protein, 565 aa, Artificial Sequence
  MRK_sH1_Con
MKVKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 11 - protein, 528 aa, Artificial Sequence
  MRK_sH1_Con
MKVKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNDECMESVKNGTYDYPKYSEESKLNREKIDGVKLESMGVYQ

SEQ ID NO: 12 - protein, 566 aa, Artificial Sequence
  Cobra_P1
MKARLLVLLCALAATDADTICIGYHANNSTDTVDTVLEKNVTVTHSVNLLEDSHNGKLCKLKGIAPLQLGKCNIAGWLLGNPECESLLSARSWSYIVETPNSENGTCYPGDFIDYEELREQLSSVSSFERFEIFPKESSWPNHNTTKGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 13 - protein, 566 aa, Artificial Sequence
  Cobra_X3
MEARLLVLLCAFAATNADTICIGYHANNSTDTVDTVLEKNVTVTHSVNLLEDSHNGKLCRLKGIAPLQLGNCSVAGWILGNPECESLFSKESWSYIAETPNPENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 14 - protein, 684 aa, Artificial Sequence
  ConH1_ferritin
MKAKLLVLLCAFTATDADTICIGYHANNSTDTVDTVLEKNVTVTHSVNLLEDSHNGKLCKLKGIAPLQLGKCNIAGWILGNPECESLISKRSWSYIVETPNSENGTCYPGDFADYEELREQLSSVSSFERFEIFPKESSWPNHNVTKGVTAACSHAGKSSFYRNLLWLTEKNGSYPKLSKSYVNNKEKEVLVLWGVHHPSNITDQRTLYQNENAYVSVVSSHYNRRFTPEIAKRPKVRGQAGRINYYWTLLEPGDTIIFEANGNLIAPWYAFALSRGFGSGIITSNAPMHECDTKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 15 - protein, 685 aa, Artificial Sequence
  ConH3_ferritin
MKTIIALSYIFCLVFAQKLPGNDNSTATLCLGHHAVPNGTLVKTITNDQIEVTNATELVQSSSTGRICDSPHRILDGTNCTLIDALLGDPHCDGFQNKEWDLFVERSKAYSNCYPYDVPDYASLRSLVASSGTLEFNNEGFNWTGVTQNGGSSACKRGSDKSFFSRLNWLHKLKYKYPALNVTMPNNDKFDKLYIWGVHHPSTDSDQTSLYVQASGRVTVSTKRSQQTVIPNIGSRPWVRGLSSRISIYWTIVKPGDILLINSTGNLIAPRGYFKIRSGKSSIMRSDAPIGTCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAIDQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 16 - protein, 684 aa, Artificial Sequence
  Merck_pH1_Con_ferritin
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 17 - protein, 683 aa, Artificial Sequence
  Merck_sH1_Con_ferritin
MKVKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNDECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 18 - protein, 684 aa, Artificial Sequence
  Cobra_P1_ferritin
MKARLLVLLCALAATDADTICIGYHANNSTDTVDTVLEKNVTVTHSVNLLEDSHNGKLCKLKGIAPLQLGKCNIAGWLLGNPECESLLSARSWSYIVETPNSENGTCYPGDFIDYEELREQLSSVSSFERFEIFPKESSWPNHNTTKGVTAACSHAGKSSFYRNLLWLTKKGGSYPKLSKSYVNNKGKEVLVLWGVHHPSTSTDQQSLYQNENAYVSVVSSNYNRRFTPEIAERPKVRGQAGRMNYYWTLLEPGDTIIFEATGNLIAPWYAFALSRGSGSGIITSNASMHECNTKCQTPQGAINSSLPFQNIHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNNLEKRMENLNKKVDDGFLDIWTYNAELLVLLENERTLDFHDSNVKNLYEKVKSQLRNNAKEIGNGCFEFYHKCDNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 19 - protein, 684 aa, Artificial Sequence
  Cobra_X3_ferritin
MEARLLVLLCAFAATNADTICIGYHANNSTDTVDTVLEKNVTVTHSVNLLEDSHNGKLCRLKGIAPLQLGNCSVAGWILGNPECESLFSKESWSYIAETPNPENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 20 - protein, 498 aa, Artificial Sequence
  Wildtype Sequence of Nucleoprotein
MASQGTKRSYEQMETDGERQNATEIRASVGKMIDGIGRFYIQMCTELKLSDYEGRLIQNSLTIERMVLSAFDERRNRYLEEHPSAGKDPKKTGGPIYKRVDGRWMRELVLYDKEEIRRIWRQANNGDDATAGLTHMMIWHSNLNDTTYQRTRALVRTGMDPRMCSLMQGSTLPRRSGAAGAAVKGIGTMVMELIRMIKRGINDRNFWRGENGRKTRSAYERMCNILKGKFQTAAQRAMMDQVRESRNPGNAEIEDLIFSARSALILRGSVAHKSCLPACVYGPAVSSGYNFEKEGYSLVGIDPFKLLQNSQVYSLIRPNENPAHKSQLVWMACHSAAFEDLRLLSFIRGTKVSPRGKLSTRGVQIASNENMDNMESSTLELRSRYWAIRTRSGGNTNQQRASAGQISVQPTFSVQRNLPFEKSTVMAAFTGNTEGRTSDMRAEIIRMMEGAKPEEVSFRGRGVFELSDEKATNPIVPSFDMSNEGSYFFGDNAEEYDN

SEQ ID NO: 21 - DNA, 573 bp, Artificial Sequence
  BHA10-2
atggagacccccgcccagctgctgttcctgctgctgctgtggctgcccgacaccaccggccacgtggtgaagaccgccacccagggcgaggtgaacgtgaccggcgtgatccccctgaccaccacccccaccggcagcgccaacaagagcaagccctactacaccggcgagcacgccaaggccaccggcaactgccccatctgggtgaagacccccctgaagctggccaacggcaccaagtacggcagcgccggcagcgccacccaggaggccatcaacaagatcaccaagaacctgaacagcctgagcgagctggaggtgaagaacctgcagaggctgagcggcgccagcgacgagacccacaacgagatcctggagctggacgagaaggtggacgacctgagggccgacaccatcagcagccagatcgagctggccgtgctgctgagcaacgagggcatcatcaacagcgaggacgagggcaccggcggcggctacatccccgaggcccccagggacggccaggcctacgtgaggaaggacggcgagtgggtgctgctgagcaccttcctg

SEQ ID NO: 22 - DNA, 480 bp, Artificial Sequence
  BHA10-3
atggagacccccgcccagctgctgttcctgctgctgctgtggctgcccgacaccaccggccacgtggtgaagaccgccacccagggcgaggtgaacgtgaccggcgtgatccccctgaccaccacccccaccggcagcgccaacaagagcaagccctactacaccggcgagcacgccaaggccaccggcaactgccccatctgggtgaagacccccctgaagctggccaacggcaccaagtacggcagcgccggcagcgccacccaggaggccatcaacaagatcaccaagaacctgaacagcctgagcgagctggaggtgaagaacctgcagaggctgagctgcgccagcgacgagacccacaactgcatcctggagctggacgagaaggtggacgacctgagggccgacaccatcagcagcctgatcgagctggccgtgctgctgagcaacgagggcatcatcaacagcgaggacgag

SEQ ID NO: 23 - DNA, 780 bp, Artificial Sequence
  NIHGen6HASS-TM
atggagacccccgcccagctgctgttcctgctgctgctgtggctgcccgacaccaccggcgacaccatctgcatcggctaccacgccaacaacagcaccgacaccgtggacaccgtgctggagaagaacgtgaccgtgacccacagcgtgaacctgggcagcggcctgaggatggtgaccggcctgaggaacatcccccagagggagaccaggggcctgttcggcgccatcgccggcttcatcgagggcggctggaccggcatggtggacggctggtacggctaccaccaccagaacgagcagggcagcggctacgccgccgaccagaagagcacccagaacgccatcaacggcatcaccaacatggtgaacagcgtgatcgagaagatgggcagcggcggcagcggcaccgacctggccgagctgctggtgctgctgctgaacgagaggaccctggacttccacgacagcaacgtgaagaacctgtacgagaaggtgaagagccagctgaagaacaacgccaaggagatcggcaacggctgcttcgagttctaccacaagtgcaacaacgagtgcatggagagcgtgaagaacggcacctacgactaccccaagtacagcgaggagagcaagctgaacagggagaagatcgaccagggcaccggcggcatcctggccatctacagcaccgtggccagcagcctggtgctgctggtgagcctgggcgccatcagcttctggatgtgcagcaacggcagcctgcagtgcagaatctgcatc

SEQ ID NO: 24 - DNA, 798 bp, Artificial Sequence
  NIHGen6HASS-TM2
atggagacccccgcccagctgctgttcctgctgctgctgtggctgcccgacaccaccggcgacaccatctgcatcggctaccacgccaacaacagcaccgacaccgtggacaccgtgctggagaagaacgtgaccgtgacccacagcgtgaacctgggcagcggcctgaggatggtgaccggcctgaggaacatcccccagagggagaccaggggcctgttcggcgccatcgccggcttcatcgagggcggctggaccggcatggtggacggctggtacggctaccaccaccagaacgagcagggcagcggctacgccgccgaccagaagagcacccagaacgccatcaacggcatcaccaacatggtgaacagcgtgatcgagaagatgggcagcggcggcagcggcaccgacctggccgagctgctggtgctgctgctgaacgagaggaccctggacttccacgacagcaacgtgaagaacctgtacgagaaggtgaagagccagctgaagaacaacgccaaggagatcggcaacggctgcttcgagttctaccacaagtgcaacaacgagtgcatggagagcgtgaagaacggcacctacgactaccccaagtacagcgaggagagcaagctgaacagggagaagatcgacggagtgaaattggaatcaatgggggtctatcagatcctggccatctacagcaccgtggccagcagcctggtgctgctggtgagcctgggcgccatcagcttctggatgtgcagcaacggcagcctgcagtgcagaatctgcatc

SEQ ID NO: 25 - DNA, 744 bp, Artificial Sequence
  NIHGen6HASS-foldon
atggagacccccgcccagctgctgttcctgctgctgctgtggctgcccgacaccaccggcgacaccatctgcatcggctaccacgccaacaacagcaccgacaccgtggacaccgtgctggagaagaacgtgaccgtgacccacagcgtgaacctgggcagcggcctgaggatggtgaccggcctgaggaacatcccccagagggagaccaggggcctgttcggcgccatcgccggcttcatcgagggcggctggaccggcatggtggacggctggtacggctaccaccaccagaacgagcagggcagcggctacgccgccgaccagaagagcacccagaacgccatcaacggcatcaccaacatggtgaacagcgtgatcgagaagatgggcagcggcggcagcggcaccgacctggccgagctgctggtgctgctgctgaacgagaggaccctggacttccacgacagcaacgtgaagaacctgtacgagaaggtgaagagccagctgaagaacaacgccaaggagatcggcaacggctgcttcgagttctaccacaagtgcaacaacgagtgcatggagagcgtgaagaacggcacctacgactaccccaagtacagcgaggagagcaagctgaacagggagaagatcgaccccggcagcggctacatccccgaggcccccagggacggccaggcctacgtgaggaaggacggcgagtgggtgctgctgagcaccttcctg

SEQ ID NO: 26 - DNA, 1698 bp, Artificial Sequence
  ConH1
atgaaggccaagctgctggtgctgctgtgcgccttcaccgccaccgacgccgacaccatctgcattggctaccatgccaacaacagcaccgacaccgtggacaccgtgctcgagaagaacgtgaccgtgacccactccgtgaatttgttggaggacagccacaacggcaagctgtgtaagctgaagggcatcgcccccctgcagctgggcaagtgcaacatcgccggctggatcttgggcaatcccgagtgcgaaagcctgatctccaagagaagctggagctacatcgtggagactcccaacagcgaaaacggcacctgctaccccggcgacttcgctgactacgaggaactgagagagcagctgagcagcgtgagcagctttgagagattcgagatcttccccaaagagagcagctggcccaaccacaacgtaaccaagggcgtgacagccgcctgcagccacgccggtaagagcagcttctacagaaacctgctgtggctgacagagaagaacggcagctaccccaagctgagcaagagctatgtgaacaacaaggagaaggaggtgctggtcctgtggggcgtacaccaccccagcaacattaccgatcagagaaccctgtaccagaacgagaatgcctacgtgagcgtggtgagcagccactacaacagaagattcacccccgagattgccaagagaccgaaagtgagaggccaggccggaagaatcaactactactggaccctgctggagcccggcgacaccatcatcttcgaggccaacggcaacctgatcgccccctggtatgccttcgccctgagcagaggcttcggaagcggcatcatcacatccaacgcccccatgcatgaatgcgacacaaagtgtcagaccccccagggcgccatcaacagcagcctgcccttccagaacgtgcaccctgtgaccatcggcgagtgccccaagtacgtgagaagcaccaagctgagaatggtgaccggcctgagaaatatccccagtatccagagcagaggcctgttcggcgccatcgccggcttcatcgagggcggctggaccggcatgatcgacggctggtacggctaccaccaccagaacgagcagggcagcggctacgccgccgaccagaagagcactcagaacgccatcaacggcatcaccaacaaggtgaacagcgtgatcgagaagatgaacacacagttcaccgccgtgggcaaagagttcaataagctcgagaagagaatggaaaacctgaacaagaaggttgacgacggtttcctggatatctggacctacaacgccgagctgcttgtgctgctggagaacgagcgtaccctggactttcacgactcgaacgtgaagaacctgtacgaaaaggtgaagtcccagctgaagaacaacgccaaagaaattggcaacggctgcttcgagttctaccacaagtgtaacaacgagtgcatggagagcgtgaagaacggcacctacgactatcccaagtacagcgaggagagcaagctaaacagagagaagattgacggcgtgaaactggagtcaatgggcgtgtaccagatcctggccatctacagcaccgtggccagcagcctcgtgctgctggtgagcctgggcgccataagcttctggatgtgtagcaacggcagcctgcagtgcagaatctgcatc

SEQ ID NO: 27 - DNA, 1698 bp, Artificial Sequence
  ConH3
atgaagaccatcatcgctctgagctacatattctgcctggtgttcgcccagaagctgcccggcaacgacaacagcaccgccaccctgtgcctgggccatcacgcagtgccaaacggcaccttagttaagaccatcaccaacgaccagatcgaggtgaccaacgccaccgagctggtgcagagcagtagcaccggcagaatctgcgacagcccccaccggatccttgacggcactaactgcaccctgatcgacgccctgctgggcgacccccactgcgacgggttccagaacaaggagtgggacctgttcgtggagagaagcaaggcctacagcaactgctacccctacgatgtgcccgactacgccagcctgagatctcttgtggctagcagcggcaccctggagttcaacaatgagggcttcaattggacaggcgtgacccagaacggcggcagcagcgcctgcaagagaggcagcgacaagagcttcttcagcagactgaactggctgcacaagctgaagtacaagtatcccgccctgaacgtgaccatgcccaataacgacaagttcgataagctgtatatttggggcgtgcaccaccccagcaccgacagcgaccagacctccctgtacgtccaggcgagcggcagagtgaccgtgagcaccaaacggagccagcaaaccgtgatccccaacatcggcagcagaccttgggtcagaggactgagcagcagaatcagcatctactggaccatcgtgaagcctggcgacatcttgctgatcaatagcaccggcaacctgatcgcccccagaggctacttcaagatcagaagcggcaagagctcaatcatgagaagcgacgcccccataggcacctgcaacagcgagtgcatcaccccgaacggcagcatccccaacgacaagcccttccagaacgtgaacagaatcacatacggcgcctgccccaggtacgtaaagcaaaacaccctgaagctggccaccggcatgagaaacgtacccgagaagcagaccagaggcatcttcggcgccatcgcgggcttcatcgagaatggctgggaaggcatggtggacggctggtacggcttcagacatcagaacagcgagggcaccggccaggccgccgacctgaaaagcacccaggccgccatcgaccagatcaacggcaagctgaacagactgatcgaaaagaccaacgagaagttccaccagatcgagaaggagttcagcgaagtggagggaagaatacaggacctggagaaatacgtggaggacaccaagatcgacctgtggtcgtacaacgccgagctgctggtggccctggaaaaccagcacaccattgacctgaccgatagcgagatgaacaagctgttcgagagaaccagaaaacagctgagagagaacgccgaggacatgggcaacggctgctttaagatctaccacaagtgcgacaacgcctgcatcggcagcatcagaaacggcacctacgaccacgacgtgtacagagacgaggccctgaacaacagattccagatcaagggcgtggagctgaagagcggctacaaggactggatcctgtggatcagcttcgccatcagctgtttcctgctttgcgtagtgctgctgggcttcatcatgtgggcctgccagaagggcaatatcagatgcaacatttgcatc

SEQ ID NO: 28 - DNA, 1698 bp, Artificial Sequence
  MRK_pH1_Con
atgaaggccatcctggtggtgctgctgtacaccttcgccacggccaacgctgacaccctgtgcatcggataccacgcgaacaacagcaccgacaccgttgacaccgtgctggagaagaacgtgaccgtgacccacagcgtgaacctcctggaagacaagcacaacggcaagctgtgcaagctgagaggcgtggcccccctgcacctgggcaagtgcaacatcgccggctggatcttaggcaaccccgagtgcgagagcctgagcaccgccagcagctggagctacattgtggagaccagcagcagcgacaacggcacctgctaccccggcgacttcatcgactacgaggagctgagagagcagctgagcagcgtgagcagcttcgagagattcgagatcttccccaagacctcaagctggcccaaccacgacagcaataagggcgtgactgccgcctgcccccacgccggcgccaagagcttctacaagaacctgatctggctggtgaagaaaggcaatagctaccccaagctgagcaagtcctatatcaacgacaagggcaaggaggtgttggttctgtggggcatccatcaccccagcaccagtgctgatcagcaaagcctgtaccaaaacgccgatgcctacgtgttcgtgggcacctcaagatacagcaagaagttcaagcccgagatcgccatcagacccaaggtgagagaccaggagggtagaatgaactactactggaccctcgtggagccgggcgacaagatcaccttcgaggccaccggcaacctggtggtgcccagatacgccttcgccatggagagaaatgccggcagcgggatcatcatctcggacacccccgtgcacgattgtaacaccacctgccagacccccaagggcgccatcaacacctccctgcccttccagaacatccaccccatcaccatcggcaagtgccccaagtacgtgaagagcaccaagctgagactggcgaccggactgagaaacgtgcccagcatccagtcaagaggcctgttcggcgccatcgccggcttcatcgagggcggctggacaggcatggtggacggctggtacggctaccaccaccagaacgagcagggcagcggatacgccgccgacctgaagagcacacaaaacgccatcgacaagatcaccaacaaggtgaacagcgtgatcgaaaagatgaacacccagttcaccgccgtgggcaaggagttcaaccacctggagaagagaatcgagaacctgaacaagaaagtggacgacggcttcctggacatctggacctacaacgccgagctgctggtcctgctggagaacgagagaaccctggactaccatgacagcaacgtgaagaacctgtacgagaaggtgagaagccagctgaagaacaacgccaaggagataggcaacggctgcttcgagttctaccacaagtgcgacaacacctgcatggagagcgtgaagaacggcacctacgactacccaaagtatagcgaggaggcaaagctgaacagagaggagatcgacggcgtgaagctggagagcaccaggatctatcaaatcctggccatatacagcaccgtggccagcagcctggtgttagtggtgagcctgggcgccatcagcttctggatgtgtagcaacggcagcctgcagtgcagaatctgcatc

SEQ ID NO: 29 - DNA, 1587 bp, Artificial Sequence
  MRK_pH1_Con
atgaaggccatcctggtggtgctgctgtacaccttcgccacggccaacgctgacaccctgtgcatcggataccacgcgaacaacagcaccgacaccgttgacaccgtgctggagaagaacgtgaccgtgacccacagcgtgaacctcctggaagacaagcacaacggcaagctgtgcaagctgagaggcgtggcccccctgcacctgggcaagtgcaacatcgccggctggatcttaggcaaccccgagtgcgagagcctgagcaccgccagcagctggagctacattgtggagaccagcagcagcgacaacggcacctgctaccccggcgacttcatcgactacgaggagctgagagagcagctgagcagcgtgagcagcttcgagagattcgagatcttccccaagacctcaagctggcccaaccacgacagcaataagggcgtgactgccgcctgcccccacgccggcgccaagagcttctacaagaacctgatctggctggtgaagaaaggcaatagctaccccaagctgagcaagtcctatatcaacgacaagggcaaggaggtgttggttctgtggggcatccatcaccccagcaccagtgctgatcagcaaagcctgtaccaaaacgccgatgcctacgtgttcgtgggcacctcaagatacagcaagaagttcaagcccgagatcgccatcagacccaaggtgagagaccaggagggtagaatgaactactactggaccctcgtggagccgggcgacaagatcaccttcgaggccaccggcaacctggtggtgcccagatacgccttcgccatggagagaaatgccggcagcgggatcatcatctcggacacccccgtgcacgattgtaacaccacctgccagacccccaagggcgccatcaacacctccctgcccttccagaacatccaccccatcaccatcggcaagtgccccaagtacgtgaagagcaccaagctgagactggcgaccggactgagaaacgtgcccagcatccagtcaagaggcctgttcggcgccatcgccggcttcatcgagggcggctggacaggcatggtggacggctggtacggctaccaccaccagaacgagcagggcagcggatacgccgccgacctgaagagcacacaaaacgccatcgacaagatcaccaacaaggtgaacagcgtgatcgaaaagatgaacacccagttcaccgccgtgggcaaggagttcaaccacctggagaagagaatcgagaacctgaacaagaaagtggacgacggcttcctggacatctggacctacaacgccgagctgctggtcctgctggagaacgagagaaccctggactaccatgacagcaacgtgaagaacctgtacgagaaggtgagaagccagctgaagaacaacgccaaggagataggcaacggctgcttcgagttctaccacaagtgcgacaacacctgcatggagagcgtgaagaacggcacctacgactacccaaagtatagcgaggaggcaaagctgaacagagaggagatcgacggcgtgaagctggagagcaccaggatctatcaa

SEQ ID NO: 30 - DNA, 1695 bp, Artificial Sequence
  MRK_sH1_Con
atgaaggtgaagctgctggtgctgctgtgtaccttcactgccacttacgccgacaccatttgcatcggctaccacgccaacaacagcaccgataccgtggacaccgtgctggagaagaacgtcaccgtgacccacagcgtgaacctgctggaggatagccataacggcaagctgtgcctgctgaagggaatcgcccccctgcagctcggcaactgcagcgtggccggctggattctgggcaaccccgagtgcgaactgctgattagcaaagagtcctggagctacatcgtggaaaccccgaatcccgagaacggcacctgctaccccggctacttcgccgactacgaagagctaagagagcagctgagtagcgtgagctcattcgagagattcgagatctttcccaaggagtctagctggcccaatcacaccgtgaccggcgtgagcgccagctgtagccacaacggcaagagcagcttctacagaaacctgctgtggctgaccggcaagaacggactgtaccctaacctgagcaagagctacgcgaacaataaggagaaggaggtgctagtgctgtggggcgtgcaccacccccccaacatcggcgaccagagagccctgtaccacaccgagaacgcctacgtgagcgtggtgagcagccactatagcagaagattcacccccgagatcgccaagagaccaaaggtgagagatcaggaaggaagaataaactactactggaccctcctggagcccggcgacaccatcatcttcgaggctaacggcaacctgatcgcccccagatacgccttcgccctgagcagaggcttcggcagcggcatcatcaccagcaatgcccccatggatgagtgcgacgccaagtgccagaccccccagggcgccatcaactcgagcctgcccttccagaatgtgcaccccgtgaccatcggcgagtgccccaagtacgtgagaagcgccaagctgagaatggtgaccggcctgagaaacatcccaagcatccagagcagagggctgttcggcgccatcgctggcttcatcgagggcggctggaccggcatggtggacggctggtacggttatcaccaccagaacgagcagggcagcggctacgccgccgaccagaagagcacccagaacgccatcaacggcattacaaacaaggtgaacagcgttatcgagaagatgaacacccaattcaccgccgtgggcaaggagttcaacaagctggagagaagaatggagaacctgaacaagaaggtggacgacggcttcctggacatctggacctacaacgccgagctgctggtgctgctggagaacgagagaaccctggacttccacgactccaacgtgaagaacttatacgagaaggtgaagagccagctgaagaacaacgccaaagaaatcggaaacggctgcttcgaattctaccacaagtgcaacgacgaatgcatggagagcgtgaagaacggaacctacgactaccccaagtacagcgaggaaagcaaactgaacagagagaagatcgacggcgtgaagttagagagcatgggcgtgtatcagatcctggccatttatagcacggtggccagcagcctggtgctgctggtgagcctgggcgccatcagcttctggatgtgcagcaacggcagcctgcagtgcagaatctgcatc

SEQ ID NO: 31 - DNA, 1584 bp, Artificial Sequence
  MRK_sH1_Con
atgaaggtgaagctgctggtgctgctgtgtaccttcactgccacttacgccgacaccatttgcatcggctaccacgccaacaacagcaccgataccgtggacaccgtgctggagaagaacgtcaccgtgacccacagcgtgaacctgctggaggatagccataacggcaagctgtgcctgctgaagggaatcgcccccctgcagctcggcaactgcagcgtggccggctggattctgggcaaccccgagtgcgaactgctgattagcaaagagtcctggagctacatcgtggaaaccccgaatcccgagaacggcacctgctaccccggctacttcgccgactacgaagagctaagagagcagctgagtagcgtgagctcattcgagagattcgagatctttcccaaggagtctagctggcccaatcacaccgtgaccggcgtgagcgccagctgtagccacaacggcaagagcagcttctacagaaacctgctgtggctgaccggcaagaacggactgtaccctaacctgagcaagagctacgcgaacaataaggagaaggaggtgctagtgctgtggggcgtgcaccacccccccaacatcggcgaccagagagccctgtaccacaccgagaacgcctacgtgagcgtggtgagcagccactatagcagaagattcacccccgagatcgccaagagaccaaaggtgagagatcaggaaggaagaataaactactactggaccctcctggagcccggcgacaccatcatcttcgaggctaacggcaacctgatcgcccccagatacgccttcgccctgagcagaggcttcggcagcggcatcatcaccagcaatgcccccatggatgagtgcgacgccaagtgccagaccccccagggcgccatcaactcgagcctgcccttccagaatgtgcaccccgtgaccatcggcgagtgccccaagtacgtgagaagcgccaagctgagaatggtgaccggcctgagaaacatcccaagcatccagagcagagggctgttcggcgccatcgctggcttcatcgagggcggctggaccggcatggtggacggctggtacggttatcaccaccagaacgagcagggcagcggctacgccgccgaccagaagagcacccagaacgccatcaacggcattacaaacaaggtgaacagcgttatcgagaagatgaacacccaattcaccgccgtgggcaaggagttcaacaagctggagagaagaatggagaacctgaacaagaaggtggacgacggcttcctggacatctggacctacaacgccgagctgctggtgctgctggagaacgagagaaccctggacttccacgactccaacgtgaagaacttatacgagaaggtgaagagccagctgaagaacaacgccaaagaaatcggaaacggctgcttcgaattctaccacaagtgcaacgacgaatgcatggagagcgtgaagaacggaacctacgactaccccaagtacagcgaggaaagcaaactgaacagagagaagatcgacggcgtgaagttagagagcatgggcgtgtatcag

SEQ ID NO: 32 - DNA, 1698 bp, Artificial Sequence
  Cobra_P1
atgaaggcccgcctcttggtgctgctgtgcgccctggcggccacagacgccgacacaatctgtatcggctaccacgccaataatagcaccgataccgtggataccgtgctcgagaagaacgtcaccgttacacactccgtgaatttactggaggacagccacaatggcaagctctgcaaactgaagggtatcgccccactccaactgggcaagtgcaacatcgcaggctggctgctgggcaaccctgagtgtgagagcctgctgagcgctagaagctggagctacatagtggagacacctaacagcgaaaacggcacatgctaccccggcgacttcatcgattacgaggaactgcgggagcagctgagtagcgtgagctcctttgagagatttgagatcttccccaaagagagcagctggcccaaccataataccaccaaaggcgtgaccgccgcttgcagtcatgcagggaaaagtagcttctaccggaacctgctctggttgaccaagaagggagggagctacccaaagttgagcaaaagctacgtgaataacaagggcaaggaggtgctcgtgctgtggggagtccaccatcccagcacatccactgatcagcagtccctgtatcagaacgaaaacgcctacgtgagtgtggtgagctctaactacaacagacggttcacccctgaaattgctgagaggccaaaggtgagaggccaggccggcagaatgaactactattggaccctcctggagcctggggacacgatcatcttcgaggcgaccgggaacctcatcgctccctggtatgccttcgccctgagcaggggcagtggcagcggaatcatcaccagcaacgccagcatgcatgagtgcaacactaaatgccagaccccccagggggccatcaacagctccctgcccttccagaacatccatcctgtgaccattggggagtgccccaagtacgtgaggtccaccaagctgaggatggtgactggactgaggaacatccccagcatccagagccgggggctgtttggcgccattgccggctttatcgagggtggctggacaggtatgattgatggctggtacggataccaccaccagaacgagcaggggagtgggtatgctgccgaccagaaatctactcagaacgccatcaatggcatcaccaataaggtgaacagcgtcatcgagaagatgaacacccagttcaccgctgtgggcaaggagttcaacaacctggaaaagcgcatggagaacctgaacaagaaggtggacgacggcttcctggacatctggacctacaacgccgagctgcttgtcctcctggagaacgagaggaccttggacttccatgacagcaatgtgaagaacctctacgagaaagtgaagagccagctgagaaacaatgctaaggagatcggcaacggctgcttcgagttttaccacaagtgcgacaatgagtgcatggagagcgtgaagaatggcacttatgactaccccaagtactcagaggagtccaaactgaatagagagaagattgatggcgtcaagctagagtccatgggcgtttaccagatcctggcaatctatagcaccgtggccagctccctggtgctgctggtgtcactgggagccatatccttctggatgtgctccaacggcagccttcagtgtagaatctgcatc

SEQ ID NO: 33 - DNA, 1698 bp, Artificial Sequence
  Cobra_X3
atggaggcccgcctgctcgtgcttctgtgcgcctttgccgccactaacgccgacaccatctgtatcggctaccacgccaacaatagtacagataccgtggacactgtgctggagaagaacgtaacagtgacacattctgtcaacctgctcgaggactctcataatggcaagctgtgccgcctgaagggcatcgcccctctgcagctgggaaattgctccgtggccggctggatcctgggcaatccggaatgcgaaagcctgttcagcaaggagagctggagctacatcgccgagacacctaaccctgagaacgggacctgctaccctggatacttcgccgactacgaagagctgcgggagcagctcagctcagtgtcatccttcgagcggttcgagatcttccccaaggagagctcttggcccaaccacaccgtgaccaagggcgtcacagcaagctgtagccacaacggcaagagctccttctatagaaacctgctgtggctgaccgagaagaacggcctgtaccccaatctgagtaagtcctacgtgaacaacaaggaaaaggaagtgctggtgctgtggggcgtgcaccacccctccaacatcggcgaccagcgcgccatctaccacactgagaatgcatacgtaagcgttgtcagctcccactatagtaggagattcacacccgagatcgctaagaggcccaaggtgagagaccaggagggcagaatcaattattactggaccctgctggagcccggagacaccattatcttcgaagctaacggcaatttgatcgccccttggtatgcctttgccctctcaaggggtttcgggagcggaattatcacctccaatgccagcatggatgagtgcgacgccaagtgccagacgcctcagggcgccattaattcctccctgcccttccagaacgtgcaccccgtgaccatcggggagtgccccaagtatgttagatccactaagctcaggatggtgacaggactgcgcaacatcccgagcattcagagcaggggcctcttcggggccattgctgggttcatcgagggcgggtggaccggcatgatcgacggctggtatggctaccaccaccagaacgagcagggcagcgggtacgctgctgaccaaaagtccacccaaaatgctatcaacggcatcaccaacaaggttaatagcgtcatcgaaaagatgaatacccagttcacagccgtgggaaaggaattcaacaagctggaacgacggatggagaacctgaataagaaggtggacgacgggttcctggacatctggacttataacgctgagctgctcgtgctgttagagaacgagagaaccctggactttcacgacagcaacgtgaagaacctgtacgagaaggtgaagtctcagctgaaaaataacgctaaggaaattggcaacgggtgcttcgaattctatcacaagtgcaacaacgaatgcatggagagtgttaagaacggaacctatgactaccccaagtacagtgaggaaagtaaactgaatagggagaagatcgacggcgtgaaactggagtccatgggggtttaccagattctggccatctatagcaccgtggccagcagcttagtgctgctggtgtccctcggcgctattagcttctggatgtgcagcaacggaagcctgcagtgtcggatatgcatc

SEQ ID NO: 34 - DNA, 2052 bp, Artificial Sequence
  ConH1_ferritin
atgaaggcgaagctccttgtgctgctctgcgcgttcaccgccaccgacgcagatacaatttgcatcggataccacgccaacaattccaccgacaccgtggacaccgttctggagaaaaacgtgacggtgacccacagcgtgaacctcctggaggatagccataacggcaagctgtgtaagctgaaaggcatcgcccccctgcagctgggaaagtgcaacattgctggatggatcctgggaaatcccgagtgtgaaagcctcattagcaaacgcagctggagctacattgtggagaccccaaattctgagaatgggacctgttaccctggcgactttgccgactacgaggagctgagagagcagttgagcagcgtcagctccttcgagagattcgaaatctttccaaaggagtcttcgtggcccaaccacaacgtgactaagggcgtcaccgcagcttgtagccacgcgggcaaatcttccttctacagaaacctactgtggctcaccgagaaaaacggcagctaccccaagctgagcaagagctacgtgaataacaaagagaaggaagtgctggtgctgtggggcgtccaccaccccagcaacatcacagaccaaagaacactctaccagaacgagaacgcctacgtgagtgtggtgtccagccattacaaccgccgattcacccccgagatcgccaaacggcccaaagtgcggggccaggccggaagaattaactactactggaccctcctggaaccaggagacaccattatcttcgaagccaatggcaatctgatcgctccctggtacgccttcgcactgtcgagagggtttggcagcggcatcatcacctccaacgccccaatgcatgaatgtgataccaagtgccagaccccacagggcgccattaacagcagcctgccattccagaacgtccatcccgtgacaatcggcgagtgtcctaagtacgtgcgctcaacgaaactgaggatggtgacaggactgagaaacattccctcaatccagagcagagggctgttcggcgccatagccggattcattgagggcggatggacaggcatgattgacggctggtatggctaccaccatcagaacgagcaaggcagtggctacgcagccgaccagaagagcacacagaacgccattaacgggatcaccaacaaggtgaatagcgtgatcgagaagatgaatacccagttcactgccgtgggtaaggagttcaacaagctggagaagcggatggagaacctcaacaagaaagtcgatgatggcttcctggacatctggacctataatgctgaactgctcgtgctacttgagaatgagaggacgcttgactttcacgactccaacgtaaaaaacctgtacgagaaggtgaagtcgcagctgaaaaataacgccaaggaaatcggcaacggctgttttgagttttaccataaatgcaataacgagtgcatggagagcgtgaagaatggcacctacgactatcccaaatactccgaggagagcaagctcaaccgggagaaaatcgatagcggcggggatatcattaagctgcttaacgagcaggtcaacaaggagatgcagtcaagcaacctttacatgagcatgagcagctggtgttacacacacagcctggacggagccggactgttcctgttcgaccacgctgcagaggaatatgagcacgctaagaagcttataattttcctcaacgagaataacgtgcccgtccagctgacctccatcagcgcccccgagcacaagtttgagggcctgacccagatcttccagaaggcctacgagcacgagcagcacatcagcgagtctatcaacaacatcgtagaccatgcaatcaagtctaaggaccacgctacatttaactttctgcaatggtacgtggctgaacaacacgaggaggaggtactgttcaaggatattctcgacaagatcgaactcatcgggaatgagaaccacggcctgtacctggccgaccagtacgtgaaaggaattgccaaatccagaaagtcc

SEQ ID NO: 35 - DNA, 2055 bp, Artificial Sequence
  ConH3_ferritin
atgaagacaatcattgccctgagctacattttttgcttagtgtttgctcagaaactgccaggcaacgataattcaacagccaccttgtgcctcggccaccacgctgtgcctaacggcactctggtgaagaccatcaccaacgaccagatcgaggtgaccaacgccacggagctggtgcagtcaagctccaccggaagaatctgcgatagcccccataggattctggatggcaccaactgcaccctgattgacgccctgctcggcgatccccactgcgacggtttccaaaacaaggagtgggacctgtttgtggagagaagcaaggcctattcaaattgctacccttacgacgtccctgattacgcctcactcaggtccctggtggccagcagcgggaccctggaattcaacaatgaggggttcaactggaccggggtgacccaaaacggcggctccagcgcctgtaagaggggcagcgacaagtccttcttctctaggctgaactggttgcacaaactgaagtacaagtaccctgcattaaacgtgaccatgcccaacaacgataaattcgacaagctgtacatctggggagtgcatcaccccagcacagactcagaccagaccagtctgtatgtgcaggcaagcgggagggtgacggtctccaccaagcggagccagcagaccgtgatccccaacatcggctccagaccatgggtcaggggcctgagcagccggatctccatctactggaccatagtgaagcctggcgacatcctgctgatcaacagcaccggcaacctcatcgcccctcgcggttacttcaagatccgtagtggcaaatcaagcatcatgagatccgacgcacccatcgggacctgcaatagcgagtgcatcacccccaacggatctatccctaatgacaagccttttcagaacgtgaaccggattacctatggtgcctgccccagatacgtgaagcagaacaccctgaagctggcgaccggcatgcgcaacgtgccggaaaagcagacccggggcatcttcggcgccattgccgggtttattgagaatggctgggaaggcatggtggatgggtggtacggctttaggcatcagaactctgagggtactggtcaggccgccgacctgaaatccacccaggccgccattgaccagattaacgggaaacttaacagactgattgagaagaccaatgagaagttccaccagatcgaaaaggaattctccgaagtggagggcaggattcaggacttagagaaatatgtggaggataccaagatcgacctgtggagctataacgccgagctgcttgtggctctggagaaccagcacaccatcgatctgaccgacagcgagatgaataagctgttcgagaggacacgcaagcagctgagggagaacgccgaggacatggggaacgggtgctttaagatctatcacaagtgcgacaatgcctgcatcgggtctatcagaaatggcacttatgatcatgacgtgtacagagatgaggccctgaataatagatttcaaattaagtccgggggtgacatcattaaactgctgaacgaacaagtgaataaagagatgcagagctctaacttgtacatgagcatgagcagttggtgctacacacactctctggacggggctggcctgttcctgttcgatcacgcagcagaggagtatgagcacgccaaaaaactgattatcttcctcaacgagaacaacgtgcccgtccagctcacctccatctcagcccccgagcacaagttcgagggcctcacccagatcttccagaaagcatatgagcatgaacagcatatcagtgaaagcatcaacaatatcgtggaccacgctattaaatcaaaggatcacgccaccttcaactttctgcagtggtatgtcgccgagcagcatgaggaggaggtgctttttaaagacatcctggacaagatcgagctgatcggcaacgaaaaccatggcctgtacctggctgaccagtatgtgaagggaattgccaagtccagaaaatcc

SEQ ID NO: 36 - DNA, 2052 bp, Artificial Sequence
  MRK_pH1_Con_ferritin
atgaaggcgattctggttgtgctgctgtacaccttcgccaccgccaacgccgacacactgtgcattggctaccacgccaataacagcacggacaccgtggacacggtgctggagaaaaacgtgaccgtgacccacagcgtgaacctgctggaggacaagcacaacggtaagttgtgcaagctcaggggagtggcaccactgcacttgggcaagtgtaatatcgctggctggatattgggaaatccagagtgcgaaagcctgagtactgcctccagctggagctacattgtggagaccagcagcagcgacaacggcacctgctaccccggcgacttcatcgactatgaggaattgagggaacagctgagttcagtttccagcttcgagcgatttgagatatttcccaagacgtcctcttggcccaaccacgacagcaacaagggcgtgacagccgcctgcccccacgccggggcgaagagcttctacaagaacctgatctggctggtgaagaagggcaacagctacccaaagctatccaagtcctatattaacgacaaaggcaaggaggtgttggtgctctggggcattcaccacccctccacctccgccgaccagcaaagtctttaccagaacgcggacgcctacgtctttgtcggcaccagcagatacagcaagaagtttaagcccgagattgctatcagacccaaggtgagagaccaggaaggcagaatgaactattattggaccctggtggaacccggcgacaaaataacattcgaagccaccgggaatctggtggtgcccagatatgcctttgccatggagcgcaatgccggcagcggcattattatctctgacacccccgtgcacgactgcaacaccacctgtcagacccctaagggggctatcaacaccagcctgcccttccagaatattcaccccatcactatcggcaagtgccccaagtacgtcaagagcacaaaactgagactggccacagggctgaggaatgtacctagcatccagtccagagggctgttcggggccatcgctggcttcatcgaaggaggctggaccggcatggtcgatggatggtacggatatcaccaccaaaacgagcaggggtcaggatacgccgctgacctgaagagcacccagaacgccatcgacaagatcaccaacaaggtgaatagcgtgatcgagaagatgaacacccagttcaccgcagtgggcaaggagttcaaccacctggagaagagaatcgagaacctgaacaagaaagtggatgacgggttcctggacatctggacctacaacgccgagcttctggtgctcttggagaatgagagaaccctggattatcatgacagcaatgtcaaaaacctctacgagaaggtgcggagccagctgaagaacaacgcaaaggagattggcaacggctgcttcgagttttatcacaagtgcgacaacacttgtatggagagcgttaagaatggcacttacgattaccccaagtactccgaggaagccaagctgaacagagaagaaatcgactccggcggcgacataatcaagctcctgaacgaacaggtgaacaaggagatgcaaagctccaacctctacatgagcatgagctcatggtgctacactcacagcctggacggagctggactgttcttgttcgaccacgcggccgaggagtacgagcacgccaagaagctcatcatcttccttaacgagaataacgtgccagtgcagctcacctccatcagcgcccccgagcataagttcgagggtctgacccaaatcttccagaaggcttacgagcatgagcagcacatcagcgagagcattaacaacatcgtggatcacgctattaaatctaaagaccacgccaccttcaacttcctgcagtggtacgtggcagaacagcacgaggaggaggtcctgttcaaggatatactggacaaaatcgagctgatcggcaacgagaaccacggcctgtacctggccgatcagtacgtcaaaggtattgccaagtctcgcaagagc

SEQ ID NO: 37 - DNA, 2049 bp, Artificial Sequence
  MRK_sH1_Con_ferritin
atgaaggtgaagctgcttgtgctgctgtgcaccttcaccgctacctacgcagacacaatctgtatcggataccacgccaataactcaaccgatacagtggacaccgtgctcgagaagaacgtgacagtgacgcacagcgtgaacctgcttgaggattcccataacggtaagctctgtctgctgaagggcatcgcccctcttcagctgggaaactgctccgttgccggctggatcctgggcaaccccgagtgtgagcttctgatcagcaaggagtcgtggtcatatatcgtggagacccctaatccagagaacggaacctgttaccccggctactttgccgactacgaggagctcagagagcagctgagcagcgtgagcagcttcgagagattcgagatcttccccaaggagagcagttggcctaatcacaccgtgaccggcgtgagcgcctcctgcagccacaacggcaagtcttccttttacagaaacctgctgtggctgacaggcaaaaacgggttgtaccctaacctgagcaagtcctatgctaacaataaggagaaggaagtcctggtgttgtggggcgttcaccatcccccaaacatcggagaccaacgcgccctatatcacactgagaacgcctacgtgagcgtggtgtcaagccactatagcagacggttcacccccgaaatcgcaaagagaccgaaggtgcgggaccaggagggaaggattaactattactggacactcctggagcccggggacactatcatctttgaagccaacgggaacctcatcgcacccaggtacgctttcgctctgtccaggggattcgggagcggtatcattacctcgaacgccccgatggatgagtgcgacgccaaatgccaaaccccccagggcgctattaactctagcctcccttttcagaacgtgcaccccgtgaccatcggagagtgccccaagtacgtgcggagcgctaagctcaggatggtgaccggcctgcggaacatcccctctatccaatccaggggtctgttcggcgccattgccggatttatcgagggcgggtggaccgggatggtggatggatggtatggataccaccatcagaatgaacaaggcagcggatacgccgccgatcagaagtcaacacaaaacgccatcaacggaattaccaacaaagtcaactccgtgatcgagaagatgaacacccagttcacggccgtgggcaaagagttcaacaagctcgagcggcgaatggagaacctcaacaagaaggtggacgatggattcctggacatctggacgtacaatgccgaactgctcgtgctgctggaaaacgagagaacactcgatttccacgacagcaacgtgaagaatctgtatgagaaggtcaaatcccagttgaagaacaacgccaaggagatcggcaatggctgtttcgagttctatcacaagtgtaatgacgagtgcatggagagcgttaagaacggcacctacgactaccccaaatacagcgaagagagcaagctgaaccgtgagaagatcgacagcggaggcgatatcatcaagctgctgaacgaacaggtgaacaaggagatgcagtccagcaatctctacatgagtatgtcctcgtggtgctacacccacagcctggatggagccggactgtttctgttcgaccacgccgccgaggagtacgagcatgccaaaaagctgatcatcttcctcaatgaaaacaacgtgcccgtgcagttgaccagcatcagcgcccccgagcataaattcgagggactgacacagatctttcagaaggcctatgagcacgagcagcacatcagcgaaagcatcaacaacatcgtggaccacgccatcaagtccaaggatcacgccaccttcaacttcctgcagtggtacgttgccgaacagcacgaggaggaggtgctgtttaaggacatcctggacaaaatcgaactgatcggaaacgagaaccatggtctgtacctcgccgaccagtacgtgaagggaatcgccaagagcaggaagtcg

SEQ ID NO: 38 - DNA, 2052 bp, Artificial Sequence
  Cobra_P1_ferritin
atgaaggccagactgttggtgctgctgtgtgcccttgccgccacagacgccgacaccatctgtatcggctaccacgctaataacagcaccgacaccgtggacacagtgcttgaaaagaacgtgacagtgacccacagcgttaacctgcttgaggactctcacaacgggaagctgtgtaaactgaaggggatcgcccctctgcagctgggcaagtgcaacatcgctggctggctgctgggaaatcccgagtgtgagagcctgctgtccgctcgtagctggagctacatagttgaaacccctaacagcgagaatggcacctgctaccctggagacttcatcgactacgaggagctcagagagcagctgagcagcgtgagctcgtttgaaagatttgagatctttcccaaggagtcctcatggcccaaccacaacactaccaaaggcgtgaccgctgcttgttcacacgctggcaaatcctccttctaccggaacctgctgtggctgaccaagaaaggcggatcctaccccaaactgagcaagtcatacgtgaataacaagggcaaagaggtgctggtgctgtggggcgtgcaccacccctccaccagcaccgatcagcaaagcctgtaccagaacgagaacgcctacgtcagcgttgtgagcagcaactacaaccggagattcacccccgagattgccgagagacctaaggtgagagggcaggctggcagaatgaactactattggactctgctggagcccggagacacaattatcttcgaggccactggcaatctgatcgcaccctggtacgccttcgccttaagcaggggcagcgggtctggaattatcacttccaatgccagcatgcacgagtgcaacaccaagtgccagaccccccagggcgccattaacagcagcctgcccttccagaacatccaccccgtcactatcggcgagtgccccaagtatgtgaggagcactaagctgaggatggtgaccgggcttagaaacatccccagcatccagtcaagaggcctattcggcgcaatagccggcttcattgaaggcggctggaccgggatgatcgatggctggtatggctatcaccatcagaacgagcaaggctccgggtacgccgccgaccagaaatccacacagaatgccatcaatggaattactaacaaggttaattccgtcatcgagaagatgaatacccagtttaccgccgtgggaaaggagttcaacaatctggagaagcggatggagaacctcaacaagaaggtagatgatggattcctcgacatctggacatacaatgctgaactgctggtgctgctcgagaacgagagaaccttagacttccacgacagcaacgtgaagaatctgtacgagaaggttaagtctcaactgagaaataacgctaaggagattggcaatggctgtttcgagttctaccacaagtgtgacaacgaatgtatggaatctgtgaagaacgggacctacgactaccccaagtacagcgaggagagcaagctgaacagagagaagatcgactcaggcggcgacatcattaagctgctgaatgagcaggttaataaggagatgcagagctccaatctgtatatgagtatgagcagctggtgttacactcactccctggacggcgccggactgttcctgttcgaccacgctgcagaggagtacgaacacgcaaaaaagctgataatctttctgaatgaaaacaacgtgcccgtccagctgacctctatttctgccccagagcacaagttcgagggcctgacacagatcttccaaaaggcctacgaacacgagcagcacatcagcgagtcaatcaacaacatagtcgatcacgccattaagtctaaggaccacgccaccttcaacttcctccagtggtatgtggccgagcagcacgaggaggaggttctttttaaggatattctcgataaaatcgagttgatcggcaacgagaatcatggcctgtacctggcagaccaatatgtgaaggggatcgccaagtcaaggaagagc

SEQ ID NO: 39 - DNA, 2052 bp, Artificial Sequence
  Cobra_X3_ferritin
atggaggccagactgctggtgctgctgtgcgccttcgccgccaccaacgcagacaccatctgcattggctaccacgccaacaacagcaccgataccgtggacacagtgctcgaaaagaacgtgacagtgactcacagcgtgaacctcctggaggacagccacaacggcaagctgtgccggctgaagggtatcgcccccttgcagctgggaaactgcagcgtggcagggtggatcttgggcaatcccgagtgcgaaagtctgttttctaaggagtcctggtcctacatcgccgagacaccgaaccccgaaaacggaacatgctatcctggctacttcgctgactacgaagagctgcgggagcagcttagctccgtctccagctttgagcggtttgagatcttcccgaaagagtctagctggcccaatcacacagtcaccaagggggtgaccgcatcctgcagccacaacggcaagtcctctttctacagaaacctgctgtggctgaccgagaaaaacgggctgtaccctaacctttccaagagctatgtcaacaacaaggagaaggaggtgctggtgctgtggggggttcaccaccccagcaacatcggagaccagagagctatctatcacaccgaaaacgcctacgtgagcgtggtgagcagccattatagcagacgcttcacccctgagattgccaaacggcccaaagtgcgggaccaggagggcagaatcaactattactggaccctcctggaacctggcgataccattatctttgaggccaacggcaacctgatcgccccatggtacgcctttgctctgagccggggctttggctcaggcatcattaccagcaacgccagcatggacgagtgcgatgccaagtgccagacaccccagggcgccatcaacagctccctgccctttcaaaatgtccatcccgtgaccatcggcgagtgtcccaagtacgtccggtccactaaactgcggatggtgaccggactcagaaatatcccaagcatccagagcagaggcctgtttggcgccatcgctggatttatcgagggaggctggactggcatgatcgatggctggtacggctatcatcatcagaacgagcagggcagcggatatgccgcagaccagaagtcgacccagaacgccatcaatggaattaccaacaaggtgaacagcgtgatcgagaagatgaacacccagttcactgccgtcggcaaggaattcaacaagctggaacgtcggatggaaaacctcaacaaaaaggtggatgacggcttcctggatatctggacctacaacgccgagctcctggtgctccttgagaacgagagaaccctcgatttccacgatagcaacgtgaaaaatctctacgaaaaggtgaagagccagctgaaaaataacgccaaggagatagggaatggctgcttcgagttctaccataagtgcaacaacgagtgcatggagagcgtcaaaaacggcacttacgattaccccaagtattcagaagagagcaaactgaacagggaaaaaattgactccggcggagacattatcaagctgctgaatgaacaggtgaacaaagagatgcagagctccaacctttacatgagcatgagcagctggtgctatacccattccctcgacggggccgggctgttcctgttcgaccatgccgctgaagaatacgagcacgccaagaaactgatcatcttcttaaacgagaacaatgtgccagtgcagctgacctcaatcagcgcccccgagcacaagttcgagggactcactcagattttccagaaggcctacgagcacgagcaacacattagcgaatccatcaacaatatcgtggaccacgccataaagagcaaggaccatgccacctttaacttccttcaatggtacgtggccgagcagcacgaggaggaggtcctgttcaaggacatcctcgacaaaatcgagctgatcggcaatgaaaaccatggcctctacctggctgaccagtatgtgaaaggtatcgctaagtcaagaaaaagc

SEQ ID NO: 40 - DNA, 1494 bp, Artificial Sequence
  Wildtype Sequence of Nucleoprotein
atggccagccagggcaccaagagaagctacgagcagatggagaccgacggcgagagacagaacgccaccgagatcagagccagcgtgggcaagatgatcgacggcatcggcagattctacatccagatgtgcaccgagctcaagctgagcgactacgagggcagactgatccagaacagcctgaccatcgaaagaatggttctgagcgccttcgacgagagaagaaacagatacctggaggagcaccccagcgccggcaaggaccccaagaagaccggcggccccatctacaagagagtggacggcagatggatgagagagctggtgctgtacgacaaggaggagatcagaagaatctggagacaggccaacaacggcgacgacgccaccgccggcctgacccacatgatgatctggcacagcaacctgaacgacaccacctaccagagaaccagagccctggtgagaaccggcatggaccccagaatgtgcagcttaatgcagggcagcaccctgcccagaagatccggcgccgctggtgccgccgtcaagggcatcggcaccatggtgatggagctgatccgcatgatcaagcgcggcatcaacgacagaaacttctggagaggcgaaaacggcagaaagaccagaagcgcctacgagagaatgtgcaacatcctgaagggcaagttccagaccgccgcccaaagagccatgatggaccaggtgagagagagcagaaaccccggcaacgccgagatcgaagacctgatcttcagcgccagatcggccctgatcctgagaggcagcgtggcccacaagagctgcctgcccgcctgcgtgtatggccccgccgtgagcagcggctacaacttcgagaaggagggctacagcctggtgggcatcgaccccttcaagctgctgcagaactctcaggtgtatagcctgatcagacccaacgagaaccccgcccacaagagccagctggtgtggatggcctgccacagcgccgccttcgaggacctgagactgctgagcttcatcagaggtaccaaggtgtcccccagaggcaagctgagcaccagaggtgtgcagatcgccagcaatgagaacatggacaatatggagagcagcaccctggagctaagaagcaggtactgggccatccggaccagaagcggcggcaataccaaccagcagagagccagcgccggccagatcagcgtgcagcccaccttcagcgtgcagagaaacctgccctttgagaagagcaccgtgatggccgccttcaccggcaacaccgagggcagaaccagcgacatgagagccgagatcatcagaatgatggagggcgccaagcccgaggaggtgagctttagaggcagaggcgtgttcgagctgagcgacgagaaggccaccaacccaattgtgcccagcttcgacatgtcgaacgagggcagctacttcttcggcgacaacgccgaggagtacgacaac

SEQ ID NO: 41 - protein, 566 aa, Artificial Sequence
  MRK_H3_consUnique
MKTIIALSYILCLVFAQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQASGRITVSTKRSQQAVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 42 - protein, 566 aa, Artificial Sequence
  MRK_H3_ConsensusA
MKTIIALSYILCLVFAQKLPGNDNSTATLCLGHHAVPNGTLVKTITNDQIEVTNATELVQSSSTGRICDSPHRILDGENCTLIDALLGDPHCDGFQNKEWDLFVERSKAYSNCYPYDVPDYASLRSLVASSGTLEFNNESFNWTGVAQNGTSYACKRGSVKSFFSRLNWLHQLKYKYPALNVTMPNNDKFDKLYIWGVHHPSTDSDQTSLYVQASGRVTVSTKRSQQTVIPNIGSRPWVRGVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 43 - protein, 566 aa, Artificial Sequence
  MRK_H3_ConsensusB
MKTIIALSYILCLVFAQKLPGNDNSTATLCLGHHAVPNGTIVKTITNDQIEVTNATELVQNSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 44 - protein, 566 aa, Artificial Sequence
  MRK_H1_cot_all
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSKSYINDKGKEVLVLWGIHHPSTTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 45 - protein, 566 aa, Artificial Sequence
  MRK_H3_cot_all
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 46 - protein, 565 aa, Artificial Sequence
  MRK_sH1_Con_v2
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 47 - protein, 558 aa, Artificial Sequence
  MRK_sH1_Con_ecto
MKVKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNDECMESVKNGTYDYPKYSEESKLNREKIDGVKLESMGVGSAGSAGYIPEAPRDGQAYVRKDGEWVLSTFL

SEQ ID NO: 48 - protein, 231 aa, Artificial Sequence
  MRK_sH1_Con_RBD
MKVKLLVLLCTFTATYAGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRG

SEQ ID NO: 49 - protein, 560 aa, Artificial Sequence
  MRK_pH1_Con_ecto
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIGSAGSAGYIPEAPRDGQAYVRKDGEWVLLSTFL

SEQ ID NO: 50 - protein, 233 aa, Artificial Sequence
  MRK_pH1_Con_RBD
MKVKLLVLLCTFTATYAGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNA

SEQ ID NO: 51 - protein, 562 aa, Artificial Sequence
  eH1HA_d5v1
METPAQLLFLLLLWLPDTTGDTICIGYHANNSTDTVDTVLEKNVTVTHSVNLLEDSHNGKLCRLKGIAPLQLGKCNIAGWLLGNPECDPLPPMKSWSYIVETPNSENGICYPGDFIDYEELREQLSSVSSFERFEIFPKGSSWPNHNTNGVTAACSHEGKNSFYRNLLWLTKKEGLYPNLENSYVNKKEKEVLVLWGIHHPSNNKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIGSAGSAGYIPEAPRDGQAYVRKDGEWVLLSTFL

SEQ ID NO: 52 - protein, 562 aa, Artificial Sequence
  eH1HA_d5v2
METPAQLLFLLLLWLPDRRGDTICIGYHANNSTDTVDTVLEKNVTVTHSVNLLEDSHNGKLCRLKGIAPLQLGKCNIAGWLLGNPECDPLPPMKSWSYIVETPNSENGICYPGDFIDYEELREQLSSVSSFERFEIFPKGSSWPNHTTNGVTAACSHEGKNSFYRNLLWLTKKEGSYPNLKNSYVNKKEKEVLVLWGIHHPSNSKEQQNLYQNENAHVSVVTSNYNRRFTPEIAERPKVRDQAGRMNYYWTLLKPGDTIIFEADGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIGSAGSAGYIPEAPRDGQAYVRKDGEWVLLSTFL

SEQ ID NO: 53 - protein, 562 aa, Artificial Sequence
  eH1HA_d5v3
METPAQLLFLLLLWLPDTTGDTICIGYHANNSTDTVDTVLEKNVTVTHSVNLLEDSHNGKLCRLKGIAPLQLGKCNIAGWLLGNPECDPLPPMKSWSYIVETPNSENGICYPGDFIDYEELREQLSSVSSFERFEIFPKGSSWPDHNTNGVTAACSHEGKNSFYRNLLWLTEKKGSYPNLKNPYVNKKEKEVLVLWGIHHPSNSKEQQNLYRNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIGSAGSAGYIPEAPRDGQAYVRKDGEWVLLSTFL

SEQ ID NO: 54 - protein, 562 aa, Artificial Sequence
  eH1HA_d5v4
METPAQLLFLLLLWLPDTTGDTICIGYHANNSTDTVDTVLEKNVTVTHSVNLLEDSHNGKLCKLKGIAPLQLGKCNIAGWLLGNPGCDPLLPVGSWSYIVETPNSENGICYPGDFIDYEELREQLSSVSSFERFKIFPKESSWPDHNTNGVTAACSHEGKNSFYRNLLWLTKKESSYPNLENSYVNKKRKEVLVLWGIHHPSNSKEQQNLYQNENAYVSVVTSNYNRRFTPEIAERPKVKGQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIGSAGSAGYIPEAPRDGQAYVRKDGEWVLLSTFL

SEQ ID NO: 55 - protein, 233 aa, Artificial Sequence
  MRK_RBS_HA129
MKVKLLVLLCTFTATYAGVAPLHLGKCNIAGWLLGNPECELLLTVSSWSYIVETSNSDNGTCYPGDFINYEELREQLSSVSSFERFEIFPKTSSWPDHETNRGVTAACPYAGANSFYRNLIWLVKKGNSYPKLSKSYVNNKGKEVLVLWGIHHPPTSTDQQSLYQNADAYVFVGSSRYSKKFKPEIAIRPKVRDQEGRMNYYWTLVEPGDKITFEATGNLVVPRYAFAMERNA

SEQ ID NO: 56 - protein, 233 aa, Artificial Sequence
  RBD1-Cal09-PC-Cb
MKVKLLVLLCTFTATYAGVAPLHLGKCNIAGWILGNPECESLSTASSWSNITETPSSDNGTCYPGDFIDYEELREQLSSVSSFERFEIFPKTSSWPNHSSNKGVTAACPHAGAKSFYKNLIWLVKKNGSYPKLNKSYINDSGKEVLVLWGIHHPSNSTDQQSLYQNADTYVFVGSSNYSKKFKPEIAIRPKVRDQEGRMNYYWTLVEPGDKITFEATGNLVVPRYAFAMERNA

SEQ ID NO: 57 - protein, 233 aa, Artificial Sequence
  RBD1-Cal09-PC
MKVKLLVLLCTFTATYAGVAPLHLGKCNIAGWILGNPECESNSTASSWSNITETPSSDNGTCYPGDFIDYEELREQLSSVSSFERFEIFPKTSSWPNHSSNKGVTAACPHAGAKSFYKNLIWLVKKNGSYPKLNKSYINDSGKEVLVLWGIHHPSNSTDQQSLYQNADTYVFVGSSNYSKKFKPEIAIRPKVRDQEGRMNYYWTLVEPGDKITFEATGNLVVPRYAFAMERNA

SEQ ID NO: 58 - protein, 233 aa, Artificial Sequence
  RBD1-Cal09
MKVKLLVLLCTFTATYAGVAPLHLGKCNIAGWILGNPECESLSTASSWSNITETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNA

SEQ ID NO: 59 - protein, 233 aa, Artificial Sequence
  MRK_RBD-Cal09-PC-Cb
MKVKLLVLLCTFTATYAGVAPLHLGKCNIAGWILGNPECESLSTASSWSYIVETPSSDNGTCYPGDFIDYEELREQLSSVSSFERFEIFPKTSSWPNHSSNKGVTAACPHAGAKSFYKNLIWLVKKNGSYPKLNKSYINDSGKEVLVLWGIHHPSNSTDQQSLYQNADTYVFVGSSNYSKKFKPEIAIRPKVRDQEGRMNYYWTLVEPGDKITFEATGNLVVPRYAFAMERNA

SEQ ID NO: 60 - protein, 233 aa, Artificial Sequence
  MRK_RBD-Cal09-PC
MKVKLLVLLCTFTATYAGVAPLHLGKCNIAGWILGNPECESNSTASSWSYIVETPSSDNGTCYPGDFIDYEELREQLSSVSSFERFEIFPKTSSWPNHSSNKGVTAACPHAGAKSFYKNLIWLVKKNGSYPKLNKSYINDSGKEVLVLWGIHHPSNSTDQQSLYQNADTYVFVGSSNYSKKFKPEIAIRPKVRDQEGRMNYYWTLVEPGDKITFEATGNLVVPRYAFAMERNA

SEQ ID NO: 61 - protein, 233 aa, Artificial Sequence
  MRKRBD-Cal09
MKVKLLVLLCTFTATYAGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNA

SEQ ID NO: 62 - protein, 565 aa, Artificial Sequence
  FLHA_PR8
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 63 - protein, 566 aa, Artificial Sequence
  FLHA_Cal09
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 64 - DNA, 699 bp, Artificial Sequence
  MRK_pH1_Con_RBD
atgaaggtgaagctgctggtgctgctgtgcaccttcaccgccacctacgccggcgtggcccctctgcacctgggcaagtgcaacatcgccggctggatcctgggcaaccctgagtgcgagagccttagcacagcctcctcctggagctacatcgtggagacgagcagcagcgataacgggacctgctaccctggcgacttcatcgactacgaggagctgagagagcagctgagcagcgtgagcagcttcgagagattcgagatcttccctaagaccagcagctggcctaaccacgacagcaacaagggcgtgaccgccgcctgcccacacgccggggccaagagcttctacaagaacctgatctggctggtgaagaagggcaacagctaccctaaactgagcaagtcctacatcaacgacaaaggcaaggaggtcctcgtgctctggggcatccaccaccctagcaccagcgccgatcagcagagcctgtaccagaacgccgacgcgtacgtgttcgtgggcaccagcagatacagcaagaagttcaagcctgagatcgccatcagacctaaggtgagggaccaggagggcagaatgaactactactggaccctggtggagcccggagataagatcacatttgaggccaccggcaacctggtggtgcctagatacgccttcgccatggagagaaacgcc

SEQ ID NO: 65 - DNA, 1680 bp, Artificial Sequence
  MRK_pH1_Con_ecto
atgaaggccatcctcgtggtgctgctgtacacctttgccaccgccaacgccgataccctgtgtatcggctaccacgccaacaacagcaccgacaccgtggatactgtcctggagaagaacgtgaccgtgacccacagcgtgaacctgctggaggacaagcacaacggcaagctgtgcaagctgagaggcgtggcccctctgcacctgggcaagtgcaacatcgccggctggatcctgggcaaccctgagtgcgagagccttagcacagcctcctcctggagctacatcgtggagacgagcagcagcgataacgggacctgctaccctggcgacttcatcgactacgaggagctgagagagcagctgagcagcgtgagcagcttcgagagattcgagatcttccctaagaccagcagctggcctaaccacgacagcaacaagggcgtgaccgccgcctgcccacacgccggggccaagagcttctacaagaacctgatctggctggtgaagaagggcaacagctaccctaaactgagcaagtcctacatcaacgacaaaggcaaggaggtcctcgtgctctggggcatccaccaccctagcaccagcgccgatcagcagagcctgtaccagaacgccgacgcgtacgtgttcgtgggcaccagcagatacagcaagaagttcaagcctgagatcgccatcagacctaaggtgagggaccaggagggcagaatgaactactactggaccctggtggagcccggagataagatcacatttgaggccaccggcaacctggtggtgcctagatacgccttcgccatggagagaaacgccggcagcggcatcatcatcagcgacacccctgtgcacgactgcaacaccacctgccagacccctaagggcgccatcaacacgagcctgcctttccagaacatccaccctatcaccatcggcaagtgccctaagtacgtgaagtcaaccaaactgagactcgccaccggcctcagaaacgtgcctagcatccagagcagaggcctcttcggcgccatcgcgggattcatcgagggcggctggaccggcatggtggacggctggtacggctaccaccatcagaacgagcagggcagcgggtacgcggccgacctcaagagcacccagaacgccatcgacaagatcaccaacaaggtgaacagcgtgatcgagaagatgaacacccagttcaccgccgtgggcaaggagttcaaccacctggagaagagaatcgagaacctgaacaagaaggtggacgacggcttcctggacatctggacctacaacgcagaactgctcgtgcttctggagaacgagagaaccctggactaccacgactccaacgtgaagaacctgtacgagaaggtgagaagccagctgaagaacaacgccaaggagatcggcaacggctgcttcgagttctaccacaagtgcgacaacacctgcatggagagcgtgaagaacggcacctacgactaccctaagtacagcgaggaggccaagctgaacagagaggagatcgacggcgtgaagctggagagcaccagaatcggctcagccgggagcgccggctacatccctgaggcccctagagacggccaggcctacgtgagaaaggacggcgagtgggtgctgctgagcaccttcctg

SEQ ID NO: 66 - DNA, 693 bp, Artificial Sequence
  MRK_sH1_Con_RBD
atgaaggtgaagctgctggtgctgctgtgcaccttcaccgccacctacgccggaatcgctcccctgcagctcggcaactgcagcgtggccggctggattctgggcaaccccgagtgcgaactgctgattagcaaagagtcctggagctacatcgtggaaaccccgaatcccgagaacggcacctgctaccccggctacttcgccgactacgaggagctaagagagcagctgagtagcgtgagctcattcgagagattcgagatctttcccaaggagtctagctggcccaatcacaccgtcaccggcgtgtccgccagctgtagccacaacggcaagagcagcttctacagaaacctgctgtggctgaccggcaagaacggactgtaccctaacctgagcaagagctacgcgaacaataaggagaaggaggtgctagtgctgtggggcgtgcaccatccgcccaacatcggcgaccagagagccctgtaccacaccgagaacgcctacgtgagcgtggtgagcagccactatagcagaagattcacccctgagatcgccaagaggccaaaggtgagagatcaggaaggaagaataaactactactggaccctcctggagcccggcgacaccatcatcttcgaggctaacggcaacctgatcgcccctagatacgccttcgccctgagcagaggc

SEQ ID NO: 67 - DNA, 1677 bp, Artificial Sequence
  MRK_sH1_Con_ecto
atgaaggtgaagctgctggtgctgctgtgtaccttcactgccacttacgccgacaccatttgcatcggctaccacgccaacaacagcaccgataccgtggacaccgtgctggagaagaacgtcaccgtgacccacagcgtgaacctgctggaggatagccataacggcaagctgtgcctgctgaagggaatcgctcccctgcagctcggcaactgcagcgtggccggctggattctgggcaaccccgagtgcgaactgctgattagcaaagagtcctggagctacatcgtggaaaccccgaatcccgagaacggcacctgctaccccggctacttcgccgactacgaggagctaagagagcagctgagtagcgtgagctcattcgagagattcgagatctttcccaaggagtctagctggcccaatcacaccgtcaccggcgtgtccgccagctgtagccacaacggcaagagcagcttctacagaaacctgctgtggctgaccggcaagaacggactgtaccctaacctgagcaagagctacgcgaacaataaggagaaggaggtgctagtgctgtggggcgtgcaccatccgcccaacatcggcgaccagagagccctgtaccacaccgagaacgcctacgtgagcgtggtgagcagccactatagcagaagattcacccctgagatcgccaagaggccaaaggtgagagatcaggaaggaagaataaactactactggaccctcctggagcccggcgacaccatcatcttcgaggctaacggcaacctgatcgcccctagatacgccttcgccctgagcagaggcttcggcagcggcatcatcaccagcaacgctcccatggacgagtgcgacgccaagtgccagaccccgcagggcgccatcaactcgagcctgcccttccagaacgtgcaccccgtgaccatcggcgagtgccccaagtacgtgagaagcgccaagctgagaatggtgaccggcctgagaaacatcccaagcatccagagcagagggctgttcggcgccatcgctggcttcatcgagggcggctggaccggcatggtggacggctggtacggttatcaccaccagaacgagcagggcagcggctacgccgccgaccagaagtccacccagaacgccatcaacggcattacaaacaaggtgaacagcgttatcgagaagatgaacacccaattcaccgccgtgggcaaggagttcaacaagctggagagaagaatggagaacctgaacaagaaggtggacgacggcttcctggacatctggacctacaacgccgaactgctggtcctgctggagaacgagagaaccctggacttccacgactccaacgtgaagaacttatacgagaaggtcaaatcccagctgaagaacaacgccaaagaaatcggaaacggctgcttcgaattctaccacaagtgcaacgacgagtgcatggagagcgtgaagaacggaacctacgactaccccaagtacagcgaggaaagcaaactgaacagagagaagatcgacggcgtgaagttagagagcatgggcgtgggcagcgccggctctgctggatacatccctgaggcccctagagacggccaggcctacgtgagaaaggacggcgagtgggtgctgctgagcaccttcctg

SEQ ID NO: 68 - DNA, 1695 bp, Artificial Sequence
  MRK_sH1_Con_v2
atgaaggtgaaactcctcgtcctgctgtgcaccttcaccgccacctacgccgataccatctgtattggctaccacgccaacaactccaccgacaccgtggataccgtgctcgagaagaacgtgaccgtgacccacagcgtgaacctgctggagaacagccacaacggcaagctgtgcctgctgaagggcatcgcgcccctgcagttgggtaactgctccgtggccggctggatcctgggcaaccctgagtgcgagctgctgatcagcaaggagagctggagctacatcgtggagaagcctaaccccgagaacggcacctgctaccctggccacttcgccgactacgaggagctgagagagcaactcagcagcgtgagcagcttcgagagattcgagatcttccctaaggagagcagctggcccaatcacactgtgaccggcgtgtccgcttcttgcagccataacggggaaagctccttctacagaaatctcctttggctgacggggaagaacggcctgtaccctaacctgagcaagagctacgccaacaacaaggagaaggaggtgctggtgctgtggggcgtgcaccaccctcctaacatcggcgaccagaaggccctgtaccacaccgagaacgcctacgtcagcgtggtgtccagccactacagcagaaagttcacccctgagatcgccaagaggcctaaggtgcgggaccaggagggcagaatcaactactactggaccctgctggagcctggcgacaccatcatcttcgaggccaacggcaacctgatcgcccctagatacgccttcgccctgagcagaggcttcggcagcggcatcatcaacagcaacgcccctatggacaagtgcgacgccaagtgccagactccgcagggcgctatcaacagctccctgcctttccagaacgtgcaccctgtgaccatcggcgagtgccctaagtacgtgagaagcgccaagctgagaatggtgaccggcctgagaaacatccctagcatccagagcagaggcctgttcggcgccatcgccgggtttatcgagggcggctggaccggcatggtggacggctggtacggctaccaccaccagaacgagcagggctccggctacgccgccgaccagaaatccacccagaacgccatcaacggcatcaccaacaaggtgaacagcgtcatcgagaagatgaacacccagttcaccgccgtgggcaaggagttcaacaagctggagagaagaatggagaacctgaacaagaaggtggacgacggcttcatcgacatctggacctacaacgccgagcttctggtgctcctggagaacgagagaaccctggacttccacgacagcaacgtgaagaacctgtacgagaaggtgaagtcccagctgaagaacaacgccaaggagatcggcaacggctgcttcgagttctaccacaagtgcaacgacgagtgcatggagagcgtgaagaacggcacctacgattaccccaagtacagcgaggagagcaagctgaacagagagaagatcgacggcgtgaagctggagagcatgggcgtgtaccagatcctggccatctactccaccgtggccagtagcctggtgctgctggtgagcctgggcgcaatcagcttctggatgtgcagcaacggcagcctgcagtgcagaatctgcatc

SEQ ID NO: 69 - DNA, 699 bp, Artificial Sequence
  MRK_RBS_HA129
atgaaggtcaaacttctcgtgctcctgtgcaccttcaccgccacctacgcgggcgtggctccgcttcacctgggcaagtgcaacatcgccggttggctgctgggtaacccagagtgcgagctactgctgaccgtgagcagctggagctacatcgtggaaaccagcaacagcgacaacggcacctgctaccctggcgacttcatcaactacgaggagctgagagagcagctcagcagcgtgtccagcttcgagagattcgagatcttccctaagactagcagctggcccgaccacgaaacaaacagaggcgtgaccgccgcttgtccatacgccggcgccaacagcttctacagaaacctgatctggctggtgaagaagggcaacagctaccctaagctgagcaagagctacgtgaacaacaagggcaaggaggtgcttgtgctgtggggcatccaccaccctcctaccagcaccgaccagcagagcctgtaccagaacgccgacgcctacgtgttcgtgggcagcagcagatacagcaagaagttcaagcctgagatcgccatcagacctaaggtgagggaccaggagggcagaatgaactactactggactctggtggagcccggcgacaagatcaccttcgaggccaccggcaacctggtggtgcctagatacgccttcgccatggagagaaacgcc

SEQ ID NO: 70 - DNA, 1698 bp, Artificial Sequence
  MRK_H1_cot_all
atgaaggccatcctggtcgtgctgctctacacattcgccaccgccaacgcagacactctgtgcatcggctaccacgccaacaacagcaccgacaccgtggataccgtgctggagaagaacgtgaccgtgacccacagcgtgaacctgctggaggacaagcacaacggcaagctgtgcaagctgagaggcgtggcccctctgcacctgggcaagtgcaacatcgccggctggatcctgggaaaccccgagtgcgagagcctgtcaaccgcctcgagctggtcctacatcgtggaaaccagcagcagcgataacgggacgtgctacccgggcgacttcatcaactacgaggagctgagagaacagctgagcagcgtcagtagcttcgagagattcgagatcttccctaagaccagcagctggcctaaccacgacagcaacaagggcgtgaccgccgcttgcccgcacgcaggcgccaagagcttctacaagaacctgatctggctggtgaagaagggcaacagctaccctaagctgagcaagagctacatcaacgacaaggggaaggaggtgctagtcctgtggggcatccatcaccctagcaccacagccgaccagcaaagcctgtaccagaacgcggacgcctacgtgttcgtcggcaccagcagatacagcaagaagttcaagcctgagatcgccatcagacctaaggtgcgagatcaggagggcagaatgaactactactggaccctggtggagcccggagacaagattactttcgaagcgaccggcaacctggtggtgcctagatacgccttcgccatggagagaaacgccggcagcggcatcatcatcagcgacacccctgtgcacgactgcaacaccacctgccagacccctaaaggcgccatcaacacaagcctgccttttcagaacatccaccctatcaccatcggcaagtgccctaagtacgtgaagtccaccaagctccgcctggcaaccggcctcaggaacgtgcctagcatccagagcagaggcctgttcggggccatagccggcttcatagagggtggctggaccggcatggttgacgggtggtacggataccatcaccagaacgagcaaggcagcggctacgccgcagacctgaagtcaacccagaacgccatcgacaagatcaccaacaaggtgaacagcgtgatcgagaagatgaacacccagttcaccgccgtgggcaaggagttcaaccacctagagaagaggatcgagaacctgaataagaaggtggacgacggcttcctggacatctggacctacaacgccgagctgctcgtcctcctggagaacgagagaaccctggactaccacgatagcaacgtgaagaacctgtacgagaaggtgagaaaccagctgaagaataacgccaaggagatcggcaacggctgcttcgagttctaccacaagtgcgacaacacctgcatggagagcgtgaagaacggcacctacgactaccctaagtacagcgaggaggccaagctgaacagagagaagatcgacggcgtgaagctggagagcaccagaatctaccagatcctggccatctacagcaccgtggccagcagcctcgtgctcgtggtgagcctgggcgccatctccttctggatgtgcagcaacggcagcctgcagtgcagaatctgcatc

SEQ ID NO: 71 - DNA, 1698 bp, Artificial Sequence
  MRK_H3_cot_all
atgaagaccatcatcgccctgagctacatcctgtgcctggtgttcgcgcagaaactccccggcaacgacaatagcactgccaccctgtgtctgggccatcacgccgtgcctaacggaaccatcgtgaagacgatcaccaacgacagaatcgaggtgaccaacgccaccgagctggtccagaattcgagcatcggcgaaatctgcgacagccctcaccagatcctggacggcgagaactgcaccctgattgacgcactgctaggcgacccacagtgtgacggcttccagaacaagaagtgggacctgttcgtggagagaagcaaggcctacagcaactgctacccttacgacgtgcctgactacgccagcctgagatccctcgtggcctccagcggcaccctcgagttcaataacgagagcttcaactggaccggagtcacccagaacgggacatccagcgcctgcatcagaagaagcaacagcagcttcttcagcagactgaactggctgacccacctgaacttcaagtaccctgccctgaacgtgaccatgcctaacaacgagcagttcgacaagctgtacatctggggcgtgcaccatcccggcaccgacaaggaccagatcttcctgtacgcccagagctccggcaggatcaccgtgagcaccaagagaagccagcaggccgtgatccctaacatcggcagcagacctagaatcagaaacatccctagcagaatcagcatctactggaccatagtgaagcccggcgacatcctgctgatcaactcgaccggcaacctgatcgctcctaggggctacttcaagatcagaagcggcaagagcagcatcatgagaagcgacgcgcccatcgggaagtgcaagtccgagtgcatcacccctaacggcagcatccccaacgacaagcctttccagaacgtgaacagaatcacctacggcgcctgccctagatacgtgaagcagagcacactgaagctggccaccggcatgaggaacgtgcctgagaagcagaccagaggcatcttcggggctattgccggcttcatcgagaacggttgggagggaatggtcgacgggtggtacggcttcagacaccagaacagcgaaggcaggggacaggccgccgacctcaagtccacccaggctgccatcgatcagatcaacgggaagctgaacagactgatcggcaagaccaacgagaagttccaccagatcgagaaggagttcagcgaggtggagggcagaatccaggacctggagaagtacgtggaggacacgaagatcgacctgtggagctacaacgcagagctgttggtggcactggagaaccagcacaccatcgacctgaccgacagcgagatgaacaagctgttcgagaagaccaagaagcagttacgagagaacgccgaggacatgggaaacggctgttttaagatctaccacaagtgcgacaacgcctgcatcgggagcatcaggaacgggacctacgaccacgacgtgtacagagacgaggccctgaacaacagattccagatcaagggcgtggagctgaagtccggctacaaggactggatcctgtggatcagcttcgccatcagctgcttcctgctgtgcgtggccctcctgggctttataatgtgggcctgccagaagggcaacatcaggtgcaacatctgcatc

SEQ ID NO: 72 - DNA, 1698 bp, Artificial Sequence
  MRK_H3_ConsensusA
atgaagaccatcatcgccctgagctacatcctgtgcctggtgttcgcgcagaaactccccggcaacgacaatagcactgccaccctgtgtctgggccatcacgccgtgcctaacggaaccctcgtgaagacgatcaccaacgaccagatcgaggtgaccaacgccaccgagctggtccagagttcgagcaccggcagaatctgcgacagccctcaccggatcctggacggcgagaactgcaccctgattgacgcactgctaggcgacccacactgtgacggcttccagaacaaggagtgggacctgttcgtggagagaagcaaggcctacagcaactgctacccttacgacgtgcctgactacgccagcctgagatccctcgtggcctccagcggcaccctcgagttcaataacgagagcttcaactggaccggagtcgcccagaacgggacatcctacgcctgcaagagaggaagcgtcaagagcttcttcagcagactgaactggctgcaccagctgaagtacaagtaccctgccctgaacgtgaccatgcctaacaacgacaagttcgacaagctgtacatctggggcgtgcaccatcccagcaccgacagcgaccagacctccctgtacgtccaggcatccggcagggtcaccgtgagcaccaagagaagccagcagaccgtgatccctaacatcggcagcagaccttgggtcagaggcgtctctagcagaatcagcatctactggaccatagtgaagcccggcgacatcctgctgatcaactcgaccggcaacctgatcgctcctaggggctacttcaagatcagaagcggcaagagcagcatcatgagaagcgacgcgcccatcgggaagtgcaactccgagtgcatcacccctaacggcagcatccccaacgacaagcctttccagaacgtgaacagaatcacctacggcgcctgccctagatacgtgaagcagaacacactgaagctggccaccggcatgaggaacgtgcctgagaagcagaccagaggcatcttcggggctattgccggcttcatcgagaacggttgggagggaatggtcgacgggtggtacggcttcagacaccagaacagcgaaggcacgggacaggccgccgacctcaagtccacccaggctgccatcaatcagatcaacgggaagctgaacagactgatcgagaagaccaacgagaagttccaccagatcgagaaggagttcagcgaggtggagggcagaatccaggacctggagaagtacgtggaggacacgaagatcgacctgtggagctacaacgcagagctgttggtggcactggagaaccagcacaccatcgacctgaccgacagcgagatgaacaagctgttcgagaggaccaggaagcagttacgagagaacgccgaggacatgggaaacggctgttttaagatctaccacaagtgcgacaacgcctgcatcgggagcatcaggaacgggacctacgaccacgacgtgtacagagacgaggccctgaacaacagattccagatcaagggcgtggagctgaagtccggctacaaggactggatcctgtggatcagcttcgccatcagctgcttcctgctgtgcgtggtcctcctgggctttataatgtgggcctgccagaagggcaacatcaggtgcaacatctgcatc

SEQ ID NO: 73 - DNA, 1698 bp, Artificial Sequence
  MRK_H3_ConsensusB
atgaagaccatcatcgccctgagctacatcctgtgcctggtgttcgcgcagaaactccccggcaacgacaatagcactgccaccctgtgtctgggccatcacgccgtgcctaacggaaccatcgtgaagacgatcaccaacgaccagatcgaggtgaccaacgccaccgagctggtccagaattcgagcaccggcgaaatctgcgacagccctcaccagatcctggacggcgagaactgcaccctgattgacgcactgctaggcgacccacagtgtgacggcttccagaacaagaagtgggacctgttcgtggagagaagcaaggcctacagcaactgctacccttacgacgtgcctgactacgccagcctgagatccctcgtggcctccagcggcaccctcgagttcaataacgagagcttcaactggaccggagtcacccagaacgggacatccagcgcctgcatcagaagaagcaacagcagcttcttcagcagactgaactggctgacccacctgaacttcaagtaccctgccctgaacgtgaccatgcctaacaacgagcagttcgacaagctgtacatctggggcgtgcaccatcccggcaccgacaaggaccagatcttcctgtacgcccagagctccggcaggatcaccgtgagcaccaagagaagccagcaggccgtgatccctaacatcggcagcagacctagaatcagaaacatccctagcagaatcagcatctactggaccatagtgaagcccggcgacatcctgctgatcaactcgaccggcaacctgatcgctcctaggggctacttcaagatcagaagcggcaagagcagcatcatgagaagcgacgcgcccatcgggaagtgcaactccgagtgcatcacccctaacggcagcatccccaacgacaagcctttccagaacgtgaacagaatcacctacggcgcctgccctagatacgtgaagcagagcacactgaagctggccaccggcatgaggaacgtgcctgagaagcagaccagaggcatcttcggggctattgccggcttcatcgagaacggttgggagggaatggtcgacgggtggtacggcttcagacaccagaacagcgaaggcaggggacaggccgccgacctcaagtccacccaggctgccatcgatcagatcaacgggaagctgaacagactgatcggcaagaccaacgagaagttccaccagatcgagaaggagttcagcgaggtggagggcagaatccaggacctggagaagtacgtggaggacacgaagatcgacctgtggagctacaacgcagagctgttggtggcactggagaaccagcacaccatcgacctgaccgacagcgagatgaacaagctgttcgagaagaccaagaagcagttacgagagaacgccgaggacatgggaaacggctgttttaagatctaccacaagtgcgacaacgcctgcatcgggagcatcaggaacgggacctacgaccacgacgtgtacagagacgaggccctgaacaacagattccagatcaagggcgtggagctgaagtccggctacaaggactggatcctgtggatcagcttcgccatcagctgcttcctgctgtgcgtggccctcctgggctttataatgtgggcctgccagaagggcaacatcaggtgcaacatctgcatc

SEQ ID NO: 74 - DNA, 1698 bp, Artificial Sequence
  MRK_H3_consUnique
atgaagaccatcatcgccctgagctacatcctgtgcctggtgttcgcgcagaaactccccggcaacgacaatagcactgccaccctgtgtctgggccatcacgccgtgcctaacggaaccatcgtgaagacgatcaccaacgaccagatcgaggtgaccaacgccaccgagctggtccagagttcgagcaccggcgaaatctgcgacagccctcaccagatcctggacggcgagaactgcaccctgattgacgcactgctaggcgacccacagtgtgacggcttccagaacaagaagtgggacctgttcgtggagagaagcaaggcctacagcaactgctacccttacgacgtgcctgactacgccagcctgagatccctcgtggcctccagcggcaccctcgagttcaataacgagagcttcaactggaccggagtcacccagaacgggacatccagcgcctgcatcagaagaagcaacagcagcttcttcagcagactgaactggctgacccacctgaacttcaagtaccctgccctgaacgtgaccatgcctaacaacgagcagttcgacaagctgtacatctggggcgtgcaccatcccggcaccgacaaggaccagatcttcctgtacgcccaggcatccggcaggatcaccgtgagcaccaagagaagccagcaggccgtgatccctaacatcggcagcagacctagagtcagaaacatccctagcagaatcagcatctactggaccatagtgaagcccggcgacatcctgctgatcaactcgaccggcaacctgatcgctcctaggggctacttcaagatcagaagcggcaagagcagcatcatgagaagcgacgcgcccatcgggaagtgcaactccgagtgcatcacccctaacggcagcatccccaacgacaagcctttccagaacgtgaacagaatcacctacggcgcctgccctagatacgtgaagcagaacacactgaagctggccaccggcatgaggaacgtgcctgagaagcagaccagaggcatcttcggggctattgccggcttcatcgagaacggttgggagggaatggtcgacgggtggtacggcttcagacaccagaacagcgaaggcaggggacaggccgccgacctcaagtccacccaggctgccatcgatcagatcaacgggaagctgaacagactgatcggcaagaccaacgagaagttccaccagatcgagaaggagttcagcgaggtggagggcagaatccaggacctggagaagtacgtggaggacacgaagatcgacctgtggagctacaacgcagagctgttggtggcactggagaaccagcacaccatcgacctgaccgacagcgagatgaacaagctgttcgagaagaccaagaagcagttacgagagaacgccgaggacatgggaaacggctgttttaagatctaccacaagtgcgacaacgcctgcatcgggagcatcaggaacgggacctacgaccacgacgtgtacagagacgaggccctgaacaacagattccagatcaagggcgtggagctgaagtccggctacaaggactggatcctgtggatcagcttcgccatcagctgcttcctgctgtgcgtggccctcctgggctttataatgtgggcctgccagaagggcaacatcaggtgcaacatctgcatc

SEQ ID NO: 75 - DNA, 699 bp, Artificial Sequence
  RBD1-Cal09-PC-Cb
atgaaggtgaagcttctcgtgctcttatgcaccttcaccgccacctacgccggcgtggctccgcttcaccttggcaagtgcaacatcgccggctggatcttgggaaaccccgagtgcgagagcttgagcaccgccagcagctggagcaacatcacggaaacccctagcagcgacaacggcacctgctaccccggcgacttcatcgactacgaggagctgcgggagcagctgagcagcgtgagcagcttcgagcggttcgagatcttccccaagaccagctcttggcccaaccacagcagcaacaagggcgtgaccgccgcctgccctcacgctggcgccaagagcttctacaagaacctgatctggctggtgaagaagaacggcagctaccccaagctgaacaagtcttacattaacgactcaggcaaggaggtgctggtcctgtggggcatccaccaccccagcaacagcaccgaccaacagagcctgtaccagaacgccgacacctacgtgttcgtgggcagcagcaactacagcaagaagttcaagcccgagatcgccatccggcccaaggtgcgggaccaggagggccggatgaactactactggaccctggtggagcctggcgacaagatcaccttcgaggccaccggcaacctggtggtgccccggtacgccttcgccatggagcggaacgcc

SEQ ID NO: 76 - DNA, 699 bp, Artificial Sequence
  RBD1-Cal09-PC
atgaaggtgaagcttctcgtgctcttatgcaccttcaccgccacctacgccggcgtggctccgcttcaccttggcaagtgcaacatcgccggctggatcttgggaaaccccgagtgcgagagcaacagcaccgccagcagctggagcaacatcacggaaacccctagcagcgacaacggcacctgctaccccggcgacttcatcgactacgaggagctgcgggagcagctgagcagcgtgagcagcttcgagcggttcgagatcttccccaagaccagctcttggcccaaccacagcagcaacaagggcgtgaccgccgcctgccctcacgctggcgccaagagcttctacaagaacctgatctggctggtgaagaagaacggcagctaccccaagctgaacaagtcttacattaacgactcaggcaaggaggtgctggtcctgtggggcatccaccaccccagcaacagcaccgaccaacagagcctgtaccagaacgccgacacctacgtgttcgtgggcagcagcaactacagcaagaagttcaagcccgagatcgccatccggcccaaggtgcgggaccaggagggccggatgaactactactggaccctggtggagcctggcgacaagatcaccttcgaggccaccggcaacctggtggtgccccggtacgccttcgccatggagcggaacgcc

SEQ ID NO: 77 - DNA, 699 bp, Artificial Sequence
  RBD1-Cal09
atgaaggtgaagcttctcgtgctcttatgcaccttcaccgccacctacgccggcgtggctccgcttcaccttggcaagtgcaacatcgccggctggatcttgggaaaccccgagtgcgagagcttgagcaccgccagcagctggagcaacatcacggaaacccctagcagcgacaacggcacctgctaccccggcgacttcatcgactacgaggagctgcgggagcagctgagcagcgtgagcagcttcgagcggttcgagatcttccccaagaccagctcttggcccaaccacgacagcaacaagggcgtgaccgccgcctgccctcacgctggcgccaagagcttctacaagaacctgatctggctggtgaagaagggcaacagctaccccaagctgtccaagtcttacattaacgacaagggcaaggaggtgctggtcctgtggggcatccaccaccccagcaccagcgccgaccaacagagcctgtaccagaacgccgacacctacgtgttcgtgggcagcagccggtacagcaagaagttcaagcccgagatcgccatccggcccaaggtgcgggaccaggagggccggatgaactactactggaccctggtggagcctggcgacaagatcaccttcgaggccaccggcaacctggtggtgccccggtacgccttcgccatggagcggaacgcc

SEQ ID NO: 78 - DNA, 699 bp, Artificial Sequence
  MRK_RBD-Cal09-PC-Cb
atgaaggtgaagcttctcgtgctcttatgcaccttcaccgccacctacgccggcgtggctccgcttcaccttggcaagtgcaacatcgccggctggatcttgggaaaccccgagtgcgagagcttgagcaccgccagcagctggagctacatcgtggaaacccctagcagcgacaacggcacctgctaccccggcgacttcatcgactacgaggagctgcgggagcagctgagcagcgtgagcagcttcgagcggttcgagatcttccccaagaccagctcttggcccaaccacagcagcaacaagggcgtgaccgccgcctgccctcacgctggcgccaagagcttctacaagaacctgatctggctggtgaagaagaacggcagctaccccaagctgaacaagtcttacattaacgactcaggcaaggaggtgctggtcctgtggggcatccaccaccccagcaacagcaccgaccaacagagcctgtaccagaacgccgacacctacgtgttcgtgggcagcagcaactacagcaagaagttcaagcccgagatcgccatccggcccaaggtgcgggaccaggagggccggatgaactactactggaccctggtggagcctggcgacaagatcaccttcgaggccaccggcaacctggtggtgccccggtacgccttcgccatggagcggaacgcc

SEQ ID NO: 79 - DNA, 699 bp, Artificial Sequence
  MRK_RBD-Cal09-PC
atgaaggtgaagcttctcgtgctcttatgcaccttcaccgccacctacgccggcgtggctccgcttcaccttggcaagtgcaacatcgccggctggatcttgggaaaccccgagtgcgagagcaacagcaccgccagcagctggagctacatcgtggaaacccctagcagcgacaacggcacctgctaccccggcgacttcatcgactacgaggagctgcgggagcagctgagcagcgtgagcagcttcgagcggttcgagatcttccccaagaccagctcttggcccaaccacagcagcaacaagggcgtgaccgccgcctgccctcacgctggcgccaagagcttctacaagaacctgatctggctggtgaagaagaacggcagctaccccaagctgaacaagtcttacattaacgactcaggcaaggaggtgctggtcctgtggggcatccaccaccccagcaacagcaccgaccaacagagcctgtaccagaacgccgacacctacgtgttcgtgggcagcagcaactacagcaagaagttcaagcccgagatcgccatccggcccaaggtgcgggaccaggagggccggatgaactactactggaccctggtggagcctggcgacaagatcaccttcgaggccaccggcaacctggtggtgccccggtacgccttcgccatggagcggaacgcc

SEQ ID NO: 80 - DNA, 699 bp, Artificial Sequence
  MRKRBD-Cal09
atgaaggtgaagcttctcgtgctcttatgcaccttcaccgccacctacgccggcgtggctccgcttcaccttggcaagtgcaacatcgccggctggatcttgggaaaccccgagtgcgagagcttgagcaccgccagcagctggagctacatcgtggaaacccctagcagcgacaacggcacctgctaccccggcgacttcatcgactacgaggagctgcgggagcagctgagcagcgtgagcagcttcgagcggttcgagatcttccccaagaccagctcttggcccaaccacgacagcaacaagggcgtgaccgccgcctgccctcacgctggcgccaagagcttctacaagaacctgatctggctggtgaagaagggcaacagctaccccaagctgtccaagtcttacattaacgacaagggcaaggaggtgctggtcctgtggggcatccaccaccccagcaccagcgccgaccaacagagcctgtaccagaacgccgacacctacgtgttcgtgggcagcagccggtacagcaagaagttcaagcccgagatcgccatccggcccaaggtgcgggaccaggagggccggatgaactactactggaccctggtggagcctggcgacaagatcaccttcgaggccaccggcaacctggtggtgccccggtacgccttcgccatggagcggaacgcc

SEQ ID NO: 81 - DNA, 1695 bp, Artificial Sequence
  FLHA_PR8
atgaaggccaatttgttggtccttctatgtgccctagccgccgccgacgccgacacaatctgcatcggatatcacgcaaacaacagcaccgacaccgtggatacggtcttggagaagaacgtgaccgtgacccattccgtgaaccttctcgaggatagccacaatggcaagctgtgtagactcaagggcattgccccgctgcagctgggaaagtgcaatattgctggctggctgttgggcaaccctgagtgtgaccctctgttaccagtgagatcttggagctatatcgtcgaaacccctaacagcgagaacggcatatgctacccaggcgacttcatcgactacgaggaactgcgcgagcagctgagctctgtgtcgagcttcgagcggttcgagatcttccctaaggaatctagctggcctaatcataacacaaatggcgttactgctgcctgtagccacgagggaaagagcagtttctaccggaatctgctgtggctgacagagaaggagggctcctaccctaagctgaagaatagctatgtgaacaagaagggcaaggaggtgctggtgctgtggggaatacaccacccacctaactcgaaggagcagcagaatctgtaccagaatgagaatgcctacgtgtccgtcgtgacctccaactacaaccggcggttcacgcctgagatcgccgagaggcctaaggtgagggaccaggccggacgcatgaactactactggaccctgctgaagcctggcgatacaatcatcttcgaggctaatggaaacctgatcgcgccaatgtacgccttcgccctgtccagaggattcggcagcggcatcatcacatccaacgcctccatgcacgaatgcaacaccaagtgccagacgcctctgggagctatcaatagcagcttgccttaccagaatatccaccctgtgaccattggagagtgtccaaagtacgtgcgcagcgcaaagctgcggatggtcacaggcctgcggaatataccttctatccagagccgaggcctgttcggtgccattgccggcttcatcgagggtggctggaccggaatgatcgacggctggtatggataccaccaccagaatgaacagggcagcggctacgccgccgatcagaagtccacccagaacgcaatcaatggtatcacaaacaaggtgaacactgtaatcgagaagatgaacatccaattcacagccgtgggcaaggagttcaataagctggagaagcggatggagaacctcaacaagaaggtggacgacggcttcctggatatctggacctacaacgcagagctgctggtgttgctggagaacgagagaaccctcgacttccatgatagcaacgttaagaacctatacgagaaggtgaagtcacagctgaagaataacgccaaggagattggcaacggctgcttcgaattctaccacaagtgcgacaacgagtgtatggagagcgtccggaatggcacctacgactatcctaagtatagcgaggagagcaagcttaatagagagaaggtcgatggcgtgaagctggagtcaatgggaatctaccagatcctggctatttattcaaccgtggcatcaagtctggtgcttctggtcagcctgggcgccatcagcttctggatgtgctccaatggcagcctgcaatgccgcatctgcata

SEQ ID NO: 82 - DNA, 1698 bp, Artificial Sequence
  FLHA_Cal09
atgaaggctatcttggtggtgttgttgtacacattcgccaccgccaacgccgacaccctctgcatcggctaccacgcgaacaattcaaccgacaccgttgacaccgtcctcgagaagaacgtgaccgtgactcatagcgtcaacctcctcgaggacaagcataacggcaagctctgtaagcttagaggagtggcccctctccacctgggcaagtgtaacattgcaggctggatcctgggcaaccctgagtgcgagagcctgtcaaccgctagcagctggagctacatcgtggaaaccccatccagcgataacggcacctgctaccctggcgatttcatcgactacgaggagctgcgcgagcagttgagcagcgtctccagcttcgagagattcgagatcttccctaagactagcagctggcctaatcatgactccaataagggcgtgacggccgcctgtcctcacgctggagccaagtcgttctacaagaacctgatctggctggtaaagaagggcaacagctacccaaagctgagcaagtcctacatcaacgacaagggcaaggaagtgctggtgctgtggggaatccatcacccaagcacctctgcggaccagcagtctctgtatcagaacgccgacacctatgtgttcgtaggctcctccagatactccaagaagttcaagccagagattgctatccgcccaaaggtgcgggatcaagagggtcgcatgaattattactggaccctggtcgagccaggcgataagatcacattcgaagccacgggaaatctggtggtgcctagatacgctttcgccatggagagaaacgccggcagcggcatcatcatatccgacacacctgtgcacgactgcaacacaacatgccagacgccaaagggagccatcaacacatctcttccattccagaacattcacccaatcacaatcggcaagtgtccaaagtacgtgaagtccaccaagcttagactggccaccggcctgcgtaacatccctagcatccagtcgagaggcctcttcggcgccatcgccggattcattgaaggtggctggaccggcatggtggacggttggtatggctaccaccaccagaacgagcagggcagcggctacgccgcggacctgaagtccacccagaacgctattgacgagatcaccaacaaggtgaacagcgtgatcgagaagatgaatacccagttcaccgccgtcggcaaggagttcaaccatctggagaagagaatcgagaacctcaacaagaaggtcgacgacggcttcctggacatttggacttacaacgctgagttgttggtgcttcttgagaatgagcggaccctggactatcacgactcaaatgtgaagaacctgtacgagaaggtgagatcccagctgaagaacaatgctaaggaaatcggcaacggctgcttcgagttctatcataagtgtgacaacacctgcatggagtctgttaagaacggcacatacgactacccgaagtactctgaggaggccaagctgaaccgagaggagatagacggcgttaagctagaaagtacaaggatctaccagatccttgccatctactccaccgtggcctccagcctggtgttggtggtgagcctgggcgccatcagcttctggatgtgcagtaacggaagcctacagtgccgaatctgcatc

SEQ ID NO: 83 - protein, 585 aa, Artificial Sequence
  MRK_B_consUnique
MKAIIVLLMVVTSNADRICTGITSSNSPHVVKTATQGEVNVTGVIPLTTTPTKSHFANLKGTRTRGKLCPDCLNCTDLDVALGRPMCVGTTPSAKASILHEVRPVTSGCFPIMHDRTKIRQLPNLLRGYEHIRLSTQNVIDAEKAPGGPYRLGTSGSCPNATSKNGFFATMAWAVPKNDNNKNATNPLTVEVPYICTEGEDQITVWGFHSDNKTQMKKLYGDSNPQKFTSSANGVTTHYVSQIGGFPDQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSICL

SEQ ID NO: 84 - protein, 584 aa, Artificial Sequence
  MRK_B_ConsensusA
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYENIRLSTQNVIDAEKAPGGPYRLGTSGSCPNATSKIGFFATMAWAVPKDNYKNATNPLTVEVPYICTEGEDQITVWGFHSDNKTQMKNLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 85 - protein, 585 aa, Artificial Sequence
  MRK_B_ConsensusB
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNVTNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 86 - protein, 585 aa, Artificial Sequence
  MRK_B_cot_AA
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYENIRLSTQNVIDAEKAPGGPYRLGTSGSCPNATSKSGFFATMAWAVPKNDNNKNATNPLTVEVPYICTEGEDQITVWGFHSDNKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 87 - protein, 585 aa, Artificial Sequence; identical to SEQ ID NO: 85
  MRK_B_COT_A

SEQ ID NO: 88 - protein, 584 aa, Artificial Sequence
  MRK_B_COT_B
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYENIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

(19) United States
(12) Patent Application Publication
(10) Pub. No.: US 2019/0314486 A1
(43) Pub. Date: Oct. 17, 2019

(54) INFLUENZA HEMAGGLUTININ PROTEIN VACCINES

(71) Applicant: Merck Sharp & Dohme Corp., Rahway, NJ (US)

(72) Inventors: Jessica Anne Flynn, Lansdale, PA (US); Lan Zhang, Chalfont, PA (US); Kerim Babaoglu, Lansdale, PA (US); Arthur Fridman, East Norriton, PA (US); David Nickle, Seattle, WA (US)

(73) Assignee: Merck Sharp & Dohme Corp., Rahway, NJ (US)

(21) Appl. No.: 16/343,272

(22) PCT Filed: Oct. 18, 2017
(86) PCT No.: PCT/US17/57107

§ 371 Date: Apr. 18, 2019

Related U.S. Application Data
(60) Provisional application No. 62/490,086, filed on Apr. 26, 2017.
(60) Provisional application No. 62/411,240, filed on Oct. 21, 2016.

(51) Int. Cl. A61K 39/145; A61P 31/16; C07K 14/11

(52) U.S. Cl. CPC A61K 39/145; A61K 2039/53; C07K 14/11; A61P 31/16

(57) ABSTRACT

The disclosure relates to influenza virus hemagglutinin protein and DNA vaccines, as well as methods of using the vaccines and compositions comprising the vaccines.

### SEQUENCE LISTING

The instant application contains a Sequence Listing which has been submitted electronically in ASCII format and is hereby incorporated by reference in its entirety. Said ASCII copy, created Oct. 10, 2017, is named 24378-US-PCT SL.txt and is 277 kilobytes in size.

### FIELD OF THE INVENTION

The present invention relates to influenza virus hemagglutinin protein and DNA vaccines, as well as methods of using the vaccines and compositions comprising the vaccines.

### BACKGROUND OF INVENTION

Influenza viruses are members of the orthomyxoviridae family, and are classified into three distinct types (A, B, and C), based on antigenic differences between their nucleoprotein (NP) and matrix (M) protein. The orthomyxoviruses are enveloped animal viruses of approximately 100 nm in diameter. The influenza virions consist of an internal ribonucleoprotein core (a helical nucleocapsid) containing a single-stranded RNA genome, and an outer lipoprotein envelope lined inside by a matrix protein (M1). The segmented genome of influenza A and B viruses consists of eight molecules (seven for influenza C virus) of linear, negative polarity, single-stranded RNAs, which encode several polypeptides including: the RNA-directed RNA polymerase proteins (PB2, PB1 and PA) and nucleoprotein (NP), which form the nucleocapsid; the matrix proteins (M1, M2, which is also a surface-exposed protein embedded in the virus membrane); two surface glycoproteins, which project from the lipoprotein envelope: hemagglutinin (HA) and neuraminidase (NA); and nonstructural proteins (NS1 and NS2). Transcription and replication of the genome takes place in the nucleus and assembly takes place at the plasma membrane.

Hemagglutinin is the major envelope glycoprotein of influenza A and B viruses, and hemagglutinin-esterase (HE) of influenza C viruses is a protein homologous to HA. The rapid evolution of the HA protein of the influenza virus results in the constant emergence of new strains, rendering the adaptive immune response of the host only partially protective to new infections. The amino acid sequence of the stem region of the hemagglutinin protein is highly conserved across types, sub-types and strains of influenza viruses and contains a site of vulnerability for group 1 viruses. Thus, an immune response directed to this region of the HA protein may protect individuals against influenza viruses from several types, sub-types and/or strains.

Ferritin, an iron storage protein found in almost all living organisms, is an example which has been extensively studied and engineered for a number of potential biochemical/biomedical purposes [Iwahori, K. U.S. Patent 2009/0233377 (2009); Meldrum, F. C. et al. Science 257, 522-523 (1992); Naitou, M. et al. U.S. Patent 2011/0038025 (2011); Yamashita, I. Biochim Biophys Acta 1800, 846-857 (2010)]. Further, the molecular architecture of ferritin, which consists of 24 subunits assembling into an octahedral cage with 432 symmetry, has the potential to display multimeric antigens on its surface.

There are eighteen known HA serotypes and nine known NA serotypes for Influenza A viruses. The identity of the different serotypes present in a viral particle typically is used to describe a virus. For example, H1N1 is an influenza virus with HA serotype H1 and NA serotype N1; H5N1 is an influenza virus with HA serotype H5 and NA serotype N1. Only H1, H2 and H3 serotypes, and N1 and N2 serotypes usually infect humans.

Influenza strains are generally species or genus specific; i.e. an influenza strain which can infect pigs (a swine influenza virus) typically does not infect humans or birds; an influenza strain which can infect birds (an avian influenza virus) typically does not infect humans or pigs; and an influenza strain which can infect humans (a human influenza virus) does not infect birds or pigs. Influenza strains, however, can mutate and become infective from one species to another. For example, a strain which only infects pigs, a swine influenza, can mutate or recombine to become a strain that can infect humans only or both pigs and humans. A flu virus commonly referred to as “swine flu” is an influenza virus strain, such as an H1N1 strain, which can infect humans and which was derived from a strain that was previously specific for pigs (i.e. a swine flu virus is a swine origin human influenza or swine derived human influenza). A flu virus commonly referred to as “bird flu” is an influenza virus strain, such as an H5N1 strain, which can infect humans and which was derived from a strain that was previously specific for birds (i.e. a bird flu virus avian origin human influenza or avian derived human influenza).

The biggest challenge for therapy and prophylaxis against influenza and other infections using traditional vaccines is the limitation of vaccines in breadth, providing protection only against closely related subtypes. In addition, the length of time required to complete current standard influenza virus vaccine production processes inhibits the rapid development and production of an adapted vaccine in a pandemic situation.

### SUMMARY OF INVENTION

The present invention provides influenza vaccines comprising at least one isolated antigenic hemagglutinin (HA) protein (SEQ ID NOs: 1-4, 8-11, 14-19, 41-61, and 83-88). In certain embodiments, the protein has at least 90%, 95%, 96%, 97.5%, 98%, 99%, 95-99% identity to a mature amino acid sequence in any one of SEQ ID NOs: 1-4, 8-11, 14-19, 41-61, and 83-88. In one embodiment, the polypeptide comprises a mature amino acid sequence that is the extracellular domain of SEQ ID NO: 8 or 10. The exact sequence of the extracellular domain can vary based on the truncation site. In one embodiment, the truncation site is any of the italic residues of SEQ ID NO:8 or 10 in Table 1. In another embodiment, the extracellular domain of SEQ ID NO: 8 or 10 can be linked to a foldon domain (GYIPEAPRDGQAYVRKDGEWVLLSTFL) through a linker.

The present invention also provides an influenza virus vaccine comprising at least two isolated antigenic polypeptides, wherein the first polypeptide comprises the mature amino acid sequence of any one of SEQ ID NOS: 1-5, 8-11, 14-19, 41-63, and 83-88, and the second polypeptide comprises the mature amino acid sequence of SEQ ID NO: 20. In certain embodiments, the protein has at least 90%, 95%, 96%, 97.5%, 98%, 99%, or 95-99% identity to a mature amino acid sequence in any one of SEQ ID NOs: 1-5, 8-11, 14-19, 41-63, and 83-88. The present invention also provides an influenza virus vaccine comprising at least two isolated antigenic polypeptides, wherein the first polypeptide comprises the mature amino acid sequence of any one of SEQ ID NO: 4 or 5, and the second polypeptide comprises the mature amino acid sequence of SEQ ID NO: 20.

The present invention also provides an influenza virus vaccine comprising an isolated polynucleotide sequence comprising SEQ ID NOs: 21-24, 28-31 34-39, or 64-81. In certain embodiments, the polynucleotide has at least 90%, 95%, 96%, 97%, 98%, 99%, 95-99% identity to any one of SEQ ID NOs: 21-24, 28-31 34-39, or 64-81. Also provided is an influenza virus vaccine comprising an isolated polynucleotide sequence which encodes an antigenic peptide having the mature amino acid sequence of any one of SEQ ID NO: 1-4, 8-11, 14-19, 41-61, and 83-88.

The present invention also provides an influenza virus vaccine comprising a first isolated polynucleotide sequence comprising SEQ ID NOs: 21-24, 28-31, 34-39, or 64-81, and a second isolated polynucleotide sequence comprising SEQ ID NO: 40. In certain embodiments, the polynucleotide has at least 90%, 95%, 96%, 97.5%, 98%, 99%, or 95-99% identity to any one of SEQ ID NOs: 21-24, 28-31, 34-39, 40, and 64-81. The present invention also provides an influenza virus vaccine comprising a first isolated polynucleotide sequence comprising SEQ ID NO: 24 or 25 and a second isolated polynucleotide sequence comprising SEQ ID NO: 40.

The present disclosure also provides antibody molecules, including full length antibodies and antibody derivatives, directed against the novel influenza virus sequences.

In some embodiments, the vaccine further comprises an aluminum adjuvant such as MMA or APA.

In some embodiments, the antigenic polypeptide comprises G430C, E438C, Q457L mutations of the Influenza B/Brisbane/60/2008 HA sequence to improve trimerization (amino acid residue number one is from the first amino acid of the N-terminal Methionine of the signal peptide for the HA sequence).

In some embodiments, the antigenic polypeptide comprises mutations I333T, M429S and L432T of the HA sequence of the Influenza B/Brisbane/60/2008 (amino acid residue number one is from the first amino acid of the N-terminal Methionine of the signal peptide for the HA sequence).

In some embodiments, the antigenic polypeptide comprises ferritin sequence of HA protein for nanoparticle formation.

In some embodiments, the antigenic polypeptide comprises the transmembrane domain of the HA sequence.

In some embodiments, the antigenic polypeptide comprises the consensus HA sequence for pandemic H1 strains.

In some embodiments, the antigenic polypeptide comprises the consensus HA sequence for seasonal H1 strains.

In some embodiments, the antigenic polypeptide is formulated with a lipid nanoparticle comprising a cationic lipid, a PEG-modified lipid, a sterol and a non-cationic lipid.

In some embodiments, at least one influenza antigenic polypeptide comprises a mutated N-linked glycosylation site.

In some embodiments, the vaccine is multivalent, and comprises at least two to ten, two, three, four or five or ten of the above antigenic polypeptides.

Some embodiments of the present disclosure provide methods of inducing an antigen specific immune response in a subject, comprising administering to the subject any of the vaccine as provided herein in an amount effective to produce an antigen-specific immune response. In some embodiments, the vaccine is a combination vaccine comprising a combination of influenza vaccines.

In some embodiments, an antigen-specific immune response comprises a T cell response or a B cell response.

In some embodiments, a method of producing an antigen-specific immune response comprises administering to a subject a single dose (no booster dose) of an influenza vaccine of the present disclosure.

In some embodiments, a method further comprises administering to the subject a second (booster) dose of an influenza vaccine. Additional doses of an influenza vaccine may be administered.

In some embodiments, an influenza vaccine is administered to a subject by intradermal injection, intramuscular injection, or by intranasal administration. In some embodiments, an influenza vaccine is administered to a subject by intramuscular injection.

Some embodiments of the present disclosure provide methods of inducing an antigen specific immune response in a subject, including administering to a subject an influenza vaccine in an effective amount to produce an antigen specific immune response in a subject. Antigen-specific immune responses in a subject may be determined, in some embodiments, by assaying for antibody titer (for titer of an antibody that binds to an influenza antigenic polypeptide) following administration to the subject of any of the influenza vaccines of the present disclosure.

In some embodiments, the vaccine is formulated in an effective amount to produce an antigen specific immune response in a subject.

In some embodiments, the vaccine immunizes the subject against Influenza for up to 1 or 2 years. In some embodiments, the vaccine immunizes the subject against Influenza for more than 2 years, more than 3 years, more than 4 years, or for 5-10 years. In one embodiment, the vaccination is yearly.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a vaccine may be administered later in life, for example, at the age of about 6 months to about 5 years, or older.

In some embodiments, the subject is a young adult between the ages of about 20 years and about 50 years (e.g., about 20, 25, 30, 35, 40, 45 or 50 years old).

In some embodiments, the subject is an elderly subject about 50-60 years old, 60 years old, about 70 years old, or older, 80 years or older, 90 years or older (e.g., about 60, 65, 70, 75, 80, 85 or 90 years old).

In some embodiments, the subject has been exposed to influenza; the subject is infected with influenza; or subject is at risk of infection by influenza.

In some embodiments, the subject is immunocompromised (has an impaired immune system, e.g., has an immune disorder or autoimmune disorder).

The details of various embodiments of the disclosure are set forth in the description below. Other features, objects, and advantages of the disclosure will be apparent from the description and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIGS. 1A-1B depict endpoint titers of pooled serum from animals vaccinated with the test vaccines. In FIG. 1A, the vaccines tested are shown on the x-axis and the binding to HA from each of the different strains of influenza is plotted as an endpoint titer. In FIG. 1B, the vaccines tested are shown on the x-axis, and the endpoint titer to NP protein is plotted as an endpoint titer.

FIG. 2 shows an examination of functional antibody response through an assessment of the ability of serum to neutralize a panel of HA-pseudotyped viruses.

FIG. 3 is a representation of cell-mediated immune responses following mRNA vaccination. Splenocytes were harvested from vaccinated mice and stimulated with a pool of overlapping NP peptides. The % of CD4 or CD8 T cells secreting one of the three cytokines (IFN-γ, IL-2, or TNF-α) is plotted.

FIG. 4 is a representation of cell-mediated immune responses following mRNA vaccination. Splenocytes were harvested from vaccinated mice and stimulated with a pool of overlapping HA peptides. The % of CD4 or CD8 T cells secreting one of the three cytokines (IFN-γ, IL-2, or TNF-α) is plotted.

FIG. 5 shows murine weight loss following challenge with a lethal dose of mouse-adapted H1N1 A/Puerto Rico/8/1934. The percentage of weight lost as compared to baseline was calculated for each animal and was averaged across the group. The group average was plotted over time in days. Error bars represent standard error of the mean. Efficacy of the NIHGen6HASS-foldon+NP combination vaccines was better than that of either the NIHGen6HASS-foldon or NP mRNA vaccine alone, regardless of antigen co-formulation or co-delivery method.

FIG. 6A depicts the endpoint titers of the pooled serum from animals vaccinated with the test vaccines. FIG. 6B shows efficacy of the test vaccines (NIHGen6HASS-foldon and NIHGen6HASS-TM2) is similar. Following challenge with a lethal dose of mouse-adapted H1N1 A/Puerto Rico/8/1934, the percentage of group weight lost as compared to baseline was calculated and plotted over time in days.

FIGS. 7A and 7B shows the endpoint neutralization titers detected in serum from vaccinated animals against a panel of 11 H1N1 influenza viruses. For each sample, the highest dilution of serum that resulted in an OD greater than the cutoff was assigned as the microneutralization titer. A value of <20 indicates lack of neutralization, even at the lowest dilution tested.

FIGS. 8A and 8B show hemagglutination inhibition (HAI) titers of each serum sample (indicated in the first column) against a panel of 11 H1N1 influenza viruses. Antisera from individual animals exposed to the same vaccine regimen were pooled and tested for their ability to inhibit agglutination of turkey red blood cells induced by multiple influenza A H1 virus strains. The highest dilution with no visible agglutination was assigned as the serum titer represented in the table. A value of <10 indicates a lack of HAI, even at the lowest serum dilution tested. Titers >40 (correlate of protection for influenza) are highlighted in gray. FIGS. 8C and 8D shows murine weight loss following challenge with a lethal dose of mouse adapted H1N1 A/Puerto Rico/8/1934. The percentage of group weight lost as compared to baseline was calculated and plotted over time in days.

FIGS. 9A and 9B show the murine survival following challenge with H1N1 Ca109. The percentage of group survival was calculated and plotted over time in days. FIGS. 9C and 9D shows murine weight loss following challenge with H1N1 Ca109. The percentage of group weight lost as compared to baseline was calculated and plotted over time in days.

FIGS. 10A and 10B show hemagglutination inhibition (HAI) titers of each serum sample (indicated in the first column) against a panel of 11 H1N1 influenza viruses. Antisera from individual animals exposed to the same vaccine regimen were pooled and tested for their ability to inhibit agglutination of turkey red blood cells induced by multiple influenza A h1 (FIG. 10A) and H3 (FIG. 10B) virus strains. The highest dilution with no visible agglutination was assigned as the serum titer represented in the table. A value of <10 indicates a lack of HAI, even at the lowest serum dilution tested. Titers >40 (correlate of protection for influenza) are highlighted in gray.

FIG. 11A shows the murine survival following challenge with H1N1 PR8. The percentage of group survival was calculated and plotted over time in days. FIG. 11B shows murine weight loss following challenge with H1N1 PR8. The percentage of group weight lost as compared to baseline was calculated and plotted over time in days. FIG. 11C shows murine survival following challenge with H3 HK68. The percentage of group survival was calculated and plotted over time in days. FIG. 11D shows murine weight loss following challenge with H3 HK68. The percentage of group weight lost as compared to baseline was calculated and plotted over time in days.

### DETAILED DESCRIPTION

“Consensus” or “consensus sequence” as used herein means a polypeptide sequence based on analysis of an alignment of multiple subtypes of a particular influenza antigen. DNA sequences that encode a consensus polypeptide sequence may be prepared. Vaccines comprising isolated proteins that comprise consensus sequences and/or DNA molecules that encode such proteins can be used to induce broad immunity against multiple subtypes or serotypes of a particular influenza antigen. Consensus influenza antigens can include influenza A consensus hemagglutinin amino acid sequences, including for example consensus H1, consensus H2, consensus H3, or influenza B consensus hemagglutinin amino acid sequences.

“RBD” as used herein means receptor binding domain.

“Isolated” polypeptides or polynucleotides are at least partially free of other biological molecules from the cells or cell cultures in which they are produced. Such biological molecules include other nucleic acids, proteins, lipids, carbohydrates, or other material such as cellular debris and growth medium. It may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the polypeptides or polynucleotides.

In some embodiments, the virus is a strain of Influenza A or Influenza B or combinations thereof. In some embodiments, the strain of Influenza A or Influenza B is associated with birds, pigs, horses, dogs, humans or non-human primates.

Some embodiments provide methods of preventing or treating influenza viral infection comprising administering to a subject any of the vaccines described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the vaccine. In some embodiments, the vaccine is administered to the subject by intradermal, intramuscular injection, subcutaneous injection, intranasal inoculation, or oral administration.

In some embodiments, the vaccine comprises at least one of the aforementioned antigenic polypeptides of the invention and at least one protein, or immunogenic fragment or variant or homolog thereof, selected from a NP protein, a NA protein, a M1 protein, a M2 protein, a NS1 protein and a NS2 protein obtained from influenza virus.

The influenza antigens provided herein can be arranged as a vaccine that causes seroconversion in vaccinated mammals and provides cross-reactivity against a broad range of seasonal strains of influenza and also pandemic strains of influenza. The seroconversion and broad cross-reactivity can be determined by measuring inhibiting titers against different hemagglutinin strains of influenza. Preferred combinations include at least two antigens from each of the influenza antigens described herein.

### Lipid Nanoparticles

As used herein, “lipid nanoparticle” or “LNP” refers to any lipid composition that can be used to deliver a product, including, but not limited to, liposomes or vesicles, wherein an aqueous volume is encapsulated by amphipathic lipid bilayers (e.g., single; unilamellar or multiple; multilamellar), or, in other embodiments, wherein the lipids coat an interior comprising a prophylactic product, or lipid aggregates or micelles, wherein the lipid encapsulated therapeutic product is contained within a relatively disordered lipid mixture. Except where noted, the lipid nanoparticle does not need to have the antigenic polypeptide incorporated therein and may be used to deliver a product when in the same formulation.

As used herein, “polyamine” means compounds having two or more amino groups. Examples include putrescine, cadaverine, spermidine, and spermine.

Unless otherwise specified, mole % refers to a mole percent of total lipids. Generally, the LNPs of the compositions of the invention are composed of one or more cationic lipids (including ionizable cationic lipids) and one or more poly(ethyleneglycol)-lipids (PEG-lipids). In certain embodiments, the LNPs further comprise one or more non-cationic lipids. The one or more non-cationic lipids can include a phospholipid, phospholipid derivative, a sterol, a fatty acid, or a combination thereof.

Cationic lipids and ionizable cationic lipids suitable for the LNPs are described herein. Ionizable cationic lipids are characterized by the weak basicity of their lipid head groups, which affects the surface charge of the lipid in a pH-dependent manner, rendering them positively charged at acidic pH but close to charge-neutral at physiologic pH. Cationic lipids are characterized by monovalent or multivalent cationic charge on their headgroups, which renders them positively charged at neutral pH. In certain embodiments, the cationic and ionizable lipid is capable of complexing with hydrophilic bioactive molecules to produce a hydrophobic complex that partitions into the organic phase of a two-phase aqueous/organic system. It is contemplated that both monovalent and polyvalent cationic lipids may be utilized to form hydrophobic complexes with bioactive molecules.

Preferred cationic and ionizable cationic lipids for use in forming the LNPs include, but are not limited to, N,N-dioleyl-N,N-dimethylammonium chloride (“DODAC”); N-(2,3dioleyloxy)propyl)-N,N,Ntrimethylammonium chloride (“DOTMA”); N,NdistearylN,N-dimethylammonium bromide (“DDAB”); N-(2,3dioleoyloxy)propyl)-N,N,N-trimethylamntonium chloride (“DODAP”); 1,2 bis (oleoyloxy)-3-(trimethylammonio) propane (DOTAP); 3-(N-(N,N-dimethylaminoethane)-carbam-oyl)cholesterol (′DC-Chol”); diheptadecylamidoglycylspermidine (“DHGS”) and N-(1,2-dimyristyloxyprop-3-yl)-N,N-dimethyl-N-hydoxyethyl ammonium bromide (“DMRIE”). Additionally, a number of commercial preparations of cationic lipids, as well as other components, are available which can be used in the present invention. These include, for example, LIPOFECTIN® (commercially available cationic lipid nanoparticles comprising DOTMA and 1,2dioleoyl-sn-3-phosphoethanolamine (“DOPE”), from GIBCOBRL, Grand Island, N.Y., USA); and LIPOFECTAMINE® (commercially available cationic lipid nanoparticles comprising N-(1-(2,3dioleyloxy)propyl)N-(2-(sperminecarboxamido)ethyl)-N,N-dimethylammonium trifluoroacetate (“DOSPA’) and (“DOPE”), from (GIBCOBRL). The following lipids are cationic and have a positive charge at below physiological pH: DODAP, DODMA, DMDMA, 1,2-DiLinoleyloxy-N,N-dimethylaminopropane (DLinDMA), 4-(2,2-diocta-9,12-dienyl-[1,3]dioxolan-4-ylmethyl)-dimethylamine, DLinKDMA (WO 2009/132131 A1), DLin-K-C2-DMA (WO2010/042877), DLin-M-C3-DMA (WO2010/146740 and/or WO2010/105209), DLin-MC3-DMA (heptatriaconta-6,9,28,31-tetraen-19-yl 4-(dimethylamino)butanoate; Jayaraman et al., 2012, Angew. Chem. Int. Ed. Engl. 51:8529-8533), 2-{4-[(3β)-cholest-5-en-3-yloxy]butoxy}-N,N-dimethyl-3-[(9Z,12Z)-octadeca-9,12-dienlyloxyl]propan-1-amine) (CLinDMA), and the like. Other cationic lipids suitable for use in the invention include, e.g., the cationic lipids described in U.S. Pat. Nos. 5,208,036, 5,264,618, 5,279,833 and 5,283,185, and U.S. Patent Application Publication Nos. 2008/0085870 and 2008/0057080. Other cationic lipids suitable for use in the invention include, e.g., Lipids E0001-E0118 or E0119-E0180 as disclosed in Table 6 (pages 112-139) of International Patent Application Publication No. WO2011/076807 (which also discloses methods of making, and methods of using these cationic lipids).

In certain aspects of this embodiment of the invention, the LNPs comprise one or more of the following ionizable cationic lipids: DLinDMA, DlinKC2DMA DLin-MC3-DMA, CLinDMA, or S-Octyl CLinDMA (See International Patent Application Publication No. WO2010/021865).

In certain aspects of this embodiment of the invention, LNPs comprise one or more ionizable cationic lipids described in International Patent Application Publication No. WO2011/022460 A1, or any pharmaceutically acceptable salt thereof, or a stereoisomer of any of the compounds or salts therein.

When structures of the same constitution differ in respect to the spatial arrangement of certain atoms or groups, they are stereoisomers, and the considerations that are significant in analyzing their interrelationships are topological. If the relationship between two stereoisomers is that of an object and its nonsuperimposable mirror image, the two structures are enantiomeric, and each structure is said to be chiral. Stereoisomers also include diastereomers, cis-trans isomers and conformational isomers. Diastereoisomers can be chiral or achiral, and are not mirror images of one another. Cis-trans isomers differ only in the positions of atoms relative to a specified plane in cases where these atoms are, or are considered as if they were, parts of a rigid structure. Conformational isomers are isomers that can be interconverted by rotations about formally single bonds. Examples of such conformational isomers include cyclohexane conformations with chair and boat conformers, carbohydrates, linear alkane conformations with staggered, eclipsed and gauche conformers, etc. See J. Org. Chem. 35, 2849 (1970).

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, enantiomers are identical except that they are non-superimposable mirror images of one another. A mixture of enantiomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the Formulas of the invention, it is understood that both the (R) and (S) configurations of the chiral carbon, and hence both enantiomers and mixtures thereof, are embraced within the Formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines (bonds to atoms below the plane). The Cahn-Inglod-Prelog system can be used to assign the (R) or (S) configuration to a chiral carbon.

When the compounds of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. In a particular embodiment when a specific absolute configuration is designated, the enantiomeric excess of depicted compounds is at least about 90%.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

The LNPs may also comprise any combination of two or more of the cationic lipids described herein. In certain aspects, the cationic lipid typically comprises from about 0.1 to about 99.9 mole % of the total lipid present in said particle. In certain aspects, the cationic lipid can comprise from about 80 to about 99.9% mole %. In other aspects, the cationic lipid comprises from about 2% to about 70%, from about 5% to about 50%, from about 10% to about 45%, from about 20% to about 99.8%, from about 30% to about 70%, from about 34% to about 59%, from about 20% to about 40%, or from about 30% to about 40% (mole %) of the total lipid present in said particle.

The LNPs described herein can further comprise a noncationic lipid, which can be any of a variety of neutral uncharged, zwitterionic or anionic lipids capable of producing a stable complex. They are preferably neutral, although they can be negatively charged. Examples of noncationic lipids useful in the present invention include phospholipid-related materials, such as natural phospholipids, synthetic phospholipid derivatives, fatty acids, sterols, and combinations thereof. Natural phospholipids include phosphatidylcholine (PC), phosphatidylethanolamine (PE), and phosphatidylglycerol (PG), phosphatidylserine (PS), phosphatidylinositol (PI), Phosphatidic acid (phosphatidate) (PA), dipalmitoylphosphatidylcholine, monoacyl-phosphatidylcholine (lyso PC), 1-palmitoyl-2-oleoyl-sn-glycero-3-phosphocholine (POPC), N-Acyl-PE, phosphoinositides, and phosphosphingolipids. Phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), and phosphatidylserine (DOPS). Fatty acids include C14:0, palmitic acid (C16:0), stearic acid (C18:0), oleic acid (C18:1), linoleic acid (C18:2), linolenic acid (C18:3), and arachidonic acid (C20:4), C20:0, C22:0 and lethicin.

In certain embodiments of the invention the non-cationic lipid is selected from lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylet-hanolamine (POPE) and dioleoyl-phosphatidylethanolamine 4-(N-maleimidomethyl)-cyclohexane-1-carboxylate (DOPE-mal). Noncationic lipids also include sterols such as cholesterol, stigmasterol or stigmastanol. Cholesterol is known in the art. See U.S. Patent Application Publication Nos: U.S. 2006/0240554 and U.S. 2008/0020058. In certain embodiments, the LNP comprise a combination of a phospholipid and a sterol.

Where present, the non-cationic lipid typically comprises from about 0.1% to about 65%, about 2% to about 65%, about 10% to about 65%, or about 25% to about 65% expressed as mole percent of the total lipid present in the LNP. The LNPs described herein further include a polyethyleneglycol (PEG) lipid conjugate (“PEG-lipid”) which may aid as a bilayer stabilizing component. The lipid component of the PEG lipid may be any non-cationic lipid described above including natural phospholipids, synthetic phospholipid derivatives, fatty acids, sterols, and combinations thereof. In certain embodiments of the invention, the PEG-lipids include, PEG coupled to dialkyloxypropyls (PEG-DAA) as described in, e.g., International Patent Application Publication No. WO 05/026372, PEG coupled to diacylglycerol (PEG-DAG) as described in, e.g., U.S. Patent Publication Nos. 20030077829 and 2005008689; PEG coupled to phosphatidylethanolamine (PE) (PEG-PE), or PEG conjugated to 1,2-Di-O-hexadecyl-sn-glyceride (PEG-DSG), or any mixture thereof (see, e.g., U.S. Pat. No. 5,885,613).

In one embodiment, the PEG-DAG conjugate is a dilaurylglycerol (C 12)-PEG conjugate, a PEG dimyristylglycerol (C14)conjugate, a PEG-dipalmitoylglycerol (C16) conjugate, a PEG-dilaurylglycamide (C12) conjugate, a PEG-dimyristylglycamide (C14) conjugate, a PEG-dipalmitoylglycamide (C 16) conjugate, or a PEG-disterylglycamide (C 18). Those of skill in the art will readily appreciate that other diacylglycerols can be used in the PEG-DAG conjugates.

In certain embodiments, PEG-lipids include, but are not limited to, PEG-dimyristolglycerol (PEG-DMG), PEG-disteryl glycerol (PEG-DSG), PEG-dipalmetoleyl, PEG-dioleyl, PEG-distearyl, PEG-diacylglycamide (PEG-DAG), PEG- dipalmitoyl phosphatidylethanolamine (PEG-DPPE), and PEG-1,2-dimyristyloxlpropyl-3-amine (PEG-c-DMA).

In certain embodiments, the PEG-lipid is PEG coupled to dimyristoylglycerol (PEG-DMG), e.g., as described in Abrams et al., 2010, Molecular Therapy 18(1):171, and U.S. Patent Application Publication Nos. US 2006/0240554 and US 2008/0020058.

In certain embodiments, the PEG-lipid, such as a PEG-DAG, PEG-cholesterol, PEG-DMB, comprises a polyethylene glycol having an average molecular weight ranging of about 500 daltons to about 10,000 daltons, of about 750 daltons to about 5,000 daltons, of about 1,000 daltons to about 5,000 daltons, of about 1,500 daltons to about 3,000 daltons or of about 2,000 daltons. In certain embodiments, the PEG-lipid comprises PEG400, PEG1500, PEG2000 or PEG5000.

The acyl groups in any of the lipids described above are preferably acyl groups derived from fatty acids having about C10 to about C24 carbon chains. In one embodiment, the acyl group is lauroyl, myristoyl, palmitoyl, stearoyl or oleoyl.

The PEG-lipid conjugate typically comprises from about 0.1% to about 15%, from about 0.5% to about 20%, from about 1.5% to about 18%, from about 4% to about 15%, from about 5% to about 12%, from about 1% to about 4%, or about 2% expressed as a mole % of the total lipid present in said particle.

In certain embodiments of the invention, the LNPs comprise one or more cationic lipids, cholesterol and 1,2-Dimyristoyl-sn-glycerol methoxypolyethylene glycol (PEG-DMG).

In certain embodiments the invention, the LNPs comprise one or more cationic lipids, cholesterol, 1,2-Distearoyl-sn-glycero-3-phosphocholine (DSPC), and 1,2-Dimyristoyl-sn-glycerol methoxypolyethylene glycol (PEG-DMG).

In certain embodiments of the invention, the LNPs comprise lipid compounds assembled within the following molar ratios:

Cationic Lipid (20-99.8 mole %)

Non-cationic lipid (0.1-65 mole %) and

PEG-DMG (0.1-20 mole %).

In certain embodiments of the invention, the LNPs comprise lipid compounds assembled within the following molar ratios:

Cationic Lipid (30-70 mole %)

Non-cationic lipid (20-65 mole %) and

PEG-DMG (1-15 mole %).

In certain aspects of this embodiment, the non-cationic lipid is cholesterol. Exemplary LNPs may include cationic lipid/cholesterol/PEG-DMG at about the following molar ratios: 58/30/10.

In certain aspects of this embodiment, the non-cationic lipid is cholesterol and DSPC. Exemplary LNPs may include cationic lipid/cholesterol/DSPC/PEG-DMG at about the following molar ratios: 59/30/10/1; 58/30/10/2; 43/41/15/1; 42/41/15/2; 40/48/10/2; 39/41/19/1; 38/41/19/2; 34/41/24/1; and 33/41/24/2.

### Preparation of LNPs

LNPs can be formed, for example, by a rapid precipitation process which entails micro-mixing the lipid components dissolved in ethanol with an aqueous solution using a confined volume mixing apparatus such as a confined volume T-mixer, a multi-inlet vortex mixer (MIVM), or a microfluidics mixer device as described below. The lipid solution contains one or more cationic lipids, one or more noncationic lipids (e.g., DSPC), PEG-DMG, and optionally cholesterol, at specific molar ratios in ethanol. The aqueous solution consists of a sodium citrate or sodium acetate buffered salt solution with pH in the range of 2-6, preferably 3.5-5.5. The two solutions are heated to a temperature in the range of 25° C.-45° C., preferably 30° C.-40° C., and then mixed in a confined volume mixer thereby instantly forming the LNP. When a confined volume T-mixer is used, the T-mixer has an internal diameter (ID) range from 0.25 to 1.0 mm. The alcohol and aqueous solutions are delivered to the inlet of the T-mixer using programmable syringe pumps, and with a total flow rate from 10-600 mL/minute. The alcohol and aqueous solutions are combined in the confined-volume mixer with a ratio in the range of 1:1 to 1:3 vol:vol, but targeting 1:1.1 to 1:2.3. The combination of ethanol volume fraction, reagent solution flow rates and t-mixer tubing ID utilized at this mixing stage has the effect of controlling the particle size of the LNPs between 30 and 300 nm. The resulting LNP suspension is twice diluted into higher pH buffers in the range of 6-8 in a sequential, multi-stage in-line mixing process. For the first dilution, the LNP suspension is mixed with a buffered solution at a higher pH (pH 6-7.5) with a mixing ratio in the range of 1:1 to 1:3 vol:vol, but targeting 1:2 vol:vol. This buffered solution is at a temperature in the range of 15-40° C., targeting 30-40° C. The resulting LNP suspension is further mixed with a buffered solution at a higher pH, e.g., 6-8 and with a mixing ratio in the range of 1:1 to 1:3 vol:vol, but targeting 1:2 vol:vol. This later buffered solution is at a temperature in the range of 15-40° C., targeting 16-25° C. The mixed LNPs are held from 30 minutes to 2 hours prior to an anion exchange filtration step. The temperature during incubation period is in the range of 15-40° C., targeting 30-40° C. After incubation, the LNP suspension is filtered through a 0.8 tm filter containing an anion exchange separation step. This process uses tubing IDs ranging from 1 mm ID to 5 mm ID and a flow rate from 10 to 2000 mL/minute. The LNPs are concentrated and diafiltered via an ultrafiltration process where the alcohol is removed and the buffer is exchanged for the final buffer solution such as phosphate buffered saline or a buffer system suitable for cryopreservation (for example containing sucrose, trehalose or combinations thereof). The ultrafiltration process uses a tangential flow filtration format (TFF). This process uses a membrane nominal molecular weight cutoff range from 30-500 KD, targeting 100 KD. The membrane format can be hollow fiber or flat sheet cassette. The TFF processes with the proper molecular weight cutoff retains the LNP in the retentate and the filtrate or permeate contains the alcohol and final buffer wastes. The TFF process is a multiple step process with an initial concentration to a lipid concentration of 20-30 mg/mL. Following concentration, the LNP suspension is diafiltered against the final buffer (for example, phosphate buffered saline (PBS) with pH 7-8, 10 mM Tris, 140 mM NaCl with pH 7-8, or 10 mM Tris, 70 mM NaCl, 5 wt % sucrose, with pH 7-8) for 5-20 volumes to remove the alcohol and perform buffer exchange. The material is then concentrated an additional 1-3 fold via ultrafiltration. The final steps of the LNP manufacturing process are to sterile filter the concentrated LNP solution into a suitable container under aseptic conditions. Sterile filtration is accomplished by passing the LNP solution through a pre-filter (Acropak 500 PES 0.45/0.8 tm capsule) and a bioburden reduction filter (Acropak 500 PES 0.2/0.8 tm capsule). Following filtration, the vialed LNP product is stored under suitable storage conditions (2° C.-8° C., or −20° C. if frozen formulation).

In some embodiments, the LNPs of the compositions provided herein have a mean geometric diameter that is less than 1000 nm. In some embodiments, the LNPs have mean geometric diameter that is greater than 50 nm but less than 500 nm. In some embodiments, the mean geometric diameter of a population of LNPs is about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, the mean geometric diameter is between 100-400 nm, 100-300 nm, 100-250 nm, or 100-200 nm. In some embodiments, the mean geometric diameter is between 60-400 nm, 60-350 nm, 60-300 nm, 60-250 nm, or 60-200 nm. In some embodiments, the mean geometric diameter is between 75-250 nm. In some embodiments, 30%, 40%, 50%, 60%, 70%, 80%, 90%, or more of the LNPs of a population of LNPs have a diameter that is less than 500 nm. In some embodiments, 10%, 20%, 30%, 40%, 50%, 60%, 70%, 80%, 90%, or more of the LNPs of a population of LNPs have a diameter that is greater than 50 nm but less than 500 nm. In some embodiments, 10%, 20%, 30%, 40%, 50%, 60%, 70%, 80%, 90%, or more of the LNPs of a population of LNPs have a diameter of about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, 10%, 20%, 30%, 40%, 50%, 60%, 70%, 80%, 90%, or more of the LNPs of a population of LNPs have a diameter that is between 100-400 nm, 100-300 nm, 100-250 nm, or 100-200 nm. In some embodiments, 10%, 20%, 30%, 40%, 50%, 60%, 70%, 80%, 90%, or more of the LNPs of a population of LNPs have a diameter that is between 60-400 nm, 60-350 nm, 60-300 nm, 60-250 nm, or 60-200 nm.

In a particular embodiment, the size of the LNPs ranges between about 1 and 1000 nm, preferably between about 10 and 500 nm, more preferably between about 100 to 300 nm, and preferably 100 nm.

### Nucleic Acids/Polynucleotides

DNA of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence.

In some embodiments, a codon-optimized sequence shares between 65% and 85% (e.g., between about 67% and about 85%, or between about 67% and about 80%) sequence identity to a naturally-occurring sequence or a wild-type sequence. In some embodiments, a codon-optimized sequence shares between 65% and 75%, or about 80% sequence identity to a naturally-occurring sequence or wild-type sequence.

### Antigens/Antigenic Polypeptides

In some embodiments, an antigenic polypeptide includes gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. Polypeptides may also comprise single chain polypeptides or multichain polypeptides, such as antibodies or insulin, and may be associated or linked to each other. Most commonly, disulfide linkages are found in multichain polypeptides. The term “polypeptide” may also apply to amino acid polymers in which at least one amino acid residue is an artificial chemical analogue of a corresponding naturally-occurring amino acid.

A “polypeptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

“Analogs” is meant to include polypeptide variants that differ by one or more amino acid alterations, for example, substitutions, additions or deletions of amino acid residues that still maintain one or more of the properties of the parent or starting polypeptide.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, 96%, 97%, 98%, 99% or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997).” Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules) and/or between polypeptide molecules. Polymeric molecules (e.g. nucleic acid molecules (e.g. DNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12, 387 (1984)), BLASTP, BLASTN, and FASTA Altschul, S. F. et al., J. Molec. Biol., 215, 403 (1990)).

### Signal Peptides

In some embodiments, antigenic polypeptides comprise a signal peptide. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. ER processing produces mature proteins, wherein the signal peptide is cleaved from precursor proteins, typically by a ER-resident signal peptidase of the host cell, or they remain uncleaved and function as a membrane anchor. As referred herein, “mature amino acid sequence” does not contain the signal peptide sequence.

### Methods of Treatment

Provided herein are compositions (e.g., pharmaceutical compositions), methods, kits and reagents for prevention and/or treatment of influenza virus in humans and other mammals. Influenza virus vaccines can be used as therapeutic or prophylactic agents. They may be used in medicine to prevent and/or treat infectious disease. In exemplary aspects, the influenza virus vaccines of the present disclosure are used to provide prophylactic protection from influenza virus. Prophylactic protection from influenza virus can be achieved following administration of an influenza virus vaccine of the present disclosure. Vaccines can be administered once, twice, three times, four times or more. It is possible, although less desirable, to administer the vaccine to an infected individual to achieve a therapeutic response. Dosing may need to be adjusted accordingly.

In some embodiments, the influenza virus vaccines of the present disclosure can be used as a method of preventing an influenza virus infection in a subject, the method comprising administering to said subject at least one influenza virus vaccine as provided herein. In some embodiments, the influenza virus vaccines of the present disclosure can be used as a method of inhibiting a primary influenza virus infection in a subject, the method comprising administering to said subject at least one influenza virus vaccine as provided herein. In some embodiments, the influenza virus vaccines of the present disclosure can be used as a method of treating an influenza virus infection in a subject, the method comprising administering to said subject at least one influenza virus vaccine as provided herein. In some embodiments, the influenza virus vaccines of the present disclosure can be used as a method of reducing an incidence of influenza virus infection in a subject, the method comprising administering to said subject at least one influenza virus vaccine as provided herein. In some embodiments, the influenza virus vaccines of the present disclosure can be used as a method of inhibiting spread of influenza virus from a first subject infected with influenza virus to a second subject not infected with influenza virus, the method comprising administering to at least one of said first subject sand said second subject at least one influenza virus vaccine as provided herein.

A method of eliciting an immune response in a subject against an influenza virus is provided in aspects of the invention. The method involves administering to the subject an influenza virus vaccine described herein, thereby inducing in the subject an immune response specific to influenza virus antigenic polypeptide or an immunogenic fragment thereof.

A prophylactically effective dose is a therapeutically effective dose that prevents infection with the virus at a clinically acceptable level. In some embodiments the therapeutically effective dose is a dose listed in a package insert for the vaccine.

### Therapeutic and Prophylactic Compositions

Provided herein are compositions (e.g., pharmaceutical compositions), methods, kits and reagents for prevention, treatment or diagnosis of influenza in humans and other mammals, for example. Influenza virus vaccines can be used as therapeutic or prophylactic agents. They may be used in medicine to prevent and/or treat infectious disease. In some embodiments, the respiratory vaccines of the present disclosure are used for the priming of immune effector cells, for example, to activate peripheral blood mononuclear cells (PBMCs) ex vivo, which are then infused (re-infused) into a subject. In some embodiments, vaccines in accordance with the present disclosure may be used for treatment of Influenza.

Influenza virus vaccines may be administered prophylactically or therapeutically as part of an active immunization scheme to healthy individuals or early in infection during the incubation phase or during active infection after onset of symptoms. In some embodiments, the amount of vaccine of the present disclosure provided to a cell, a tissue or a subject may be an amount effective for immune prophylaxis.

Influenza virus vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In some embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, influenza virus vaccines may be administered intramuscularly, intradermally, or intranasally, similarly to the administration of inactivated vaccines known in the art. In some embodiments, influenza virus vaccines are administered intramuscularly.

Influenza virus vaccines may be utilized in various settings depending on the prevalence of the infection or the degree or level of unmet medical need. As a non-limiting example, the Vaccines may be utilized to treat and/or prevent a variety of influenzas. Vaccines have superior properties in that they produce much larger antibody titers and produce responses early than commercially available anti-viral agents/compositions.

Provided herein are pharmaceutical compositions including influenza virus vaccines optionally in combination with one or more pharmaceutically acceptable excipients.

Influenza virus vaccines may be formulated or administered alone or in conjunction with one or more other components. For instance, Influenza virus vaccines (vaccine compositions) may comprise other components including, but not limited to, adjuvants.

In some embodiments, influenza vaccines do not include an adjuvant (they are adjuvant free).

Aluminium has long been shown to stimulate the immune response against co-administered antigens, primarily by stimulating a TH2 response. It is preferred that the aluminium adjuvant of the compositions provided herein is not in the form of an aluminium precipitate. Aluminium-precipitated vaccines may increase the immune response to a target antigen, but have been shown to be highly heterogeneous preparations and have had inconsistent results {see Lindblad E. B. Immunology and Cell Biology 82: 497-505 (2004)). Aluminium-adsorbed vaccines, in contrast, can be preformed in a standardized manner, which is an essential characteristic of vaccine preparations for administration into humans. Moreover, it is thought that physical adsorption of a desired antigen onto the aluminium adjuvant has an important role in adjuvant function, perhaps in part by allowing a slower clearing from the injection site or by allowing a more efficient uptake of antigen by antigen presenting cells.

The aluminium adjuvant of the present invention may be in the form of aluminium hydroxide (Al(OH)₃), aluminium phosphate (AlPO₄), aluminium hydroxyphosphate, amorphous aluminium hydroxyphosphate sulfate (AAHS) or so-called “alum” (KA1(S04)-12H20) {see Klein et al, Analysis of aluminium hydroxyphosphate vaccine adjuvants by (27)A1 MAS NMR., J. Pharm. Sci. 89(3): 311-21 (2000)). In exemplary embodiments of the invention provided herein, the aluminium adjuvant is aluminium hydroxyphosphate or AAHS. The ratio of phosphate to aluminium in the aluminium adjuvant can range from 0 to 1.3. In preferred embodiments of this aspect of the invention, the phosphate to aluminium ratio is within the range of 0.1 to 0.70. In particularly preferred embodiments, the phosphate to aluminium ratio is within the range of 0.2 to 0.50. APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a target aggregate particle size in the range of 2-8 tm. The product is then diafiltered against physiological saline and steam sterilized. See, e.g., International Patent Application Publication No. WO2013/078102.

In some embodiments of the invention, the aluminium adjuvant is in the form of AAHS (referred to interchangeably herein as Merck aluminium adjuvant (MAA)). MAA carries zero charge at neutral pH, while AlOH carries a net positive charge and AlPO₄ typically carries a net negative charge at neutral pH.

One of skill in the art will be able to determine an optimal dosage of aluminium adjuvant that is both safe and effective at increasing the immune response to the targeted antigenic polypeptides. For a discussion of the safety profile of aluminium, as well as amounts of aluminium included in FDA-licensed vaccines, see Baylor et al., Vaccine 20: S18-S23 (2002). Generally, an effective and safe dose of aluminium adjuvant varies from 150 to 600 μg/dose (300 to 1200 μg/mL concentration). In specific embodiments of the formulations and compositions of the present invention, there is between 200 and 300 μg aluminium adjuvant per dose of vaccine. In alternative embodiments of the formulations and compositions of the present invention, there is between 300 and 500 μg aluminium adjuvant per dose of vaccine.

Influenza virus vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety). In some embodiments, influenza virus vaccines are administered to humans, human patients or subjects.

Formulations of the influenza vaccine compositions described herein may be prepared by any method known or hereafter developed in the art of pharmacology. In general, such preparatory methods include the step of bringing the active ingredient (e.g., polypeptide or polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Modes of Vaccine Administration

Influenza vaccines may be administered by any route which results in a therapeutically effective outcome. These include, but are not limited, to intradermal, intramuscular, intranasal and/or subcutaneous administration. The present disclosure provides methods comprising administering vaccines to a subject in need thereof. The exact amount required will vary from subject to subject, depending on the species, age, and general condition of the subject, the severity of the disease, the particular composition, its mode of administration, its mode of activity, and the like. Influenza vaccines compositions are typically formulated in dosage unit form for ease of administration and uniformity of dosage. It will be understood, however, that the total daily usage of vaccine compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, influenza vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see, e.g., the range of unit doses described in International Publication No WO2013078199, the contents of which are herein incorporated by reference in their entirety). The desired dosage may be delivered three times a day, two times a day, once a day, every other day, every third day, every week, every two weeks, every three weeks, every four weeks, every 2 months, every three months, every 6 months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, influenza vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, influenza vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

An influenza vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, intranasal and subcutaneous).

### Influenza Virus Vaccine Formulations and Methods of Use

Some aspects of the present disclosure provide formulations of the influenza vaccine, wherein the vaccine is formulated in an effective amount to produce an antigen specific immune response in a subject (e.g., production of antibodies specific to an influenza antigenic polypeptide). “An effective amount” is a dose of a vaccine effective to produce an antigen-specific immune response. Also provided herein are methods of inducing an antigen-specific immune response in a subject.

In some embodiments, the antigen-specific immune response is characterized by measuring an anti- influenza antigenic polypeptide antibody titer produced in a subject administered an influenza vaccine as provided herein. An antibody titer is a measurement of the amount of antibodies within a subject, for example, antibodies that are specific to a particular antigen (e.g., an influenza antigenic polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the influenza vaccine.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### Examples

The first underlined sequence for each of the amino acid sequences listed in Table 1, indicates a signal or secretory sequence, which may be substituted by an alternative sequence that achieves the same or similar function, or the signal or secretory sequence may be deleted. Other underlined sequences for the amino acid sequences listed in Table 1, indicates a foldon sequence, which is a heterologous sequence that naturally trimerizes, to bring 3 HA stems together in a trimer; a ferritin; or transmembrane sequence. Such foldon, ferritin or transmembrane sequence may be substituted by an alternative sequence, which achieves the same or similar function.

TABLE 1
Antigenic Polypeptide Sequences

| Name | Sequence | SEQ ID NO: |
|---|---|---|
| BHA10-2: HA10 version for | METPAQLLFLLLLWLPDTTGHVVKTATQGEVNVT | 1 |
| Influenza | GVIPLTTTPTGSANKSKPYYTGEHAKATGNCPIW | |
| B/Brisbane/60/2008 strain, | VKTPLKLANGTKYGSAGSATQEAINKITKNLNSL | |
| with exposed hydrophobic | SELEVKNLQRLSGASDETHNEILELDEKVDDLRA | |
| residues mutated I333T, | DTISSQIELAVLLSNEGIINSEDEGTGGGYIPEA | |
| M432S, L435T | PRDGQAYVRKDGEWVLLSTFL | |
| (bold/underlined) and foldon | | |
| sequence (second underlined) | | |
| BHA10-3: BHA10-2 without | METPAQLLFLLLLWLPDTTGHVVKTATQGEVNVT | 2 |
| GTGG linker or foldon | GVIPLTTTPTGSANKSKPYYTGEHAKATGNCPIW | |
| domain, with G430C, | VKTPLKLANGTKYGSAGSATQEAINKITKNLNSL | |
| E438C, Q457L mutations | SELEVKNLQRLSCASDETHNCILELDEKVDDLRA | |
| (bold) for trimerization | DTISSLIELAVLLSNEGIINSEDE | |
| NIHGen6HASS-TM: Gen6 | METPAQLLFLLLLWLPDTTGDTICIGYHANNSTD | 3 |
| HASS construct without | TVDTVLEKNVTVTHSVNLGSGLRMVTGLRNIPQR | |
| foldon or ferritin, linker | ETRGLFGAIAGFIEGGWTGMVDGWYGYHHQNEQG | |
| (bold) with transmembrane | SGYAADQKSTQNAINGITNMVNSVIEKMGSGGSG | |
| domain (second underlined), | TDLAELLVLLLNERTLDFHDSNVKNLYEKVKSQL | |
| version 1 | KNNAKEIGNGCFETYHKCNNECMESVKNGTYDYP | |
| | KYSEESKLNREKIDQGTGGILAIYSTVASSLVLL | |
| | VSLGAISFWMCSNGSLQCRICI | |
| NIHGen6HASS-TM2: Gen6 | METPAQLLFLLLLWLPDTTGDTICIGYHANNSTD | 4 |
| HASSconstruct without | TVDTVLEKNVTVTHSVNLGSGLRMVTGLRNIPQR | |
| foldon or ferritin, linker | ETRGLFGAIAGFIEGGWTGMVDGWYGYHHQNEQG | |
| (bold), with transmembrane | SGYAADQKSTQNAINGITNMVNSVIEKMGSGGSG | |
| domain (second underlined), | TDLAELLVLLLNERTLDFHDSNVKNLYEKVKSQL | |
| version 2 | KNNAKEIGNGCFETYHKCNNECMESVKNGTYDYP | |
| | KYSEESKLNREKIDGVKLESMGVYQILAIYSTVA | |
| | SSLVLLVSLGAISFWMCSNGSLQCRICI | |
| NIHGen6HASS-foldon: | METPAQLLFLLLLWLPDTTGDTICIGYHANNSTD | 5 |
| Gen6 HASSconstruct with | TVDTVLEKNVTVTHSVNLGSGLRMVTGLRNIPQR | |
| foldon sequence (second | ETRGLFGAIAGFIEGGWTGMVDGWYGYHHQNEQG | |
| underlined) | SGYAADQKSTQNAINGITNMVNSVIEKMGSGGSG | |
| | TDLAELLVLLLNERTLDFHDSNVKNLYEKVKSQL | |
| | KNNAKEIGNGCFETYHKCNNECMESVKNGTYDYP | |
| | KYSEESKLNREKIDPGSGYIPEAPRDGQAYVRKD | |
| | GEWVLLSTFL | |
| ConH1: consensus HA | MKAKLLVLLCAFTATDADTICIGYHANNSTDTVD | 6 |
| sequence for subtype H1 | TVLEKNVTVTHSVNLLEDSHNGKLCKLKGIAPLQ | |
| with transmembrane domain | LGKCNIAGWILGNPECESLISKRSWSYIVETPNS | |
| (second underlined) | ENGTCYPGDFADYEELREQLSSVSSFERFEIFPK | |
| | ESSWPNHNVTKGVTAACSHAGKSSFYRNLLWLTE | |
| | KNGSYPKLSKSYVNNKEKEVLVLWGVHHPSNITD | |
| | QRTLYQNENAYVSVVSSHYNRRFTPEIAKRPKVR | |
| | GQAGRINYYWTLLEPGDTIIFEANGNLIAPWYAF | |
| | ALSRGFGSGITTSNAPMHECDTKCQTPQGAINSS | |
| | LPFQNVHPVTIGECPKYVRSTKLRMVTGLRNIPS | |
| | IQSRGLFGAIAGFIEGGWTGMIDGWYGYHHQNEQ | |
| | GSGYAADQKSTQNAINGITNKVNSVIEKMNTQFT | |
| | AVGKEFNKLEKRMENLNKKVDDGFLDIWTYNAEL | |
| | LVLLENERTLDFHDSNVKNLYEKVKSQLKNNAKE | |
| | IGNGCFEFYHKCNNECMESVKNGTYDYPKYSEES | |
| | KLNREKIDGVKLESMGVYQILAIYSTVASSLVLL | |
| | VSLGAISFWMCSNGSLQCRICI | |
| ConH3: consensus HA | MKTIIALSYIFCLVFAQKLPGNDNSTATLCLGHH | 7 |
| sequence for subtype H3 | AVPNGTLVKTITNDQIEVTNATELVQSSSTGRIC | |
| with transmembrane domain | DSPHRILDGTNCTLIDALLGDPHCDGFQNKEWDL | |
| (second underlined) | FVERSKAYSNCYPYDVPDYASLRSLVASSGTLEF | |
| | NNEGFNWTGVTQNGGSSACKRGSDKSFFSRLNWL | |
| | HKLKYKYPALNVTMPNNDKFDKLYIWGVHHPSTD | |
| | SDQTSLYVQASGRVTVSTKRSQQTVIPNIGSRPW | |
| | VRGLSSRISIYWTIVKPGDILLINSTGNLIAPRG | |
| | YFKIRSGKSSIMRSDAPIGTCNSECITPNGSIPN | |
| | DKPFQNVNRITYGACPRYVKQNTLKLATGMRNVP | |
| | EKQTRGIFGAIAGFIENGWEGMVDGWYGFRHQNS | |
| | EGTGQAADLKSTQAAIDQINGKLNRLIEKTNEKF | |
| | HQIEKEFSEVEGRIQDLEKYVEDTKIDLWSYNAE | |
| | LLVALENQHTIDLTDSEMNKLFERTRKQLRENAE | |
| | DMGNGCFKIYHKCDNACIGSIRNGTYDHDVYRDE | |
| | ALNNRFQIKGVELKSGYKDWILWISFAISCFLLC | |
| | VVLLGFIMWACQKGNIRCNICI | |
| MRK_pH1_Con: consensus | MKAILVVLLYTFATANADTLCIGYHANNSTDTVD | 8 |
| HA sequence for pandemic | TVLKNVTVTHSVNLLEDKHNGKLCKLRGVAPLHL | |
| H1 strains, includes | GKCNIAGWILGNPECESLSTASSWSYIVETSSSD | |
| transmembrane sequence | NGTCYPGDFIDYEELREQLSSVSSFERFEIFPKT | |
| (second underlined) | SSWPNHDSNKGVTAACPHAGAKSFYKNLIWLVKK | |
| | GNSYPKLSKSYINDKGKEVLVLWGIHHPSTSADQ | |
| | QSLYQNADAYVFVGTSRYSKKFKPEIAIRPKVRD | |
| | QEGRMNYYWTLVEPGDKITFEATGNLVVPRYAFA | |
| | MERNAGSGIIISDTPVHDCNTTCQTPKGAINTSL | |
| | PFQNIHPITIGKCPKYVKSTKLRLATGLRNVPSI | |
| | QSRGLFGAIAGFIEGGWTGMVDGWYGYHHQNEQG | |
| | SGYAADLKSTQNAIDKITNKVNSVIEKMNTQFTA | |
| | VGKEFNHLEKRIENLNKKVDDGFLDIWTYNAELL | |
| | VLLENERTLDYHDSNVKNLYEKVRSQLKNNAKEI | |
| | GNGCFEFYHKCDNTCMESVKNGTYDYPKYSEEAK | |
| | LNREEIDGVKLESTRIYQILAIYSTVASSLVLVV | |
| | SLGAISFWMCSNGSLQCRICI | |
| MRK_pH1_Con: consensus | MKAILVVLLYTFATANADTLCIGYHANNSTDTVD | 9 |
| HA sequence for pandemic | TVLKNVTVTHSVNLLEDKHNGKLCKLRGVAPLHL | |
| H1 strains, extracellular | GKCNIAGWILGNPECESLSTASSWSYIVETSSSD | |
| domain (italics indicate | NGTCYPGDFIDYEELREQLSSVSSFERFEIFPKT | |
| other truncation sites for | SSWPNHDSNKGVTAACPHAGAKSFYKNLIWLVKK | |
| extracellular domain) | GNSYPKLSKSYINDKGKEVLVLWGIHHPSTSADQ | |
| | QSLYQNADAYVFVGTSRYSKKFKPEIAIRPKVRD | |
| | QEGRMNYYWTLVEPGDKITFEATGNLVVPRYAFA | |
| | MERNAGSGIIISDTPVHDCNTTCQTPKGAINTSL | |
| | PFQNIHPITIGKCPKYVKSTKLRLATGLRNVPSI | |
| | QSRGLFGAIAGFIEGGWTGMVDGWYGYHHQNEQG | |
| | SGYAADLKSTQNAIDKITNKVNSVIEKMNTQFTA | |
| | VGKEFNHLEKRIENLNKKVDDGFLDIWTYNAELL | |
| | VLLENERTLDYHDSNVKNLYEKVRSQLKNNAKEI | |
| | GNGCFEFYHKCDNTCMESVKNGTYDYPKYSEEAK | |
| | LNREEIDGVKLESTRIYQ | |
| MRK_sH1_Con: consensus | MKVKLLVLLCTFTATYADTICIGYHANNSTDTVD | 10 |
| HA sequence for seasonal | TVLEKNVTVTHSVNLLEDSHNGKLCLLKGIAPLQ | |
| H1 strains, includes | LGNCSVAGWILGNPECELLISKESWSYIVETPNP | |
| transmembrane sequence | ENGTCYPGYFADYEELREQLSSVSSFERFEIFPK | |
| (second underlined) | ESSWPNHTVTGVSASCSHNGKSSFYRNLLWLTGK | |
| | NGLYPNLSKSYANNKEKEVLVLWGVHHPPNIGDQ | |
| | RALYHTENAYVSVVSSHYSRRFTPEIAKRPKVRD | |
| | QEGRINYYWTLLEPGDTIIFEANGNLIAPRYAFA | |
| | LSRGFGSGIITSNAPMDECDAKCQTPQGAINSSL | |
| | PFQNVHPVTIGECPKYVRSAKLRMVTGLRNIPSI | |
| | QSRGLFGAIAGFIEGGWTGMVDGWYGYHHQNEQG | |
| | SGYAADQKSTQNAINGITNKVNSVIEKMNTQFTA | |
| | VGKEFNKLERRMENLNKKVDDGFLDIWTYNAELL | |
| | VLLENERTLDFHDSNVKNLYEKVKSQLKNNAKEI | |
| | GNGCFEFYHKCNDECMESVKNGTYDYPKYSEESK | |
| | LNREKIDGVKLESMGVYQILAIYSTVASSLVLLV | |
| | SLGAISFWMCSNGSLQCRICI | |
| MRK_sH1_Con: consensus | MKVKLLVLLCTFTATYADTICIGYHANNSTDTVD | 11 |
| HA sequence for seasonal | TVLEKNVTVTHSVNLLEDSHNGKLCLLKGIAPLQ | |
| H1 strains, extracellular | LGNCSVAGWILGNPECELLISKESWSYIVETPNP | |
| domain (italics indicate | ENGTCYPGYFADYEELREQLSSVSSFERFEIFPK | |
| other truncation sites for | ESSWPNHTVTGVSASCSHNGKSSFYRNLLWLTGK | |
| extracellular domain) | NGLYPNLSKSYANNKEKEVLVLWGVHHPPNIGDQ | |
| | RALYHTENAYVSVVSSHYSRRFTPEIAKRPKVRD | |
| | QEGRINYYWTLLEPGDTIIFEANGNLIAPRYAFA | |
| | LSRGFGSGIITSNAPMDECDAKCQTPQGAINSSL | |
| | PFQNVHPVTIGECPKYVRSAKLRMVTGLRNIPSI | |
| | QSRGLFGAIAGFIEGGWTGMVDGWYGYHHQNEQG | |
| | SGYAADQKSTQNAINGITNKVNSVIEKMNTQFTA | |
| | VGKEFNKLERRMENLNKKVDDGFLDIWTYNAELL | |
| | VLLENERTLDFHDSNVKNLYEKVKSQLKNNAKEI | |
| | GNGCFEFYHKCNDECMESVKNGTYDYPKYSEESK | |
| | LNREKIDGVKLESMGVYQ | |
| Cobra_P1: consensus HA | MKARLLVLLCALAATDADTICIGYHANNSTDTVD | 12 |
| sequence P1 for H1 subtype | TVLEKNVTVTHSVNLLEDSHNGKLCKLKGIAPLQ | |
| with transmembrane domain | LGKCNIAGWLLGNPECESLLSARSWSYIVETPNS | |
| (second underlined) | ENGTCYPGDFIDYEELREQLSSVSSFERFEIFPK | |
| | ESSWPNHNTTKGVTAACSHAGKSSFYRNLLWLIK | |
| | KGGSYPKLSKSYVNNKGKEVLVLWGVHHPSTSTD | |
| | QQSLYQNENAYVSVVSSNYNRRFTPEIAERPKVR | |
| | GQAGRMNYYWTLLEPGDTIIFEATGNLIAPWYAF | |
| | ALSRGSGSGIITSNASMHECNTKCQTPQGAINSS | |
| | LPFQNIHPVTIGECPKYVRSTKLRMVTGLRNIPS | |
| | IQSRGLFGAIAGFIEGGWTGMIDGWYGYHHQNEQ | |
| | GSGYAADQKSTQNAINGITNKVNSVIEKMNTQFT | |
| | AVGKEFNNLEKRMENLNKKVDDGFLDIWTYNAEL | |
| | LVLLENERTLDFHDSNVKNLYEKVKSQLRNNAKE | |
| | IGNGCFETYHKCDNECMESVKNGTYDYPKYSEES | |
| | KLNREKIDGVKLESMGVYQILAIYSTVASSLVLL | |
| | VSLGAISFWMCSNGSLQCRICI | |
| Cobra_X3: consensus HA | MEARLLVLLCAFAATNADTICIGYHANNSTDTVD | 13 |
| sequence X3 for H1 subtype | TVLEKNVTVTHSVNLLEDSHNGKLCRLKGIAPLQ | |
| with transmembrane domain | LGNCSVAGWILGNPECESLFSKESWSYIAETPNP | |
| (second underlined) | ENGTCYPGYFADYEELREQLSSVSSFERFEIFPK | |
| | ESSWPNHTVTKGVTASCSHNGKSSFYRNLLWLTE | |
| | KNGLYPNLSKSYVNNKEKEVLVLWGVHHPSNIGD | |
| | QRAIYHTENAYVSVVSSHYSRRFTPEIAKRPKVR | |
| | DQEGRINYYWTLLEPGDTIIFEANGNLIAPWYAF | |
| | ALSRGFGSGIITSNASMDECDAKCQTPQGAINSS | |
| | LPFQNVHPVTIGECPKYVRSTKLRMVTGLRNIPS | |
| | IQSRGLFGAIAGFIEGGWTGMIDGWYGYHHQNEQ | |
| | GSGYAADQKSTQNAINGITNKVNSVIEKMNTQFT | |
| | AVGKEFNKLERRMENLNKKVDDGFLDIWTYNAEL | |
| | LVLLENERTLDFHDSNVKNLYEKVKSQLKNNAKE | |
| | IGNGCFEFYHKCNNECMESVKNGTYDYPKYSEES | |
| | KLNREKIDGVKLESMGVYQILAIYSTVASSLVLL | |
| | VSLGAISFWMCSNGSLQCRICI | |
| ConH1_ferritin: consensus | MKAKLLVLLCAFTATDADTICIGYHANNSTDTVD | 14 |
| HA sequence for subtype | TVLEKNVTVTHSVNLLEDSHNGKLCKLKGIAPLQ | |
| H1, linker (bold), with | LGKCNIAGWILGNPECESLISKRSWSYIVETPNS | |
| ferritin for particle | ENGTCYPGDFADYEELREQLSSVSSFERFEIFPK | |
| formation (second | ESSWPNHNVTKGVTAACSHAGKSSFYRNLLWLTE | |
| underlined) | KNGSYPKLSKSYVNNKEKEVLVLWGVHHPSNITD | |
| | QRTLYQNENAYVSVVSSHYNRRFTPEIAKRPKVR | |
| | GQAGRINYYWTLLEPGDTIIFEANGNLIAPWYAF | |
| | ALSRGFGSGITTSNAPMHECDTKCQTPQGAINSS | |
| | LPFQNVHPVTIGECPKYVRSTKLRMVTGLRNIPS | |
| | IQSRGLFGAIAGFIEGGWTGMIDGWYGYHHQNEQ | |
| | GSGYAADQKSTQNAINGITNKVNSVIEKMNTQFT | |
| | AVGKEFNKLEKRMENLNKKVDDGFLDIWTYNAEL | |
| | LVLLENERTLDFHDSNVKNLYEKVKSQLKNNAKE | |
| | IGNGCFEFYHKCNNECMESVKNGTYDYPKYSEES | |
| | KLNREKIDSGGDIIKLLNEQVNKEMQSSNLYMSM | |
| | SSWCYTHSLDGAGLFLFDHAAEEYEHAKKLIIFL | |
| | NENNVPVQLTSISAPEHKEEGLTQIFQKAYEHEQ | |
| | HISESINNIVDHAIKSKDHATFNFLQWYVAEQHE | |
| | EEVLFKDILDKIELIGNENHGLYLADQYVKGIAK | |
| | SRKS | |
| ConH3_ferritin: consensus | MKTIIALSYIFCLVFAQKLPGNDNSTATLCLGHH | 15 |
| HA sequence for subtype | AVPNGTLVKTITNDQIEVTNATELVQSSSTGRIC | |
| H3, linker (bold), with | DSPHRILDGTNCTLIDALLGDPHCDGFQNKEWDL | |
| ferritin for particle | FVERSKAYSNCYPYDVPDYASLRSLVASSGTLEF | |
| formation (second | NNEGFNWTGVTQNGGSSACKRGSDKSFFSRLNWL | |
| underlined) | HKLKYKYPALNVTMPNNDKFDKLYIWGVHHPSTD | |
| | SDQTSLYVQASGRVTVSTKRSQQTVIPNIGSRPW | |
| | VRGLSSRISIYWTIVKPGDILLINSTGNLIAPRG | |
| | YFKIRSGKSSIMRSDAPIGTCNSECITPNGSIPN | |
| | DKPFQNVNRITYGACPRYVKQNTLKLATGMRNVP | |
| | EKQTRGIFGAIAGFIENGWEGMVDGWYGFRHQNS | |
| | EGTGQAADLKSTQAAIDQINGKLNRLIEKTNEKF | |
| | HQIEKEFSEVEGRIQDLEKYVEDTKIDLWSYNAE | |
| | LLVALENQHTIDLTDSEMNKLFERTRKQLRENAE | |
| | DMGNGCFKIYHKCDNACIGSIRNGTYDHDVYRDE | |
| | ALNNRFQIKSGGDIIKLLNEQVNKEMQSSNLYMS | |
| | MSSWCYTHSLDGAGLFLFDHAAEEYEHAKKLIIF | |
| | LNENNVPVQLTSISAPEHKFEGLTQIFQKAYEHE | |
| | QHISESINNIVDHAIKSKDHATFNFLQWYVAEQH | |
| | EEEVLFKDILDKIELIGNENHGLYLADQYVKGIA | |
| | KSRKS | |
| Merck_pH1_Con_ferritin: | MKAILVVLLYTFATANADTLCIGYHANNSTDTVD | 16 |
| consensus HA sequence for | TVLEKNVTVTHSVNLLEDKHNGKLCKLRGVAPLH | |
| pandemic H1 strains, linker | LGKCNIAGWILGNPECESLSTASSWSYIVETSSS | |
| (bold), with ferritin for | DNGTCYPGDFIDYEELREQLSSVSSFERFEIFPK | |
| particle formation (second | TSSWPNHDSNKGVTAACPHAGAKSFYKNLIWLVK | |
| underlined) | KGNSYPKLSKSYINDKGKEVLVLWGIHHPSTSAD | |
| | QQSLYQNADAYVFVGTSRYSKKFKPEIAIRPKVR | |
| | DQEGRMNYYWTLVEPGDKITFEATGNLVVPRYAF | |
| | AMERNAGSGIIISDTPVHDCNTTCQTPKGAINTS | |
| | LPFQNIHPITIGKCPKYVKSTKLRLATGLRNVPS | |
| | IQSRGLFGAIAGFIEGGWTGMVDGWYGYHHQNEQ | |
| | GSGYAADLKSTQNAIDKITNKVNSVIEKMNTQFT | |
| | AVGKEFNHLEKRIENLNKKVDDGFLDIWTYNAEL | |
| | LVLLENERTLDYHDSNVKNLYEKVRSQLKNNAKE | |
| | IGNGCFEFYHKCDNTCMESVKNGTYDYPKYSEEA | |
| | KLNREEIDSGGDIIKLLNEQVNKEMQSSNLYMSM | |
| | SSWCYTHSLDGAGLFLFDHAAEEYEHAKKLIIFL | |
| | NENNVPVQLTSISAPEHKFEGLTQIFQKAYEHEQ | |
| | HISESINNIVDHAIKSKDHATFNFLQWYVAEQHE | |
| | EEVLFKDILDKIELIGNENHGLYLADQYVKGIAK | |
| | SRKS | |
| Merck_sH1_Con_ferritin: | MKVKLLVLLCTFTATYADTICIGYHANNSTDTVD | 17 |
| consensus HA sequence for | TVLEKNVTVTHSVNLLEDSHNGKLCLLKGIAPLQ | |
| seasonal H1 strains, linker | LGNCSVAGWILGNPECELLISKESWSYIVETPNP | |
| (bold), with ferritin for | ENGTCYPGYFADYEELREQLSSVSSFERFEIFPK | |
| particle formation (second | ESSWPNHTVTGVSASCSHNGKSSFYRNLLWLTGK | |
| underlined) | NGLYPNLSKSYANNKEKEVLVLWGVHHPPNIGDQ | |
| | RALYHTENAYVSVVSSHYSRRFTPEIAKRPKVRD | |
| | QEGRINYYWTLLEPGDTIIFEANGNLIAPRYAFA | |
| | LSRGFGSGIITSNAPMDECDAKCQTPQGAINSSL | |
| | PFQNVHPVTIGECPKYVRSAKLRMVTGLRNIPSI | |
| | QSRGLFGAIAGFIEGGWTGMVDGWYGYHHQNEQG | |
| | SGYAADQKSTQNAINGITNKVNSVIEKMNTQFTA | |
| | VGKEFNKLERRMENLNKKVDDGFLDIWTYNAELL | |
| | VLLENERTLDFHDSNVKNLYEKVKSQLKNNAKEI | |
| | GNGCFEFYHKCNDECMESVKNGTYDYPKYSEESK | |
| | LNREKIDSGGDIIKLLNEQVNKEMQSSNLYMSMS | |
| | SWCYTHSLDGAGLFLFDHAAEEYEHAKKLIIFLN | |
| | ENNVPVQLTSISAPEHKFEGLTQIFQKAYEHEQH | |
| | ISESINNIVDHAIKSKDHATFNFLQWYVAEQHEE | |
| | EVLFKDILDKIELIGNENHGLYLADQYVKGIAKS | |
| | RKS | |
| Cobra_P1_ferritin: | MKARLLVLLCALAATDADTICIGYHANNSTDTVD | 18 |
| consensus HA sequence P1 | TVLEKNVTVTHSVNLLEDSHNGKLCKLKGIAPLQ | |
| for H1 subtype, linker | LGKCNIAGWLLGNPECESLLSARSWSYIVETPNS | |
| (bold), with ferritin for | ENGTCYPGDFIDYEELREQLSSVSSFERFEIFPK | |
| particle formation (second | ESSWPNHNTTKGVTAACSHAGKSSFYRNLLWLIK | |
| underlined) | KGGSYPKLSKSYVNNKGKEVLVLWGVHHPSTSTD | |
| | QQSLYQNENAYVSVVSSNYNRRFTPEIAERPKVR | |
| | GQAGRMNYYWTLLEPGDTIIFEATGNLIAPWYAF | |
| | ALSRGSGSGIITSNASMHECNTKCQTPQGAINSS | |
| | LPFQNIHPVTIGECPKYVRSTKLRMVTGLRNIPS | |
| | IQSRGLFGAIAGFIEGGWTGMIDGWYGYHHQNEQ | |
| | GSGYAADQKSTQNAINGITNKVNSVIEKMNTQFT | |
| | AVGKEFNNLEKRMENLNKKVDDGFLDIWTYNAEL | |
| | LVLLENERTLDFHDSNVKNLYEKVKSQLRNNAKE | |
| | IGNGCFETYHKCDNECMESVKNGTYDYPKYSEES | |
| | KLNREKIDSGGDIIKLLNEQVNKEMQSSNLYMSM | |
| | SSWCYTHSLDGAGLFLFDHAAEEYEHAKKLIIFL | |
| | NENNVPVQLTSISAPEHKFEGLTQIFQKAYEHEQ | |
| | HISESINNIVDHAIKSKDHATFNFLQWYVAEQHE | |
| | EEVLFKDILDKIELIGNENHGLYLADQYVKGIAK | |
| | SRKS | |
| Cobra_X3_ferritin: | MEARLLVLLCAFAATNADTICIGYHANNSTDTVD | 19 |
| consensus HA sequence X3 | TVLEKNVTVTHSVNLLEDSHNGKLCRLKGIAPLQ | |
| for H1 subtype, linker | LGNCSVAGWILGNPECESLFSKESWSYIAETPNP | |
| (bold), with ferritin for | ENGTCYPGYFADYEELREQLSSVSSFERFEIFPK | |
| particle formation (second | ESSWPNHTVTKGVTASCSHNGKSSFYRNLLWLTE | |
| underlined) | KNGLYPNLSKSYVNNKEKEVLVLWGVHHPSNIGD | |
| | QRAIYHTENAYVSVVSSHYSRRFTPEIAKRPKVR | |
| | DQEGRINYYWTLLEPGDTIIFEANGNLIAPWYAF | |
| | ALSRGFGSGIITSNASMDECDAKCQTPQGAINSS | |
| | LPFQNVHPVTIGECPKYVRSTKLRMVTGLRNIPS | |
| | IQSRGLFGAIAGFIEGGWTGMIDGWYGYHHQNEQ | |
| | GSGYAADQKSTQNAINGITNKVNSVIEKMNTQFT | |
| | AVGKEFNKLERRMENLNKKVDDGFLDIWTYNAEL | |
| | LVLLENERTLDFHDSNVKNLYEKVKSQLKNNAKE | |
| | IGNGCFEFYHKCNNECMESVKNGTYDYPKYSEES | |
| | KLNREKIDSGGDIIKLLNEQVNKEMQSSNLYMSM | |
| | SSWCYTHSLDGAGLFLFDHAAEEYEHAKKLIIFL | |
| | NENNVPVQLTSISAPEHKFEGLTQIFQKAYEHEQ | |
| | HISESINNIVDHAIKSKDHATFNFLQWYVAEQHE | |
| | EEVLFKDILDKIELIGNENHGLYLADQYVKGIAK | |
| | SRKS | |
| NP: Wildtype sequence of | MASQGTKRSYEQMETDGERQNATEIRASVGKMID | 20 |
| nucleoprotein | GIGRFYIQMCTELKLSDYEGRLIQNSLTIERMVL | |
| | SAFDERRNRYLEEHPSAGKDPKKTGGPIYKRVDG | |
| | RWMRELVLYDKEEIRRIWRQANNGDDATAGLTHM | |
| | MIWHSNLNDTTYQRTRALVRTGMDPRMCSLMQGS | |
| | TLPRRSGAAGAAVKGIGTMVMELIRMIKRGINDR | |
| | NFWRGENGRKTRSAYERMCNILKGKFQTAAQRAM | |
| | MDQVRESRNPGNAEIEDLIFSARSALILRGSVAH | |
| | KSCLPACVYGPAVSSGYNFEKEGYSLVGIDPFKL | |
| | LQNSQVYSLIRPNENPAHKSQLVWMACHSAAFED | |
| | LRLLSFIRGTKVSPRGKLSTRGVQIASNENMDNM | |
| | ESSTLELRSRYWAIRTRSGGNTNQQRASAGQISV | |
| | QPTFSVQRNLPFEKSTVMAAFTGNTEGRTSDMRA | |
| | EIIRMMEGAKPEEVSFRGRGVFELSDEKATNPIV | |
| | PSFDMSNEGSYFFGDNAEEYDN | |
| MRK_H3_consUnique: | MKTIIALSYILCLVFAQKLPGNDNSTATLCLGHH | 41 |
| consensus sequence for | AVPNGTIVKTITNDQIEVTNATELVQSSSTGEIC | |
| subtype H3 with | DSPHQILDGENCTLIDALLGDPQCDGFQNKKWDL | |
| transmembrane domain | FVERSKAYSNCYPYDVPDYASLRSLVASSGTLEF | |
| (second underlined), italics | NNESFNWTGVTQNGTSSACIRRSNSSFFSRLNWL | |
| indicate possible truncation | THLNFKYPALNVTMPNNEQFDKLYIWGVHHPGTD | |
| sites for extracellular | KDQIFLYAQASGRITVSTKRSQQAVIPNIGSRPR | |
| domain | VRNIPSRISIYWTIVKPGDILLINSTGNLIAPRG | |
| | YFKIRSGKSSIMRSDAPIGKCNSECITPNGSIPN | |
| | DKPFQNVNRITYGACPRYVKQNTLKLATGMRNVP | |
| | EKQTRGIFGAIAGFIENGWEGMVDGWYGFRHQNS | |
| | EGRGQAADLKSTQAAIDQINGKLNRLIGKTNEKF | |
| | HQIEKEFSEVEGRIQDLEKYVEDTKIDLWSYNAE | |
| | LLVALENQHTIDLTDSEMNKLFEKTKKQLRENAE | |
| | DMGNGCFKIYHKCDNACIGSIRNGTYDHDVYRDE | |
| | ALNNRFQIKGVELKSGYKDWILWISFAISCFLLC | |
| | VALLGFIMWACQKGNIRCNICI | |
| MRK_H3_ConsensusA: | MKTIIALSYILCLVFAQKLPGNDNSTATLCLGHH | 42 |
| consensus sequence for | AVPNGTLVKTITNDQIEVTNATELVQSSSTGRIC | |
| subtype H3, cluster A with | DSPHRILDGENCTLIDALLGDPHCDGFQNKEWDL | |
| transmembrane domain | FVERSKAYSNCYPYDVPDYASLRSLVASSGTLEF | |
| (second underlined), italics | NNESFNWTGVAQNGTSYACKRGSVKSFFSRLNWL | |
| indicate possible truncation | HQLKYKYPALNVTMPNNDKFDKLYIWGVHHPSTD | |
| sites for extracellular | SDQTSLYVQASGRVTVSTKRSQQTVIPNIGSRPW | |
| domain | VRGVSSRISIYWTIVKPGDILLINSTGNLIAPRG | |
| | YFKIRSGKSSIMRSDAPIGKCNSECITPNGSIPN | |
| | DKPFQNVNRITYGACPRYVKQNTLKLATGMRNVP | |
| | EKQTRGIFGAIAGFIENGWEGMVDGWYGFRHQNS | |
| | EGTGQAADLKSTQAAINQINGKLNRLIEKTNEKF | |
| | HQIEKEFSEVEGRIQDLEKYVEDTKIDLWSYNAE | |
| | LLVALENQHTIDLTDSEMNKLFERTRKQLRENAE | |
| | DMGNGCFKIYHKCDNACIGSIRNGTYDHDVYRDE | |
| | ALNNRFQIKGVELKSGYKDWILWISFAISCFLLC | |
| | VVLLGFIMWACQKGNIRCNICI | |
| MRK_H3_ConsensusB: | MKTIIALSYILCLVFAQKLPGNDNSTATLCLGHH | 43 |
| consensus sequence for | AVPNGTIVKTITNDQIEVTNATELVQNSSTGEIC | |
| subtype H3, cluster B with | DSPHQILDGENCTLIDALLGDPQCDGFQNKKWDL | |
| transmembrane domain | FVERSKAYSNCYPYDVPDYASLRSLVASSGTLEF | |
| (second underlined), italics | NNESFNWTGVTQNGTSSACIRRSNSSFFSRLNWL | |
| indicate possible truncation | THLNFKYPALNVTMPNNEQFDKLYIWGVHHPGTD | |
| sites for extracellular | KDQIFLYAQSSGRITVSTKRSQQAVIPNIGSRPR | |
| domain | IRNIPSRISIYWTIVKPGDILLINSTGNLIAPRG | |
| | YFKIRSGKSSIMRSDAPIGKCNSECITPNGSIPN | |
| | DKPFQNVNRITYGACPRYVKQSTLKLATGMRNVP | |
| | EKQTRGIFGAIAGFIENGWEGMVDGWYGFRHQNS | |
| | EGRGQAADLKSTQAAIDQINGKLNRLIGKTNEKF | |
| | HQIEKEFSEVEGRIQDLEKYVEDTKIDLWSYNAE | |
| | LLVALENQHTIDLTDSEMNKLFEKTKKQLRENAE | |
| | DMGNGCFKIYHKCDNACIGSIRNGTYDHDVYRDE | |
| | ALNNRFQIKGVELKSGYKDWILWISFAISCFLLC | |
| | VALLGFIMWACQKGNIRCNICI | |
| MRK_H1_cot_all: “center | MKAILVVLLYTFATANADTLCIGYHANNSTDTVD | 44 |
| of tree” sequence for | TVLEKNVTVTHSVNLLEDKHNGKLCKLRGVAPLH | |
| subtype H1 with | LGKCNIAGWILGNPECESLSTASSWSYIVETSSS | |
| transmembrane domain | DNGTCYPGDFINYEELREQLSSVSSFERFEIFPK | |
| (second underlined), italics | TSSWPNHDSNKGVTAACPHAGAKSFYKNLIWLVK | |
| indicate possible truncation | KGNSYPKLSKSYINDKGKEVLVLWGIHHPSTTAD | |
| sites for extracellular | QQSLYQNADAYVFVGTSRYSKKFKPEIAIRPKVR | |
| domain | DQEGRMNYYWTLVEPGDKITFEATGNLVVPRYAF | |
| | AMERNAGSGIIISDTPVHDCNTTCQTPKGAINTS | |
| | LPFQNIHPITIGKCPKYVKSTKLRLATGLRNVPS | |
| | IQSRGLFGAIAGFIEGGWTGMVDGWYGYHHQNEQ | |
| | GSGYAADLKSTQNAIDKITNKVNSVIEKMNTQFT | |
| | AVGKEFNHLEKRIENLNKKVDDGFLDIWTYNAEL | |
| | LVLLENERTLDYHDSNVKNLYEKVRNQLKNNAKE | |
| | IGNGCFEFYHKCDNTCMESVKNGTYDYPKYSEEA | |
| | KLNREKIDGVKLESTRIYQILAIYSTVASSLVLV | |
| | VSLGAISFWMCSNGSLQCRICI | |
| MRK_H3_cot_all: “center | MKTIIALSYILCLVFAQKLPGNDNSTATLCLGHH | 45 |
| of tree” sequence for | AVPNGTIVKTITNDRIEVTNATELVQNSSIGEIC | |
| subtype H3 with | DSPHQILDGENCTLIDALLGDPQCDGFQNKKWDL | |
| transmembrane domain | FVERSKAYSNCYPYDVPDYASLRSLVASSGTLEF | |
| (second underlined), italics | NNESFNWTGVTQNGTSSACIRRSNSSFFSRLNWL | |
| indicate possible truncation | THLNFKYPALNVTMPNNEQFDKLYIWGVHHPGTD | |
| sites for extracellular | KDQIFLYAQSSGRITVSTKRSQQAVIPNIGSRPR | |
| domain | IRNIPSRISIYWTIVKPGDILLINSTGNLIAPRG | |
| | YFKIRSGKSSIMRSDAPIGKCKSECITPNGSIPN | |
| | DKPFQNVNRITYGACPRYVKQSTLKLATGMRNVP | |
| | EKQTRGIFGAIAGFIENGWEGMVDGWYGFRHQNS | |
| | EGRGQAADLKSTQAAIDQINGKLNRLIGKTNEKF | |
| | HQIEKEFSEVEGRIQDLEKYVEDTKIDLWSYNAE | |
| | LLVALENQHTIDLTDSEMNKLFEKTKKQLRENAE | |
| | DMGNGCFKIYHKCDNACIGSIRNGTYDHDVYRDE | |
| | ALNNRFQIKGVELKSGYKDWILWISFAISCFLLC | |
| | VALLGFIMWACQKGNIRCNICI | |
| MRK_sH1_Con_v2: | MKVKLLVLLCTFTATYADTICIGYHANNSTDTVD | 46 |
| consensus sequence of HA | TVLEKNVTVTHSVNLLENSHNGKLCLLKGIAPLQ | |
| subtype H1, includes | LGNCSVAGWILGNPECELLISKESWSYIVEKPNP | |
| transmembrane sequence | ENGTCYPGHFADYEELREQLSSVSSFERFEIFPK | |
| (second underlined), italics | ESSWPNHTVTGVSASCSHNGESSFYRNLLWLTGK | |
| indicate possible truncation | NGLYPNLSKSYANNKEKEVLVLWGVHHPPNIGDQ | |
| sites for extracellular | KALYHTENAYVSVVSSHYSRKFTPEIAKRPKVRD | |
| domain | QEGRINYYWTLLEPGDTIIFEANGNLIAPRYAFA | |
| | LSRGFGSGIINSNAPMDKCDAKCQTPQGAINSSL | |
| | PFQNVHPVTIGECPKYVRSAKLRMVTGLRNIPSI | |
| | QSRGLFGAIAGFIEGGWTGMVDGWYGYHHQNEQG | |
| | SGYAADQKSTQNAINGITNKVNSVIEKMNTQFTA | |
| | VGKEFNKLERRMENLNKKVDDGFIDIWTYNAELL | |
| | VLLENERTLDFHDSNVKNLYEKVKSQLKNNAKEI | |
| | GNGCFETYHKCNDECMESVKNGTYDYPKYSEESK | |
| | LNREKIDGVKLESMGVYQILAIYSTVASSLVLLV | |
| | SLGAISFWMCSNGSLQCRICI | |
| MRK_sH1_Con_ecto: ecto | MKVKLLVLLCTFTATYADTICIGYHANNSTDTVD | 47 |
| domain of consensus sH1 | TVLEKNVTVTHSVNLLEDSHNGKLCLLKGIAPLQ | |
| sequence (without | LGNCSVAGWILGNPECELLISKESWSYIVETPNP | |
| transmembrane domain) | ENGTCYPGYFADYEELREQLSSVSSFERFEIFPK | |
| with foldon sequence | ESSWPNHTVTGVSASCSHNGKSSFYRNLLWLTGK | |
| (second underlined), linker | NGLYPNLSKSYANNKEKEVLVLWGVHHPPNIGDQ | |
| (bold), italics indicate other | RALYHTENAYVSVVSSHYSRRFTPEIAKRPKVRD | |
| truncation sites for | QEGRINYYWTLLEPGDTIIFEANGNLIAPRYAFA | |
| extracellular domain. | LSRGFGSGIITSNAPMDECDAKCQTPQGAINSSL | |
| Linker sequence (GSAGSA) | PFQNVHPVTIGECPKYVRSAKLRMVTGLRNIPSI | |
| in bold | QSRGLFGAIAGFIEGGWTGMVDGWYGYHHQNEQG | |
| | SGYAADQKSTQNAINGITNKVNSVIEKMNTQFTA | |
| | VGKEFNKLERRMENLNKKVDDGFLDIWTYNAELL | |
| | VLLENERTLDFHDSNVKNLYEKVKSQLKNNAKEI | |
| | GNGCFEFYHKCNDECMESVKNGTYDYPKYSEESK | |
| | LNREKIDGVKLESMGVGSAGSAGYIPEAPRDGQA | |
| | YVRKDGEWVLSTFL | |
| MRK_sH1_Con_RBD: | MKVKLLVLLCTFTATYAGIAPLQLGNCSVAGWIL | 48 |
| receptor binding domain | GNPECELLISKESWSYIVETPNPENGTCYPGYFA | |
| (RBD) of consensus sH1 | DYEELREQLSSVSSFERFEIFPKESSWPNHTVTG | |
| sequence | VSASCSHNGKSSFYRNLLWLTGKNGLYPNLSKSY | |
| | ANNKEKEVLVLWGVHHPPNIGDQRALYHTENAYV | |
| | SVVSSHYSRRFTPEIAKRPKVRDQEGRINYYWTL | |
| | LEPGDTIIFEANGNLIAPRYAFALSRG | |
| MRK_pH1_Con_ecto: ecto | MKAILVVLLYTFATANADTLCIGYHANNSTDTVD | 49 |
| domain of consensus pH1 | TVLEKNVTVTHSVNLLEDKHNGKLCKLRGVAPLH | |
| sequence (without | LGKCNIAGWILGNPECESLSTASSWSYIVETSSS | |
| transmembrane domain) | DNGTCYPGDFIDYEELREQLSSVSSFERFEIFPK | |
| with foldon sequence | TSSWPNHDSNKGVTAACPHAGAKSFYKNLIWLVK | |
| (second underlined), linker | KGNSYPKLSKSYINDKGKEVLVLWGIHHPSTSAD | |
| (bold), italics indicate other | QQSLYQNADAYVFVGTSRYSKKFKPEIAIRPKVR | |
| truncation sites for | DQEGRMNYYWTLVEPGDKITFEATGNLVVPRYAF | |
| extracellular domain. | AMERNAGSGIIISDTPVHDCNTTCQTPKGAINTS | |
| Linker Sequence in bold | LPFQNIHPITIGKCPKYVKSTKLRLATGLRNVPS | |
| | IQSRGLFGAIAGFIEGGWTGMVDGWYGYHHQNEQ | |
| | GSGYAADLKSTQNAIDKITNKVNSVIEKMNTQFT | |
| | AVGKEFNHLEKRIENLNKKVDDGFLDIWTYNAEL | |
| | LVLLENERTLDYHDSNVKNLYEKVRSQLKNNAKE | |
| | IGNGCFEFYHKCDNTCMESVKNGTYDYPKYSEEA | |
| | KLNREEIDGVKLESTRIGSAGSAGYIPEAPRDGQ | |
| | AYVREDGEWVLLSTFL | |
| MRK_pH1_Con_RBD: | MKVKLLVLLCTFTATYAGVAPLHLGKCNIAGWIL | 50 |
| receptor binding domain | GNPECESLSTASSWSYIVETSSSDNGTCYPGDFI | |
| (RBD) domain of consensus | DYEELREQLSSVSSFERFEIFPKTSSWPNHDSNK | |
| pH1 sequence | GVTAACPHAGAKSFYKNLIWLVKKGNSYPKLSKS | |
| | YINDKGKEVLVLWGIHHPSTSADQQSLYQNADAY | |
| | VFVGTSRYSKKFKPEIAIRPKVRDQEGRMNYYWT | |
| | LVEPGDKITFEATGNLVVPRYAFAMERNA | |
| eH1HA_d5v1: | METPAQLLFLLLLWLPDTTGDTICIGYHANNSTD | 51 |
| linker (bold) with foldon | TVDTVLEKNVTVTHSVNLLEDSHNGKLCRLKGIA | |
| sequence (second | PLQLGKCNIAGWLLGNPECDPLPPMKSWSYIVET | |
| underlined) | PNSENGICYPGDFIDYEELREQLSSVSSFERFEI | |
| Includes the following | FPKGSSWPNHNTNGVTAACSHEGKNSFYRNLLWL | |
| mutations (in bold) | TKKEGLYPNLENSYVNKKEKEVLVLWGIHHPSNN | |
| L75P | KEQQNLYQNENAYVSVVTSNYNRRFTPEIAERPK | |
| V77M | VRDQAGRMNYYWTLLKPGDTIIFEANGNLIAPMY | |
| R78K | AFALSRGFGSGIITSNASMHECNTKCQTPLGAIN | |
| E124G | SSLPYQNIHPVTIGECPKYVRSAKLRMVTGLRNI | |
| G173E | PSIQSRGLFGAIAGFIEGGWTGMIDGWYGYHHQN | |
| S145N | EQGSGYAADQKSTQNAINGITNKVNTVIEKMNIQ | |
| S160L | FTAVGKEFNKLEKRMENLNKKVDDGFLDIWTYNA | |
| K165E | ELLVLLENERTLDFHDSNVKNLYEKVKSQLKNNA | |
| S188N | KEIGNGCFEFYHKCDNECMESVRNGTYDYPKYSE | |
| E156K | ESKLNREKVDGVKLESMGIGSAGSAGYIPEAPRD | |
| | GQAYVRKDGEWVLLSTFL | |
| eH1HA_d5v2: | METPAQLLFLLLLWLPDRRGDTICIGYHANNSTD | 52 |
| linker (bold) with foldon | TVDTVLEKNVTVTHSVNLLEDSHNGKLCRLKGIA | |
| sequence (second | PLQLGKCNIAGWLLGNPECDPLPPMKSWSYIVET | |
| underlined) | PNSENGICYPGDFIDYEELREQLSSVSSFERFEI | |
| Includes the following | FPKGSSWPNHTTNGVTAACSHEGKNSFYRNLLWL | |
| mutations (in bold) | TKKEGSYPNLKNSYVNKKEKEVLVLWGIHHPSNS | |
| L75P | KEQQNLYQNENAHVSVVTSNYNRRFTPEIAERPK | |
| V77M | VRDQAGRMNYYWTLLKPGDTIIFEADGNLIAPMY | |
| R78K | AFALSRGFGSGIITSNASMHECNTKCQTPLGAIN | |
| E124G; | SSLPYQNIHPVTIGECPKYVRSAKLRMVTGLRNI | |
| G173E; | PSIQSRGLFGAIAGFIEGGWTGMIDGWYGYHHQN | |
| S145N; | EQGSGYAADQKSTQNAINGITNKVNTVIEKMNIQ | |
| N248D | FTAVGKEFNKLEKRMENLNKKVDDGFLDIWTYNA | |
| N131T(glyc) | ELLVLLENERTLDFHDSNVKNLYEKVKSQLKNNA | |
| Y201H; | KEIGNGCFEFYHKCDNECMESVRNGTYDYPKYSE | |
| E156K | ESKLNREKVDGVKLESMGIGSAGSAGYIPEAPRD | |
| | GQAYVRKDGEWVLLSTFL | |
| eH1HA_d5v3: | METPAQLLFLLLLWLPDTTGDTICIGYHANNSTD | 53 |
| linker (bold) with foldon | TVDTVLEKNVTVTHSVNLLEDSHNGKLCRLKGIA | |
| sequence (second | PLQLGKCNIAGWLLGNPECDPLPPMKSWSYIVET | |
| underlined) | PNSENGICYPGDFIDYEELREQLSSVSSFERFEI | |
| Includes the following | FPKGSSWPDHNTNGVTAACSHEGKNSFYRNLLWL | |
| mutations (in bold) | TEKKGSYPNLKNPYVNKKEKEVLVLWGIHHPSNS | |
| L75P | KEQQNLYRNENAYVSVVTSNYNRRFTPEIAERPK | |
| V77M | VRDQAGRMNYYWTLLKPGDTIIFEANGNLIAPMY | |
| R78K | AFALSRGFGSGIITSNASMHECNTKCQTPLGAIN | |
| E124G | SSLPYQNIHPVTIGECPKYVRSAKLRMVTGLRNI | |
| G173E | PSIQSRGLFGAIAGFIEGGWTGMIDGWYGYHHQN | |
| S145N | EQGSGYAADQKSTQNAINGITNKVNTVIEKMNIQ | |
| N129D | FTAVGKEFNKLEKRMENLNKKVDDGFLDIWTYNA | |
| E158K | ELLVLLENERTLDFHDSNVKNLYEKVKSQLKNNA | |
| S167P | KEIGNGCFEFYHKCDNECMESVRNGTYDYPKYSE | |
| Q196R | ESKLNREKVDGVKLESMGIGSAGSAGYIPEAPRD | |
| | GQAYVRKDGEWVLLSTFL | |
| eH1HA_d5v4: | METPAQLLFLLLLWLPDTTGDTICIGYHANNSTD | 54 |
| linker (bold) with foldon | TVDTVLEKNVTVTHSVNLLEDSHNGKLCKLKGIA | |
| sequence (second | PLQLGKCNIAGWLLGNPGCDPLLPVGSWSYIVET | |
| underlined) | PNSENGICYPGDFIDYEELREQLSSVSSFERFKI | |
| Includes the following | FPKESSWPDHNTNGVTAACSHEGKNSFYRNLLWL | |
| mutations (in bold) | TKKESSYPNLENSYVNKKRKEVLVLWGIHHPSNS | |
| R47K | KEQQNLYQNENAYVSVVTSNYNRRFTPEIAERPK | |
| R78G | VKGQAGRMNYYWTLLKPGDTIIFEANGNLIAPMY | |
| E119K | AFALSRGFGSGIITSNASMHECNTKCQTPLGAIN | |
| G173R | SSLPYQNIHPVTIGECPKYVRSAKLRMVTGLRNI | |
| R224K | PSIQSRGLFGAIAGFIEGGWTGMIDGWYGYHHQN | |
| E70G | EQGSGYAADQKSTQNAINGITNKVNTVIEKMNIQ | |
| S145N | FTAVGKEFNKLEKRMENLNKKVDDGFLDIWTYNA | |
| D225G | ELLVLLENERTLDFHDSNVKNLYEKVKSQLKNNA | |
| N129D | KEIGNGCFEFYHKCDNECMESVRNGTYDYPKYSE | |
| K165E | ESKLNREKVDGVKLESMGIGSAGSAGYIPEAPRD | |
| E156K | GQAYVRKDGEWVLLSTFL | |
| G159S | | |
| MRK_RBS_HA129 | MKVKLLVLLCTFTATYAGVAPLHLGKCNIAGWLL | 55 |
| | GNPECELLLTVSSWSYIVETSNSDNGTCYPGDFI | |
| | NYEELREQLSSVSSFERFEIFPKTSSWPDHETNR | |
| | GVTAACPYAGANSFYRNLIWLVKKGNSYPKLSKS | |
| | YVNNKGKEVLVLWGIHHPPTSTDQQSLYQNADAY | |
| | VFVGSSRYSKKFKPEIAIRPKVRDQEGRMNYYWT | |
| | LVEPGDKITFEATGNLVVPRYAFAMERNA | |
| RBD1-Cal09-PC-Cb | MKVKLLVLLCTFTATYAGVAPLHLGKCNIAGWIL | 56 |
| 6 glycosylation sites to | GNPECESLSTASSWSNITETPSSDNGTCYPGDFI | |
| allow access to the Cb | DYEELREQLSSVSSFERFEIFPKTSSWPNHSSNK | |
| epitope | GVTAACPHAGAKSFYKNLIWLVKKNGSYPKLNKS | |
| | YINDSGKEVLVLWGIHHPSNSTDQQSLYQNADTY | |
| | VFVGSSNYSKKFKPEIAIRPKVRDQEGRMNYYWT | |
| | LVEPGDKITFEATGNLVVPRYAFAMERNA | |
| RBD1-Cal09-PC | MKVKLLVLLCTFTATYAGVAPLHLGKCNIAGWIL | 57 |
| 7 added glycosylation sites | GNPECESNSTASSWSNITETPSSDNGTCYPGDFI | |
| | DYEELREQLSSVSSFERFEIFPKTSSWPNHSSNK | |
| | GVTAACPHAGAKSFYKNLIWLVKKNGSYPKLNKS | |
| | YINDSGKEVLVLWGIHHPSNSTDQQSLYQNADTY | |
| | VFVGSSNYSKKFKPEIAIRPKVRDQEGRMNYYWT | |
| | LVEPGDKITFEATGNLVVPRYAFAMERNA | |
| RBD1-Cal09 | MKVKLLVLLCTFTATYAGVAPLHLGKCNIAGWIL | 58 |
| | GNPECESLSTASSWSNITETPSSDNGTCYPGDFI | |
| | DYEELREQLSSVSSFERFEIFPKTSSWPNHDSNK | |
| | GVTAACPHAGAKSFYKNLIWLVKKGNSYPKLSKS | |
| | YINDKGKEVLVLWGIHHPSTSADQQSLYQNADTY | |
| | VFVGSSRYSKKFKPEIAIRPKVRDQEGRMNYYWT | |
| | LVEPGDKITFEATGNLVVPRYAFAMERNA | |
| MRK RBD-Cal09-PC-Cb | MKVKLLVLLCTFTATYAGVAPLHLGKCNIAGWIL | 59 |
| | GNPECESLSTASSWSYIVETPSSDNGTCYPGDFI | |
| | DYEELREQLSSVSSFERFEIFPKTSSWPNHSSNK | |
| | GVTAACPHAGAKSFYKNLIWLVKKNGSYPKLNKS | |
| | YINDSGKEVLVLWGIHHPSNSTDQQSLYQNADTY | |
| | VFVGSSNYSKKFKPEIAIRPKVRDQEGRMNYYWT | |
| | LVEPGDKITFEATGNLVVPRYAFAMERNA | |
| MRK_RBD-Cal09-PC | MKVKLLVLLCTFTATYAGVAPLHLGKCNIAGWIL | 60 |
| | GNPECESNSTASSWSYIVETPSSDNGTCYPGDFI | |
| | DYEELREQLSSVSSFERFEIFPKTSSWPNHSSNK | |
| | GVTAACPHAGAKSFYKNLIWLVKKNGSYPKLNKS | |
| | YINDSGKEVLVLWGIHHPSNSTDQQSLYQNADTY | |
| | VFVGSSNYSKKFKPEIAIRPKVRDQEGRMNYYWT | |
| | LVEPGDKITFEATGNLVVPRYAFAMERNA | |
| MRKRBD-Cal09 | MKVKLLVLLCTFTATYAGVAPLHLGKCNIAGWIL | 61 |
| | GNPECESLSTASSWSYIVETPSSDNGTCYPGDFI | |
| | DYEELREQLSSVSSFERFEIFPKTSSWPNHDSNK | |
| | GVTAACPHAGAKSFYKNLIWLVKKGNSYPKLSKS | |
| | YINDKGKEVLVLWGIHHPSTSADQQSLYQNADTY | |
| | VFVGSSRYSKKFKPEIAIRPKVRDQEGRMNYYWT | |
| | LVEPGDKITFEATGNLVVPRYAFAMERNA | |
| FLHA_PR8 | MKANLLVLLCALAAADADTICIGYHANNSTDTVD | 62 |
| includes transmembrane | TVLEKNVTVTHSVNLLEDSHNGKLCRLKGIAPLQ | |
| sequence (second | LGKCNIAGWLLGNPECDPLLPVRSWSYIVETPNS | |
| underlined), italics indicate | ENGICYPGDFIDYEELREQLSSVSSFERFEIFPK | |
| possible truncation sites for | ESSWPNHNTNGVTAACSHEGKSSFYRNLLWLTEK | |
| extracellular domain | EGSYPKLKNSYVNKKGKEVLVLWGIHHPPNSKEQ | |
| | QNLYQNENAYVSVVTSNYNRRFTPEIAERPKVRD | |
| | QAGRMNYYWTLLKPGDTIIFEANGNLIAPMYAFA | |
| | LSRGFGSGIITSNASMHECNTKCQTPLGAINSSL | |
| | PYQNIHPVTIGECPKYVRSAKLRMVTGLRNIPSI | |
| | QSRGLFGAIAGFIEGGWTGMIDGWYGYHHQNEQG | |
| | SGYAADQKSTQNAINGITNKVNTVIEKMNIQFTA | |
| | VGKEFNKLEKRMENLNKKVDDGFLDIWTYNAELL | |
| | VLLENERTLDFHDSNVKNLYEKVKSQLKNNAKEI | |
| | GNGCFEFYHKCDNECMESVRNGTYDYPKYSEESK | |
| | LNREKVDGVKLESMGIYQILAIYSTVASSLVLLV | |
| | SLGAISFWMCSNGSLQCRICI | |
| FLHA_Cal09 | MKAILVVLLYTFATANADTLCIGYHANNSTDTVD | 63 |
| includes transmembrane | TVLEKNVTVTHSVNLLEDKHNGKLCKLRGVAPLH | |
| sequence (second | LGKCNIAGWILGNPECESLSTASSWSYIVETPSS | |
| underlined), italics indicate | DNGTCYPGDFIDYEELREQLSSVSSFERFEIFPK | |
| possible truncation sites for | TSSWPNHDSNKGVTAACPHAGAKSFYKNLIWLVK | |
| extracellular domain | KGNSYPKLSKSYINDKGKEVLVLWGIHHPSTSAD | |
| | QQSLYQNADTYVFVGSSRYSKKFKPEIAIRPKVR | |
| | DQEGRMNYYWTLVEPGDKITFEATGNLVVPRYAF | |
| | AMERNAGSGIIISDTPVHDCNTTCQTPKGAINTS | |
| | LPFQNIHPITIGKCPKYVKSTKLRLATGLRNIPS | |
| | IQSRGLFGAIAGFIEGGWTGMVDGWYGYHHQNEQ | |
| | GSGYAADLKSTQNAIDEITNKVNSVIEKMNTQFT | |
| | AVGKEFNHLEKRIENLNKKVDDGFLDIWTYNAEL | |
| | LVLLENERTLDYHDSNVKNLYEKVRSQLKNNAKE | |
| | IGNGCFEIYHKCDNTCMESVKNGTYDYPKYSEEA | |
| | KLNREEIDGVKLESTRIYQILAIYSTVASSLVLV | |
| | VSLGAISFWMCSNGSLQCRICI | |
| MRK_B_consUnique: | MKAIIVLLMVVTSNADRICTGITSSNSPHVVKTA | 83 |
| consensus sequence for | TQGEVNVTGVIPLTTTPTKSHFANLKGTRTRGKL | |
| type B | CPDCLNCTDLDVALGRPMCVGTTPSAKASILHEV | |
| | RPVTSGCFPIMHDRTKIRQLPNLLRGYEHIRLST | |
| | QNVIDAEKAPGGPYRLGTSGSCPNATSKNGFFAT | |
| | MAWAVPKNDNNKNATNPLTVEVPYICTEGEDQIT | |
| | VWGFHSDNKTQMKKLYGDSNPQKFTSSANGVTTH | |
| | YVSQIGGFPDQTEDGGLPQSGRIVVDYMVQKPGK | |
| | TGTIVYQRGVLLPQKVWCASGRSKVIKGSLPLIG | |
| | EADCLHEKYGGLNKSKPYYTGEHAKAIGNCPIWV | |
| | KTPLKLANGTKYRPPAKLLKERGFFGAIAGFLEG | |
| | GWEGMIAGWHGYTSHGAHGVAVAADLKSTQEAIN | |
| | KITKNLNSLSELEVKNLQRLSGAMDELHNEILEL | |
| | DEKVDDLRADTISSQIELAVLLSNEGIINSEDEH | |
| | LLALERKLKKMLGPSAVDIGNGCFETKHKCNQTC | |
| | LDRIAAGTFNAGEFSLPTFDSLNITAASLNDDGL | |
| | DNHTILLYYSTAASSLAVTLMIAIFIVYMVSRDN | |
| | VSCSICL | |
| MRK_B_ConsensusA: | MKAIIVLLMVVTSNADRICTGITSSNSPHVVKTA | 84 |
| consensus sequence for | TQGEVNVTGVIPLTTTPTKSYFANLKGTRTRGKL | |
| type B, cluster A | CPDCLNCTDLDVALGRPMCVGTTPSAKASILHEV | |
| | RPVTSGCFPIMHDRTKIRQLPNLLRGYENIRLST | |
| | QNVIDAEKAPGGPYRLGTSGSCPNATSKIGFFAT | |
| | MAWAVPKDNYKNATNPLTVEVPYICTEGEDQITV | |
| | WGFHSDNKTQMKNLYGDSNPQKFTSSANGVTTHY | |
| | VSQIGDFPDQTEDGGLPQSGRIVVDYMMQKPGKT | |
| | GTIVYQRGVLLPQKVWCASGRSKVIKGSLPLIGE | |
| | ADCLHEKYGGLNKSKPYYTGEHAKAIGNCPIWVK | |
| | TPLKLANGTKYRPPAKLLKERGFFGAIAGFLEGG | |
| | WEGMIAGWHGYTSHGAHGVAVAADLKSTQEAINK | |
| | ITKNLNSLSELEVKNLQRLSGAMDELHNEILELD | |
| | EKVDDLRADTISSQIELAVLLSNEGIINSEDEHL | |
| | LALERKLKKMLGPSAVDIGNGCFETKHKCNQTCL | |
| | DRIAAGTFNAGEFSLPTFDSLNITAASLNDDGLD | |
| | NHTILLYYSTAASSLAVTLMLAIFIVYMVSRDNV | |
| | SCSICL | |
| MRK_B_ConsensusB: | MKAIIVLLMVVTSNADRICTGITSSNSPHVVKTA | 85 |
| consensus sequence for | TQGEVNVTGVIPLTTTPTKSHFANLKGTETRGKL | |
| type B, cluster B | CPKCLNCTDLDVALGRPKCTGKIPSARVSILHEV | |
| | RPVTSGCFPIMHDRTKIRQLPNLLRGYEHIRLST | |
| | HNVINAENAPGGPYKIGTSGSCPNVTNGNGFFAT | |
| | MAWAVPKNDKNKTATNPLTIEVPYICTEGEDQIT | |
| | VWGFHSDNETQMAKLYGDSKPQKFTSSANGVTTH | |
| | YVSQIGGFPNQTEDGGLPQSGRIVVDYMVQKSGK | |
| | TGTITYQRGILLPQKVWCASGRSKVIKGSLPLIG | |
| | EADCLHEKYGGLNKSKPYYTGEHAKAIGNCPIWV | |
| | KTPLKLANGTKYRPPAKLLKERGFFGAIAGFLEG | |
| | GWEGMIAGWHGYTSHGAHGVAVAADLKSTQEAIN | |
| | KITKNLNSLSELEVKNLQRLSGAMDELHNEILEL | |
| | DEKVDDLRADTISSQIELAVLLSNEGIINSEDEH | |
| | LLALERKLKKMLGPSAVEIGNGCFETKHKCNQTC | |
| | LDRIAAGTFDAGEFSLPTFDSLNITAASLNDDGL | |
| | DNHTILLYYSTAASSLAVTLMIAIFVVYMVSRDN | |
| | VSCSICL | |
| MRK_B_cot_AA | MKAIIVLLMVVTSNADRICTGITSSNSPHVVKTA | 86 |
| “Center of tree” sequence | TQGEVNVTGVIPLTTTPTKSYFANLKGTRTRGKL | |
| for type B | CPDCLNCTDLDVALGRPMCVGTTPSAKASILHEV | |
| | RPVTSGCFPIMHDRTKIRQLPNLLRGYENIRLST | |
| | QNVIDAEKAPGGPYRLGTSGSCPNATSKSGFFAT | |
| | MAWAVPKNDNNKNATNPLTVEVPYICTEGEDQIT | |
| | VWGFHSDNKTQMKNLYGDSNPQKFTSSANGVTTH | |
| | YVSQIGGFPDQTEDGGLPQSGRIVVDYMMQKPGK | |
| | TGTIVYQRGVLLPQKVWCASGRSKVIKGSLPLIG | |
| | EADCLHEKYGGLNKSKPYYTGEHAKAIGNCPIWV | |
| | KTPLKLANGTKYRPPAKLLKERGFFGAIAGFLEG | |
| | GWEGMIAGWHGYTSHGAHGVAVAADLKSTQEAIN | |
| | KITKNLNSLSELEVKNLQRLSGAMDELHNEILEL | |
| | DEKVDDLRADTISSQIELAVLLSNEGIINSEDEH | |
| | LLALERKLKKMLGPSAVDIGNGCFETKHKCNQTC | |
| | LDRIAAGTFNAGEFSLPTFDSLNITAASLNDDGL | |
| | DNHTILLYYSTAASSLAVTLMLAIFIVYMVSRDN | |
| | VSCSICL | |
| MRK_B_COT_A | MKAIIVLLMVVTSNADRICTGITSSNSPHVVKTA | 87 |
| “center of tree” sequence | TQGEVNVTGVIPLTTTPTKSHFANLKGTETRGKL | |
| for type B, cluster A | CPKCLNCTDLDVALGRPKCTGKIPSARVSILHEV | |
| | RPVTSGCFPIMHDRTKIRQLPNLLRGYEHIRLST | |
| | HNVINAENAPGGPYKIGTSGSCPNVTNGNGFFAT | |
| | MAWAVPKNDKNKTATNPLTIEVPYICTEGEDQIT | |
| | VWGFHSDNETQMAKLYGDSKPQKFTSSANGVTTH | |
| | YVSQIGGFPNQTEDGGLPQSGRIVVDYMVQKSGK | |
| | TGTITYQRGILLPQKVWCASGRSKVIKGSLPLIG | |
| | EADCLHEKYGGLNKSKPYYTGEHAKAIGNCPIWV | |
| | KTPLKLANGTKYRPPAKLLKERGFFGAIAGFLEG | |
| | GWEGMIAGWHGYTSHGAHGVAVAADLKSTQEAIN | |
| | KITKNLNSLSELEVKNLQRLSGAMDELHNEILEL | |
| | DEKVDDLRADTISSQIELAVLLSNEGIINSEDEH | |
| | LLALERKLKKMLGPSAVEIGNGCFETKHKCNQTC | |
| | LDRIAAGTFDAGEFSLPTFDSLNITAASLNDDGL | |
| | DNHTILLYYSTAASSLAVTLMIAIFVVYMVSRDN | |
| | VSCSICL | |
| MRK_B_COT_B | MKAIIVLLMVVTSNADRICTGITSSNSPHVVKTA | 88 |
| “center of tree” sequence | TQGEVNVTGVIPLTTTPTKSYFANLKGTRTRGKL | |
| for type B, cluster B | CPDCLNCTDLDVALGRPMCVGTTPSAKASILHEV | |
| | RPVTSGCFPIMHDRTKIRQLPNLLRGYENIRLST | |
| | QNVIDAEKAPGGPYRLGTSGSCPNATSKIGFFAT | |
| | MAWAVPKDNYKNATNPLTVEVPYICTEGEDQITV | |
| | WGFHSDNKTQMKSLYGDSNPQKFTSSANGVTTHY | |
| | VSQIGDFPDQTEDGGLPQSGRIVVDYMMQKPGKT | |
| | GTIVYQRGVLLPQKVWCASGRSKVIKGSLPLIGE | |
| | ADCLHEKYGGLNKSKPYYTGEHAKAIGNCPIWVK | |
| | TPLKLANGTKYRPPAKLLKERGFFGAIAGFLEGG | |
| | WEGMIAGWHGYTSHGAHGVAVAADLKSTQEAINK | |
| | ITKNLNSLSELEVKNLQRLSGAMDELHNEILELD | |
| | EKVDDLRADTISSQIELAVLLSNEGIINSEDEHL | |
| | LALERKLKKMLGPSAVDIGNGCFETKHKCNQTCL | |
| | DRIAAGTFNAGEFSLPTFDSLNITAASLNDDGLD | |
| | NHTILLYYSTAASSLAVTLMLAIFIVYMVSRDNV | |
| | SCSICL | |

TABLE 2
DNA Sequences

| Name | DNA Sequence | SEQ ID NO: |
|---|---|---|
| BHA10-2: HA10 version for | ATGGAGACCCCCGCCCAGCTGCTGTTCCTGCTGC | 21 |
| Influenza | TGCTGTGGCTGCCCGACACCACCGGCCACGTGGT | |
| B/Brisbane/60/2008 strain, | GAAGACCGCCACCCAGGGCGAGGTGAACGTGACC | |
| with exposed hydrophobic | GGCGTGATCCCCCTGACCACCACCCCCACCGGCA | |
| residues mutated I333T, | GCGCCAACAAGAGCAAGCCCTACTACACCGGCGA | |
| M432S, L435T | GCACGCCAAGGCCACCGGCAACTGCCCCATCTGG | |
| (bold/underlined) and foldon | GTGAAGACCCCCCTGAAGCTGGCCAACGGCACCA | |
| sequence (second | AGTACGGCAGCGCCGGCAGCGCCACCCAGGAGGC | |
| underlined) | CATCAACAAGATCACCAAGAACCTGAACAGCCTG | |
| | AGCGAGCTGGAGGTGAAGAACCTGCAGAGGCTGA | |
| | GCGGCGCCAGCGACGAGACCCACAACGAGATCCT | |
| | GGAGCTGGACGAGAAGGTGGACGACCTGAGGGCC | |
| | GACACCATCAGCAGCCAGATCGAGCTGGCCGTGC | |
| | TGCTGAGCAACGAGGGCATCATCAACAGCGAGGA | |
| | CGAGGGCACCGGCGGCGGCTACATCCCCGAGGCC | |
| | CCCAGGGACGGCCAGGCCTACGTGAGGAAGGACG | |
| | GCGAGTGGGTGCTGCTGAGCACCTTCCTG | |
| BHA10-3: BHA10-2 without | ATGGAGACCCCCGCCCAGCTGCTGTTCCTGCTGC | 22 |
| GTGG linker or foldon | TGCTGTGGCTGCCCGACACCACCGGCCACGTGGT | |
| domain, with G430C, | GAAGACCGCCACCCAGGGCGAGGTGAACGTGACC | |
| E438C, Q457L mutations | GGCGTGATCCCCCTGACCACCACCCCCACCGGCA | |
| (bold) for trimerization | GCGCCAACAAGAGCAAGCCCTACTACACCGGCGA | |
| | GCACGCCAAGGCCACCGGCAACTGCCCCATCTGG | |
| | GTGAAGACCCCCCTGAAGCTGGCCAACGGCACCA | |
| | AGTACGGCAGCGCCGGCAGCGCCACCCAGGAGGC | |
| | CATCAACAAGATCACCAAGAACCTGAACAGCCTG | |
| | AGCGAGCTGGAGGTGAAGAACCTGCAGAGGCTGA | |
| | GCTGCGCCAGCGACGAGACCCACAACTGCATCCT | |
| | GGAGCTGGACGAGAAGGTGGACGACCTGAGGGCC | |
| | GACACCATCAGCAGCCTGATCGAGCTGGCCGTGC | |
| | TGCTGAGCAACGAGGGCATCATCAACAGCGAGGA | |
| | CGAG | |
| NIHGen6HASS-TM: Gen6 | ATGGAGACCCCCGCCCAGCTGCTGTTCCTGCTGC | 23 |
| HA SS construct without | TGCTGTGGCTGCCCGACACCACCGGCGACACCAT | |
| foldon or ferritin, linker | CTGCATCGGCTACCACGCCAACAACAGCACCGAC | |
| (bold) with transmembrane | ACCGTGGACACCGTGCTGGAGAAGAACGTGACCG | |
| domain (second underlined), | TGACCCACAGCGTGAACCTGGGCAGCGGCCTGAG | |
| version 1 | GATGGTGACCGGCCTGAGGAACATCCCCCAGAGG | |
| | GAGACCAGGGGCCTGTTCGGCGCCATCGCCGGCT | |
| | TCATCGAGGGCGGCTGGACCGGCATGGTGGACGG | |
| | CTGGTACGGCTACCACCACCAGAACGAGCAGGGC | |
| | AGCGGCTACGCCGCCGACCAGAAGAGCACCCAGA | |
| | ACGCCATCAACGGCATCACCAACATGGTGAACAG | |
| | CGTGATCGAGAAGATGGGCAGCGGCGGCAGCGGC | |
| | ACCGACCTGGCCGAGCTGCTGGTGCTGCTGCTGA | |
| | ACGAGAGGACCCTGGACTTCCACGACAGCAACGT | |
| | GAAGAACCTGTACGAGAAGGTGAAGAGCCAGCTG | |
| | AAGAACAACGCCAAGGAGATCGGCAACGGCTGCT | |
| | TCGAGTTCTACCACAAGTGCAACAACGAGTGCAT | |
| | GGAGAGCGTGAAGAACGGCACCTACGACTACCCC | |
| | AAGTACAGCGAGGAGAGCAAGCTGAACAGGGAGA | |
| | AGATCGACCAGGGCACCGGCGGCATCCTGGCCAT | |
| | CTACAGCACCGTGGCCAGCAGCCTGGTGCTGCTG | |
| | GTGAGCCTGGGCGCCATCAGCTTCTGGATGTGCA | |
| | GCAACGGCAGCCTGCAGTGCAGAATCTGCATC | |
| NIHGen6HASS-TM2: Gen6 | ATGGAGACCCCCGCCCAGCTGCTGTTCCTGCTGC | 24 |
| HA SS construct without | TGCTGTGGCTGCCCGACACCACCGGCGACACCAT | |
| foldon or ferritin, linker | CTGCATCGGCTACCACGCCAACAACAGCACCGAC | |
| (bold), with transmembrane | ACCGTGGACACCGTGCTGGAGAAGAACGTGACCG | |
| domain (second underlined), | TGACCCACAGCGTGAACCTGGGCAGCGGCCTGAG | |
| version 2 | GATGGTGACCGGCCTGAGGAACATCCCCCAGAGG | |
| | GAGACCAGGGGCCTGTTCGGCGCCATCGCCGGCT | |
| | TCATCGAGGGCGGCTGGACCGGCATGGTGGACGG | |
| | CTGGTACGGCTACCACCACCAGAACGAGCAGGGC | |
| | AGCGGCTACGCCGCCGACCAGAAGAGCACCCAGA | |
| | ACGCCATCAACGGCATCACCAACATGGTGAACAG | |
| | CGTGATCGAGAAGATGGGCAGCGGCGGCAGCGGC | |
| | ACCGACCTGGCCGAGCTGCTGGTGCTGCTGCTGA | |
| | ACGAGAGGACCCTGGACTTCCACGACAGCAACGT | |
| | GAAGAACCTGTACGAGAAGGTGAAGAGCCAGCTG | |
| | AAGAACAACGCCAAGGAGATCGGCAACGGCTGCT | |
| | TCGAGTTCTACCACAAGTGCAACAACGAGTGCAT | |
| | GGAGAGCGTGAAGAACGGCACCTACGACTACCCC | |
| | AAGTACAGCGAGGAGAGCAAGCTGAACAGGGAGA | |
| | AGATCGACGGAGTGAAATTGGAATCAATGGGGGT | |
| | CTATCAGATCCTGGCCATCTACAGCACCGTGGCC | |
| | AGCAGCCTGGTGCTGCTGGTGAGCCTGGGCGCCA | |
| | TCAGCTTCTGGATGTGCAGCAACGGCAGCCTGCA | |
| | GTGCAGAATCTGCATC | |
| NIHGen6HASS-foldon | ATGGAGACCCCCGCCCAGCTGCTGTTCCTGCTGC | 25 |
| Gen6 HA SS construct with | TGCTGTGGCTGCCCGACACCACCGGCGACACCAT | |
| foldon sequence (second | CTGCATCGGCTACCACGCCAACAACAGCACCGAC | |
| underlined) | ACCGTGGACACCGTGCTGGAGAAGAACGTGACCG | |
| | TGACCCACAGCGTGAACCTGGGCAGCGGCCTGAG | |
| | GATGGTGACCGGCCTGAGGAACATCCCCCAGAGG | |
| | GAGACCAGGGGCCTGTTCGGCGCCATCGCCGGCT | |
| | TCATCGAGGGCGGCTGGACCGGCATGGTGGACGG | |
| | CTGGTACGGCTACCACCACCAGAACGAGCAGGGC | |
| | AGCGGCTACGCCGCCGACCAGAAGAGCACCCAGA | |
| | ACGCCATCAACGGCATCACCAACATGGTGAACAG | |
| | CGTGATCGAGAAGATGGGCAGCGGCGGCAGCGGC | |
| | ACCGACCTGGCCGAGCTGCTGGTGCTGCTGCTGA | |
| | ACGAGAGGACCCTGGACTTCCACGACAGCAACGT | |
| | GAAGAACCTGTACGAGAAGGTGAAGAGCCAGCTG | |
| | AAGAACAACGCCAAGGAGATCGGCAACGGCTGCT | |
| | TCGAGTTCTACCACAAGTGCAACAACGAGTGCAT | |
| | GGAGAGCGTGAAGAACGGCACCTACGACTACCCC | |
| | AAGTACAGCGAGGAGAGCAAGCTGAACAGGGAGA | |
| | AGATCGACCCCGGCAGCGGCTACATCCCCGAGGC | |
| | CCCCAGGGACGGCCAGGCCTACGTGAGGAAGGAC | |
| | GGCGAGTGGGTGCTGCTGAGCACCTTCCTG | |
| ConH1: consensus HA | ATGAAGGCCAAGCTGCTGGTGCTGCTGTGCGCCT | 26 |
| sequence for subtype H1, | TCACCGCCACCGACGCCGACACCATCTGCATTGG | |
| with transmembrane domain | CTACCATGCCAACAACAGCACCGACACCGTGGAC | |
| (second underlined) | ACCGTGCTCGAGAAGAACGTGACCGTGACCCACT | |
| | CCGTGAATTTGTTGGAGGACAGCCACAACGGCAA | |
| | GCTGTGTAAGCTGAAGGGCATCGCCCCCCTGCAG | |
| | CTGGGCAAGTGCAACATCGCCGGCTGGATCTTGG | |
| | GCAATCCCGAGTGCGAAAGCCTGATCTCCAAGAG | |
| | AAGCTGGAGCTACATCGTGGAGACTCCCAACAGC | |
| | GAAAACGGCACCTGCTACCCCGGCGACTTCGCTG | |
| | ACTACGAGGAACTGAGAGAGCAGCTGAGCAGCGT | |
| | GAGCAGCTTTGAGAGATTCGAGATCTTCCCCAAA | |
| | GAGAGCAGCTGGCCCAACCACAACGTAACCAAGG | |
| | GCGTGACAGCCGCCTGCAGCCACGCCGGTAAGAG | |
| | CAGCTTCTACAGAAACCTGCTGTGGCTGACAGAG | |
| | AAGAACGGCAGCTACCCCAAGCTGAGCAAGAGCT | |
| | ATGTGAACAACAAGGAGAAGGAGGTGCTGGTCCT | |
| | GTGGGGCGTACACCACCCCAGCAACATTACCGAT | |
| | CAGAGAACCCTGTACCAGAACGAGAATGCCTACG | |
| | TGAGCGTGGTGAGCAGCCACTACAACAGAAGATT | |
| | CACCCCCGAGATTGCCAAGAGACCGAAAGTGAGA | |
| | GGCCAGGCCGGAAGAATCAACTACTACTGGACCC | |
| | TGCTGGAGCCCGGCGACACCATCATCTTCGAGGC | |
| | CAACGGCAACCTGATCGCCCCCTGGTATGCCTTC | |
| | GCCCTGAGCAGAGGCTTCGGAAGCGGCATCATCA | |
| | CATCCAACGCCCCCATGCATGAATGCGACACAAA | |
| | GTGTCAGACCCCCCAGGGCGCCATCAACAGCAGC | |
| | CTGCCCTTCCAGAACGTGCACCCTGTGACCATCG | |
| | GCGAGTGCCCCAAGTACGTGAGAAGCACCAAGCT | |
| | GAGAATGGTGACCGGCCTGAGAAATATCCCCAGT | |
| | ATCCAGAGCAGAGGCCTGTTCGGCGCCATCGCCG | |
| | GCTTCATCGAGGGCGGCTGGACCGGCATGATCGA | |
| | CGGCTGGTACGGCTACCACCACCAGAACGAGCAG | |
| | GGCAGCGGCTACGCCGCCGACCAGAAGAGCACTC | |
| | AGAACGCCATCAACGGCATCACCAACAAGGTGAA | |
| | CAGCGTGATCGAGAAGATGAACACACAGTTCACC | |
| | GCCGTGGGCAAAGAGTTCAATAAGCTCGAGAAGA | |
| | GAATGGAAAACCTGAACAAGAAGGTTGACGACGG | |
| | TTTCCTGGATATCTGGACCTACAACGCCGAGCTG | |
| | CTTGTGCTGCTGGAGAACGAGCGTACCCTGGACT | |
| | TTCACGACTCGAACGTGAAGAACCTGTACGAAAA | |
| | GGTGAAGTCCCAGCTGAAGAACAACGCCAAAGAA | |
| | ATTGGCAACGGCTGCTTCGAGTTCTACCACAAGT | |
| | GTAACAACGAGTGCATGGAGAGCGTGAAGAACGG | |
| | CACCTACGACTATCCCAAGTACAGCGAGGAGAGC | |
| | AAGCTAAACAGAGAGAAGATTGACGGCGTGAAAC | |
| | TGGAGTCAATGGGCGTGTACCAGATCCTGGCCAT | |
| | CTACAGCACCGTGGCCAGCAGCCTCGTGCTGCTG | |
| | GTGAGCCTGGGCGCCATAAGCTTCTGGATGTGTA | |
| | GCAACGGCAGCCTGCAGTGCAGAATCTGCATC | |
| ConH3: consensus HA | ATGAAGACCATCATCGCTCTGAGCTACATATTCT | 27 |
| sequence for subtype H3, | GCCTGGTGTTCGCCCAGAAGCTGCCCGGCAACGA | |
| with transmembrane domain | CAACAGCACCGCCACCCTGTGCCTGGGCCATCAC | |
| (second underlined) | GCAGTGCCAAACGGCACCTTAGTTAAGACCATCA | |
| | CCAACGACCAGATCGAGGTGACCAACGCCACCGA | |
| | GCTGGTGCAGAGCAGTAGCACCGGCAGAATCTGC | |
| | GACAGCCCCCACCGGATCCTTGACGGCACTAACT | |
| | GCACCCTGATCGACGCCCTGCTGGGCGACCCCCA | |
| | CTGCGACGGGTTCCAGAACAAGGAGTGGGACCTG | |
| | TTCGTGGAGAGAAGCAAGGCCTACAGCAACTGCT | |
| | ACCCCTACGATGTGCCCGACTACGCCAGCCTGAG | |
| | ATCTCTTGTGGCTAGCAGCGGCACCCTGGAGTTC | |
| | AACAATGAGGGCTTCAATTGGACAGGCGTGACCC | |
| | AGAACGGCGGCAGCAGCGCCTGCAAGAGAGGCAG | |
| | CGACAAGAGCTTCTTCAGCAGACTGAACTGGCTG | |
| | CACAAGCTGAAGTACAAGTATCCCGCCCTGAACG | |
| | TGACCATGCCCAATAACGACAAGTTCGATAAGCT | |
| | GTATATTTGGGGCGTGCACCACCCCAGCACCGAC | |
| | AGCGACCAGACCTCCCTGTACGTCCAGGCGAGCG | |
| | GCAGAGTGACCGTGAGCACCAAACGGAGCCAGCA | |
| | AACCGTGATCCCCAACATCGGCAGCAGACCTTGG | |
| | GTCAGAGGACTGAGCAGCAGAATCAGCATCTACT | |
| | GGACCATCGTGAAGCCTGGCGACATCTTGCTGAT | |
| | CAATAGCACCGGCAACCTGATCGCCCCCAGAGGC | |
| | TACTTCAAGATCAGAAGCGGCAAGAGCTCAATCA | |
| | TGAGAAGCGACGCCCCCATAGGCACCTGCAACAG | |
| | CGAGTGCATCACCCCGAACGGCAGCATCCCCAAC | |
| | GACAAGCCCTTCCAGAACGTGAACAGAATCACAT | |
| | ACGGCGCCTGCCCCAGGTACGTAAAGCAAAACAC | |
| | CCTGAAGCTGGCCACCGGCATGAGAAACGTACCC | |
| | GAGAAGCAGACCAGAGGCATCTTCGGCGCCATCG | |
| | CGGGCTTCATCGAGAATGGCTGGGAAGGCATGGT | |
| | GGACGGCTGGTACGGCTTCAGACATCAGAACAGC | |
| | GAGGGCACCGGCCAGGCCGCCGACCTGAAAAGCA | |
| | CCCAGGCCGCCATCGACCAGATCAACGGCAAGCT | |
| | GAACAGACTGATCGAAAAGACCAACGAGAAGTTC | |
| | CACCAGATCGAGAAGGAGTTCAGCGAAGTGGAGG | |
| | GAAGAATACAGGACCTGGAGAAATACGTGGAGGA | |
| | CACCAAGATCGACCTGTGGTCGTACAACGCCGAG | |
| | CTGCTGGTGGCCCTGGAAAACCAGCACACCATTG | |
| | ACCTGACCGATAGCGAGATGAACAAGCTGTTCGA | |
| | GAGAACCAGAAAACAGCTGAGAGAGAACGCCGAG | |
| | GACATGGGCAACGGCTGCTTTAAGATCTACCACA | |
| | AGTGCGACAACGCCTGCATCGGCAGCATCAGAAA | |
| | CGGCACCTACGACCACGACGTGTACAGAGACGAG | |
| | GCCCTGAACAACAGATTCCAGATCAAGGGCGTGG | |
| | AGCTGAAGAGCGGCTACAAGGACTGGATCCTGTG | |
| | GATCAGCTTCGCCATCAGCTGTTTCCTGCTTTGC | |
| | GTAGTGCTGCTGGGCTTCATCATGTGGGCCTGCC | |
| | AGAAGGGCAATATCAGATGCAACATTTGCATC | |
| MRK_pH1_Con: consensus | ATGAAGGCCATCCTGGTGGTGCTGCTGTACACCT | 28 |
| HA sequence for pandemic | TCGCCACGGCCAACGCTGACACCCTGTGCATCGG | |
| H1 strains, includes | ATACCACGCGAACAACAGCACCGACACCGTTGAC | |
| transmembrane sequence | ACCGTGCTGGAGAAGAACGTGACCGTGACCCACA | |
| (second underlined) | GCGTGAACCTCCTGGAAGACAAGCACAACGGCAA | |
| | GCTGTGCAAGCTGAGAGGCGTGGCCCCCCTGCAC | |
| | CTGGGCAAGTGCAACATCGCCGGCTGGATCTTAG | |
| | GCAACCCCGAGTGCGAGAGCCTGAGCACCGCCAG | |
| | CAGCTGGAGCTACATTGTGGAGACCAGCAGCAGC | |
| | GACAACGGCACCTGCTACCCCGGCGACTTCATCG | |
| | ACTACGAGGAGCTGAGAGAGCAGCTGAGCAGCGT | |
| | GAGCAGCTTCGAGAGATTCGAGATCTTCCCCAAG | |
| | ACCTCAAGCTGGCCCAACCACGACAGCAATAAGG | |
| | GCGTGACTGCCGCCTGCCCCCACGCCGGCGCCAA | |
| | GAGCTTCTACAAGAACCTGATCTGGCTGGTGAAG | |
| | AAAGGCAATAGCTACCCCAAGCTGAGCAAGTCCT | |
| | ATATCAACGACAAGGGCAAGGAGGTGTTGGTTCT | |
| | GTGGGGCATCCATCACCCCAGCACCAGTGCTGAT | |
| | CAGCAAAGCCTGTACCAAAACGCCGATGCCTACG | |
| | TGTTCGTGGGCACCTCAAGATACAGCAAGAAGTT | |
| | CAAGCCCGAGATCGCCATCAGACCCAAGGTGAGA | |
| | GACCAGGAGGGTAGAATGAACTACTACTGGACCC | |
| | TCGTGGAGCCGGGCGACAAGATCACCTTCGAGGC | |
| | CACCGGCAACCTGGTGGTGCCCAGATACGCCTTC | |
| | GCCATGGAGAGAAATGCCGGCAGCGGGATCATCA | |
| | TCTCGGACACCCCCGTGCACGATTGTAACACCAC | |
| | CTGCCAGACCCCCAAGGGCGCCATCAACACCTCC | |
| | CTGCCCTTCCAGAACATCCACCCCATCACCATCG | |
| | GCAAGTGCCCCAAGTACGTGAAGAGCACCAAGCT | |
| | GAGACTGGCGACCGGACTGAGAAACGTGCCCAGC | |
| | ATCCAGTCAAGAGGCCTGTTCGGCGCCATCGCCG | |
| | GCTTCATCGAGGGCGGCTGGACAGGCATGGTGGA | |
| | CGGCTGGTACGGCTACCACCACCAGAACGAGCAG | |
| | GGCAGCGGATACGCCGCCGACCTGAAGAGCACAC | |
| | AAAACGCCATCGACAAGATCACCAACAAGGTGAA | |
| | CAGCGTGATCGAAAAGATGAACACCCAGTTCACC | |
| | GCCGTGGGCAAGGAGTTCAACCACCTGGAGAAGA | |
| | GAATCGAGAACCTGAACAAGAAAGTGGACGACGG | |
| | CTTCCTGGACATCTGGACCTACAACGCCGAGCTG | |
| | CTGGTCCTGCTGGAGAACGAGAGAACCCTGGACT | |
| | ACCATGACAGCAACGTGAAGAACCTGTACGAGAA | |
| | GGTGAGAAGCCAGCTGAAGAACAACGCCAAGGAG | |
| | ATAGGCAACGGCTGCTTCGAGTTCTACCACAAGT | |
| | GCGACAACACCTGCATGGAGAGCGTGAAGAACGG | |
| | CACCTACGACTACCCAAAGTATAGCGAGGAGGCA | |
| | AAGCTGAACAGAGAGGAGATCGACGGCGTGAAGC | |
| | TGGAGAGCACCAGGATCTATCAAATCCTGGCCAT | |
| | ATACAGCACCGTGGCCAGCAGCCTGGTGTTAGTG | |
| | GTGAGCCTGGGCGCCATCAGCTTCTGGATGTGTA | |
| | GCAACGGCAGCCTGCAGTGCAGAATCTGCATC | |
| MRK_pH1_Con: consensus | ATGAAGGCCATCCTGGTGGTGCTGCTGTACACCT | 29 |
| HA sequence for pandemic | TCGCCACGGCCAACGCTGACACCCTGTGCATCGG | |
| H1 strains, extracellular | ATACCACGCGAACAACAGCACCGACACCGTTGAC | |
| domain (italics indicate | ACCGTGCTGGAGAAGAACGTGACCGTGACCCACA | |
| other truncation sites for | GCGTGAACCTCCTGGAAGACAAGCACAACGGCAA | |
| extracellular domain) | GCTGTGCAAGCTGAGAGGCGTGGCCCCCCTGCAC | |
| | CTGGGCAAGTGCAACATCGCCGGCTGGATCTTAG | |
| | GCAACCCCGAGTGCGAGAGCCTGAGCACCGCCAG | |
| | CAGCTGGAGCTACATTGTGGAGACCAGCAGCAGC | |
| | GACAACGGCACCTGCTACCCCGGCGACTTCATCG | |
| | ACTACGAGGAGCTGAGAGAGCAGCTGAGCAGCGT | |
| | GAGCAGCTTCGAGAGATTCGAGATCTTCCCCAAG | |
| | ACCTCAAGCTGGCCCAACCACGACAGCAATAAGG | |
| | GCGTGACTGCCGCCTGCCCCCACGCCGGCGCCAA | |
| | GAGCTTCTACAAGAACCTGATCTGGCTGGTGAAG | |
| | AAAGGCAATAGCTACCCCAAGCTGAGCAAGTCCT | |
| | ATATCAACGACAAGGGCAAGGAGGTGTTGGTTCT | |
| | GTGGGGCATCCATCACCCCAGCACCAGTGCTGAT | |
| | CAGCAAAGCCTGTACCAAAACGCCGATGCCTACG | |
| | TGTTCGTGGGCACCTCAAGATACAGCAAGAAGTT | |
| | CAAGCCCGAGATCGCCATCAGACCCAAGGTGAGA | |
| | GACCAGGAGGGTAGAATGAACTACTACTGGACCC | |
| | TCGTGGAGCCGGGCGACAAGATCACCTTCGAGGC | |
| | CACCGGCAACCTGGTGGTGCCCAGATACGCCTTC | |
| | GCCATGGAGAGAAATGCCGGCAGCGGGATCATCA | |
| | TCTCGGACACCCCCGTGCACGATTGTAACACCAC | |
| | CTGCCAGACCCCCAAGGGCGCCATCAACACCTCC | |
| | CTGCCCTTCCAGAACATCCACCCCATCACCATCG | |
| | GCAAGTGCCCCAAGTACGTGAAGAGCACCAAGCT | |
| | GAGACTGGCGACCGGACTGAGAAACGTGCCCAGC | |
| | ATCCAGTCAAGAGGCCTGTTCGGCGCCATCGCCG | |
| | GCTTCATCGAGGGCGGCTGGACAGGCATGGTGGA | |
| | CGGCTGGTACGGCTACCACCACCAGAACGAGCAG | |
| | GGCAGCGGATACGCCGCCGACCTGAAGAGCACAC | |
| | AAAACGCCATCGACAAGATCACCAACAAGGTGAA | |
| | CAGCGTGATCGAAAAGATGAACACCCAGTTCACC | |
| | GCCGTGGGCAAGGAGTTCAACCACCTGGAGAAGA | |
| | GAATCGAGAACCTGAACAAGAAAGTGGACGACGG | |
| | CTTCCTGGACATCTGGACCTACAACGCCGAGCTG | |
| | CTGGTCCTGCTGGAGAACGAGAGAACCCTGGACT | |
| | ACCATGACAGCAACGTGAAGAACCTGTACGAGAA | |
| | GGTGAGAAGCCAGCTGAAGAACAACGCCAAGGAG | |
| | ATAGGCAACGGCTGCTTCGAGTTCTACCACAAGT | |
| | GCGACAACACCTGCATGGAGAGCGTGAAGAACGG | |
| | CACCTACGACTACCCAAAGTATAGCGAGGAGGCA | |
| | AAGCTGAACAGAGAGGAGATCGACGGCGTGAAGC | |
| | TGGAGAGCACCAGGATCTATCAA | |
| MRK_sH1_Con: consensus | ATGAAGGTGAAGCTGCTGGTGCTGCTGTGTACCT | 30 |
| HA sequence for seasonal | TCACTGCCACTTACGCCGACACCATTTGCATCGG | |
| H1 strains, includes | CTACCACGCCAACAACAGCACCGATACCGTGGAC | |
| transmembrane sequence | ACCGTGCTGGAGAAGAACGTCACCGTGACCCACA | |
| (second underlined) | GCGTGAACCTGCTGGAGGATAGCCATAACGGCAA | |
| | GCTGTGCCTGCTGAAGGGAATCGCCCCCCTGCAG | |
| | CTCGGCAACTGCAGCGTGGCCGGCTGGATTCTGG | |
| | GCAACCCCGAGTGCGAACTGCTGATTAGCAAAGA | |
| | GTCCTGGAGCTACATCGTGGAAACCCCGAATCCC | |
| | GAGAACGGCACCTGCTACCCCGGCTACTTCGCCG | |
| | ACTACGAAGAGCTAAGAGAGCAGCTGAGTAGCGT | |
| | GAGCTCATTCGAGAGATTCGAGATCTTTCCCAAG | |
| | GAGTCTAGCTGGCCCAATCACACCGTGACCGGCG | |
| | TGAGCGCCAGCTGTAGCCACAACGGCAAGAGCAG | |
| | CTTCTACAGAAACCTGCTGTGGCTGACCGGCAAG | |
| | AACGGACTGTACCCTAACCTGAGCAAGAGCTACG | |
| | CGAACAATAAGGAGAAGGAGGTGCTAGTGCTGTG | |
| | GGGCGTGCACCACCCCCCCAACATCGGCGACCAG | |
| | AGAGCCCTGTACCACACCGAGAACGCCTACGTGA | |
| | GCGTGGTGAGCAGCCACTATAGCAGAAGATTCAC | |
| | CCCCGAGATCGCCAAGAGACCAAAGGTGAGAGAT | |
| | CAGGAAGGAAGAATAAACTACTACTGGACCCTCC | |
| | TGGAGCCCGGCGACACCATCATCTTCGAGGCTAA | |
| | CGGCAACCTGATCGCCCCCAGATACGCCTTCGCC | |
| | CTGAGCAGAGGCTTCGGCAGCGGCATCATCACCA | |
| | GCAATGCCCCCATGGATGAGTGCGACGCCAAGTG | |
| | CCAGACCCCCCAGGGCGCCATCAACTCGAGCCTG | |
| | CCCTTCCAGAATGTGCACCCCGTGACCATCGGCG | |
| | AGTGCCCCAAGTACGTGAGAAGCGCCAAGCTGAG | |
| | AATGGTGACCGGCCTGAGAAACATCCCAAGCATC | |
| | CAGAGCAGAGGGCTGTTCGGCGCCATCGCTGGCT | |
| | TCATCGAGGGCGGCTGGACCGGCATGGTGGACGG | |
| | CTGGTACGGTTATCACCACCAGAACGAGCAGGGC | |
| | AGCGGCTACGCCGCCGACCAGAAGAGCACCCAGA | |
| | ACGCCATCAACGGCATTACAAACAAGGTGAACAG | |
| | CGTTATCGAGAAGATGAACACCCAATTCACCGCC | |
| | GTGGGCAAGGAGTTCAACAAGCTGGAGAGAAGAA | |
| | TGGAGAACCTGAACAAGAAGGTGGACGACGGCTT | |
| | CCTGGACATCTGGACCTACAACGCCGAGCTGCTG | |
| | GTGCTGCTGGAGAACGAGAGAACCCTGGACTTCC | |
| | ACGACTCCAACGTGAAGAACTTATACGAGAAGGT | |
| | GAAGAGCCAGCTGAAGAACAACGCCAAAGAAATC | |
| | GGAAACGGCTGCTTCGAATTCTACCACAAGTGCA | |
| | ACGACGAATGCATGGAGAGCGTGAAGAACGGAAC | |
| | CTACGACTACCCCAAGTACAGCGAGGAAAGCAAA | |
| | CTGAACAGAGAGAAGATCGACGGCGTGAAGTTAG | |
| | AGAGCATGGGCGTGTATCAGATCCTGGCCATTTA | |
| | TAGCACGGTGGCCAGCAGCCTGGTGCTGCTGGTG | |
| | AGCCTGGGCGCCATCAGCTTCTGGATGTGCAGCA | |
| | ACGGCAGCCTGCAGTGCAGAATCTGCATC | |
| MRK_sH1_Con: consensus | ATGAAGGTGAAGCTGCTGGTGCTGCTGTGTACCT | 31 |
| HA sequence for seasonal | TCACTGCCACTTACGCCGACACCATTTGCATCGG | |
| H1 strains, extracellular | CTACCACGCCAACAACAGCACCGATACCGTGGAC | |
| domain (italics indicate | ACCGTGCTGGAGAAGAACGTCACCGTGACCCACA | |
| other truncation sites for | GCGTGAACCTGCTGGAGGATAGCCATAACGGCAA | |
| extracellular domain) | GCTGTGCCTGCTGAAGGGAATCGCCCCCCTGCAG | |
| | CTCGGCAACTGCAGCGTGGCCGGCTGGATTCTGG | |
| | GCAACCCCGAGTGCGAACTGCTGATTAGCAAAGA | |
| | GTCCTGGAGCTACATCGTGGAAACCCCGAATCCC | |
| | GAGAACGGCACCTGCTACCCCGGCTACTTCGCCG | |
| | ACTACGAAGAGCTAAGAGAGCAGCTGAGTAGCGT | |
| | GAGCTCATTCGAGAGATTCGAGATCTTTCCCAAG | |
| | GAGTCTAGCTGGCCCAATCACACCGTGACCGGCG | |
| | TGAGCGCCAGCTGTAGCCACAACGGCAAGAGCAG | |
| | CTTCTACAGAAACCTGCTGTGGCTGACCGGCAAG | |
| | AACGGACTGTACCCTAACCTGAGCAAGAGCTACG | |
| | CGAACAATAAGGAGAAGGAGGTGCTAGTGCTGTG | |
| | GGGCGTGCACCACCCCCCCAACATCGGCGACCAG | |
| | AGAGCCCTGTACCACACCGAGAACGCCTACGTGA | |
| | GCGTGGTGAGCAGCCACTATAGCAGAAGATTCAC | |
| | CCCCGAGATCGCCAAGAGACCAAAGGTGAGAGAT | |
| | CAGGAAGGAAGAATAAACTACTACTGGACCCTCC | |
| | TGGAGCCCGGCGACACCATCATCTTCGAGGCTAA | |
| | CGGCAACCTGATCGCCCCCAGATACGCCTTCGCC | |
| | CTGAGCAGAGGCTTCGGCAGCGGCATCATCACCA | |
| | GCAATGCCCCCATGGATGAGTGCGACGCCAAGTG | |
| | CCAGACCCCCCAGGGCGCCATCAACTCGAGCCTG | |
| | CCCTTCCAGAATGTGCACCCCGTGACCATCGGCG | |
| | AGTGCCCCAAGTACGTGAGAAGCGCCAAGCTGAG | |
| | AATGGTGACCGGCCTGAGAAACATCCCAAGCATC | |
| | CAGAGCAGAGGGCTGTTCGGCGCCATCGCTGGCT | |
| | TCATCGAGGGCGGCTGGACCGGCATGGTGGACGG | |
| | CTGGTACGGTTATCACCACCAGAACGAGCAGGGC | |
| | AGCGGCTACGCCGCCGACCAGAAGAGCACCCAGA | |
| | ACGCCATCAACGGCATTACAAACAAGGTGAACAG | |
| | CGTTATCGAGAAGATGAACACCCAATTCACCGCC | |
| | GTGGGCAAGGAGTTCAACAAGCTGGAGAGAAGAA | |
| | TGGAGAACCTGAACAAGAAGGTGGACGACGGCTT | |
| | CCTGGACATCTGGACCTACAACGCCGAGCTGCTG | |
| | GTGCTGCTGGAGAACGAGAGAACCCTGGACTTCC | |
| | ACGACTCCAACGTGAAGAACTTATACGAGAAGGT | |
| | GAAGAGCCAGCTGAAGAACAACGCCAAAGAAATC | |
| | GGAAACGGCTGCTTCGAATTCTACCACAAGTGCA | |
| | ACGACGAATGCATGGAGAGCGTGAAGAACGGAAC | |
| | CTACGACTACCCCAAGTACAGCGAGGAAAGCAAA | |
| | CTGAACAGAGAGAAGATCGACGGCGTGAAGTTAG | |
| | AGAGCATGGGCGTGTATCAG | |
| Cobra_P1: consensus HA | ATGAAGGCCCGCCTCTTGGTGCTGCTGTGCGCCC | 32 |
| sequence P1 for H1 subtype, | TGGCGGCCACAGACGCCGACACAATCTGTATCGG | |
| with transmembrane domain | CTACCACGCCAATAATAGCACCGATACCGTGGAT | |
| (second underlined) | ACCGTGCTCGAGAAGAACGTCACCGTTACACACT | |
| | CCGTGAATTTACTGGAGGACAGCCACAATGGCAA | |
| | GCTCTGCAAACTGAAGGGTATCGCCCCACTCCAA | |
| | CTGGGCAAGTGCAACATCGCAGGCTGGCTGCTGG | |
| | GCAACCCTGAGTGTGAGAGCCTGCTGAGCGCTAG | |
| | AAGCTGGAGCTACATAGTGGAGACACCTAACAGC | |
| | GAAAACGGCACATGCTACCCCGGCGACTTCATCG | |
| | ATTACGAGGAACTGCGGGAGCAGCTGAGTAGCGT | |
| | GAGCTCCTTTGAGAGATTTGAGATCTTCCCCAAA | |
| | GAGAGCAGCTGGCCCAACCATAATACCACCAAAG | |
| | GCGTGACCGCCGCTTGCAGTCATGCAGGGAAAAG | |
| | TAGCTTCTACCGGAACCTGCTCTGGTTGACCAAG | |
| | AAGGGAGGGAGCTACCCAAAGTTGAGCAAAAGCT | |
| | ACGTGAATAACAAGGGCAAGGAGGTGCTCGTGCT | |
| | GTGGGGAGTCCACCATCCCAGCACATCCACTGAT | |
| | CAGCAGTCCCTGTATCAGAACGAAAACGCCTACG | |
| | TGAGTGTGGTGAGCTCTAACTACAACAGACGGTT | |
| | CACCCCTGAAATTGCTGAGAGGCCAAAGGTGAGA | |
| | GGCCAGGCCGGCAGAATGAACTACTATTGGACCC | |
| | TCCTGGAGCCTGGGGACACGATCATCTTCGAGGC | |
| | GACCGGGAACCTCATCGCTCCCTGGTATGCCTTC | |
| | GCCCTGAGCAGGGGCAGTGGCAGCGGAATCATCA | |
| | CCAGCAACGCCAGCATGCATGAGTGCAACACTAA | |
| | ATGCCAGACCCCCCAGGGGGCCATCAACAGCTCC | |
| | CTGCCCTTCCAGAACATCCATCCTGTGACCATTG | |
| | GGGAGTGCCCCAAGTACGTGAGGTCCACCAAGCT | |
| | GAGGATGGTGACTGGACTGAGGAACATCCCCAGC | |
| | ATCCAGAGCCGGGGGCTGTTTGGCGCCATTGCCG | |
| | GCTTTATCGAGGGTGGCTGGACAGGTATGATTGA | |
| | TGGCTGGTACGGATACCACCACCAGAACGAGCAG | |
| | GGGAGTGGGTATGCTGCCGACCAGAAATCTACTC | |
| | AGAACGCCATCAATGGCATCACCAATAAGGTGAA | |
| | CAGCGTCATCGAGAAGATGAACACCCAGTTCACC | |
| | GCTGTGGGCAAGGAGTTCAACAACCTGGAAAAGC | |
| | GCATGGAGAACCTGAACAAGAAGGTGGACGACGG | |
| | CTTCCTGGACATCTGGACCTACAACGCCGAGCTG | |
| | CTTGTCCTCCTGGAGAACGAGAGGACCTTGGACT | |
| | TCCATGACAGCAATGTGAAGAACCTCTACGAGAA | |
| | AGTGAAGAGCCAGCTGAGAAACAATGCTAAGGAG | |
| | ATCGGCAACGGCTGCTTCGAGTTTTACCACAAGT | |
| | GCGACAATGAGTGCATGGAGAGCGTGAAGAATGG | |
| | CACTTATGACTACCCCAAGTACTCAGAGGAGTCC | |
| | AAACTGAATAGAGAGAAGATTGATGGCGTCAAGC | |
| | TAGAGTCCATGGGCGTTTACCAGATCCTGGCAAT | |
| | CTATAGCACCGTGGCCAGCTCCCTGGTGCTGCTG | |
| | GTGTCACTGGGAGCCATATCCTTCTGGATGTGCT | |
| | CCAACGGCAGCCTTCAGTGTAGAATCTGCATC | |
| Cobra_X3: consensus HA | ATGGAGGCCCGCCTGCTCGTGCTTCTGTGCGCCT | 33 |
| sequence X3 for H1 | TTGCCGCCACTAACGCCGACACCATCTGTATCGG | |
| subtype, with | CTACCACGCCAACAATAGTACAGATACCGTGGAC | |
| transmembrane domain | ACTGTGCTGGAGAAGAACGTAACAGTGACACATT | |
| (second underlined) | CTGTCAACCTGCTCGAGGACTCTCATAATGGCAA | |
| | GCTGTGCCGCCTGAAGGGCATCGCCCCTCTGCAG | |
| | CTGGGAAATTGCTCCGTGGCCGGCTGGATCCTGG | |
| | GCAATCCGGAATGCGAAAGCCTGTTCAGCAAGGA | |
| | GAGCTGGAGCTACATCGCCGAGACACCTAACCCT | |
| | GAGAACGGGACCTGCTACCCTGGATACTTCGCCG | |
| | ACTACGAAGAGCTGCGGGAGCAGCTCAGCTCAGT | |
| | GTCATCCTTCGAGCGGTTCGAGATCTTCCCCAAG | |
| | GAGAGCTCTTGGCCCAACCACACCGTGACCAAGG | |
| | GCGTCACAGCAAGCTGTAGCCACAACGGCAAGAG | |
| | CTCCTTCTATAGAAACCTGCTGTGGCTGACCGAG | |
| | AAGAACGGCCTGTACCCCAATCTGAGTAAGTCCT | |
| | ACGTGAACAACAAGGAAAAGGAAGTGCTGGTGCT | |
| | GTGGGGCGTGCACCACCCCTCCAACATCGGCGAC | |
| | CAGCGCGCCATCTACCACACTGAGAATGCATACG | |
| | TAAGCGTTGTCAGCTCCCACTATAGTAGGAGATT | |
| | CACACCCGAGATCGCTAAGAGGCCCAAGGTGAGA | |
| | GACCAGGAGGGCAGAATCAATTATTACTGGACCC | |
| | TGCTGGAGCCCGGAGACACCATTATCTTCGAAGC | |
| | TAACGGCAATTTGATCGCCCCTTGGTATGCCTTT | |
| | GCCCTCTCAAGGGGTTTCGGGAGCGGAATTATCA | |
| | CCTCCAATGCCAGCATGGATGAGTGCGACGCCAA | |
| | GTGCCAGACGCCTCAGGGCGCCATTAATTCCTCC | |
| | CTGCCCTTCCAGAACGTGCACCCCGTGACCATCG | |
| | GGGAGTGCCCCAAGTATGTTAGATCCACTAAGCT | |
| | CAGGATGGTGACAGGACTGCGCAACATCCCGAGC | |
| | ATTCAGAGCAGGGGCCTCTTCGGGGCCATTGCTG | |
| | GGTTCATCGAGGGCGGGTGGACCGGCATGATCGA | |
| | CGGCTGGTATGGCTACCACCACCAGAACGAGCAG | |
| | GGCAGCGGGTACGCTGCTGACCAAAAGTCCACCC | |
| | AAAATGCTATCAACGGCATCACCAACAAGGTTAA | |
| | TAGCGTCATCGAAAAGATGAATACCCAGTTCACA | |
| | GCCGTGGGAAAGGAATTCAACAAGCTGGAACGAC | |
| | GGATGGAGAACCTGAATAAGAAGGTGGACGACGG | |
| | GTTCCTGGACATCTGGACTTATAACGCTGAGCTG | |
| | CTCGTGCTGTTAGAGAACGAGAGAACCCTGGACT | |
| | TTCACGACAGCAACGTGAAGAACCTGTACGAGAA | |
| | GGTGAAGTCTCAGCTGAAAAATAACGCTAAGGAA | |
| | ATTGGCAACGGGTGCTTCGAATTCTATCACAAGT | |
| | GCAACAACGAATGCATGGAGAGTGTTAAGAACGG | |
| | AACCTATGACTACCCCAAGTACAGTGAGGAAAGT | |
| | AAACTGAATAGGGAGAAGATCGACGGCGTGAAAC | |
| | TGGAGTCCATGGGGGTTTACCAGATTCTGGCCAT | |
| | CTATAGCACCGTGGCCAGCAGCTTAGTGCTGCTG | |
| | GTGTCCCTCGGCGCTATTAGCTTCTGGATGTGCA | |
| | GCAACGGAAGCCTGCAGTGTCGGATATGCATC | |
| ConH1_ferritin: consensus | ATGAAGGCGAAGCTCCTTGTGCTGCTCTGCGCGT | 34 |
| HA sequence for subtype | TCACCGCCACCGACGCAGATACAATTTGCATCGG | |
| H1, with ferritin (second | ATACCACGCCAACAATTCCACCGACACCGTGGAC | |
| underlined) for particle | ACCGTTCTGGAGAAAAACGTGACGGTGACCCACA | |
| formation | GCGTGAACCTCCTGGAGGATAGCCATAACGGCAA | |
| | GCTGTGTAAGCTGAAAGGCATCGCCCCCCTGCAG | |
| | CTGGGAAAGTGCAACATTGCTGGATGGATCCTGG | |
| | GAAATCCCGAGTGTGAAAGCCTCATTAGCAAACG | |
| | CAGCTGGAGCTACATTGTGGAGACCCCAAATTCT | |
| | GAGAATGGGACCTGTTACCCTGGCGACTTTGCCG | |
| | ACTACGAGGAGCTGAGAGAGCAGTTGAGCAGCGT | |
| | CAGCTCCTTCGAGAGATTCGAAATCTTTCCAAAG | |
| | GAGTCTTCGTGGCCCAACCACAACGTGACTAAGG | |
| | GCGTCACCGCAGCTTGTAGCCACGCGGGCAAATC | |
| | TTCCTTCTACAGAAACCTACTGTGGCTCACCGAG | |
| | AAAAACGGCAGCTACCCCAAGCTGAGCAAGAGCT | |
| | ACGTGAATAACAAAGAGAAGGAAGTGCTGGTGCT | |
| | GTGGGGCGTCCACCACCCCAGCAACATCACAGAC | |
| | CAAAGAACACTCTACCAGAACGAGAACGCCTACG | |
| | TGAGTGTGGTGTCCAGCCATTACAACCGCCGATT | |
| | CACCCCCGAGATCGCCAAACGGCCCAAAGTGCGG | |
| | GGCCAGGCCGGAAGAATTAACTACTACTGGACCC | |
| | TCCTGGAACCAGGAGACACCATTATCTTCGAAGC | |
| | CAATGGCAATCTGATCGCTCCCTGGTACGCCTTC | |
| | GCACTGTCGAGAGGGTTTGGCAGCGGCATCATCA | |
| | CCTCCAACGCCCCAATGCATGAATGTGATACCAA | |
| | GTGCCAGACCCCACAGGGCGCCATTAACAGCAGC | |
| | CTGCCATTCCAGAACGTCCATCCCGTGACAATCG | |
| | GCGAGTGTCCTAAGTACGTGCGCTCAACGAAACT | |
| | GAGGATGGTGACAGGACTGAGAAACATTCCCTCA | |
| | ATCCAGAGCAGAGGGCTGTTCGGCGCCATAGCCG | |
| | GATTCATTGAGGGCGGATGGACAGGCATGATTGA | |
| | CGGCTGGTATGGCTACCACCATCAGAACGAGCAA | |
| | GGCAGTGGCTACGCAGCCGACCAGAAGAGCACAC | |
| | AGAACGCCATTAACGGGATCACCAACAAGGTGAA | |
| | TAGCGTGATCGAGAAGATGAATACCCAGTTCACT | |
| | GCCGTGGGTAAGGAGTTCAACAAGCTGGAGAAGC | |
| | GGATGGAGAACCTCAACAAGAAAGTCGATGATGG | |
| | CTTCCTGGACATCTGGACCTATAATGCTGAACTG | |
| | CTCGTGCTACTTGAGAATGAGAGGACGCTTGACT | |
| | TTCACGACTCCAACGTAAAAAACCTGTACGAGAA | |
| | GGTGAAGTCGCAGCTGAAAAATAACGCCAAGGAA | |
| | ATCGGCAACGGCTGTTTTGAGTTTTACCATAAAT | |
| | GCAATAACGAGTGCATGGAGAGCGTGAAGAATGG | |
| | CACCTACGACTATCCCAAATACTCCGAGGAGAGC | |
| | AAGCTCAACCGGGAGAAAATCGATAGCGGCGGGG | |
| | ATATCATTAAGCTGCTTAACGAGCAGGTCAACAA | |
| | GGAGATGCAGTCAAGCAACCTTTACATGAGCATG | |
| | AGCAGCTGGTGTTACACACACAGCCTGGACGGAG | |
| | CCGGACTGTTCCTGTTCGACCACGCTGCAGAGGA | |
| | ATATGAGCACGCTAAGAAGCTTATAATTTTCCTC | |
| | AACGAGAATAACGTGCCCGTCCAGCTGACCTCCA | |
| | TCAGCGCCCCCGAGCACAAGTTTGAGGGCCTGAC | |
| | CCAGATCTTCCAGAAGGCCTACGAGCACGAGCAG | |
| | CACATCAGCGAGTCTATCAACAACATCGTAGACC | |
| | ATGCAATCAAGTCTAAGGACCACGCTACATTTAA | |
| | CTTTCTGCAATGGTACGTGGCTGAACAACACGAG | |
| | GAGGAGGTACTGTTCAAGGATATTCTCGACAAGA | |
| | TCGAACTCATCGGGAATGAGAACCACGGCCTGTA | |
| | CCTGGCCGACCAGTACGTGAAAGGAATTGCCAAA | |
| | TCCAGAAAGTCC | |
| ConH3_ferritin: consensus | ATGAAGACAATCATTGCCCTGAGCTACATTTTTT | 35 |
| HA sequence for subtype | GCTTAGTGTTTGCTCAGAAACTGCCAGGCAACGA | |
| H3, with ferritin (second | TAATTCAACAGCCACCTTGTGCCTCGGCCACCAC | |
| underlined) for particle | GCTGTGCCTAACGGCACTCTGGTGAAGACCATCA | |
| formation | CCAACGACCAGATCGAGGTGACCAACGCCACGGA | |
| | GCTGGTGCAGTCAAGCTCCACCGGAAGAATCTGC | |
| | GATAGCCCCCATAGGATTCTGGATGGCACCAACT | |
| | GCACCCTGATTGACGCCCTGCTCGGCGATCCCCA | |
| | CTGCGACGGTTTCCAAAACAAGGAGTGGGACCTG | |
| | TTTGTGGAGAGAAGCAAGGCCTATTCAAATTGCT | |
| | ACCCTTACGACGTCCCTGATTACGCCTCACTCAG | |
| | GTCCCTGGTGGCCAGCAGCGGGACCCTGGAATTC | |
| | AACAATGAGGGGTTCAACTGGACCGGGGTGACCC | |
| | AAAACGGCGGCTCCAGCGCCTGTAAGAGGGGCAG | |
| | CGACAAGTCCTTCTTCTCTAGGCTGAACTGGTTG | |
| | CACAAACTGAAGTACAAGTACCCTGCATTAAACG | |
| | TGACCATGCCCAACAACGATAAATTCGACAAGCT | |
| | GTACATCTGGGGAGTGCATCACCCCAGCACAGAC | |
| | TCAGACCAGACCAGTCTGTATGTGCAGGCAAGCG | |
| | GGAGGGTGACGGTCTCCACCAAGCGGAGCCAGCA | |
| | GACCGTGATCCCCAACATCGGCTCCAGACCATGG | |
| | GTCAGGGGCCTGAGCAGCCGGATCTCCATCTACT | |
| | GGACCATAGTGAAGCCTGGCGACATCCTGCTGAT | |
| | CAACAGCACCGGCAACCTCATCGCCCCTCGCGGT | |
| | TACTTCAAGATCCGTAGTGGCAAATCAAGCATCA | |
| | TGAGATCCGACGCACCCATCGGGACCTGCAATAG | |
| | CGAGTGCATCACCCCCAACGGATCTATCCCTAAT | |
| | GACAAGCCTTTTCAGAACGTGAACCGGATTACCT | |
| | ATGGTGCCTGCCCCAGATACGTGAAGCAGAACAC | |
| | CCTGAAGCTGGCGACCGGCATGCGCAACGTGCCG | |
| | GAAAAGCAGACCCGGGGCATCTTCGGCGCCATTG | |
| | CCGGGTTTATTGAGAATGGCTGGGAAGGCATGGT | |
| | GGATGGGTGGTACGGCTTTAGGCATCAGAACTCT | |
| | GAGGGTACTGGTCAGGCCGCCGACCTGAAATCCA | |
| | CCCAGGCCGCCATTGACCAGATTAACGGGAAACT | |
| | TAACAGACTGATTGAGAAGACCAATGAGAAGTTC | |
| | CACCAGATCGAAAAGGAATTCTCCGAAGTGGAGG | |
| | GCAGGATTCAGGACTTAGAGAAATATGTGGAGGA | |
| | TACCAAGATCGACCTGTGGAGCTATAACGCCGAG | |
| | CTGCTTGTGGCTCTGGAGAACCAGCACACCATCG | |
| | ATCTGACCGACAGCGAGATGAATAAGCTGTTCGA | |
| | GAGGACACGCAAGCAGCTGAGGGAGAACGCCGAG | |
| | GACATGGGGAACGGGTGCTTTAAGATCTATCACA | |
| | AGTGCGACAATGCCTGCATCGGGTCTATCAGAAA | |
| | TGGCACTTATGATCATGACGTGTACAGAGATGAG | |
| | GCCCTGAATAATAGATTTCAAATTAAGTCCGGGG | |
| | GTGACATCATTAAACTGCTGAACGAACAAGTGAA | |
| | TAAAGAGATGCAGAGCTCTAACTTGTACATGAGC | |
| | ATGAGCAGTTGGTGCTACACACACTCTCTGGACG | |
| | GGGCTGGCCTGTTCCTGTTCGATCACGCAGCAGA | |
| | GGAGTATGAGCACGCCAAAAAACTGATTATCTTC | |
| | CTCAACGAGAACAACGTGCCCGTCCAGCTCACCT | |
| | CCATCTCAGCCCCCGAGCACAAGTTCGAGGGCCT | |
| | CACCCAGATCTTCCAGAAAGCATATGAGCATGAA | |
| | CAGCATATCAGTGAAAGCATCAACAATATCGTGG | |
| | ACCACGCTATTAAATCAAAGGATCACGCCACCTT | |
| | CAACTTTCTGCAGTGGTATGTCGCCGAGCAGCAT | |
| | GAGGAGGAGGTGCTTTTTAAAGACATCCTGGACA | |
| | AGATCGAGCTGATCGGCAACGAAAACCATGGCCT | |
| | GTACCTGGCTGACCAGTATGTGAAGGGAATTGCC | |
| | AAGTCCAGAAAATCC | |
| MRK_pH1_Con_ferritin: | ATGAAGGCGATTCTGGTTGTGCTGCTGTACACCT | 36 |
| consensus HA sequence for | TCGCCACCGCCAACGCCGACACACTGTGCATTGG | |
| pandemic H1 strains, with | CTACCACGCCAATAACAGCACGGACACCGTGGAC | |
| ferritin (second underlined) | ACGGTGCTGGAGAAAAACGTGACCGTGACCCACA | |
| for particle formation | GCGTGAACCTGCTGGAGGACAAGCACAACGGTAA | |
| | GTTGTGCAAGCTCAGGGGAGTGGCACCACTGCAC | |
| | TTGGGCAAGTGTAATATCGCTGGCTGGATATTGG | |
| | GAAATCCAGAGTGCGAAAGCCTGAGTACTGCCTC | |
| | CAGCTGGAGCTACATTGTGGAGACCAGCAGCAGC | |
| | GACAACGGCACCTGCTACCCCGGCGACTTCATCG | |
| | ACTATGAGGAATTGAGGGAACAGCTGAGTTCAGT | |
| | TTCCAGCTTCGAGCGATTTGAGATATTTCCCAAG | |
| | ACGTCCTCTTGGCCCAACCACGACAGCAACAAGG | |
| | GCGTGACAGCCGCCTGCCCCCACGCCGGGGCGAA | |
| | GAGCTTCTACAAGAACCTGATCTGGCTGGTGAAG | |
| | AAGGGCAACAGCTACCCAAAGCTATCCAAGTCCT | |
| | ATATTAACGACAAAGGCAAGGAGGTGTTGGTGCT | |
| | CTGGGGCATTCACCACCCCTCCACCTCCGCCGAC | |
| | CAGCAAAGTCTTTACCAGAACGCGGACGCCTACG | |
| | TCTTTGTCGGCACCAGCAGATACAGCAAGAAGTT | |
| | TAAGCCCGAGATTGCTATCAGACCCAAGGTGAGA | |
| | GACCAGGAAGGCAGAATGAACTATTATTGGACCC | |
| | TGGTGGAACCCGGCGACAAAATAACATTCGAAGC | |
| | CACCGGGAATCTGGTGGTGCCCAGATATGCCTTT | |
| | GCCATGGAGCGCAATGCCGGCAGCGGCATTATTA | |
| | TCTCTGACACCCCCGTGCACGACTGCAACACCAC | |
| | CTGTCAGACCCCTAAGGGGGCTATCAACACCAGC | |
| | CTGCCCTTCCAGAATATTCACCCCATCACTATCG | |
| | GCAAGTGCCCCAAGTACGTCAAGAGCACAAAACT | |
| | GAGACTGGCCACAGGGCTGAGGAATGTACCTAGC | |
| | ATCCAGTCCAGAGGGCTGTTCGGGGCCATCGCTG | |
| | GCTTCATCGAAGGAGGCTGGACCGGCATGGTCGA | |
| | TGGATGGTACGGATATCACCACCAAAACGAGCAG | |
| | GGGTCAGGATACGCCGCTGACCTGAAGAGCACCC | |
| | AGAACGCCATCGACAAGATCACCAACAAGGTGAA | |
| | TAGCGTGATCGAGAAGATGAACACCCAGTTCACC | |
| | GCAGTGGGCAAGGAGTTCAACCACCTGGAGAAGA | |
| | GAATCGAGAACCTGAACAAGAAAGTGGATGACGG | |
| | GTTCCTGGACATCTGGACCTACAACGCCGAGCTT | |
| | CTGGTGCTCTTGGAGAATGAGAGAACCCTGGATT | |
| | ATCATGACAGCAATGTCAAAAACCTCTACGAGAA | |
| | GGTGCGGAGCCAGCTGAAGAACAACGCAAAGGAG | |
| | ATTGGCAACGGCTGCTTCGAGTTTTATCACAAGT | |
| | GCGACAACACTTGTATGGAGAGCGTTAAGAATGG | |
| | CACTTACGATTACCCCAAGTACTCCGAGGAAGCC | |
| | AAGCTGAACAGAGAAGAAATCGACTCCGGCGGCG | |
| | ACATAATCAAGCTCCTGAACGAACAGGTGAACAA | |
| | GGAGATGCAAAGCTCCAACCTCTACATGAGCATG | |
| | AGCTCATGGTGCTACACTCACAGCCTGGACGGAG | |
| | CTGGACTGTTCTTGTTCGACCACGCGGCCGAGGA | |
| | GTACGAGCACGCCAAGAAGCTCATCATCTTCCTT | |
| | AACGAGAATAACGTGCCAGTGCAGCTCACCTCCA | |
| | TCAGCGCCCCCGAGCATAAGTTCGAGGGTCTGAC | |
| | CCAAATCTTCCAGAAGGCTTACGAGCATGAGCAG | |
| | CACATCAGCGAGAGCATTAACAACATCGTGGATC | |
| | ACGCTATTAAATCTAAAGACCACGCCACCTTCAA | |
| | CTTCCTGCAGTGGTACGTGGCAGAACAGCACGAG | |
| | GAGGAGGTCCTGTTCAAGGATATACTGGACAAAA | |
| | TCGAGCTGATCGGCAACGAGAACCACGGCCTGTA | |
| | CCTGGCCGATCAGTACGTCAAAGGTATTGCCAAG | |
| | TCTCGCAAGAGC | |
| MRK_sH1_Con_ferritin: | ATGAAGGTGAAGCTGCTTGTGCTGCTGTGCACCT | 37 |
| consensus HA sequence for | TCACCGCTACCTACGCAGACACAATCTGTATCGG | |
| seasonal H1 strains, with | ATACCACGCCAATAACTCAACCGATACAGTGGAC | |
| ferritin (second underlined) | ACCGTGCTCGAGAAGAACGTGACAGTGACGCACA | |
| for particle formation | GCGTGAACCTGCTTGAGGATTCCCATAACGGTAA | |
| | GCTCTGTCTGCTGAAGGGCATCGCCCCTCTTCAG | |
| | CTGGGAAACTGCTCCGTTGCCGGCTGGATCCTGG | |
| | GCAACCCCGAGTGTGAGCTTCTGATCAGCAAGGA | |
| | GTCGTGGTCATATATCGTGGAGACCCCTAATCCA | |
| | GAGAACGGAACCTGTTACCCCGGCTACTTTGCCG | |
| | ACTACGAGGAGCTCAGAGAGCAGCTGAGCAGCGT | |
| | GAGCAGCTTCGAGAGATTCGAGATCTTCCCCAAG | |
| | GAGAGCAGTTGGCCTAATCACACCGTGACCGGCG | |
| | TGAGCGCCTCCTGCAGCCACAACGGCAAGTCTTC | |
| | CTTTTACAGAAACCTGCTGTGGCTGACAGGCAAA | |
| | AACGGGTTGTACCCTAACCTGAGCAAGTCCTATG | |
| | CTAACAATAAGGAGAAGGAAGTCCTGGTGTTGTG | |
| | GGGCGTTCACCATCCCCCAAACATCGGAGACCAA | |
| | CGCGCCCTATATCACACTGAGAACGCCTACGTGA | |
| | GCGTGGTGTCAAGCCACTATAGCAGACGGTTCAC | |
| | CCCCGAAATCGCAAAGAGACCGAAGGTGCGGGAC | |
| | CAGGAGGGAAGGATTAACTATTACTGGACACTCC | |
| | TGGAGCCCGGGGACACTATCATCTTTGAAGCCAA | |
| | CGGGAACCTCATCGCACCCAGGTACGCTTTCGCT | |
| | CTGTCCAGGGGATTCGGGAGCGGTATCATTACCT | |
| | CGAACGCCCCGATGGATGAGTGCGACGCCAAATG | |
| | CCAAACCCCCCAGGGCGCTATTAACTCTAGCCTC | |
| | CCTTTTCAGAACGTGCACCCCGTGACCATCGGAG | |
| | AGTGCCCCAAGTACGTGCGGAGCGCTAAGCTCAG | |
| | GATGGTGACCGGCCTGCGGAACATCCCCTCTATC | |
| | CAATCCAGGGGTCTGTTCGGCGCCATTGCCGGAT | |
| | TTATCGAGGGCGGGTGGACCGGGATGGTGGATGG | |
| | ATGGTATGGATACCACCATCAGAATGAACAAGGC | |
| | AGCGGATACGCCGCCGATCAGAAGTCAACACAAA | |
| | ACGCCATCAACGGAATTACCAACAAAGTCAACTC | |
| | CGTGATCGAGAAGATGAACACCCAGTTCACGGCC | |
| | GTGGGCAAAGAGTTCAACAAGCTCGAGCGGCGAA | |
| | TGGAGAACCTCAACAAGAAGGTGGACGATGGATT | |
| | CCTGGACATCTGGACGTACAATGCCGAACTGCTC | |
| | GTGCTGCTGGAAAACGAGAGAACACTCGATTTCC | |
| | ACGACAGCAACGTGAAGAATCTGTATGAGAAGGT | |
| | CAAATCCCAGTTGAAGAACAACGCCAAGGAGATC | |
| | GGCAATGGCTGTTTCGAGTTCTATCACAAGTGTA | |
| | ATGACGAGTGCATGGAGAGCGTTAAGAACGGCAC | |
| | CTACGACTACCCCAAATACAGCGAAGAGAGCAAG | |
| | CTGAACCGTGAGAAGATCGACAGCGGAGGCGATA | |
| | TCATCAAGCTGCTGAACGAACAGGTGAACAAGGA | |
| | GATGCAGTCCAGCAATCTCTACATGAGTATGTCC | |
| | TCGTGGTGCTACACCCACAGCCTGGATGGAGCCG | |
| | GACTGTTTCTGTTCGACCACGCCGCCGAGGAGTA | |
| | CGAGCATGCCAAAAAGCTGATCATCTTCCTCAAT | |
| | GAAAACAACGTGCCCGTGCAGTTGACCAGCATCA | |
| | GCGCCCCCGAGCATAAATTCGAGGGACTGACACA | |
| | GATCTTTCAGAAGGCCTATGAGCACGAGCAGCAC | |
| | ATCAGCGAAAGCATCAACAACATCGTGGACCACG | |
| | CCATCAAGTCCAAGGATCACGCCACCTTCAACTT | |
| | CCTGCAGTGGTACGTTGCCGAACAGCACGAGGAG | |
| | GAGGTGCTGTTTAAGGACATCCTGGACAAAATCG | |
| | AACTGATCGGAAACGAGAACCATGGTCTGTACCT | |
| | CGCCGACCAGTACGTGAAGGGAATCGCCAAGAGC | |
| | AGGAAGTCG | |
| Cobra_P1_ferritin: | ATGAAGGCCAGACTGTTGGTGCTGCTGTGTGCCC | 38 |
| consensus HA sequence P1 | TTGCCGCCACAGACGCCGACACCATCTGTATCGG | |
| for H1 subtype, with ferritin | CTACCACGCTAATAACAGCACCGACACCGTGGAC | |
| second underlined) for | ACAGTGCTTGAAAAGAACGTGACAGTGACCCACA | |
| particle formation | GCGTTAACCTGCTTGAGGACTCTCACAACGGGAA | |
| | GCTGTGTAAACTGAAGGGGATCGCCCCTCTGCAG | |
| | CTGGGCAAGTGCAACATCGCTGGCTGGCTGCTGG | |
| | GAAATCCCGAGTGTGAGAGCCTGCTGTCCGCTCG | |
| | TAGCTGGAGCTACATAGTTGAAACCCCTAACAGC | |
| | GAGAATGGCACCTGCTACCCTGGAGACTTCATCG | |
| | ACTACGAGGAGCTCAGAGAGCAGCTGAGCAGCGT | |
| | GAGCTCGTTTGAAAGATTTGAGATCTTTCCCAAG | |
| | GAGTCCTCATGGCCCAACCACAACACTACCAAAG | |
| | GCGTGACCGCTGCTTGTTCACACGCTGGCAAATC | |
| | CTCCTTCTACCGGAACCTGCTGTGGCTGACCAAG | |
| | AAAGGCGGATCCTACCCCAAACTGAGCAAGTCAT | |
| | ACGTGAATAACAAGGGCAAAGAGGTGCTGGTGCT | |
| | GTGGGGCGTGCACCACCCCTCCACCAGCACCGAT | |
| | CAGCAAAGCCTGTACCAGAACGAGAACGCCTACG | |
| | TCAGCGTTGTGAGCAGCAACTACAACCGGAGATT | |
| | CACCCCCGAGATTGCCGAGAGACCTAAGGTGAGA | |
| | GGGCAGGCTGGCAGAATGAACTACTATTGGACTC | |
| | TGCTGGAGCCCGGAGACACAATTATCTTCGAGGC | |
| | CACTGGCAATCTGATCGCACCCTGGTACGCCTTC | |
| | GCCTTAAGCAGGGGCAGCGGGTCTGGAATTATCA | |
| | CTTCCAATGCCAGCATGCACGAGTGCAACACCAA | |
| | GTGCCAGACCCCCCAGGGCGCCATTAACAGCAGC | |
| | CTGCCCTTCCAGAACATCCACCCCGTCACTATCG | |
| | GCGAGTGCCCCAAGTATGTGAGGAGCACTAAGCT | |
| | GAGGATGGTGACCGGGCTTAGAAACATCCCCAGC | |
| | ATCCAGTCAAGAGGCCTATTCGGCGCAATAGCCG | |
| | GCTTCATTGAAGGCGGCTGGACCGGGATGATCGA | |
| | TGGCTGGTATGGCTATCACCATCAGAACGAGCAA | |
| | GGCTCCGGGTACGCCGCCGACCAGAAATCCACAC | |
| | AGAATGCCATCAATGGAATTACTAACAAGGTTAA | |
| | TTCCGTCATCGAGAAGATGAATACCCAGTTTACC | |
| | GCCGTGGGAAAGGAGTTCAACAATCTGGAGAAGC | |
| | GGATGGAGAACCTCAACAAGAAGGTAGATGATGG | |
| | ATTCCTCGACATCTGGACATACAATGCTGAACTG | |
| | CTGGTGCTGCTCGAGAACGAGAGAACCTTAGACT | |
| | TCCACGACAGCAACGTGAAGAATCTGTACGAGAA | |
| | GGTTAAGTCTCAACTGAGAAATAACGCTAAGGAG | |
| | ATTGGCAATGGCTGTTTCGAGTTCTACCACAAGT | |
| | GTGACAACGAATGTATGGAATCTGTGAAGAACGG | |
| | GACCTACGACTACCCCAAGTACAGCGAGGAGAGC | |
| | AAGCTGAACAGAGAGAAGATCGACTCAGGCGGCG | |
| | ACATCATTAAGCTGCTGAATGAGCAGGTTAATAA | |
| | GGAGATGCAGAGCTCCAATCTGTATATGAGTATG | |
| | AGCAGCTGGTGTTACACTCACTCCCTGGACGGCG | |
| | CCGGACTGTTCCTGTTCGACCACGCTGCAGAGGA | |
| | GTACGAACACGCAAAAAAGCTGATAATCTTTCTG | |
| | AATGAAAACAACGTGCCCGTCCAGCTGACCTCTA | |
| | TTTCTGCCCCAGAGCACAAGTTCGAGGGCCTGAC | |
| | ACAGATCTTCCAAAAGGCCTACGAACACGAGCAG | |
| | CACATCAGCGAGTCAATCAACAACATAGTCGATC | |
| | ACGCCATTAAGTCTAAGGACCACGCCACCTTCAA | |
| | CTTCCTCCAGTGGTATGTGGCCGAGCAGCACGAG | |
| | GAGGAGGTTCTTTTTAAGGATATTCTCGATAAAA | |
| | TCGAGTTGATCGGCAACGAGAATCATGGCCTGTA | |
| | CCTGGCAGACCAATATGTGAAGGGGATCGCCAAG | |
| | TCAAGGAAGAGC | |
| Cobra_X3_ferritin: | ATGGAGGCCAGACTGCTGGTGCTGCTGTGCGCCT | 39 |
| consensus HA sequence X3 | TCGCCGCCACCAACGCAGACACCATCTGCATTGG | |
| for H1 subtype, with ferritin | CTACCACGCCAACAACAGCACCGATACCGTGGAC | |
| (second underlined) for | ACAGTGCTCGAAAAGAACGTGACAGTGACTCACA | |
| particle formation | GCGTGAACCTCCTGGAGGACAGCCACAACGGCAA | |
| | GCTGTGCCGGCTGAAGGGTATCGCCCCCTTGCAG | |
| | CTGGGAAACTGCAGCGTGGCAGGGTGGATCTTGG | |
| | GCAATCCCGAGTGCGAAAGTCTGTTTTCTAAGGA | |
| | GTCCTGGTCCTACATCGCCGAGACACCGAACCCC | |
| | GAAAACGGAACATGCTATCCTGGCTACTTCGCTG | |
| | ACTACGAAGAGCTGCGGGAGCAGCTTAGCTCCGT | |
| | CTCCAGCTTTGAGCGGTTTGAGATCTTCCCGAAA | |
| | GAGTCTAGCTGGCCCAATCACACAGTCACCAAGG | |
| | GGGTGACCGCATCCTGCAGCCACAACGGCAAGTC | |
| | CTCTTTCTACAGAAACCTGCTGTGGCTGACCGAG | |
| | AAAAACGGGCTGTACCCTAACCTTTCCAAGAGCT | |
| | ATGTCAACAACAAGGAGAAGGAGGTGCTGGTGCT | |
| | GTGGGGGGTTCACCACCCCAGCAACATCGGAGAC | |
| | CAGAGAGCTATCTATCACACCGAAAACGCCTACG | |
| | TGAGCGTGGTGAGCAGCCATTATAGCAGACGCTT | |
| | CACCCCTGAGATTGCCAAACGGCCCAAAGTGCGG | |
| | GACCAGGAGGGCAGAATCAACTATTACTGGACCC | |
| | TCCTGGAACCTGGCGATACCATTATCTTTGAGGC | |
| | CAACGGCAACCTGATCGCCCCATGGTACGCCTTT | |
| | GCTCTGAGCCGGGGCTTTGGCTCAGGCATCATTA | |
| | CCAGCAACGCCAGCATGGACGAGTGCGATGCCAA | |
| | GTGCCAGACACCCCAGGGCGCCATCAACAGCTCC | |
| | CTGCCCTTTCAAAATGTCCATCCCGTGACCATCG | |
| | GCGAGTGTCCCAAGTACGTCCGGTCCACTAAACT | |
| | GCGGATGGTGACCGGACTCAGAAATATCCCAAGC | |
| | ATCCAGAGCAGAGGCCTGTTTGGCGCCATCGCTG | |
| | GATTTATCGAGGGAGGCTGGACTGGCATGATCGA | |
| | TGGCTGGTACGGCTATCATCATCAGAACGAGCAG | |
| | GGCAGCGGATATGCCGCAGACCAGAAGTCGACCC | |
| | AGAACGCCATCAATGGAATTACCAACAAGGTGAA | |
| | CAGCGTGATCGAGAAGATGAACACCCAGTTCACT | |
| | GCCGTCGGCAAGGAATTCAACAAGCTGGAACGTC | |
| | GGATGGAAAACCTCAACAAAAAGGTGGATGACGG | |
| | CTTCCTGGATATCTGGACCTACAACGCCGAGCTC | |
| | CTGGTGCTCCTTGAGAACGAGAGAACCCTCGATT | |
| | TCCACGATAGCAACGTGAAAAATCTCTACGAAAA | |
| | GGTGAAGAGCCAGCTGAAAAATAACGCCAAGGAG | |
| | ATAGGGAATGGCTGCTTCGAGTTCTACCATAAGT | |
| | GCAACAACGAGTGCATGGAGAGCGTCAAAAACGG | |
| | CACTTACGATTACCCCAAGTATTCAGAAGAGAGC | |
| | AAACTGAACAGGGAAAAAATTGACTCCGGCGGAG | |
| | ACATTATCAAGCTGCTGAATGAACAGGTGAACAA | |
| | AGAGATGCAGAGCTCCAACCTTTACATGAGCATG | |
| | AGCAGCTGGTGCTATACCCATTCCCTCGACGGGG | |
| | CCGGGCTGTTCCTGTTCGACCATGCCGCTGAAGA | |
| | ATACGAGCACGCCAAGAAACTGATCATCTTCTTA | |
| | AACGAGAACAATGTGCCAGTGCAGCTGACCTCAA | |
| | TCAGCGCCCCCGAGCACAAGTTCGAGGGACTCAC | |
| | TCAGATTTTCCAGAAGGCCTACGAGCACGAGCAA | |
| | CACATTAGCGAATCCATCAACAATATCGTGGACC | |
| | ACGCCATAAAGAGCAAGGACCATGCCACCTTTAA | |
| | CTTCCTTCAATGGTACGTGGCCGAGCAGCACGAG | |
| | GAGGAGGTCCTGTTCAAGGACATCCTCGACAAAA | |
| | TCGAGCTGATCGGCAATGAAAACCATGGCCTCTA | |
| | CCTGGCTGACCAGTATGTGAAAGGTATCGCTAAG | |
| | TCAAGAAAAAGC | |
| NP: Wildtype sequence of | ATGGCCAGCCAGGGCACCAAGAGAAGCTACGAGC | 40 |
| nucleoprotein | AGATGGAGACCGACGGCGAGAGACAGAACGCCAC | |
| | CGAGATCAGAGCCAGCGTGGGCAAGATGATCGAC | |
| | GGCATCGGCAGATTCTACATCCAGATGTGCACCG | |
| | AGCTCAAGCTGAGCGACTACGAGGGCAGACTGAT | |
| | CCAGAACAGCCTGACCATCGAAAGAATGGTTCTG | |
| | AGCGCCTTCGACGAGAGAAGAAACAGATACCTGG | |
| | AGGAGCACCCCAGCGCCGGCAAGGACCCCAAGAA | |
| | GACCGGCGGCCCCATCTACAAGAGAGTGGACGGC | |
| | AGATGGATGAGAGAGCTGGTGCTGTACGACAAGG | |
| | AGGAGATCAGAAGAATCTGGAGACAGGCCAACAA | |
| | CGGCGACGACGCCACCGCCGGCCTGACCCACATG | |
| | ATGATCTGGCACAGCAACCTGAACGACACCACCT | |
| | ACCAGAGAACCAGAGCCCTGGTGAGAACCGGCAT | |
| | GGACCCCAGAATGTGCAGCTTAATGCAGGGCAGC | |
| | ACCCTGCCCAGAAGATCCGGCGCCGCTGGTGCCG | |
| | CCGTCAAGGGCATCGGCACCATGGTGATGGAGCT | |
| | GATCCGCATGATCAAGCGCGGCATCAACGACAGA | |
| | AACTTCTGGAGAGGCGAAAACGGCAGAAAGACCA | |
| | GAAGCGCCTACGAGAGAATGTGCAACATCCTGAA | |
| | GGGCAAGTTCCAGACCGCCGCCCAAAGAGCCATG | |
| | ATGGACCAGGTGAGAGAGAGCAGAAACCCCGGCA | |
| | ACGCCGAGATCGAAGACCTGATCTTCAGCGCCAG | |
| | ATCGGCCCTGATCCTGAGAGGCAGCGTGGCCCAC | |
| | AAGAGCTGCCTGCCCGCCTGCGTGTATGGCCCCG | |
| | CCGTGAGCAGCGGCTACAACTTCGAGAAGGAGGG | |
| | CTACAGCCTGGTGGGCATCGACCCCTTCAAGCTG | |
| | CTGCAGAACTCTCAGGTGTATAGCCTGATCAGAC | |
| | CCAACGAGAACCCCGCCCACAAGAGCCAGCTGGT | |
| | GTGGATGGCCTGCCACAGCGCCGCCTTCGAGGAC | |
| | CTGAGACTGCTGAGCTTCATCAGAGGTACCAAGG | |
| | TGTCCCCCAGAGGCAAGCTGAGCACCAGAGGTGT | |
| | GCAGATCGCCAGCAATGAGAACATGGACAATATG | |
| | GAGAGCAGCACCCTGGAGCTAAGAAGCAGGTACT | |
| | GGGCCATCCGGACCAGAAGCGGCGGCAATACCAA | |
| | CCAGCAGAGAGCCAGCGCCGGCCAGATCAGCGTG | |
| | CAGCCCACCTTCAGCGTGCAGAGAAACCTGCCCT | |
| | TTGAGAAGAGCACCGTGATGGCCGCCTTCACCGG | |
| | CAACACCGAGGGCAGAACCAGCGACATGAGAGCC | |
| | GAGATCATCAGAATGATGGAGGGCGCCAAGCCCG | |
| | AGGAGGTGAGCTTTAGAGGCAGAGGCGTGTTCGA | |
| | GCTGAGCGACGAGAAGGCCACCAACCCAATTGTG | |
| | CCCAGCTTCGACATGTCGAACGAGGGCAGCTACT | |
| | TCTTCGGCGACAACGCCGAGGAGTACGACAAC | |
| MRK_pH1_Con_RBD | ATGAAGGTGAAGCTGCTGGTGCTGCTGTGCACCT | 64 |
| Receptor Binding Domain | TCACCGCCACCTACGCCGGCGTGGCCCCTCTGCA | |
| of consensus pH1 sequence. | CCTGGGCAAGTGCAACATCGCCGGCTGGATCCTG | |
| | GGCAACCCTGAGTGCGAGAGCCTTAGCACAGCCT | |
| | CCTCCTGGAGCTACATCGTGGAGACGAGCAGCAG | |
| | CGATAACGGGACCTGCTACCCTGGCGACTTCATC | |
| | GACTACGAGGAGCTGAGAGAGCAGCTGAGCAGCG | |
| | TGAGCAGCTTCGAGAGATTCGAGATCTTCCCTAA | |
| | GACCAGCAGCTGGCCTAACCACGACAGCAACAAG | |
| | GGCGTGACCGCCGCCTGCCCACACGCCGGGGCCA | |
| | AGAGCTTCTACAAGAACCTGATCTGGCTGGTGAA | |
| | GAAGGGCAACAGCTACCCTAAACTGAGCAAGTCC | |
| | TACATCAACGACAAAGGCAAGGAGGTCCTCGTGC | |
| | TCTGGGGCATCCACCACCCTAGCACCAGCGCCGA | |
| | TCAGCAGAGCCTGTACCAGAACGCCGACGCGTAC | |
| | GTGTTCGTGGGCACCAGCAGATACAGCAAGAAGT | |
| | TCAAGCCTGAGATCGCCATCAGACCTAAGGTGAG | |
| | GGACCAGGAGGGCAGAATGAACTACTACTGGACC | |
| | CTGGTGGAGCCCGGAGATAAGATCACATTTGAGG | |
| | CCACCGGCAACCTGGTGGTGCCTAGATACGCCTT | |
| | CGCCATGGAGAGAAACGCC | |
| MRK_pH1_Con_ecto: ecto | ATGAAGGCCATCCTCGTGGTGCTGCTGTACACCT | 65 |
| domain of consensus pH1 | TTGCCACCGCCAACGCCGATACCCTGTGTATCGG | |
| sequence (without | CTACCACGCCAACAACAGCACCGACACCGTGGAT | |
| transmembrane domain) | ACTGTCCTGGAGAAGAACGTGACCGTGACCCACA | |
| with foldon sequence | GCGTGAACCTGCTGGAGGACAAGCACAACGGCAA | |
| (second underlined), linker | GCTGTGCAAGCTGAGAGGCGTGGCCCCTCTGCAC | |
| (bold) | CTGGGCAAGTGCAACATCGCCGGCTGGATCCTGG | |
| | GCAACCCTGAGTGCGAGAGCCTTAGCACAGCCTC | |
| | CTCCTGGAGCTACATCGTGGAGACGAGCAGCAGC | |
| | GATAACGGGACCTGCTACCCTGGCGACTTCATCG | |
| | ACTACGAGGAGCTGAGAGAGCAGCTGAGCAGCGT | |
| | GAGCAGCTTCGAGAGATTCGAGATCTTCCCTAAG | |
| | ACCAGCAGCTGGCCTAACCACGACAGCAACAAGG | |
| | GCGTGACCGCCGCCTGCCCACACGCCGGGGCCAA | |
| | GAGCTTCTACAAGAACCTGATCTGGCTGGTGAAG | |
| | AAGGGCAACAGCTACCCTAAACTGAGCAAGTCCT | |
| | ACATCAACGACAAAGGCAAGGAGGTCCTCGTGCT | |
| | CTGGGGCATCCACCACCCTAGCACCAGCGCCGAT | |
| | CAGCAGAGCCTGTACCAGAACGCCGACGCGTACG | |
| | TGTTCGTGGGCACCAGCAGATACAGCAAGAAGTT | |
| | CAAGCCTGAGATCGCCATCAGACCTAAGGTGAGG | |
| | GACCAGGAGGGCAGAATGAACTACTACTGGACCC | |
| | TGGTGGAGCCCGGAGATAAGATCACATTTGAGGC | |
| | CACCGGCAACCTGGTGGTGCCTAGATACGCCTTC | |
| | GCCATGGAGAGAAACGCCGGCAGCGGCATCATCA | |
| | TCAGCGACACCCCTGTGCACGACTGCAACACCAC | |
| | CTGCCAGACCCCTAAGGGCGCCATCAACACGAGC | |
| | CTGCCTTTCCAGAACATCCACCCTATCACCATCG | |
| | GCAAGTGCCCTAAGTACGTGAAGTCAACCAAACT | |
| | GAGACTCGCCACCGGCCTCAGAAACGTGCCTAGC | |
| | ATCCAGAGCAGAGGCCTCTTCGGCGCCATCGCGG | |
| | GATTCATCGAGGGCGGCTGGACCGGCATGGTGGA | |
| | CGGCTGGTACGGCTACCACCATCAGAACGAGCAG | |
| | GGCAGCGGGTACGCGGCCGACCTCAAGAGCACCC | |
| | AGAACGCCATCGACAAGATCACCAACAAGGTGAA | |
| | CAGCGTGATCGAGAAGATGAACACCCAGTTCACC | |
| | GCCGTGGGCAAGGAGTTCAACCACCTGGAGAAGA | |
| | GAATCGAGAACCTGAACAAGAAGGTGGACGACGG | |
| | CTTCCTGGACATCTGGACCTACAACGCAGAACTG | |
| | CTCGTGCTTCTGGAGAACGAGAGAACCCTGGACT | |
| | ACCACGACTCCAACGTGAAGAACCTGTACGAGAA | |
| | GGTGAGAAGCCAGCTGAAGAACAACGCCAAGGAG | |
| | ATCGGCAACGGCTGCTTCGAGTTCTACCACAAGT | |
| | GCGACAACACCTGCATGGAGAGCGTGAAGAACGG | |
| | CACCTACGACTACCCTAAGTACAGCGAGGAGGCC | |
| | AAGCTGAACAGAGAGGAGATCGACGGCGTGAAGC | |
| | TGGAGAGCACCAGAATCGGCTCAGCCGGGAGCGC | |
| | CGGCTACATCCCTGAGGCCCCTAGAGACGGCCAG | |
| | GCCTACGTGAGAAAGGACGGCGAGTGGGTGCTGC | |
| | TGAGCACCTTCCTG | |
| MRK_sH1_Con_RBD: | ATGAAGGTGAAGCTGCTGGTGCTGCTGTGCACCT | 66 |
| receptor binding domain | TCACCGCCACCTACGCCGGAATCGCTCCCCTGCA | |
| (RBD) of consensus sH1 | GCTCGGCAACTGCAGCGTGGCCGGCTGGATTCTG | |
| sequence | GGCAACCCCGAGTGCGAACTGCTGATTAGCAAAG | |
| | AGTCCTGGAGCTACATCGTGGAAACCCCGAATCC | |
| | CGAGAACGGCACCTGCTACCCCGGCTACTTCGCC | |
| | GACTACGAGGAGCTAAGAGAGCAGCTGAGTAGCG | |
| | TGAGCTCATTCGAGAGATTCGAGATCTTTCCCAA | |
| | GGAGTCTAGCTGGCCCAATCACACCGTCACCGGC | |
| | GTGTCCGCCAGCTGTAGCCACAACGGCAAGAGCA | |
| | GCTTCTACAGAAACCTGCTGTGGCTGACCGGCAA | |
| | GAACGGACTGTACCCTAACCTGAGCAAGAGCTAC | |
| | GCGAACAATAAGGAGAAGGAGGTGCTAGTGCTGT | |
| | GGGGCGTGCACCATCCGCCCAACATCGGCGACCA | |
| | GAGAGCCCTGTACCACACCGAGAACGCCTACGTG | |
| | AGCGTGGTGAGCAGCCACTATAGCAGAAGATTCA | |
| | CCCCTGAGATCGCCAAGAGGCCAAAGGTGAGAGA | |
| | TCAGGAAGGAAGAATAAACTACTACTGGACCCTC | |
| | CTGGAGCCCGGCGACACCATCATCTTCGAGGCTA | |
| | ACGGCAACCTGATCGCCCCTAGATACGCCTTCGC | |
| | CCTGAGCAGAGGC | |
| MRK_sH1_Con_ecto: ecto | ATGAAGGTGAAGCTGCTGGTGCTGCTGTGTACCT | 67 |
| domain of consensus sH1 | TCACTGCCACTTACGCCGACACCATTTGCATCGG | |
| sequence (without | CTACCACGCCAACAACAGCACCGATACCGTGGAC | |
| transmembrane domain) | ACCGTGCTGGAGAAGAACGTCACCGTGACCCACA | |
| with foldon sequence | GCGTGAACCTGCTGGAGGATAGCCATAACGGCAA | |
| (second underlined), linker | GCTGTGCCTGCTGAAGGGAATCGCTCCCCTGCAG | |
| (bold) | CTCGGCAACTGCAGCGTGGCCGGCTGGATTCTGG | |
| | GCAACCCCGAGTGCGAACTGCTGATTAGCAAAGA | |
| | GTCCTGGAGCTACATCGTGGAAACCCCGAATCCC | |
| | GAGAACGGCACCTGCTACCCCGGCTACTTCGCCG | |
| | ACTACGAGGAGCTAAGAGAGCAGCTGAGTAGCGT | |
| | GAGCTCATTCGAGAGATTCGAGATCTTTCCCAAG | |
| | GAGTCTAGCTGGCCCAATCACACCGTCACCGGCG | |
| | TGTCCGCCAGCTGTAGCCACAACGGCAAGAGCAG | |
| | CTTCTACAGAAACCTGCTGTGGCTGACCGGCAAG | |
| | AACGGACTGTACCCTAACCTGAGCAAGAGCTACG | |
| | CGAACAATAAGGAGAAGGAGGTGCTAGTGCTGTG | |
| | GGGCGTGCACCATCCGCCCAACATCGGCGACCAG | |
| | AGAGCCCTGTACCACACCGAGAACGCCTACGTGA | |
| | GCGTGGTGAGCAGCCACTATAGCAGAAGATTCAC | |
| | CCCTGAGATCGCCAAGAGGCCAAAGGTGAGAGAT | |
| | CAGGAAGGAAGAATAAACTACTACTGGACCCTCC | |
| | TGGAGCCCGGCGACACCATCATCTTCGAGGCTAA | |
| | CGGCAACCTGATCGCCCCTAGATACGCCTTCGCC | |
| | CTGAGCAGAGGCTTCGGCAGCGGCATCATCACCA | |
| | GCAACGCTCCCATGGACGAGTGCGACGCCAAGTG | |
| | CCAGACCCCGCAGGGCGCCATCAACTCGAGCCTG | |
| | CCCTTCCAGAACGTGCACCCCGTGACCATCGGCG | |
| | AGTGCCCCAAGTACGTGAGAAGCGCCAAGCTGAG | |
| | AATGGTGACCGGCCTGAGAAACATCCCAAGCATC | |
| | CAGAGCAGAGGGCTGTTCGGCGCCATCGCTGGCT | |
| | TCATCGAGGGCGGCTGGACCGGCATGGTGGACGG | |
| | CTGGTACGGTTATCACCACCAGAACGAGCAGGGC | |
| | AGCGGCTACGCCGCCGACCAGAAGTCCACCCAGA | |
| | ACGCCATCAACGGCATTACAAACAAGGTGAACAG | |
| | CGTTATCGAGAAGATGAACACCCAATTCACCGCC | |
| | GTGGGCAAGGAGTTCAACAAGCTGGAGAGAAGAA | |
| | TGGAGAACCTGAACAAGAAGGTGGACGACGGCTT | |
| | CCTGGACATCTGGACCTACAACGCCGAACTGCTG | |
| | GTCCTGCTGGAGAACGAGAGAACCCTGGACTTCC | |
| | ACGACTCCAACGTGAAGAACTTATACGAGAAGGT | |
| | CAAATCCCAGCTGAAGAACAACGCCAAAGAAATC | |
| | GGAAACGGCTGCTTCGAATTCTACCACAAGTGCA | |
| | ACGACGAGTGCATGGAGAGCGTGAAGAACGGAAC | |
| | CTACGACTACCCCAAGTACAGCGAGGAAAGCAAA | |
| | CTGAACAGAGAGAAGATCGACGGCGTGAAGTTAG | |
| | AGAGCATGGGCGTGGGCAGCGCCGGCTCTGCTGG | |
| | ATACATCCCTGAGGCCCCTAGAGACGGCCAGGCC | |
| | TACGTGAGAAAGGACGGCGAGTGGGTGCTGCTGA | |
| | GCACCTTCCTG | |
| MRK_sH1_Con_v2: | ATGAAGGTGAAACTCCTCGTCCTGCTGTGCACCT | 68 |
| consensus sequence of HA | TCACCGCCACCTACGCCGATACCATCTGTATTGG | |
| subtype H1, includes | CTACCACGCCAACAACTCCACCGACACCGTGGAT | |
| transmembrane sequence | ACCGTGCTCGAGAAGAACGTGACCGTGACCCACA | |
| (second underlined) | GCGTGAACCTGCTGGAGAACAGCCACAACGGCAA | |
| | GCTGTGCCTGCTGAAGGGCATCGCGCCCCTGCAG | |
| | TTGGGTAACTGCTCCGTGGCCGGCTGGATCCTGG | |
| | GCAACCCTGAGTGCGAGCTGCTGATCAGCAAGGA | |
| | GAGCTGGAGCTACATCGTGGAGAAGCCTAACCCC | |
| | GAGAACGGCACCTGCTACCCTGGCCACTTCGCCG | |
| | ACTACGAGGAGCTGAGAGAGCAACTCAGCAGCGT | |
| | GAGCAGCTTCGAGAGATTCGAGATCTTCCCTAAG | |
| | GAGAGCAGCTGGCCCAATCACACTGTGACCGGCG | |
| | TGTCCGCTTCTTGCAGCCATAACGGGGAAAGCTC | |
| | CTTCTACAGAAATCTCCTTTGGCTGACGGGGAAG | |
| | AACGGCCTGTACCCTAACCTGAGCAAGAGCTACG | |
| | CCAACAACAAGGAGAAGGAGGTGCTGGTGCTGTG | |
| | GGGCGTGCACCACCCTCCTAACATCGGCGACCAG | |
| | AAGGCCCTGTACCACACCGAGAACGCCTACGTCA | |
| | GCGTGGTGTCCAGCCACTACAGCAGAAAGTTCAC | |
| | CCCTGAGATCGCCAAGAGGCCTAAGGTGCGGGAC | |
| | CAGGAGGGCAGAATCAACTACTACTGGACCCTGC | |
| | TGGAGCCTGGCGACACCATCATCTTCGAGGCCAA | |
| | CGGCAACCTGATCGCCCCTAGATACGCCTTCGCC | |
| | CTGAGCAGAGGCTTCGGCAGCGGCATCATCAACA | |
| | GCAACGCCCCTATGGACAAGTGCGACGCCAAGTG | |
| | CCAGACTCCGCAGGGCGCTATCAACAGCTCCCTG | |
| | CCTTTCCAGAACGTGCACCCTGTGACCATCGGCG | |
| | AGTGCCCTAAGTACGTGAGAAGCGCCAAGCTGAG | |
| | AATGGTGACCGGCCTGAGAAACATCCCTAGCATC | |
| | CAGAGCAGAGGCCTGTTCGGCGCCATCGCCGGGT | |
| | TTATCGAGGGCGGCTGGACCGGCATGGTGGACGG | |
| | CTGGTACGGCTACCACCACCAGAACGAGCAGGGC | |
| | TCCGGCTACGCCGCCGACCAGAAATCCACCCAGA | |
| | ACGCCATCAACGGCATCACCAACAAGGTGAACAG | |
| | CGTCATCGAGAAGATGAACACCCAGTTCACCGCC | |
| | GTGGGCAAGGAGTTCAACAAGCTGGAGAGAAGAA | |
| | TGGAGAACCTGAACAAGAAGGTGGACGACGGCTT | |
| | CATCGACATCTGGACCTACAACGCCGAGCTTCTG | |
| | GTGCTCCTGGAGAACGAGAGAACCCTGGACTTCC | |
| | ACGACAGCAACGTGAAGAACCTGTACGAGAAGGT | |
| | GAAGTCCCAGCTGAAGAACAACGCCAAGGAGATC | |
| | GGCAACGGCTGCTTCGAGTTCTACCACAAGTGCA | |
| | ACGACGAGTGCATGGAGAGCGTGAAGAACGGCAC | |
| | CTACGATTACCCCAAGTACAGCGAGGAGAGCAAG | |
| | CTGAACAGAGAGAAGATCGACGGCGTGAAGCTGG | |
| | AGAGCATGGGCGTGTACCAGATCCTGGCCATCTA | |
| | CTCCACCGTGGCCAGTAGCCTGGTGCTGCTGGTG | |
| | AGCCTGGGCGCAATCAGCTTCTGGATGTGCAGCA | |
| | ACGGCAGCCTGCAGTGCAGAATCTGCATC | |
| MRK_RBS_HA129 | ATGAAGGTCAAACTTCTCGTGCTCCTGTGCACCT | 69 |
| | TCACCGCCACCTACGCGGGCGTGGCTCCGCTTCA | |
| | CCTGGGCAAGTGCAACATCGCCGGTTGGCTGCTG | |
| | GGTAACCCAGAGTGCGAGCTACTGCTGACCGTGA | |
| | GCAGCTGGAGCTACATCGTGGAAACCAGCAACAG | |
| | CGACAACGGCACCTGCTACCCTGGCGACTTCATC | |
| | AACTACGAGGAGCTGAGAGAGCAGCTCAGCAGCG | |
| | TGTCCAGCTTCGAGAGATTCGAGATCTTCCCTAA | |
| | GACTAGCAGCTGGCCCGACCACGAAACAAACAGA | |
| | GGCGTGACCGCCGCTTGTCCATACGCCGGCGCCA | |
| | ACAGCTTCTACAGAAACCTGATCTGGCTGGTGAA | |
| | GAAGGGCAACAGCTACCCTAAGCTGAGCAAGAGC | |
| | TACGTGAACAACAAGGGCAAGGAGGTGCTTGTGC | |
| | TGTGGGGCATCCACCACCCTCCTACCAGCACCGA | |
| | CCAGCAGAGCCTGTACCAGAACGCCGACGCCTAC | |
| | GTGTTCGTGGGCAGCAGCAGATACAGCAAGAAGT | |
| | TCAAGCCTGAGATCGCCATCAGACCTAAGGTGAG | |
| | GGACCAGGAGGGCAGAATGAACTACTACTGGACT | |
| | CTGGTGGAGCCCGGCGACAAGATCACCTTCGAGG | |
| | CCACCGGCAACCTGGTGGTGCCTAGATACGCCTT | |
| | CGCCATGGAGAGAAACGCC | |
| MRK_H1_cot_all | ATGAAGGCCATCCTGGTCGTGCTGCTCTACACAT | 70 |
| “center of tree” sequence | TCGCCACCGCCAACGCAGACACTCTGTGCATCGG | |
| for subtype H1 with | CTACCACGCCAACAACAGCACCGACACCGTGGAT | |
| transmembrane domain | ACCGTGCTGGAGAAGAACGTGACCGTGACCCACA | |
| (second underlined) | GCGTGAACCTGCTGGAGGACAAGCACAACGGCAA | |
| | GCTGTGCAAGCTGAGAGGCGTGGCCCCTCTGCAC | |
| | CTGGGCAAGTGCAACATCGCCGGCTGGATCCTGG | |
| | GAAACCCCGAGTGCGAGAGCCTGTCAACCGCCTC | |
| | GAGCTGGTCCTACATCGTGGAAACCAGCAGCAGC | |
| | GATAACGGGACGTGCTACCCGGGCGACTTCATCA | |
| | ACTACGAGGAGCTGAGAGAACAGCTGAGCAGCGT | |
| | CAGTAGCTTCGAGAGATTCGAGATCTTCCCTAAG | |
| | ACCAGCAGCTGGCCTAACCACGACAGCAACAAGG | |
| | GCGTGACCGCCGCTTGCCCGCACGCAGGCGCCAA | |
| | GAGCTTCTACAAGAACCTGATCTGGCTGGTGAAG | |
| | AAGGGCAACAGCTACCCTAAGCTGAGCAAGAGCT | |
| | ACATCAACGACAAGGGGAAGGAGGTGCTAGTCCT | |
| | GTGGGGCATCCATCACCCTAGCACCACAGCCGAC | |
| | CAGCAAAGCCTGTACCAGAACGCGGACGCCTACG | |
| | TGTTCGTCGGCACCAGCAGATACAGCAAGAAGTT | |
| | CAAGCCTGAGATCGCCATCAGACCTAAGGTGCGA | |
| | GATCAGGAGGGCAGAATGAACTACTACTGGACCC | |
| | TGGTGGAGCCCGGAGACAAGATTACTTTCGAAGC | |
| | GACCGGCAACCTGGTGGTGCCTAGATACGCCTTC | |
| | GCCATGGAGAGAAACGCCGGCAGCGGCATCATCA | |
| | TCAGCGACACCCCTGTGCACGACTGCAACACCAC | |
| | CTGCCAGACCCCTAAAGGCGCCATCAACACAAGC | |
| | CTGCCTTTTCAGAACATCCACCCTATCACCATCG | |
| | GCAAGTGCCCTAAGTACGTGAAGTCCACCAAGCT | |
| | CCGCCTGGCAACCGGCCTCAGGAACGTGCCTAGC | |
| | ATCCAGAGCAGAGGCCTGTTCGGGGCCATAGCCG | |
| | GCTTCATAGAGGGTGGCTGGACCGGCATGGTTGA | |
| | CGGGTGGTACGGATACCATCACCAGAACGAGCAA | |
| | GGCAGCGGCTACGCCGCAGACCTGAAGTCAACCC | |
| | AGAACGCCATCGACAAGATCACCAACAAGGTGAA | |
| | CAGCGTGATCGAGAAGATGAACACCCAGTTCACC | |
| | GCCGTGGGCAAGGAGTTCAACCACCTAGAGAAGA | |
| | GGATCGAGAACCTGAATAAGAAGGTGGACGACGG | |
| | CTTCCTGGACATCTGGACCTACAACGCCGAGCTG | |
| | CTCGTCCTCCTGGAGAACGAGAGAACCCTGGACT | |
| | ACCACGATAGCAACGTGAAGAACCTGTACGAGAA | |
| | GGTGAGAAACCAGCTGAAGAATAACGCCAAGGAG | |
| | ATCGGCAACGGCTGCTTCGAGTTCTACCACAAGT | |
| | GCGACAACACCTGCATGGAGAGCGTGAAGAACGG | |
| | CACCTACGACTACCCTAAGTACAGCGAGGAGGCC | |
| | AAGCTGAACAGAGAGAAGATCGACGGCGTGAAGC | |
| | TGGAGAGCACCAGAATCTACCAGATCCTGGCCAT | |
| | CTACAGCACCGTGGCCAGCAGCCTCGTGCTCGTG | |
| | GTGAGCCTGGGCGCCATCTCCTTCTGGATGTGCA | |
| | GCAACGGCAGCCTGCAGTGCAGAATCTGCATC | |
| MRK_H3_cot_all | ATGAAGACCATCATCGCCCTGAGCTACATCCTGT | 71 |
| “center of tree” sequence | GCCTGGTGTTCGCGCAGAAACTCCCCGGCAACGA | |
| for subtype H3 with | CAATAGCACTGCCACCCTGTGTCTGGGCCATCAC | |
| transmembrane domain | GCCGTGCCTAACGGAACCATCGTGAAGACGATCA | |
| (second underlined) | CCAACGACAGAATCGAGGTGACCAACGCCACCGA | |
| | GCTGGTCCAGAATTCGAGCATCGGCGAAATCTGC | |
| | GACAGCCCTCACCAGATCCTGGACGGCGAGAACT | |
| | GCACCCTGATTGACGCACTGCTAGGCGACCCACA | |
| | GTGTGACGGCTTCCAGAACAAGAAGTGGGACCTG | |
| | TTCGTGGAGAGAAGCAAGGCCTACAGCAACTGCT | |
| | ACCCTTACGACGTGCCTGACTACGCCAGCCTGAG | |
| | ATCCCTCGTGGCCTCCAGCGGCACCCTCGAGTTC | |
| | AATAACGAGAGCTTCAACTGGACCGGAGTCACCC | |
| | AGAACGGGACATCCAGCGCCTGCATCAGAAGAAG | |
| | CAACAGCAGCTTCTTCAGCAGACTGAACTGGCTG | |
| | ACCCACCTGAACTTCAAGTACCCTGCCCTGAACG | |
| | TGACCATGCCTAACAACGAGCAGTTCGACAAGCT | |
| | GTACATCTGGGGCGTGCACCATCCCGGCACCGAC | |
| | AAGGACCAGATCTTCCTGTACGCCCAGAGCTCCG | |
| | GCAGGATCACCGTGAGCACCAAGAGAAGCCAGCA | |
| | GGCCGTGATCCCTAACATCGGCAGCAGACCTAGA | |
| | ATCAGAAACATCCCTAGCAGAATCAGCATCTACT | |
| | GGACCATAGTGAAGCCCGGCGACATCCTGCTGAT | |
| | CAACTCGACCGGCAACCTGATCGCTCCTAGGGGC | |
| | TACTTCAAGATCAGAAGCGGCAAGAGCAGCATCA | |
| | TGAGAAGCGACGCGCCCATCGGGAAGTGCAAGTC | |
| | CGAGTGCATCACCCCTAACGGCAGCATCCCCAAC | |
| | GACAAGCCTTTCCAGAACGTGAACAGAATCACCT | |
| | ACGGCGCCTGCCCTAGATACGTGAAGCAGAGCAC | |
| | ACTGAAGCTGGCCACCGGCATGAGGAACGTGCCT | |
| | GAGAAGCAGACCAGAGGCATCTTCGGGGCTATTG | |
| | CCGGCTTCATCGAGAACGGTTGGGAGGGAATGGT | |
| | CGACGGGTGGTACGGCTTCAGACACCAGAACAGC | |
| | GAAGGCAGGGGACAGGCCGCCGACCTCAAGTCCA | |
| | CCCAGGCTGCCATCGATCAGATCAACGGGAAGCT | |
| | GAACAGACTGATCGGCAAGACCAACGAGAAGTTC | |
| | CACCAGATCGAGAAGGAGTTCAGCGAGGTGGAGG | |
| | GCAGAATCCAGGACCTGGAGAAGTACGTGGAGGA | |
| | CACGAAGATCGACCTGTGGAGCTACAACGCAGAG | |
| | CTGTTGGTGGCACTGGAGAACCAGCACACCATCG | |
| | ACCTGACCGACAGCGAGATGAACAAGCTGTTCGA | |
| | GAAGACCAAGAAGCAGTTACGAGAGAACGCCGAG | |
| | GACATGGGAAACGGCTGTTTTAAGATCTACCACA | |
| | AGTGCGACAACGCCTGCATCGGGAGCATCAGGAA | |
| | CGGGACCTACGACCACGACGTGTACAGAGACGAG | |
| | GCCCTGAACAACAGATTCCAGATCAAGGGCGTGG | |
| | AGCTGAAGTCCGGCTACAAGGACTGGATCCTGTG | |
| | GATCAGCTTCGCCATCAGCTGCTTCCTGCTGTGC | |
| | GTGGCCCTCCTGGGCTTTATAATGTGGGCCTGCC | |
| | AGAAGGGCAACATCAGGTGCAACATCTGCATC | |
| MRK_H3_ConsensusA: | ATGAAGACCATCATCGCCCTGAGCTACATCCTGT | 72 |
| consensus sequence for | GCCTGGTGTTCGCGCAGAAACTCCCCGGCAACGA | |
| subtype H3, cluster A with | CAATAGCACTGCCACCCTGTGTCTGGGCCATCAC | |
| transmembrane domain | GCCGTGCCTAACGGAACCCTCGTGAAGACGATCA | |
| (second underlined) | CCAACGACCAGATCGAGGTGACCAACGCCACCGA | |
| | GCTGGTCCAGAGTTCGAGCACCGGCAGAATCTGC | |
| | GACAGCCCTCACCGGATCCTGGACGGCGAGAACT | |
| | GCACCCTGATTGACGCACTGCTAGGCGACCCACA | |
| | CTGTGACGGCTTCCAGAACAAGGAGTGGGACCTG | |
| | TTCGTGGAGAGAAGCAAGGCCTACAGCAACTGCT | |
| | ACCCTTACGACGTGCCTGACTACGCCAGCCTGAG | |
| | ATCCCTCGTGGCCTCCAGCGGCACCCTCGAGTTC | |
| | AATAACGAGAGCTTCAACTGGACCGGAGTCGCCC | |
| | AGAACGGGACATCCTACGCCTGCAAGAGAGGAAG | |
| | CGTCAAGAGCTTCTTCAGCAGACTGAACTGGCTG | |
| | CACCAGCTGAAGTACAAGTACCCTGCCCTGAACG | |
| | TGACCATGCCTAACAACGACAAGTTCGACAAGCT | |
| | GTACATCTGGGGCGTGCACCATCCCAGCACCGAC | |
| | AGCGACCAGACCTCCCTGTACGTCCAGGCATCCG | |
| | GCAGGGTCACCGTGAGCACCAAGAGAAGCCAGCA | |
| | GACCGTGATCCCTAACATCGGCAGCAGACCTTGG | |
| | GTCAGAGGCGTCTCTAGCAGAATCAGCATCTACT | |
| | GGACCATAGTGAAGCCCGGCGACATCCTGCTGAT | |
| | CAACTCGACCGGCAACCTGATCGCTCCTAGGGGC | |
| | TACTTCAAGATCAGAAGCGGCAAGAGCAGCATCA | |
| | TGAGAAGCGACGCGCCCATCGGGAAGTGCAACTC | |
| | CGAGTGCATCACCCCTAACGGCAGCATCCCCAAC | |
| | GACAAGCCTTTCCAGAACGTGAACAGAATCACCT | |
| | ACGGCGCCTGCCCTAGATACGTGAAGCAGAACAC | |
| | ACTGAAGCTGGCCACCGGCATGAGGAACGTGCCT | |
| | GAGAAGCAGACCAGAGGCATCTTCGGGGCTATTG | |
| | CCGGCTTCATCGAGAACGGTTGGGAGGGAATGGT | |
| | CGACGGGTGGTACGGCTTCAGACACCAGAACAGC | |
| | GAAGGCACGGGACAGGCCGCCGACCTCAAGTCCA | |
| | CCCAGGCTGCCATCAATCAGATCAACGGGAAGCT | |
| | GAACAGACTGATCGAGAAGACCAACGAGAAGTTC | |
| | CACCAGATCGAGAAGGAGTTCAGCGAGGTGGAGG | |
| | GCAGAATCCAGGACCTGGAGAAGTACGTGGAGGA | |
| | CACGAAGATCGACCTGTGGAGCTACAACGCAGAG | |
| | CTGTTGGTGGCACTGGAGAACCAGCACACCATCG | |
| | ACCTGACCGACAGCGAGATGAACAAGCTGTTCGA | |
| | GAGGACCAGGAAGCAGTTACGAGAGAACGCCGAG | |
| | GACATGGGAAACGGCTGTTTTAAGATCTACCACA | |
| | AGTGCGACAACGCCTGCATCGGGAGCATCAGGAA | |
| | CGGGACCTACGACCACGACGTGTACAGAGACGAG | |
| | GCCCTGAACAACAGATTCCAGATCAAGGGCGTGG | |
| | AGCTGAAGTCCGGCTACAAGGACTGGATCCTGTG | |
| | GATCAGCTTCGCCATCAGCTGCTTCCTGCTGTGC | |
| | GTGGTCCTCCTGGGCTTTATAATGTGGGCCTGCC | |
| | AGAAGGGCAACATCAGGTGCAACATCTGCATC | |
| MRK_H3_ConsensusB: | ATGAAGACCATCATCGCCCTGAGCTACATCCTGT | 73 |
| consensus sequence for | GCCTGGTGTTCGCGCAGAAACTCCCCGGCAACGA | |
| subtype H3, cluster B with | CAATAGCACTGCCACCCTGTGTCTGGGCCATCAC | |
| transmembrane domain | GCCGTGCCTAACGGAACCATCGTGAAGACGATCA | |
| (second underlined) | CCAACGACCAGATCGAGGTGACCAACGCCACCGA | |
| | GCTGGTCCAGAATTCGAGCACCGGCGAAATCTGC | |
| | GACAGCCCTCACCAGATCCTGGACGGCGAGAACT | |
| | GCACCCTGATTGACGCACTGCTAGGCGACCCACA | |
| | GTGTGACGGCTTCCAGAACAAGAAGTGGGACCTG | |
| | TTCGTGGAGAGAAGCAAGGCCTACAGCAACTGCT | |
| | ACCCTTACGACGTGCCTGACTACGCCAGCCTGAG | |
| | ATCCCTCGTGGCCTCCAGCGGCACCCTCGAGTTC | |
| | AATAACGAGAGCTTCAACTGGACCGGAGTCACCC | |
| | AGAACGGGACATCCAGCGCCTGCATCAGAAGAAG | |
| | CAACAGCAGCTTCTTCAGCAGACTGAACTGGCTG | |
| | ACCCACCTGAACTTCAAGTACCCTGCCCTGAACG | |
| | TGACCATGCCTAACAACGAGCAGTTCGACAAGCT | |
| | GTACATCTGGGGCGTGCACCATCCCGGCACCGAC | |
| | AAGGACCAGATCTTCCTGTACGCCCAGAGCTCCG | |
| | GCAGGATCACCGTGAGCACCAAGAGAAGCCAGCA | |
| | GGCCGTGATCCCTAACATCGGCAGCAGACCTAGA | |
| | ATCAGAAACATCCCTAGCAGAATCAGCATCTACT | |
| | GGACCATAGTGAAGCCCGGCGACATCCTGCTGAT | |
| | CAACTCGACCGGCAACCTGATCGCTCCTAGGGGC | |
| | TACTTCAAGATCAGAAGCGGCAAGAGCAGCATCA | |
| | TGAGAAGCGACGCGCCCATCGGGAAGTGCAACTC | |
| | CGAGTGCATCACCCCTAACGGCAGCATCCCCAAC | |
| | GACAAGCCTTTCCAGAACGTGAACAGAATCACCT | |
| | ACGGCGCCTGCCCTAGATACGTGAAGCAGAGCAC | |
| | ACTGAAGCTGGCCACCGGCATGAGGAACGTGCCT | |
| | GAGAAGCAGACCAGAGGCATCTTCGGGGCTATTG | |
| | CCGGCTTCATCGAGAACGGTTGGGAGGGAATGGT | |
| | CGACGGGTGGTACGGCTTCAGACACCAGAACAGC | |
| | GAAGGCAGGGGACAGGCCGCCGACCTCAAGTCCA | |
| | CCCAGGCTGCCATCGATCAGATCAACGGGAAGCT | |
| | GAACAGACTGATCGGCAAGACCAACGAGAAGTTC | |
| | CACCAGATCGAGAAGGAGTTCAGCGAGGTGGAGG | |
| | GCAGAATCCAGGACCTGGAGAAGTACGTGGAGGA | |
| | CACGAAGATCGACCTGTGGAGCTACAACGCAGAG | |
| | CTGTTGGTGGCACTGGAGAACCAGCACACCATCG | |
| | ACCTGACCGACAGCGAGATGAACAAGCTGTTCGA | |
| | GAAGACCAAGAAGCAGTTACGAGAGAACGCCGAG | |
| | GACATGGGAAACGGCTGTTTTAAGATCTACCACA | |
| | AGTGCGACAACGCCTGCATCGGGAGCATCAGGAA | |
| | CGGGACCTACGACCACGACGTGTACAGAGACGAG | |
| | GCCCTGAACAACAGATTCCAGATCAAGGGCGTGG | |
| | AGCTGAAGTCCGGCTACAAGGACTGGATCCTGTG | |
| | GATCAGCTTCGCCATCAGCTGCTTCCTGCTGTGC | |
| | GTGGCCCTCCTGGGCTTTATAATGTGGGCCTGCC | |
| | AGAAGGGCAACATCAGGTGCAACATCTGCATC | |
| MRK_H3_consUnique: | ATGAAGACCATCATCGCCCTGAGCTACATCCTGT | 74 |
| consensus sequence for | GCCTGGTGTTCGCGCAGAAACTCCCCGGCAACGA | |
| subtype H3 with | CAATAGCACTGCCACCCTGTGTCTGGGCCATCAC | |
| transmembrane domain | GCCGTGCCTAACGGAACCATCGTGAAGACGATCA | |
| (second underlined) | CCAACGACCAGATCGAGGTGACCAACGCCACCGA | |
| | GCTGGTCCAGAGTTCGAGCACCGGCGAAATCTGC | |
| | GACAGCCCTCACCAGATCCTGGACGGCGAGAACT | |
| | GCACCCTGATTGACGCACTGCTAGGCGACCCACA | |
| | GTGTGACGGCTTCCAGAACAAGAAGTGGGACCTG | |
| | TTCGTGGAGAGAAGCAAGGCCTACAGCAACTGCT | |
| | ACCCTTACGACGTGCCTGACTACGCCAGCCTGAG | |
| | ATCCCTCGTGGCCTCCAGCGGCACCCTCGAGTTC | |
| | AATAACGAGAGCTTCAACTGGACCGGAGTCACCC | |
| | AGAACGGGACATCCAGCGCCTGCATCAGAAGAAG | |
| | CAACAGCAGCTTCTTCAGCAGACTGAACTGGCTG | |
| | ACCCACCTGAACTTCAAGTACCCTGCCCTGAACG | |
| | TGACCATGCCTAACAACGAGCAGTTCGACAAGCT | |
| | GTACATCTGGGGCGTGCACCATCCCGGCACCGAC | |
| | AAGGACCAGATCTTCCTGTACGCCCAGGCATCCG | |
| | GCAGGATCACCGTGAGCACCAAGAGAAGCCAGCA | |
| | GGCCGTGATCCCTAACATCGGCAGCAGACCTAGA | |
| | GTCAGAAACATCCCTAGCAGAATCAGCATCTACT | |
| | GGACCATAGTGAAGCCCGGCGACATCCTGCTGAT | |
| | CAACTCGACCGGCAACCTGATCGCTCCTAGGGGC | |
| | TACTTCAAGATCAGAAGCGGCAAGAGCAGCATCA | |
| | TGAGAAGCGACGCGCCCATCGGGAAGTGCAACTC | |
| | CGAGTGCATCACCCCTAACGGCAGCATCCCCAAC | |
| | GACAAGCCTTTCCAGAACGTGAACAGAATCACCT | |
| | ACGGCGCCTGCCCTAGATACGTGAAGCAGAACAC | |
| | ACTGAAGCTGGCCACCGGCATGAGGAACGTGCCT | |
| | GAGAAGCAGACCAGAGGCATCTTCGGGGCTATTG | |
| | CCGGCTTCATCGAGAACGGTTGGGAGGGAATGGT | |
| | CGACGGGTGGTACGGCTTCAGACACCAGAACAGC | |
| | GAAGGCAGGGGACAGGCCGCCGACCTCAAGTCCA | |
| | CCCAGGCTGCCATCGATCAGATCAACGGGAAGCT | |
| | GAACAGACTGATCGGCAAGACCAACGAGAAGTTC | |
| | CACCAGATCGAGAAGGAGTTCAGCGAGGTGGAGG | |
| | GCAGAATCCAGGACCTGGAGAAGTACGTGGAGGA | |
| | CACGAAGATCGACCTGTGGAGCTACAACGCAGAG | |
| | CTGTTGGTGGCACTGGAGAACCAGCACACCATCG | |
| | ACCTGACCGACAGCGAGATGAACAAGCTGTTCGA | |
| | GAAGACCAAGAAGCAGTTACGAGAGAACGCCGAG | |
| | GACATGGGAAACGGCTGTTTTAAGATCTACCACA | |
| | AGTGCGACAACGCCTGCATCGGGAGCATCAGGAA | |
| | CGGGACCTACGACCACGACGTGTACAGAGACGAG | |
| | GCCCTGAACAACAGATTCCAGATCAAGGGCGTGG | |
| | AGCTGAAGTCCGGCTACAAGGACTGGATCCTGTG | |
| | GATCAGCTTCGCCATCAGCTGCTTCCTGCTGTGC | |
| | GTGGCCCTCCTGGGCTTTATAATGTGGGCCTGCC | |
| | AGAAGGGCAACATCAGGTGCAACATCTGCATC | |
| RBD1-Cal09-PC-Cb | ATGAAGGTGAAGCTTCTCGTGCTCTTATGCACCT | 75 |
| 6 glycosylation sites to | TCACCGCCACCTACGCCGGCGTGGCTCCGCTTCA | |
| allow access to the Cb | CCTTGGCAAGTGCAACATCGCCGGCTGGATCTTG | |
| epitope | GGAAACCCCGAGTGCGAGAGCTTGAGCACCGCCA | |
| | GCAGCTGGAGCAACATCACGGAAACCCCTAGCAG | |
| | CGACAACGGCACCTGCTACCCCGGCGACTTCATC | |
| | GACTACGAGGAGCTGCGGGAGCAGCTGAGCAGCG | |
| | TGAGCAGCTTCGAGCGGTTCGAGATCTTCCCCAA | |
| | GACCAGCTCTTGGCCCAACCACAGCAGCAACAAG | |
| | GGCGTGACCGCCGCCTGCCCTCACGCTGGCGCCA | |
| | AGAGCTTCTACAAGAACCTGATCTGGCTGGTGAA | |
| | GAAGAACGGCAGCTACCCCAAGCTGAACAAGTCT | |
| | TACATTAACGACTCAGGCAAGGAGGTGCTGGTCC | |
| | TGTGGGGCATCCACCACCCCAGCAACAGCACCGA | |
| | CCAACAGAGCCTGTACCAGAACGCCGACACCTAC | |
| | GTGTTCGTGGGCAGCAGCAACTACAGCAAGAAGT | |
| | TCAAGCCCGAGATCGCCATCCGGCCCAAGGTGCG | |
| | GGACCAGGAGGGCCGGATGAACTACTACTGGACC | |
| | CTGGTGGAGCCTGGCGACAAGATCACCTTCGAGG | |
| | CCACCGGCAACCTGGTGGTGCCCCGGTACGCCTT | |
| | CGCCATGGAGCGGAACGCC | |
| RBD1-Cal09-PC | ATGAAGGTGAAGCTTCTCGTGCTCTTATGCACCT | 76 |
| 7 added glycosylation sites | TCACCGCCACCTACGCCGGCGTGGCTCCGCTTCA | |
| | CCTTGGCAAGTGCAACATCGCCGGCTGGATCTTG | |
| | GGAAACCCCGAGTGCGAGAGCAACAGCACCGCCA | |
| | GCAGCTGGAGCAACATCACGGAAACCCCTAGCAG | |
| | CGACAACGGCACCTGCTACCCCGGCGACTTCATC | |
| | GACTACGAGGAGCTGCGGGAGCAGCTGAGCAGCG | |
| | TGAGCAGCTTCGAGCGGTTCGAGATCTTCCCCAA | |
| | GACCAGCTCTTGGCCCAACCACAGCAGCAACAAG | |
| | GGCGTGACCGCCGCCTGCCCTCACGCTGGCGCCA | |
| | AGAGCTTCTACAAGAACCTGATCTGGCTGGTGAA | |
| | GAAGAACGGCAGCTACCCCAAGCTGAACAAGTCT | |
| | TACATTAACGACTCAGGCAAGGAGGTGCTGGTCC | |
| | TGTGGGGCATCCACCACCCCAGCAACAGCACCGA | |
| | CCAACAGAGCCTGTACCAGAACGCCGACACCTAC | |
| | GTGTTCGTGGGCAGCAGCAACTACAGCAAGAAGT | |
| | TCAAGCCCGAGATCGCCATCCGGCCCAAGGTGCG | |
| | GGACCAGGAGGGCCGGATGAACTACTACTGGACC | |
| | CTGGTGGAGCCTGGCGACAAGATCACCTTCGAGG | |
| | CCACCGGCAACCTGGTGGTGCCCCGGTACGCCTT | |
| | CGCCATGGAGCGGAACGCC | |
| RBD1-Cal09 | ATGAAGGTGAAGCTTCTCGTGCTCTTATGCACCT | 77 |
| | TCACCGCCACCTACGCCGGCGTGGCTCCGCTTCA | |
| | CCTTGGCAAGTGCAACATCGCCGGCTGGATCTTG | |
| | GGAAACCCCGAGTGCGAGAGCTTGAGCACCGCCA | |
| | GCAGCTGGAGCAACATCACGGAAACCCCTAGCAG | |
| | CGACAACGGCACCTGCTACCCCGGCGACTTCATC | |
| | GACTACGAGGAGCTGCGGGAGCAGCTGAGCAGCG | |
| | TGAGCAGCTTCGAGCGGTTCGAGATCTTCCCCAA | |
| | GACCAGCTCTTGGCCCAACCACGACAGCAACAAG | |
| | GGCGTGACCGCCGCCTGCCCTCACGCTGGCGCCA | |
| | AGAGCTTCTACAAGAACCTGATCTGGCTGGTGAA | |
| | GAAGGGCAACAGCTACCCCAAGCTGTCCAAGTCT | |
| | TACATTAACGACAAGGGCAAGGAGGTGCTGGTCC | |
| | TGTGGGGCATCCACCACCCCAGCACCAGCGCCGA | |
| | CCAACAGAGCCTGTACCAGAACGCCGACACCTAC | |
| | GTGTTCGTGGGCAGCAGCCGGTACAGCAAGAAGT | |
| | TCAAGCCCGAGATCGCCATCCGGCCCAAGGTGCG | |
| | GGACCAGGAGGGCCGGATGAACTACTACTGGACC | |
| | CTGGTGGAGCCTGGCGACAAGATCACCTTCGAGG | |
| | CCACCGGCAACCTGGTGGTGCCCCGGTACGCCTT | |
| | CGCCATGGAGCGGAACGCC | |
| MRK_RBD-Cal09-PC-Cb | ATGAAGGTGAAGCTTCTCGTGCTCTTATGCACCT | 78 |
| | TCACCGCCACCTACGCCGGCGTGGCTCCGCTTCA | |
| | CCTTGGCAAGTGCAACATCGCCGGCTGGATCTTG | |
| | GGAAACCCCGAGTGCGAGAGCTTGAGCACCGCCA | |
| | GCAGCTGGAGCTACATCGTGGAAACCCCTAGCAG | |
| | CGACAACGGCACCTGCTACCCCGGCGACTTCATC | |
| | GACTACGAGGAGCTGCGGGAGCAGCTGAGCAGCG | |
| | TGAGCAGCTTCGAGCGGTTCGAGATCTTCCCCAA | |
| | GACCAGCTCTTGGCCCAACCACAGCAGCAACAAG | |
| | GGCGTGACCGCCGCCTGCCCTCACGCTGGCGCCA | |
| | AGAGCTTCTACAAGAACCTGATCTGGCTGGTGAA | |
| | GAAGAACGGCAGCTACCCCAAGCTGAACAAGTCT | |
| | TACATTAACGACTCAGGCAAGGAGGTGCTGGTCC | |
| | TGTGGGGCATCCACCACCCCAGCAACAGCACCGA | |
| | CCAACAGAGCCTGTACCAGAACGCCGACACCTAC | |
| | GTGTTCGTGGGCAGCAGCAACTACAGCAAGAAGT | |
| | TCAAGCCCGAGATCGCCATCCGGCCCAAGGTGCG | |
| | GGACCAGGAGGGCCGGATGAACTACTACTGGACC | |
| | CTGGTGGAGCCTGGCGACAAGATCACCTTCGAGG | |
| | CCACCGGCAACCTGGTGGTGCCCCGGTACGCCTT | |
| | CGCCATGGAGCGGAACGCC | |
| MRK_RBD-Cal09-PC | ATGAAGGTGAAGCTTCTCGTGCTCTTATGCACCT | 79 |
| | TCACCGCCACCTACGCCGGCGTGGCTCCGCTTCA | |
| | CCTTGGCAAGTGCAACATCGCCGGCTGGATCTTG | |
| | GGAAACCCCGAGTGCGAGAGCAACAGCACCGCCA | |
| | GCAGCTGGAGCTACATCGTGGAAACCCCTAGCAG | |
| | CGACAACGGCACCTGCTACCCCGGCGACTTCATC | |
| | GACTACGAGGAGCTGCGGGAGCAGCTGAGCAGCG | |
| | TGAGCAGCTTCGAGCGGTTCGAGATCTTCCCCAA | |
| | GACCAGCTCTTGGCCCAACCACAGCAGCAACAAG | |
| | GGCGTGACCGCCGCCTGCCCTCACGCTGGCGCCA | |
| | AGAGCTTCTACAAGAACCTGATCTGGCTGGTGAA | |
| | GAAGAACGGCAGCTACCCCAAGCTGAACAAGTCT | |
| | TACATTAACGACTCAGGCAAGGAGGTGCTGGTCC | |
| | TGTGGGGCATCCACCACCCCAGCAACAGCACCGA | |
| | CCAACAGAGCCTGTACCAGAACGCCGACACCTAC | |
| | GTGTTCGTGGGCAGCAGCAACTACAGCAAGAAGT | |
| | TCAAGCCCGAGATCGCCATCCGGCCCAAGGTGCG | |
| | GGACCAGGAGGGCCGGATGAACTACTACTGGACC | |
| | CTGGTGGAGCCTGGCGACAAGATCACCTTCGAGG | |
| | CCACCGGCAACCTGGTGGTGCCCCGGTACGCCTT | |
| | CGCCATGGAGCGGAACGCC | |
| MRKRBD-Cal09 | ATGAAGGTGAAGCTTCTCGTGCTCTTATGCACCT | 80 |
| | TCACCGCCACCTACGCCGGCGTGGCTCCGCTTCA | |
| | CCTTGGCAAGTGCAACATCGCCGGCTGGATCTTG | |
| | GGAAACCCCGAGTGCGAGAGCTTGAGCACCGCCA | |
| | GCAGCTGGAGCTACATCGTGGAAACCCCTAGCAG | |
| | CGACAACGGCACCTGCTACCCCGGCGACTTCATC | |
| | GACTACGAGGAGCTGCGGGAGCAGCTGAGCAGCG | |
| | TGAGCAGCTTCGAGCGGTTCGAGATCTTCCCCAA | |
| | GACCAGCTCTTGGCCCAACCACGACAGCAACAAG | |
| | GGCGTGACCGCCGCCTGCCCTCACGCTGGCGCCA | |
| | AGAGCTTCTACAAGAACCTGATCTGGCTGGTGAA | |
| | GAAGGGCAACAGCTACCCCAAGCTGTCCAAGTCT | |
| | TACATTAACGACAAGGGCAAGGAGGTGCTGGTCC | |
| | TGTGGGGCATCCACCACCCCAGCACCAGCGCCGA | |
| | CCAACAGAGCCTGTACCAGAACGCCGACACCTAC | |
| | GTGTTCGTGGGCAGCAGCCGGTACAGCAAGAAGT | |
| | TCAAGCCCGAGATCGCCATCCGGCCCAAGGTGCG | |
| | GGACCAGGAGGGCCGGATGAACTACTACTGGACC | |
| | CTGGTGGAGCCTGGCGACAAGATCACCTTCGAGG | |
| | CCACCGGCAACCTGGTGGTGCCCCGGTACGCCTT | |
| | CGCCATGGAGCGGAACGCC | |
| FLHA_PR8 | ATGAAGGCCAATTTGTTGGTCCTTCTATGTGCCC | 81 |
| includes transmembrane | TAGCCGCCGCCGACGCCGACACAATCTGCATCGG | |
| sequence (second | ATATCACGCAAACAACAGCACCGACACCGTGGAT | |
| underlined) | ACGGTCTTGGAGAAGAACGTGACCGTGACCCATT | |
| | CCGTGAACCTTCTCGAGGATAGCCACAATGGCAA | |
| | GCTGTGTAGACTCAAGGGCATTGCCCCGCTGCAG | |
| | CTGGGAAAGTGCAATATTGCTGGCTGGCTGTTGG | |
| | GCAACCCTGAGTGTGACCCTCTGTTACCAGTGAG | |
| | ATCTTGGAGCTATATCGTCGAAACCCCTAACAGC | |
| | GAGAACGGCATATGCTACCCAGGCGACTTCATCG | |
| | ACTACGAGGAACTGCGCGAGCAGCTGAGCTCTGT | |
| | GTCGAGCTTCGAGCGGTTCGAGATCTTCCCTAAG | |
| | GAATCTAGCTGGCCTAATCATAACACAAATGGCG | |
| | TTACTGCTGCCTGTAGCCACGAGGGAAAGAGCAG | |
| | TTTCTACCGGAATCTGCTGTGGCTGACAGAGAAG | |
| | GAGGGCTCCTACCCTAAGCTGAAGAATAGCTATG | |
| | TGAACAAGAAGGGCAAGGAGGTGCTGGTGCTGTG | |
| | GGGAATACACCACCCACCTAACTCGAAGGAGCAG | |
| | CAGAATCTGTACCAGAATGAGAATGCCTACGTGT | |
| | CCGTCGTGACCTCCAACTACAACCGGCGGTTCAC | |
| | GCCTGAGATCGCCGAGAGGCCTAAGGTGAGGGAC | |
| | CAGGCCGGACGCATGAACTACTACTGGACCCTGC | |
| | TGAAGCCTGGCGATACAATCATCTTCGAGGCTAA | |
| | TGGAAACCTGATCGCGCCAATGTACGCCTTCGCC | |
| | CTGTCCAGAGGATTCGGCAGCGGCATCATCACAT | |
| | CCAACGCCTCCATGCACGAATGCAACACCAAGTG | |
| | CCAGACGCCTCTGGGAGCTATCAATAGCAGCTTG | |
| | CCTTACCAGAATATCCACCCTGTGACCATTGGAG | |
| | AGTGTCCAAAGTACGTGCGCAGCGCAAAGCTGCG | |
| | GATGGTCACAGGCCTGCGGAATATACCTTCTATC | |
| | CAGAGCCGAGGCCTGTTCGGTGCCATTGCCGGCT | |
| | TCATCGAGGGTGGCTGGACCGGAATGATCGACGG | |
| | CTGGTATGGATACCACCACCAGAATGAACAGGGC | |
| | AGCGGCTACGCCGCCGATCAGAAGTCCACCCAGA | |
| | ACGCAATCAATGGTATCACAAACAAGGTGAACAC | |
| | TGTAATCGAGAAGATGAACATCCAATTCACAGCC | |
| | GTGGGCAAGGAGTTCAATAAGCTGGAGAAGCGGA | |
| | TGGAGAACCTCAACAAGAAGGTGGACGACGGCTT | |
| | CCTGGATATCTGGACCTACAACGCAGAGCTGCTG | |
| | GTGTTGCTGGAGAACGAGAGAACCCTCGACTTCC | |
| | ATGATAGCAACGTTAAGAACCTATACGAGAAGGT | |
| | GAAGTCACAGCTGAAGAATAACGCCAAGGAGATT | |
| | GGCAACGGCTGCTTCGAATTCTACCACAAGTGCG | |
| | ACAACGAGTGTATGGAGAGCGTCCGGAATGGCAC | |
| | CTACGACTATCCTAAGTATAGCGAGGAGAGCAAG | |
| | CTTAATAGAGAGAAGGTCGATGGCGTGAAGCTGG | |
| | AGTCAATGGGAATCTACCAGATCCTGGCTATTTA | |
| | TTCAACCGTGGCATCAAGTCTGGTGCTTCTGGTC | |
| | AGCCTGGGCGCCATCAGCTTCTGGATGTGCTCCA | |
| | ATGGCAGCCTGCAATGCCGCATCTGCATA | |
| FLHA_Cal09 | ATGAAGGCTATCTTGGTGGTGTTGTTGTACACAT | 82 |
| | TCGCCACCGCCAACGCCGACACCCTCTGCATCGG | |
| | CTACCACGCGAACAATTCAACCGACACCGTTGAC | |
| | ACCGTCCTCGAGAAGAACGTGACCGTGACTCATA | |
| | GCGTCAACCTCCTCGAGGACAAGCATAACGGCAA | |
| | GCTCTGTAAGCTTAGAGGAGTGGCCCCTCTCCAC | |
| | CTGGGCAAGTGTAACATTGCAGGCTGGATCCTGG | |
| | GCAACCCTGAGTGCGAGAGCCTGTCAACCGCTAG | |
| | CAGCTGGAGCTACATCGTGGAAACCCCATCCAGC | |
| | GATAACGGCACCTGCTACCCTGGCGATTTCATCG | |
| | ACTACGAGGAGCTGCGCGAGCAGTTGAGCAGCGT | |
| | CTCCAGCTTCGAGAGATTCGAGATCTTCCCTAAG | |
| | ACTAGCAGCTGGCCTAATCATGACTCCAATAAGG | |
| | GCGTGACGGCCGCCTGTCCTCACGCTGGAGCCAA | |
| | GTCGTTCTACAAGAACCTGATCTGGCTGGTAAAG | |
| | AAGGGCAACAGCTACCCAAAGCTGAGCAAGTCCT | |
| | ACATCAACGACAAGGGCAAGGAAGTGCTGGTGCT | |
| | GTGGGGAATCCATCACCCAAGCACCTCTGCGGAC | |
| | CAGCAGTCTCTGTATCAGAACGCCGACACCTATG | |
| | TGTTCGTAGGCTCCTCCAGATACTCCAAGAAGTT | |
| | CAAGCCAGAGATTGCTATCCGCCCAAAGGTGCGG | |
| | GATCAAGAGGGTCGCATGAATTATTACTGGACCC | |
| | TGGTCGAGCCAGGCGATAAGATCACATTCGAAGC | |
| | CACGGGAAATCTGGTGGTGCCTAGATACGCTTTC | |
| | GCCATGGAGAGAAACGCCGGCAGCGGCATCATCA | |
| | TATCCGACACACCTGTGCACGACTGCAACACAAC | |
| | ATGCCAGACGCCAAAGGGAGCCATCAACACATCT | |
| | CTTCCATTCCAGAACATTCACCCAATCACAATCG | |
| | GCAAGTGTCCAAAGTACGTGAAGTCCACCAAGCT | |
| | TAGACTGGCCACCGGCCTGCGTAACATCCCTAGC | |
| | ATCCAGTCGAGAGGCCTCTTCGGCGCCATCGCCG | |
| | GATTCATTGAAGGTGGCTGGACCGGCATGGTGGA | |
| | CGGTTGGTATGGCTACCACCACCAGAACGAGCAG | |
| | GGCAGCGGCTACGCCGCGGACCTGAAGTCCACCC | |
| | AGAACGCTATTGACGAGATCACCAACAAGGTGAA | |
| | CAGCGTGATCGAGAAGATGAATACCCAGTTCACC | |
| | GCCGTCGGCAAGGAGTTCAACCATCTGGAGAAGA | |
| | GAATCGAGAACCTCAACAAGAAGGTCGACGACGG | |
| | CTTCCTGGACATTTGGACTTACAACGCTGAGTTG | |
| | TTGGTGCTTCTTGAGAATGAGCGGACCCTGGACT | |
| | ATCACGACTCAAATGTGAAGAACCTGTACGAGAA | |
| | GGTGAGATCCCAGCTGAAGAACAATGCTAAGGAA | |
| | ATCGGCAACGGCTGCTTCGAGTTCTATCATAAGT | |
| | GTGACAACACCTGCATGGAGTCTGTTAAGAACGG | |
| | CACATACGACTACCCGAAGTACTCTGAGGAGGCC | |
| | AAGCTGAACCGAGAGGAGATAGACGGCGTTAAGC | |
| | TAGAAAGTACAAGGATCTACCAGATCCTTGCCAT | |
| | CTACTCCACCGTGGCCTCCAGCCTGGTGTTGGTG | |
| | GTGAGCCTGGGCGCCATCAGCTTCTGGATGTGCA | |
| | GTAACGGAAGCCTACAGTGCCGAATCTGCATC | |

### Example 1: Mouse Immunogenicity and Efficacy Studies

### Study #1

This study was designed to test the immunogenicity and efficacy in mice of a combination of candidate influenza virus vaccines. Animals tested were 6-8 week old female BALB/c mice obtained from Charles River Laboratories. Test vaccines included the following mRNAs formulated in an LNP (comprised of a cationic lipid, a sterol, a phospholipid and a peg-lipid).: NIHGen6HASS-foldon mRNA (based on Yassine et al. Nat. Med. 2015 September; 21(9): 1065-70), an mRNA encoding the nucleoprotein NP from an H3N2 strain, or one of several combinations of NIHGen6HASS-foldon and NP mRNAs. Several methods of vaccine antigen co-delivery were tested including: mixing individual mRNAs prior to formulation with LNP (co-form), formulation of individual mRNAs prior to mixing (mix ind LNPs), and formulating mRNAs individually and injecting distal sites (opposite legs) (ind LNPs remote). Control animals were vaccinated with an RNA encoding the ectodomain of HA from H1N1 A/Puerto Rico/8/1934 (eH1HA, positive control) or empty LNP (to control for effects of the LNP) or were not vaccinated (naive).

At week 0 and week 3, animals were immunized intramuscularly (IM) with a total volume of 100 μL of each test vaccine, which was administered in a 50 μL immunization to each quadricep. Candidate influenza virus vaccines evaluated in this study were described above and are outlined in the table below. Sera were collected from all animals two weeks after the second dose. At week 6, spleens were harvested from a subset of the animals (n=4). The remaining animals (n=6) were challenged intranasally while sedated with a mixture of Ketamine and Xylazine with a lethal dose of mouse-adapted influenza virus strain H1N1 A/Puerto Rico/8/1934. Mortality was recorded and individual mouse weight was assessed daily for 20 days post-infection.

| Group # | Antigen | Antigen dose | Formulation | Volume, Route |
|---|---|---|---|---|
| 1 | NIHGen6HASS-foldon | 5 ug | LNP | 100 ul, i.m. |
| | RNA | | | |
| 2 | NP RNA | 5 ug | LNP | 100 ul, i.m. |
| 3 | NIHGen6HASS-foldon | 5 ug of each | LNP | 100 ul, i.m. |
| | RNA + NP RNA | RNA mixed, | | |
| | | then formulated | | |
| 4 | NIHGen6HASS-foldon | 5 ug of each | LNP | 100 ul, i.m. |
| | RNA + NP RNA | RNA | | |
| | | formulated, | | |
| | | then mixed | | |
| 5 | NIHGen6HASS-foldon | 5 ug of each | LNP | 100 ul, i.m. |
| | RNA + NP RNA | RNA | | |
| | | formulated and | | |
| | | injected into | | |
| | | separate legs | | |
| 6 | eH1HA RNA | 10 ug | LNP | 100 ul, i.m. |
| 7 | LNP | 0 ug | LNP | 100 ul, i.m. |
| 8 | Naïve | 0 ug | None | None |

To test the sera for the presence of antibodies capable of binding to hemagglutinin (HA) from a wide variety of influenza strains or nucleoprotein (NP), ELISA plates were coated with 100 ng of the following recombinant proteins obtained from Sino Biological Inc.: Influenza A H1N1 (A/New Caledonia/20/99) HA, cat #11683-V08H; Influenza A H3N2 (A/Aichi/2/1968) HA, cat #11707-V08H; Influenza A H1N1 (A/California/04/2009) HA, cat #11055-V08H; Influenza A H1N1 (A/Puerto Rico/8/34) HA, cat #11684-V08H; Influenza A H1N1 (A/Brisbane/59/2007) HA, cat #11052-V08H; Influenza A H2N2 (A/Japan/305/1957) HA, cat #11088-V08H; Influenza A H7N9 (A/Anhui/1/2013) HA, cat #40103-V08H, Influenza A H3N2 (A/Moscow/10/99) HA, cat #40154-V08 and Influenza A H3N2 (A/Aichi/2/1968) Nucleoprotein, cat #40207-V08B. After coating, the plates were washed, blocked with Phosphate Buffered Saline with 0.05% Tween-20 (PBST)+3% milk, and 100 μL of control antibodies or sera from immunized mice (diluted in PBST+3% milk) were added to the top well of each plate and serially diluted. Plates were sealed and incubated at room temperature for 2 hours. Plates were washed, and goat anti-mouse IgG (H+L)-HRP conjugate (Novex, diluted 1:2000 in PBST/3% milk) was added to each well containing mouse sera. Plates were incubated at room temperature for 1 hr, washed, and incubated with TMB substrate (Thermo Scientific). The color was allowed to develop for approximately 10 minutes and then quenched with 100 μL of 2N sulfuric acid. The plates were read at 450 nM on a microplate reader. Endpoint titers (2.5-fold above background) were calculated.

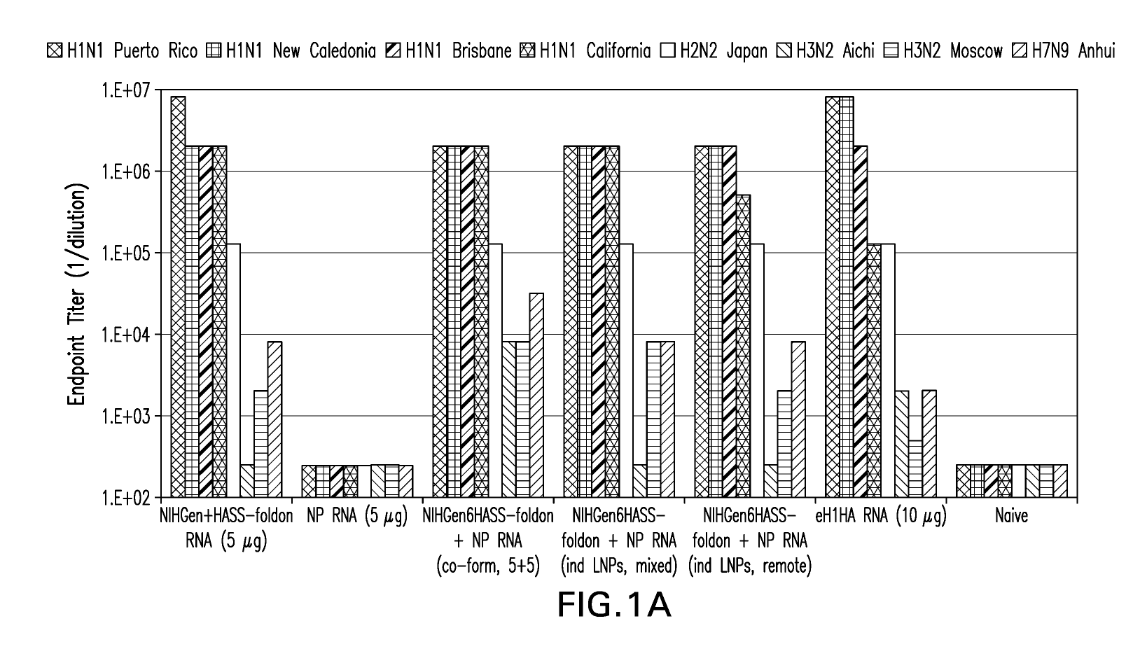

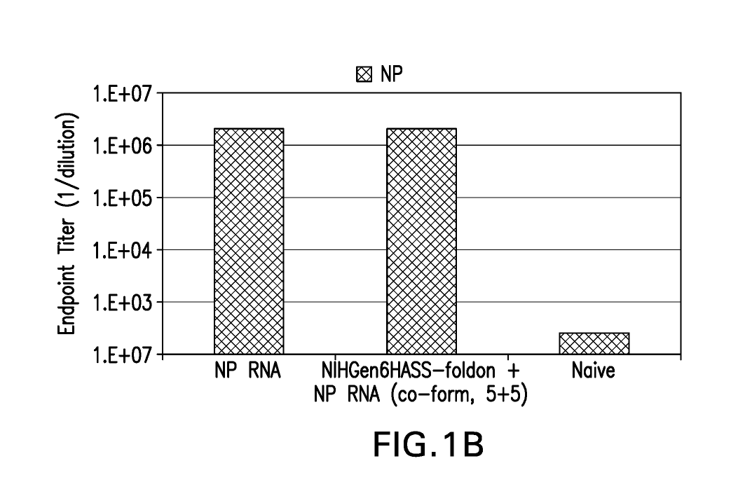

FIG. 1 depicts the endpoint titers of the pooled serum from animals vaccinated with the test vaccines. The vaccines tested are shown on the x-axis of FIG. 1A and the binding to HA from each of the different strains of influenza is plotted. The NIHGen6HASS-foldon mRNA vaccine elicited high titers of antibodies that bound all H1, H2 and H7 HAs tested. Combining the NIHGen6HASS-foldon mRNA with one that encodes NP did not negatively affect the observed anti-HA response, regardless of the method of mRNA co-formulation or co-delivery. In serum collected from identical groups from a separate study, a robust antibody response to NP protein was also detected in serum from animals vaccinated with NP mRNA containing vaccines, either NP alone or co-formulated with NIHGen6HASS-foldon mRNA (FIG. 1B).

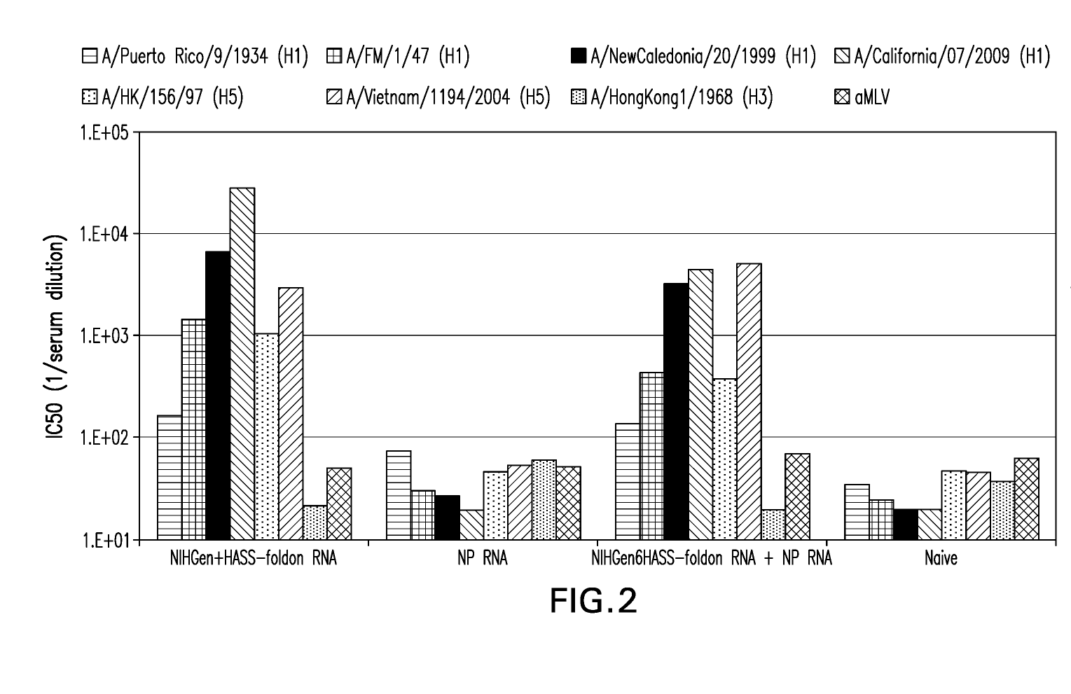

To probe the functional antibody response, the ability of serum to neutralize a panel of HA-pseudotyped viruses was assessed (FIG. 2). Briefly, 293 cells were co-transfected with a replication-defective retroviral vector containing a firefly luciferase gene, an expression vector encoding a human airway serine protease, and expression vectors encoding influenza hemagglutinin (HA) and neuraminidase (NA) proteins. The resultant pseudoviruses were harvested from the culture supernatant, filtered, and titered. Serial dilutions of serum were incubated in 96 well plates at 37° C. for one hour with pseudovirus stocks (30,000-300,000 relative light units per well) before 293 cells were added to each well. The cultures were incubated at 37° C. for 72 hours, luciferase substrate and cell lysing reagents were added, and relative light units (RLU) were measured on a luminometer. Neutralization titers are expressed as the reciprocal of the serum dilution that inhibited 50% of pseudovirus infection (IC50).

For each sample tested (listed along the x-axis), each bar represents the IC50 for neutralization of a different virus pseudotype. While the serum from naive or NP RNA vaccinated mice was unable to inhibit pseudovirus infection, the serum from mice vaccinated with NIHGen6HASS-foldon mRNA or with a combination of NIHGen6HASS-foldon and NP mRNAs neutralized, to a similar extent, all H1 and H5 virus pseudotypes tested.

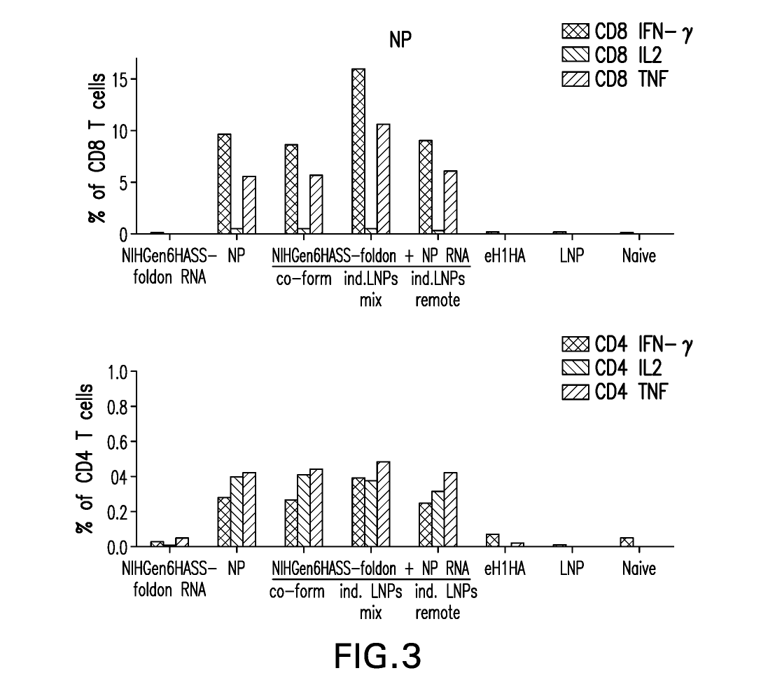

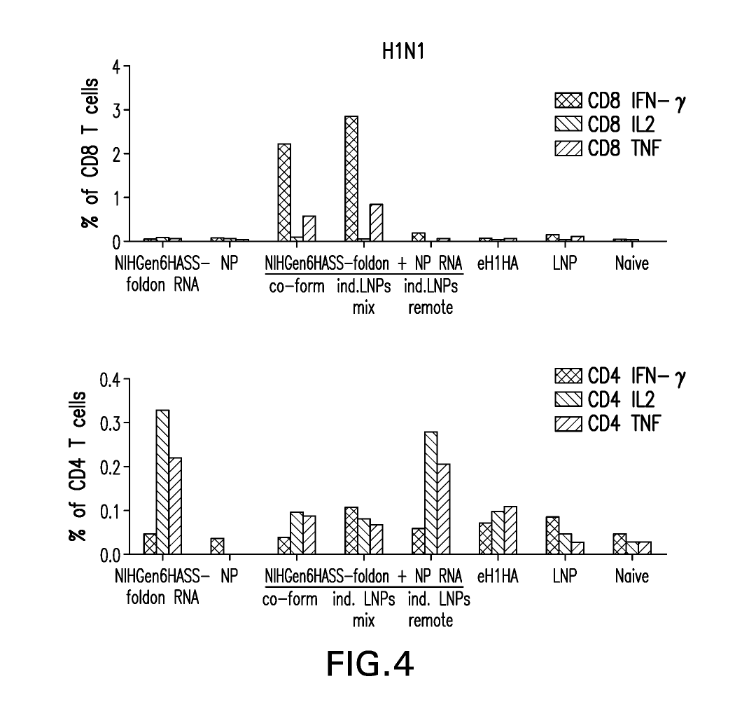

Three weeks after the administration of the second vaccine dose, spleens were harvested from a subset of animals in each group and splenocytes from animals in the same group were pooled. Splenic lymphocytes were stimulated with a pool of HA or NP peptides, and IFN-γ, IL-2 or TNF-α production was measured by intracellular staining and flow cytometry. FIG. 3 is a representation of responses following stimulation with a pool of NP peptides, and FIG. 4 is a representation of responses following stimulation with a pool of H1 HA peptides. Following vaccination with NP mRNA, either in the presence or absence of NIHGen6HASS-foldon mRNA, antigen-specific CD4 and CD8 T cells were found in the spleen. Following vaccination with NIHGen6HASS-foldon RNA or delivery of NIHGen6HASS-foldon and NP RNAs to distal injections sites (ind. LNPs remote), only HA-specific CD4 cells were observed. However, when NIHGen6HASS-foldon and NP RNAs were co-administered to the same injection site (co-form, ind. LNPs mix), an HA-specific CD8 T cell response was detected.

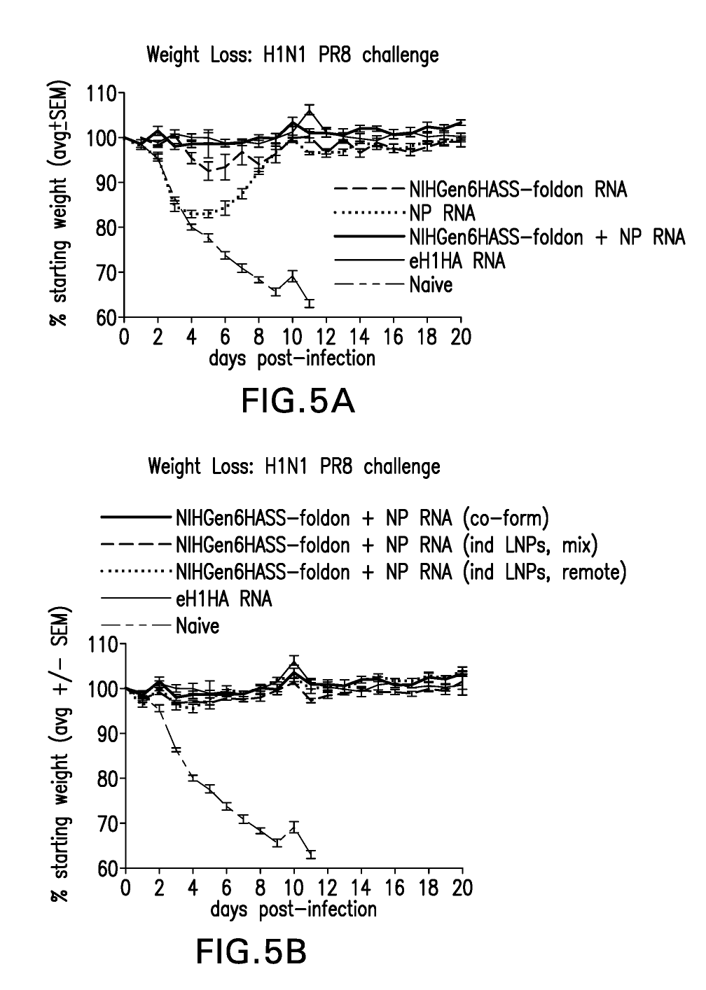

Following lethal challenge with mouse-adapted H1N1 A/Puerto Rico/8/1934, all naive animals succumbed to infection by day 12 post-infection (FIG. 5). In contrast, all animals vaccinated with NIHGen6HASS-foldon mRNA, NP mRNA, any combination of NIHGen6HASS-foldon and NP mRNAs, or eH1HA mRNA survived the challenge. As seen in FIG. 5A, although there was no mortality, mice that were vaccinated with an H3N2 NP mRNA and challenged with H1N1 virus lost a significant amount (˜15%) of weight prior to recovery. Those vaccinated with NIHGen6HASS-foldon RNA also lost ˜5% body weight. In contrast, mice vaccinated with a combination of NIHGen6HASS-foldon and NP mRNAs appeared to be completely protected from lethal influenza virus challenge, similar to those vaccinated with mRNA expressing an HA antigen homologous to that of the challenge virus (eH1HA). Although the cell-mediated immune responses varied, the vaccine efficacy was similar with all co-formulation and co-delivery methods assessed (FIG. 5B).

### Study #2

This study was designed to test the immunogenicity and efficacy in mice of a candidate influenza virus vaccine. Animals tested were 6-8 week old female BALB/c mice obtained from Charles River Laboratories. Test vaccines included the following mRNAs formulated in an LNP (comprised of a cationic lipid, a sterol, a phospholipid and a peg-lipid). LNP: NIHGen6HASS-foldon mRNA (based on Yassine et al. Nat. Med. 2015 September; 21(9): 1065-70) and NIHGen6HASS-TM2 mRNA, an RNA expressing HA stem fused to the native influenza transmembrane domain. Control animals were vaccinated with an mRNA encoding the ectodomain of the HA from H1N1 A/Puerto Rico/8/1934 (eH1HA, positive control) or were not vaccinated (naive).

At week 0 and week 3, animals were immunized intramuscularly (IM) with a total 5 volume of 100 μL of each test vaccine, which was administered in a 50 μL immunization to each quadricep. Candidate influenza virus vaccines evaluated in this study were described above and outlined in the table below. Sera were collected from all animals two weeks after the second dose. At week 6, all animals were challenged intranasally while sedated with a mixture of Ketamine and Xylazine with a lethal dose of mouse-adapted influenza virus strain H1N1 A/Puerto Rico/8/1934. Mortality was recorded and group mouse weight was assessed daily for 20 days post-infection.

| | | Antigen | | Volume, |
|---|---|---|---|---|
| Group # | Antigen | dose | Formulation | Route |
| 1 | NIHGen6HASS-foldon | 5 ug | LNP | 100 ul, i.m. |
| | RNA | | | |
| 2 | NIHGen6HASS-foldon- | 5 ug | LNP | 100 ul, i.m. |
| | TM2 RNA | | | |
| 3 | eH1HA RNA | 10 ug | LNP | 100 ul, i.m. |
| 4 | Naïve | 0 ug | None | None |

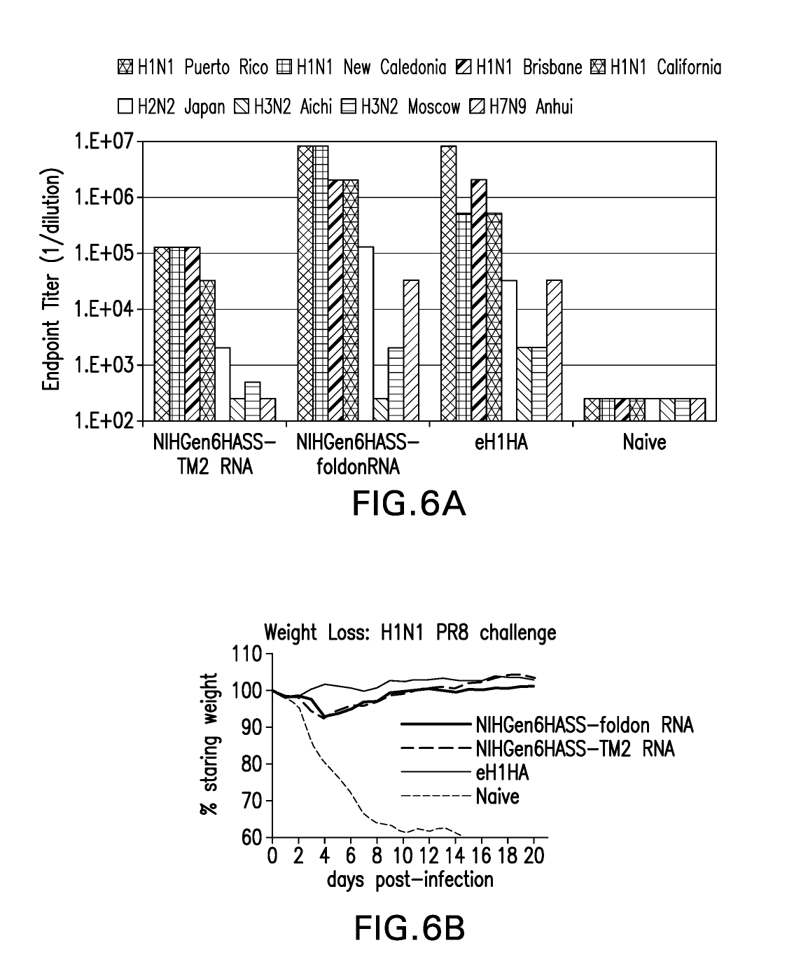

To test the sera for the presence of antibody capable of binding to hemagglutinin (HA) from a wide variety of influenza strains, ELISA plates were coated with 100 ng of the following recombinant HAs obtained from Sino Biological Inc.: Influenza A H1N1 (A/New Caledonia/20/99), cat #11683-V08H; Influenza A H3N2 (A/Aichi/2/1968), cat #11707-V08H; Influenza A H1N1 (A/California/04/2009), cat #11055-V8H; Influenza A H1N1 (A/Puerto Rico/8/34), cat #11684-V08H; Influenza A H1N1 (A/Brisbane/59/2007), cat #11052-V08H; Influenza A H2N2 (A/Japan/305/1957), cat #11088-V08H; Influenza A H7N9 (A/Anhui/1/2013), cat #40103-V08H and Influenza A H3N2 (A/Moscow/10/99), cat #40154-V08. The ELISA assay was performed and endpoint titers were calculated as described above. FIG. 6A depicts the endpoint titers of the pooled serum from animals vaccinated with the test vaccines. The vaccines tested are shown on the x-axis and the binding to HA from each of the different strains of influenza is plotted. The NIHGen6HASS-foldon mRNA vaccine elicited high titers of antibodies that bound all H1, H2 and H7 HAs tested. The binding titers from NIHGen6HASS-TM2 mRNA vaccinated mice were reduced as compared to those from NIHGen6HASS-foldon mRNA vaccinated mice.

Following lethal challenge with mouse-adapted H1N1 A/Puerto Rico/8/1934, all naive animals succumbed to infection by day 15 post-infection (FIG. 6B). In contrast, all animals vaccinated with NIHGen6HASS-foldon mRNA, NIHGen6HASS-TM2 mRNA, or eH1HA RNA survived the challenge. As shown in FIG. 6B, in spite of reduced HA binding titers, the efficacy of the NIHGen6HAS S-TM2 vaccine was equivalent to that of the NIHGen6HASS-foldon vaccine.

### Study #3

In this example, animal studies and assays were carried out to evaluate the immune response to influenza virus consensus hemagglutinin (HA) vaccine antigens delivered using an mRNA/LNP platform. The purpose of this study was to evaluate the ability of consensus HA mRNA vaccine antigens to elicit cross-reactive immune responses in the mouse.

To generate consensus HA sequences MRK_sH1_Con and MRK_pH1_Con, 2415 influenza A serotype H1 HA sequences were obtained from the NIAID Influenza Research Database (IRD) (Squires et al., Influenza Other Respir Viruses. 2012 November; 6(6): 404-416.) through the web site at http://www.fludb.org. After removal of duplicate sequences and lab strains, 2385 entries remained, including 1735 H1 sequences from pandemic H1N1 strains (pH1N1) and 650 from seasonal H1N1 strains (sH1N1). Pandemic and seasonal H1 sequences were separately aligned, and a consensus sequence was generated for each group using the Matlab 9.0 Bioinformatics toolbox (MathWorks, Natick, Mass.). Sequence profiles were generated for both groups separately using a modified Seq2Logo program (Thomsen et al., Nucleic Acids Res. 2012 July; 40 (Web Server issue):W281-7).

Animals tested were 6-8 week old female BALB/c mice obtained from Charles River Laboratories. Test vaccines included the following mRNAs formulated in an LNP (comprised of a cationic lipid, a sterol, a phospholipid and a peg-lipid).: ConH1 and ConH3 (based on Webby et al., PLoS One. 2015 Oct. 15; 10(10):e0140702.); Cobra P1 and Cobra X3 (based on Carter et al., J Virol. 2016 Apr. 14; 90(9):4720-34); MRK_pH1_Con and MRK_sH1_Con (pandemic and seasonal consensus sequences described above); and each of the above mentioned six antigens with a ferritin fusion sequence for potential particle formation.

Controls included: an LNP (comprised of a cationic lipid, a sterol, a phospholipid and a peg-lipid; control for effects of LNP); Naive (unvaccinated animals); and vaccination with eH1HA RNA, which encodes the ectodomain of HA from strain H1N1 A/PR/8/34 (positive control for the virus challenge).

At week 0 and week 3, animals were immunized intramuscularly (IM) with a total volume of 100 μL of each test vaccine, which was administered in a 50 μL immunization to each quadricep. Candidate influenza virus vaccines evaluated in this study were described above and are outlined in the table below. Sera were collected from all animals two weeks after the second dose (week 5).

| Group # | Antigen | Antigen dose | Formulation | Volume, Route |
|---|---|---|---|---|
| 1 | Con_H1 RNA | 10 ug | LNP | 100 ul, i.m. |
| 2 | Con_H3 RNA | 10 ug | LNP | 100 ul, i.m. |
| 3 | MRK_pH1_Con RNA | 10 ug | LNP | 100 ul, i.m. |
| 4 | MRK_sH1_Con RNA | 10 ug | LNP | 100 ul, i.m. |
| 5 | Cobra_P1 RNA | 10 ug | LNP | 100 ul, i.m. |
| 6 | Cobra_X3 RNA | 10 ug | LNP | 100 ul, i.m. |
| 7 | ConH1_ferritin RNA | 10 ug | LNP | 100 ul, i.m. |
| 8 | ConH3_ferritin RNA | 10 ug | LNP | 100 ul, i.m. |
| 9 | MRK_pH1_Con_ferritin RNA | 10 ug | LNP | 100 ul, i.m. |
| 10 | MRK_sH1_Con_ferritin RNA | 10 ug | LNP | 100 ul, i.m. |
| 11 | Cobra_P1_ferritin RNA | 10 ug | LNP | 100 ul, i.m. |
| 12 | Cobra_X3_ferritin RNA | 10 ug | LNP | 100 ul, i.m. |
| 13 | eH1HA | 10 ug | LNP | 100 ul, i.m. |
| 14 | LNP | 0 ug | LNP | 100 ul, i.m. |
| 15 | Naïve | 0 ug | None | None |

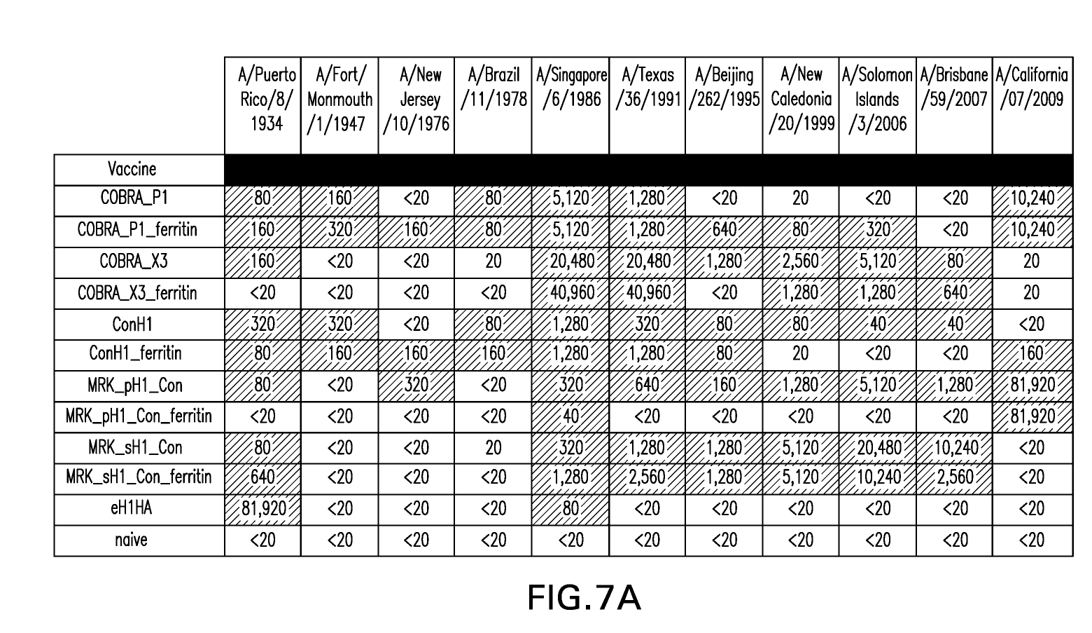

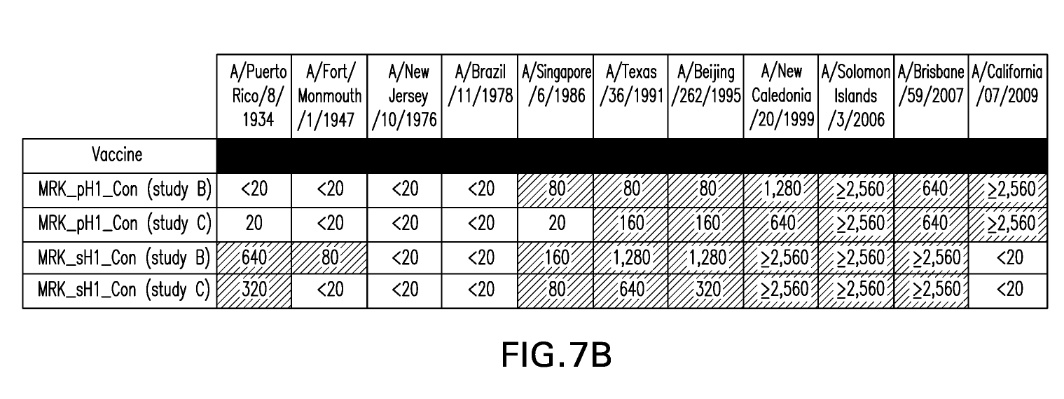

To assess the breadth of the serum neutralizing activity elicited by the consensus HA antigens, neutralization assays were performed using a panel of H1N1 influenza viruses (FIG. 7A). Briefly, Madin-Darby Canine Kidney (MDCK) cells were seeded in 96-well plates with complete media (DMEM containing Glutamax®, 50 μL/mL gentamycin and 10% FBS) and incubated overnight at 37° C. Duplicate serial dilutions of heat inactivated (35-45 min at 56° C.) serum samples as well as virus were added to cells, and cultures were incubated at 37° C. for 1 hour. Complete media was then replaced with trypsin-containing medium. Cell cultures were incubated at 37° C. for 2 days then fixed with 80% acetone and air-dried before plates were washed with PBS containing 0.1% Tween-20. An ELISA based assay was performed to detect influenza NP protein on the fixed plates. As expected, serum from mice immunized with eH1HA RNA, which encodes the ectodomain of HA from strain H1N1 A/PR/8/34, was only able to robustly neutralize a matched influenza strain (A/Puerto Rico/8/1934). In contrast, serum from mice immunized with the consensus H1 HA antigens was able to neutralize multiple diverse H1N1 strains isolated between 1934 and 2009. This observation was repeated in at least two additional independent studies in which an LNP containing a different cationic lipid nanoparticle (“LNP2”), was used to deliver the mRNA vaccines (FIG. 7B). With the exception of MRK_pH1_Con_ferritin, the ferritin fusion constructs induced at least similar, if not more potent, broadly neutralizing antibody titers as compared to the parental constructs (FIG. 7A).

### Study #4

This study was designed to test the immunogenicity and efficacy in mice of candidate influenza virus vaccines. Animals tested were 6-8 week old female BALB/c mice obtained from Charles River Laboratories. Test vaccines included the following mRNAs formulated in a cationic LNP: MRK_pH1_Con and MRK_sH1_Con (pandemic and seasonal consensus sequences described in study #3), MRK_sH1_Con_v2 (seasonal consensus sequence derived from a different H1N1 sequence database), MRK_pH1_Con ecto and MRK_sH1_Con ecto (soluble ectodomain of pandemic and seasonal consensus sequences) and MRK_pH1_Con_RBD and MRK_sH 1_Con_RBD (receptor binding domain of pandemic and seasonal consensus sequences, details on preparation of the constructs were described below in the next sections). A vaccine combining mRNA encoding MRK_pH1_Con and mRNA encoding the nucleoprotein NP from an H3N2 strain was also assessed. Control animals were vaccinated with an mRNA encoding the ectodomain of the HA from H1N1 A/Puerto Rico/8/1934 (eH1HA, positive control), empty LNP, or were not vaccinated (naive).

At week 0 and week 3, animals were immunized intramuscularly (IM) with a total volume of 100 μL of each test vaccine, which was administered in a 50 μL immunization to each quadricep. Candidate influenza virus vaccines evaluated in this study were described above and outlined in the table below. Sera were collected from all animals two weeks after the second dose. At week 6, all animals were challenged intranasally while sedated with a mixture of Ketamine and Xylazine with a lethal dose of mouse-adapted influenza virus strain H1N1 A/Puerto Rico/8/1934. Mortality was recorded and group mouse weight was assessed daily for 20 days post-infection.

| Group # | Antigen | Antigen dose | Formulation | Volume, Route |
|---|---|---|---|---|
| 1 | MRK_sH1_Con RNA | 5 ug | LNP2 | 100 ul, i.m. |
| 2 | MRK_sH1_Con_v2 | 5 ug | LNP2 | 100 ul, i.m. |
| | RNA | | | |
| 3 | MRK_sH1_Con_ecto | 5 ug | LNP2 | 100 ul, i.m. |
| | RNA | | | |
| 4 | MRK sH1_Con_RBD | 5 ug | LNP2 | 100 ul, i.m. |
| | RNA | | | |
| 5 | MRK_pH1_Con_RNA | 5 ug | LNP2 | 100 ul, i.m. |
| 6 | MRK_pH1_Con_RNA + | 5 ug of each | LNP2 | 100 ul, i.m. |
| | NP RNA | RNA | | |
| | | formulated, | | |
| | | then mixed | | |
| 7 | MRK_pH1_Con_ecto | 5 ug | LNP2 | 100 ul, i.m. |
| | RNA | | | |
| 8 | MRK_pH1_Con_RBD | 5 ug | LNP2 | 100 ul, i.m. |
| | RNA | | | |
| 9 | eH1HA RNA | 5 ug | LNP2 | 100 ul, i.m. |
| 10 | Empty LNP2 | 0 ug | LNP2 | 100 ul, i.m. |
| 11 | Naïve | 0 ug | None | None |

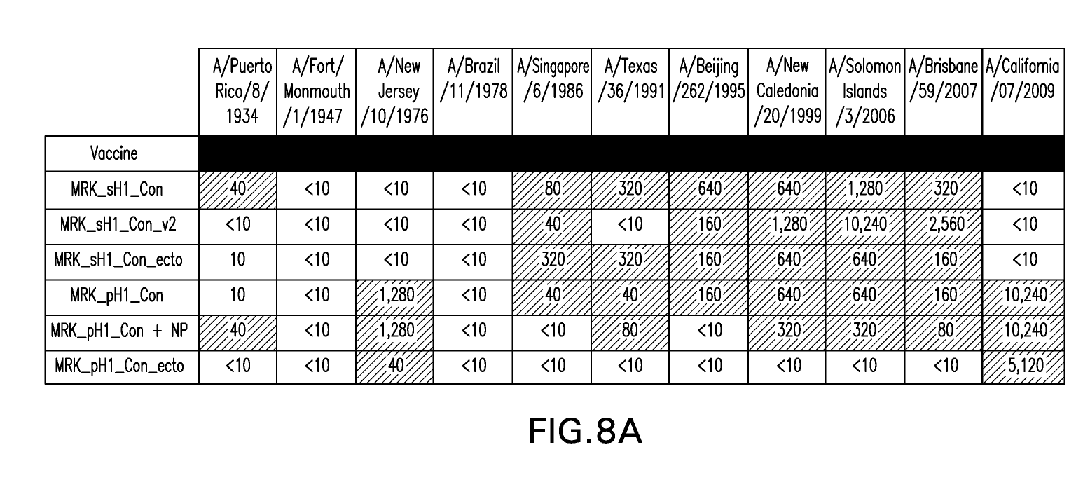

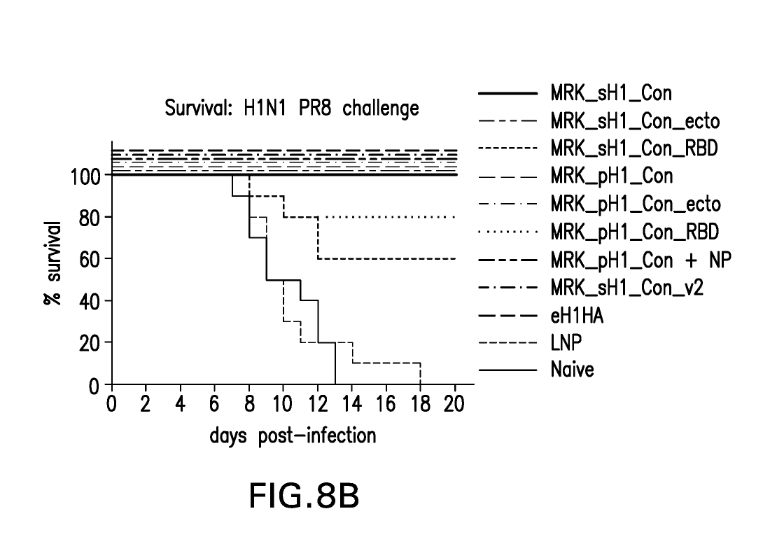

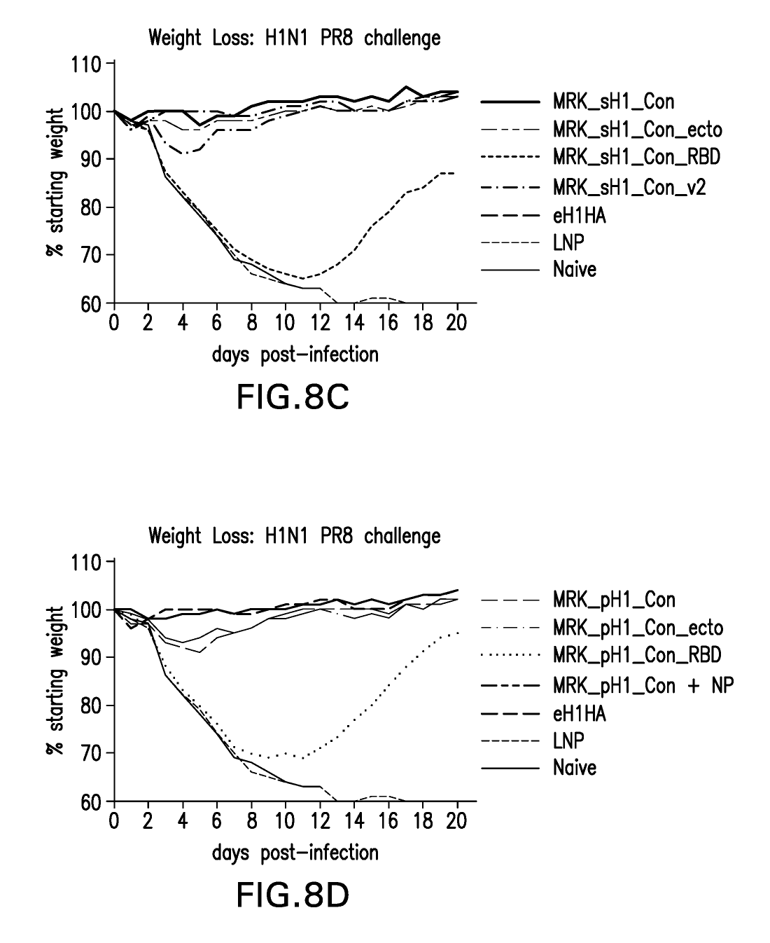

To assess the breadth of the serum activity elicited by the consensus HA antigens, hemagglutination inhibition assays (HAI) were performed using a panel of H1N1 influenza viruses (FIG. 8A). Briefly, serum samples were treated with receptor destroying enzyme (RDE) for 18-20 hrs at 37° C. before inactivation at 56° C. for 35-45 min. RDE-treated sera was then serially diluted in a 96 well plate and mixed with 4 hemagglutinating units of virus. An equal volume of 0.5% turkey red blood cells was added to each well, and plates were incubated at room temperature for 30 min. The highest dilution with no visible agglutination was assigned as the serum titer. With the exception of the pH1_Con_ecto mRNA vaccine, serum from mice immunized with mRNAs encoding full-length or the soluble ectodomain of consensus HAs were able to inhibit hemagglutination of red blood cells mediated by multiple diverse H1N1 strains isolated between 1934 and 2009. HAI titers were similar from mice immunized with pH1_Con vaccine with or without addition of NP vaccine.

All mice immunized with mRNAs encoding full-length or the soluble ectodomain of consensus H1 HAs survived a lethal PR8 virus challenge, while all naive mice and those vaccinated with empty LNP succumbed to challenge (FIG. 8B). Mice immunized with mRNAs encoding the receptor binding domain of consensus H1 HAs were partially protected: 80% survival for mice immunized with MRK_pH1_Con_RBD and 60% survival for those vaccinated with MRK_sH1_Con_RBD (FIG. 8B). Additionally, mice immunized with MRK_sH1_Con mRNA showed no weight loss post-challenge, in contrast to mice vaccinated with MRK_sH1_Con_v2, MRK_pH1_Con or MRK_pH1_Con_ecto mRNAs which lost, on average, between 7 and 10% of their body weight before recovering fully (FIG. 8C and FIG. 8D). As observed with the NIHGen6HASS-foldon+NP combination mRNA vaccine described in study #1, the MRK_pH1_Con+NP combination mRNA vaccine protected mice better than the MRK_pH1_Con mRNA vaccine alone. Mice in the MRK_pH1_Con+NP group were completely protected from lethal influenza virus challenge and lost no weight post-challenge, similar to mice vaccinated with mRNA expressing an HA antigen homologous to that of the challenge virus (eH1HA) (FIG. 8D).

### Study #5

In this example, two animal studies (A and B) were carried out to assess the ability of candidate influenza mRNA vaccine antigens to elicit cross-protective immune responses in the mouse.

Animals tested were 7-9 week old female BALB/c mice obtained from Envigo. Test vaccines included the following mRNAs formulated in a cationic LNP: MRK_pH1_Con and MRK_sH1_Con (pandemic and seasonal consensus sequences described in study #3); NIHGen6HASS-foldon, NP, a combination of NIHGen6HASS-foldon and NP vaccines, and a combination of NIHGen6HASS-TM2 and NP vaccines. Controls included: empty LNP (control for effects of LNP); Naive (unvaccinated animals); and vaccination with FL_Cal09 mRNA, which encodes the full length HA from strain H1N1 A/California/07/09 (positive control for the virus challenge).

At week 0 and week 3, animals were immunized intramuscularly (IM) with a total volume of 100 μL of each test vaccine, which was administered in a 50 μL immunization to each quadricep. Candidate influenza virus vaccines evaluated in this study were described above and are outlined in the table below. Sera were collected from all animals two weeks after the second dose (week 5). At week 6, the animals were challenged intranasally while sedated with a mixture of Ketamine and Xylazine with a lethal dose of influenza virus strain H1N1 A/California/07/2009 (Cal09). Mortality was recorded and mouse weight was assessed daily for 21 days post-infection. Mice at less than 80% of their pre-challenge weight were humanely euthanized.

| Group | | Antigen | | Volume, |
|---|---|---|---|---|
| # | Antigen | dose | Formulation | Route |
| 1 | MRK_pH1_Con RNA | 5 ug | LNP2 | 100 ul, i.m. |
| 2 | MRK_sH1_Con RNA | 5 ug | LNP2 | 100 ul, i.m. |
| 3 | NIHGen6HASS-foldon RNA | 5 ug | LNP2 | 100 ul, i.m. |
| 4 | NP RNA | 5 ug | LNP2 | 100 ul, i.m. |
| 5 | NIHGen6HASS-foldon | 5 ug each | LNP2 | 100 ul, i.m. |
| | RNA + NP RNA | | | |
| 6 | NIHGen6HASS-TM2 | 5 ug each | LNP2 | |
| | RNA + NP RNA | | | |
| 7 | FL_Cal09 RNA | 10 ug | LNP2 | 100 ul, i.m. |
| 8 | Empty LNP2 | 0 ug | LNP2 | 100 ul, i.m. |
| 9 | Naïve | 0 ug | None | None |

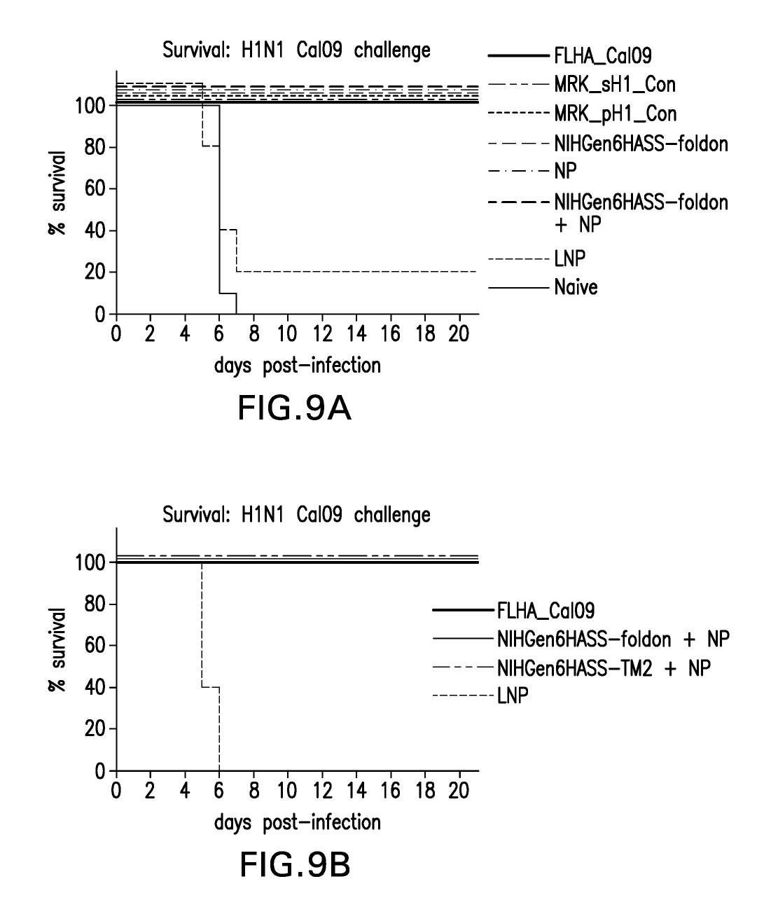

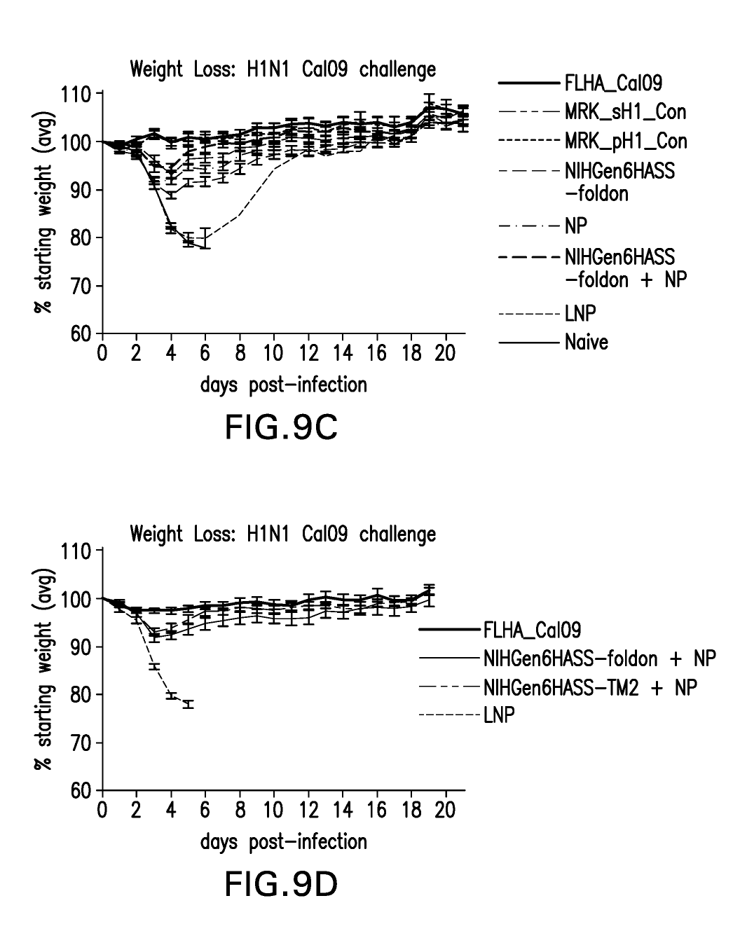

Following lethal challenge with H1N1 A/California/07/2009, all naive animals succumbed to infection by day 7 post-infection (FIG. 9A). Between 80 and 100% of mice vaccinated with empty LNP2 also succumbed to infection (FIG. 9A for study A and FIG. 9B for study B). In contrast, all animals vaccinated with candidate vaccines survived the challenge (FIGS. 9A and 9B). Although there was no mortality, mice vaccinated with an mRNA encoding NIHGen6HASS-foldon or NP lost a significant amount of body weight, approximately 10% on average, prior to recovery (FIG. 9C). Mice vaccinated with a combination of NIHGen6HASS-foldon and NP mRNAs appeared to be better protected from lethal Cal09 virus challenge, and the group lost, on average, only approximately 5% body weight. In study B, mice vaccinated with a combination of NIHGen6HASS-TM2 and NP mRNAs showed a similar pattern of weight loss and recovery (FIG. 9D). Consistent with the high serum neutralizing titers to the Cal09 strain observed in previously described studies (FIGS. 7A and 7B), mice immunized with MRK_pH1_Con mRNA survived the lethal Cal09 virus challenge and lost no weight post-infection. In contrast, mice vaccinated with MRK_sH1_Con mRNA, which does not induce neutralizing titers to Cal09 (FIGS. 7A and 7B), lost, on average, approximately 5% of their body weight post-infection, suggesting that partial protection may be mediated by mechanism(s) other than virus neutralization.

### Study #6

This study was designed to test the immunogenicity and efficacy in mice of candidate influenza virus vaccines. Animals tested were 6-8 week old female BALB/c mice obtained from Charles River Laboratories. Test vaccines included the following mRNAs formulated in a cationic LNP: MRK_H1_cot_all, MRK_H3_cot_all, MRK_H3 con_all, MRK_H3_Consensus A and MRK_H3_Consensus B. Consensus H3 sequences were generated similarly as described previously for consensus H1 sequences. COT sequences for H1 and H3 subtypes were generated as described below in the next section. Control animals were vaccinated with an mRNA encoding the HA from H1N1 A/Puerto Rico/8/1934 (FLHA_PR8, positive control for PR8 infection), vaccinated with empty LNP, infected with a nonlethal dose of mouse-adapted H3 A/Hong Kong/1/1968, or were not vaccinated (naive).

At week 0 and week 3, animals were immunized intramuscularly (IM) with a total volume of 100 μL of each test vaccine, which was administered in a 50 μL immunization to each quadricep. Candidate influenza virus vaccines evaluated in this study were described above and outlined in the table below. Sera were collected from all animals two weeks after the second dose. At week 6, all animals were challenged intranasally while sedated with a mixture of Ketamine and Xylazine with a lethal dose of mouse-adapted influenza virus strain H1N1 A/Puerto Rico/8/1934 (PR8) or H3 A/Hong Kong/1/1968 (HK68). Mortality was recorded and group mouse weight was assessed daily for 20 days post-infection.

| | | Antigen | | Volume, |
|---|---|---|---|---|
| Group # | Antigen | dose | Formulation | Route |
| 1 | FLHA_PR8 RNA | 5 ug | LNP2 | 100 ul, i.m. |
| 2 | MRK_H1_cot_all RNA | 10 ug | LNP2 | 100 ul, i.m. |
| 3 | MRK_H3_cot_all RNA | 10 ug | LNP2 | 100 ul, i.m. |
| 4 | MRK_H3_con_all RNA | 10 ug | LNP2 | 100 ul, i.m. |
| 5 | MRK_H3_Consensus A | 10 ug | LNP2 | 100 ul, i.m. |
| | RNA | | | |
| 6 | MRK_H3_Consensus B | 10 ug | LNP2 | 100 ul, i.m. |
| | RNA | | | |
| 7 | Empty LNP2 | 0 ug | LNP2 | 100 ul, i.m. |
| 8 | Mouse-adapted H3 | 0.1 LD90 | None | 20 ul, i.n. |
| | A/Hong Kong/1/1968 | | | |
| | virus | | | |
| 9 | Naïve | 0 ug | None | None |

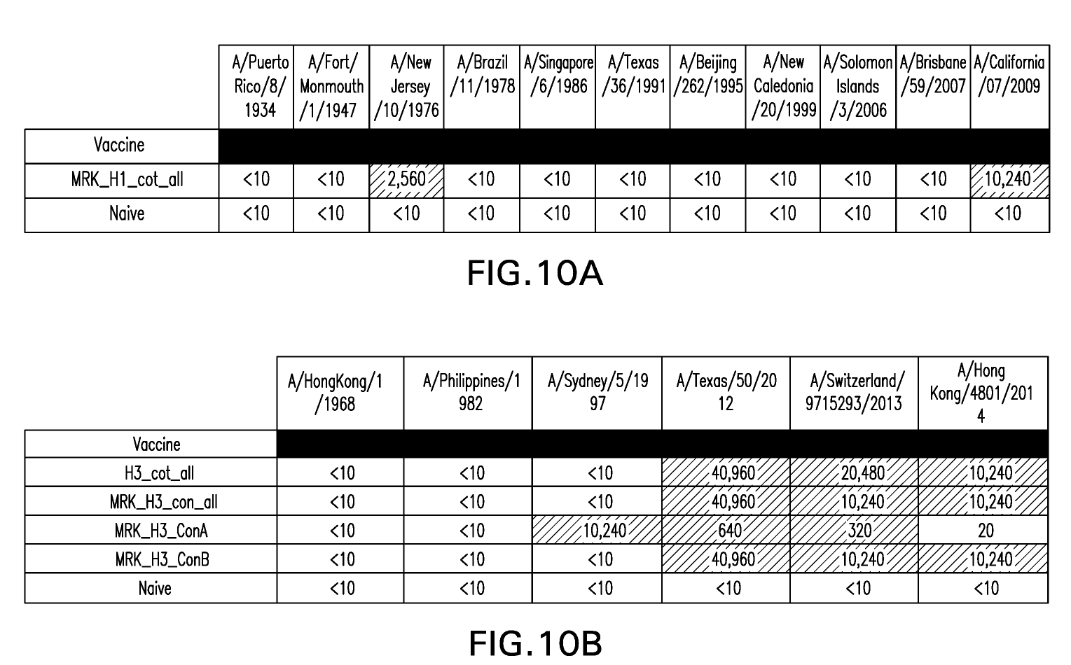

To assess the breadth of the serum activity elicited by the antigens, hemagglutination inhibition assays (HAI) were performed using a panel of H1N1 and H3N2 influenza viruses (FIGS. 10A and 10B). Briefly, serum samples were treated with receptor destroying enzyme (RDE) for 18-20 hrs at 37° C. before inactivation at 56° C. for 35-45 min. RDE-treated sera was then serially diluted in a 96 well plate and mixed with 4 hemagglutinating units of virus. An equal volume of 0.5% turkey red blood cells was added to each well, and plates were incubated at room temperature for 30 min. The highest dilution with no visible agglutination was assigned as the serum titer. While the MRK_H1_cot_all mRNA vaccine elicited titers to only two viruses in the H1 HAI panel (FIG. 10A), the MRK_H3_cot_all, MRK_H3_con_all, MRK_H3_Consensus A and MRK_H3_Consensus B mRNAs induced high HAI titers to multiple H3 strains isolated between 1997 and 2014 (FIG. 10B).

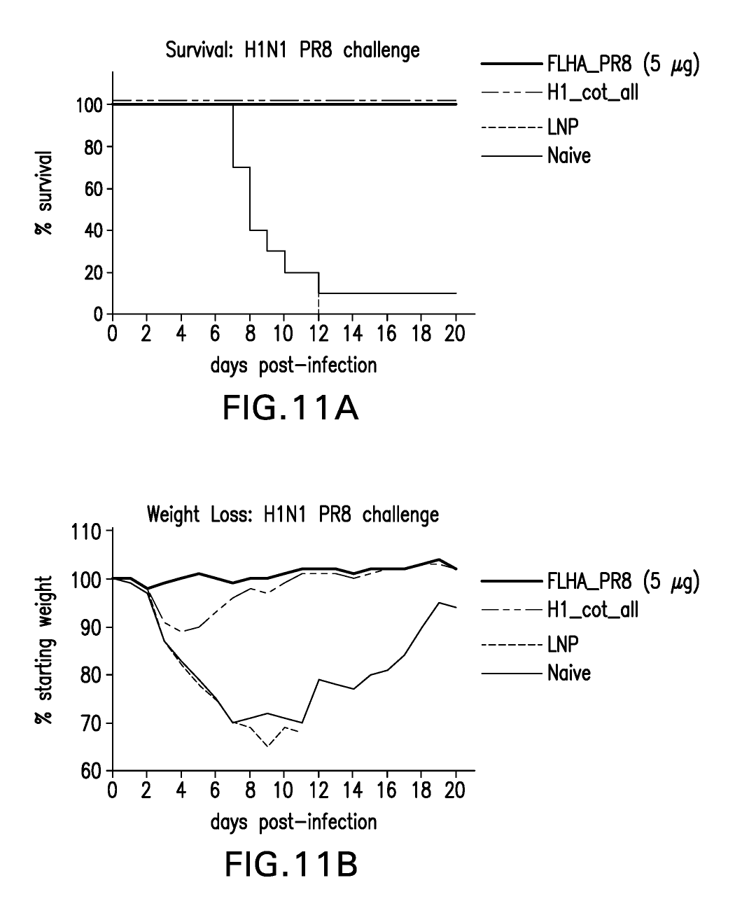

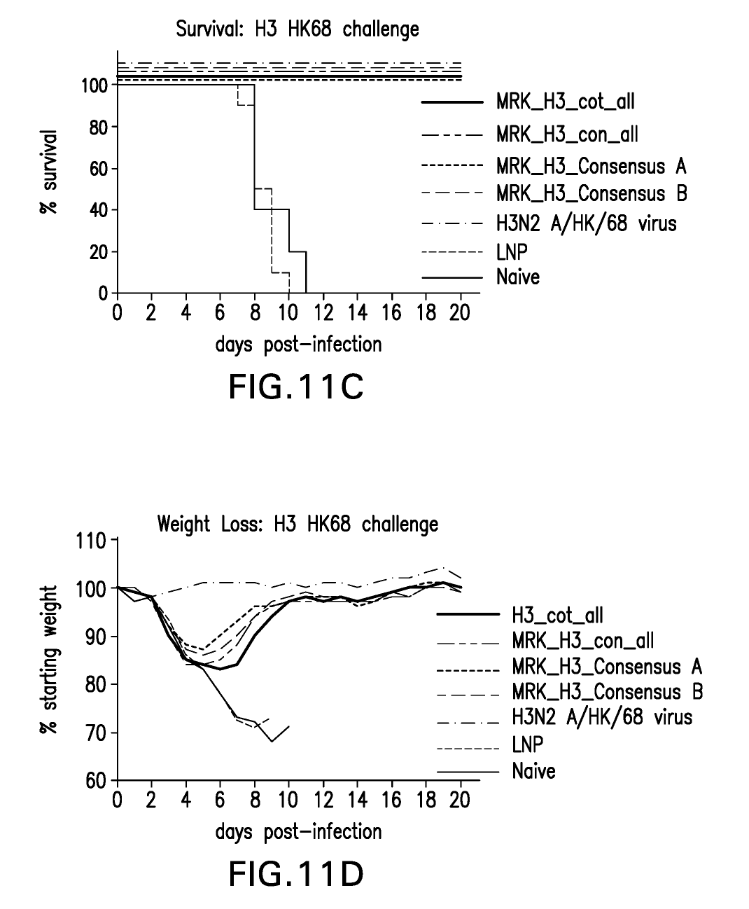

Although mice immunized with MRK_H1_cot_all mRNA did not have detectable HAI titers to the PR8 virus, they were partially protected from lethal challenge with PR8 virus. In contrast to naive or LNP2 vaccinated mice, all MRK_H1_cot_all mRNA immunized mice survived challenge (FIG. 11A), though they lost, on average, approximately 10% of their body weight post-infection (FIG. 11B). Similarly, mice vaccinated with any of the H3 COT or consensus mRNAs tested survived challenge with a lethal dose of HK68 virus (FIG. 11C) but lost between 10 and 15% or their body weight post-infection (FIG. 11D).

### Preparation of COT Constructs

The “Center of Tree” or COT is the point on the phylogenetic tree that represents the minimum of a metric of evolutionary distance. The COT minimizes the evolutionary distance to all sampled circulating strains, while still residing on an evolutionary path to better capture the biological properties of circulating viruses (D.C. Nickle et al., Consensus and ancestral state HIV vaccines, Science 299, 1515 (Mar. 7, 2003)). To prepare the COT sequences described herein, hemagglutinin DNA sequences of all of Influenza A collected after 2010 were downloaded from the Influenza Research Database (fludb.org), with H1 genotype forming data set 1 and H3 genotype forming data set 2. Any DNA sequence with aberrant/ambiguous nucleotides was removed. The initial trees for each data set where estimated by aligning the remaining sequences for each data set using the software package MUSCLE (R. C. Edgar, MUSCLE: a multiple sequence alignment method with reduced time and space complexity. BMC bioinformatics 5, 113 (Aug. 19, 2004)). Next the alignment was used to estimate a Maximum Likelihood (ML) phylogeny in the software package PhyML under a GTR+G+I model of nucleotide evolution. (S. Guindon et al., New algorithms and methods to estimate maximum-likelihood phylogenies: assessing the performance of PhyML 3.0. Systematic biology 59, 307 (May, 2010)). The estimated ML trees where re-rooted with the COT algorithm defined above—giving rise to a node called COT. The ML state for each and every node including the COT node in the trees was estimated using ML methods under the same model of nucleotide evolution estimated during the initial tree estimation. Because the COT is a node, we can parse out the COT node state DNA sequences from the list of node sequences found throughout the trees. Corresponding protein sequences were derived based on the COT DNA sequences described above.

### Preparation of Receptor Binding Domain Constructs

The following was used as a template sequence for all designs: A/California/4/2009(H1N1) (gi:2278098301UniProtKB:C3W5S1). The Receptor Binding Domain (RBD) was defined as residues 63-278 based on structural analysis (DuBois et al., J. Virology January 2011, p. 865). The 6 constructs are divided into two types: MRK_RBD which uses the native sequence as a template, and RBD1 which adds an additional glycosylation site at position 97 to increase polarity in an exposed hydrophobic patch. The MRK_RBD-Cal09-PC, MRK_RBD-Cal09-PC-Cb, RBD1-Cal09-PC and RBD1-Cal09-PC-Cb constructs are RBDs containing inserted glycosylation recognition motifs to result in a hyper-glycosylated form of the protein upon post-translational modification by the cell (Eggink et al., J. Virology January 2014 (volume 88 number 1) p. 699).

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety.

1. An influenza virus vaccine, comprising at least one isolated antigenic polypeptide:
wherein the at least one antigenic polypeptide comprises an amino acid sequence that has at least 90% identity to a mature amino acid sequence in any one of SEQ ID NOs: 1-4, 8-11, 14-19, 41-61, and 83-88.

2. (canceled)

3. (canceled)

4. The vaccine of claim 1, wherein the polypeptide has conservative mutations compared to a mature amino acid sequence in any one of SEQ ID NOs: 1-4, 8-11, 14-19, 41-61, and 83-88.

5. The vaccine of claim 1, wherein the polypeptide comprises the mature amino acid sequence that is identified in any one of SEQ ID NOs: 1-4, 8-11, 14-19, 41-61, and 83-88.

6. (canceled)

7. The vaccine of claim 1, wherein the polypeptide comprises a mature amino acid sequence that is the extracellular domain of SEQ ID NO: 8 or 10.

8. An influenza virus vaccine comprising two isolated antigenic polypeptides, wherein the first polypeptide comprises an amino acid sequence that has at least 90% identity to the mature amino acid sequence of any one of SEQ ID NOs: 1-5, 8-11, 14-19, 41-63, and 83-88 and the second polypeptide comprises an amino acid sequence that has at least 90% identity to the mature amino acid sequence of SEQ ID NO: 20.

9. (canceled)

10. (canceled)

11. The vaccine of claim 8, wherein the first and second polypeptide has conservative mutations compared to the mature amino acid sequence in any one of SEQ ID NOs: 1-5, 8-11, 14-19, 41-63, 83-88 and 20.

12. The vaccine of claim 8, wherein the first polypeptide comprises the mature amino acid sequence in SEQ ID NO: 4, and the second polypeptide comprises the mature amino acid sequence in SEQ ID NO: 20.

13. (canceled)

14. The vaccine of claim 1, wherein the vaccine is multivalent.

15. The vaccine of claim 1 formulated in an effective amount to produce an antigen-specific immune response.

16. The vaccine of claim 1, formulated with a Lipid Nanoparticle (LNP) comprising one or more cationic lipids and a poly(ethyleneglycol)-lipid (PEG-lipid).

17. The vaccine of claim 16, wherein the one or more cationic lipids are ionizable cationic lipids.

18. The vaccine of claim 17, wherein the ionizable cationic lipids are selected from DLinDMA; DlinKC2DMA; DLin-MC3-DMA; CLinDMA; S-Octyl CLinDMA;
(2S)-1-{7-[(3β)-cholest-5-en-3-yloxy]heptyloxy}-3-[(4Z)-dec-4-en-1-yloxy]-N,N-dimethylpropan-2-amine;
(2R)-1-{4-[(3β)-cholest-5-en-3-yloxy]butoxy}-3-[(4Z)-dec-4-en-1-yloxy]-N,N-dimethylpropan-2-amine;
1-[(2R)-1-{4-[(3β)-cholest-5-en-3-yloxy]butoxy}-3-(octyloxy)propan-2-yl]guanidine;
1-[(2R)-1-{7-[(3β)-cholest-5-en-3-yloxy]heptyloxy}-N,N-dimethyl-3-[(9 Z,12Z)-octadeca-9,12-dien-1-yloxy]propan-2-amine;
1-[(2R)-1-{4-[(3β)-cholest-5-en-3-yloxy]butoxy}-N,N-dimethyl-3-[(9 Z,12Z)-octadeca-9,12-dien-1-yloxy]propan-2-amine;
(2S)-1-({6-[(3β))-cholest-5-en-3-yloxy]hexyl}oxy)-N,N-dimethyl-3-[(9 Z)-octadec-9-en-1-yloxy]propan-2-amine;
(3β)-3-[6-{[(2S)-3-[(9Z)-octadec-9-en-1-yloxyl]-2-(pyrrolidin-1-yl)propyl]oxy}hexyl)oxy]cholest-5-ene;
(2R)-1-{4-[(3β)-cholest-5-en-3-yloxy]butoxy}-3-(octyloxy)propan-2-amine;
(2R)-1-({8-[(3β)-cholest-5-en-3-yloxy]octyl)}oxy)-N,N-dimethyl-3-(pentyloxy)propan-2-amine;
(2R)-1-({8-[(3β)-cholest-5-en-3-yloxy]octyl)}oxy)-3-(heptyloxy)-N,N-dimethylpropan-2-amine;
(2R)-1-({8-[(3β)-cholest-5-en-3-yloxy]octyl})oxy)-N,N-dimethyl-3-[(2Z)-pent-2-en-1-yloxy]propan-2-amine;
(2S)-1-butoxy-3-({8-[(3β)-cholest-5-en-3-yloxy]octyl})oxy)-N,N-dimethylpropan-2-amine;
(2S-1-({8-[(3(3)-cholest-5-en-3-yloxy]octyl}oxy)-3-[2,2,3,3,4,4,5,5,6,6,7,7,8,8,9,9-hexadecafluorononyl)oxy]-N,N-dimethylpropan-2-amine;
2-amino-2-{[(9Z,12Z)-octadeca-9,12-dien-1-yloxy]methyl}propane-1,3-diol;
2-amino-3-((9-(((3S,10R,13R)-10,13-dimethyl-17-(6-methylheptan-2-yl)-2,3,4,7,8,9,10,11,12,13,14,15,16,17-tetradecahydro-1H-cyclopenta[a]phenanthren-3-yl)oxy)nonyl)oxy)-2-((((9Z,12Z)-octadeca-9,12-dien-1-yl)oxy)methyl)propan-1-ol;
2-amino-3-((6-(((3S,10R,13R)-10,13-dimethyl-17-(6-methylheptan-2-yl)-2,3,4,7,8,9,10,11,12,13,14,15,16,17-tetradecahydro-1H-cyclopenta[a]phenanthren-3-yl)oxy)hexyl)oxy)-2-((((Z)-octadec-9-en-1-yl)oxy)methyl)propan-1-ol;
(20Z,23Z)-N,N-dimethylnonacosa-20,23-dien-10-amine;
(17Z,20Z)-N,N-dimethylhexacosa-17,20-dien-9-amine;
(16Z,19Z)-N,N-dimethylpentacosa-16,19-dien-8-amine;
(12Z,15Z)-N,N-dimethylhenicosa-12,15-dien-4-amine;
(14Z,17Z)-N,N-dimethyltricosa-14,17-dien-6-amine;
(15Z,18Z)-N,N-dimethyltetracosa-15,18-dien-7-amine;
(18Z,21Z)-N,N-dimethylheptacosa-18,21-dien-10-amine;
(15Z,18Z)-N,N-dimethyltetracosa-15,18-dien-5-amine;
(14Z,17Z)-N,N-dimethyltricosa-14,17-dien-4-amine;
(19Z,22Z)-N,N-dimethyloctacosa-19,22-dien-9-amine;
(18Z,21Z)-N,N-dimethylheptacosa-18,21-dien-8-amine;
(17Z,20Z)-N,N-dimethylhexacosa-17,20-dien-7-amine;
(16Z,19Z)-N,N-dimethylpentacosa-16,19-dien-6-amine;
(22Z,25Z)-N,N-dimethylhentriaconta-22,25-dien-10-amine;
(21Z,24Z)-N,N-dimethyltriaconta-21,24-dien-9-amine;
(18Z)-N,N-dimethylheptacos-18-en-10-amine;
(17Z)-N,N-dimethylhexacos-17-en-9-amine;
(19Z,22Z)-N,N-dimethyloctacosa-19,22-dien-7-amine;
N,N-dimethylheptacosan-10-amine;
(20Z,23Z)-N-ethyl-N-methylnonacosa-20,23-dien-10-amine;
1-[(11Z,14Z)-1-nonylicosa-11,14-dien-1-yl]pyrrolidine;
(20Z)-N,N-dimethylheptacos-20-en-10-amine;
(15Z)-N,N-dimethylheptacos-15-en-10-amine;
(14Z)-N,N-dimethylnonacos-14-en-10-amine;
(17Z)-N,N-dimethylnonacos-17-en-10-amine;
(24Z)-N,N-dimethyltritriacont-24-en-10-amine;
(20Z)-N,N-dimethylnonacos-20-en-10-amine;
(22Z)-N,N-dimethylhentriacont-22-en-10-amine;
(16Z)-N,N-dimethylpentacos-16-en-8-amine;
(12Z,15Z)-N,N-dimethyl-2-nonylhenicosa-12,15-dien-1-amine;
(13Z,16Z)-N,N-dimethyl-3-nonyldocosa-13,16-dien-1-amine;
N,N-dimethyl-1-[(1S,2R)-2-octylcyclopropyl]heptadecan-8-amine;
1-[(1S,2R)-2-hexylcyclopropyl]-N,N-dimethylnonadecan-10-amine;
N,N-dimethyl-1-[(1S,2R)-2-octylcyclopropyl]nonadecan-10-amine;
N,N-dimethyl-21-[(1S,2R)-2-octyl cyclopropyl]henicosan-10-amine;
N,N-dimethyl-1-[(1S,2S)-2-{[(1R,2R)-2-pentylcyclopropyl]methyl} cyclopropyl]nonadecan-10-amine;
N,N-dimethyl-1-[(1S,2R)-2-octylcyclopropyl]hexadecan-8-amine;
N,N-dimethyl-1-[(1R,2S)-2-undecylcyclopropyl]tetradecan-5-amine;
N,N-dimethyl-3-{7-[(1S,2R)-2-octylcyclopropyl]heptyl} dodecan-1-amine;
1-[(1R,2S)-2-heptylcyclopropyl]-N,N-dimethyloctadecan-9-amine;
1-[(1S,2R)-2-decylcyclopropyl]-N,N-dimethylpentadecan-6-amine;
N,N-dimethyl-1-[(1S,2R)-2-octylcyclopropyl]pentadecan-8-amine; and
(11E,20Z,23Z)-N,N-dimethylnonacosa-11,20,23-trien-10-amine;
or a pharmaceutically acceptable salt thereof, or a stereoisomer of any of the foregoing.

19. (canceled)

20. The vaccine of claim 17, which comprises 30-75 mole % ionizable cationic lipid and 0.1-20 mole % PEG-lipid.

21. The vaccine of claim 17, further comprising one or more non-cationic lipids selected from a phospholipid, a phospholipid derivative, a fatty acid, a sterol, or a combination thereof.

22. The vaccine of claim 21, wherein the sterol is cholesterol, stigmasterol or stigmastanol.

23. The vaccine of claim 21, wherein the phospholipid is selected from phosphatidylserine, 1,2-Distearoyl-sn-glycero-3-phosphocholine (DSPC), 1,2-dipalmitoleoyl-sn-glycero-3-phosphocholine, 1,2-dimyristoyl-sn-glycero-3-phosphocholine (DMPC), dilauroylphosphatidylcholine (DLPC), 1,2-dieicosenoyl-sn-glycero-3-phosphocholine, and 1,2-dioleoyl-sn-glycero-3-phosphocholine (DOPC).

24. The vaccine of claim 23, wherein the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG- dipalmitoyl phosphatidylethanolamine (PEG-DPPE), or PEG-1,2-dimyristyloxlpropyl-3-amine (PEG-c-DMA).

25. The vaccine of claim 24, which comprises 20-99.8 mole % ionizable cationic lipids, 0.1-65 mole % non-cationic lipids, and 0.1-20 mole % PEG-lipid.

26. The vaccine of claim 25, wherein the non-cationic lipids comprise a mixture of cholesterol and DSPC.

27. The vaccine of claim 16, which comprises 34-59 mole % ionizable cationic lipids selected from the group consisting of (2S)-1-({6-[(3β)-cholest-5-en-3-yloxy]hexyl}oxy)-N,N-dimethyl-3-[(9 Z)-octadec-9-en-1-yloxy]propan-2-amine; (13Z,16Z)-N,N-dimethyl-3-nonyldocosa-13,16-dien-1-amine; and N,N-dimethyl-1-[(1S,2R)-2-octylcyclopropyl]heptadecan-8-amine, 30-48 mole % cholesterol, 10-24% DSPC and 1-2 mole % PEG-DMG.

28. (canceled)

29. A method of inducing an immune response in a subject, the method comprising administering to the subject the vaccine of claim 1 in an amount effective to produce an antigen-specific immune response in the subject.

30.-36. (canceled)

37. A method of inducing cross-reactivity against a variety of influenza strains in a mammal, the method comprising administering to the mammal in need thereof the vaccine of claim 1.

38. (canceled)
